SEQUENCE LISTING (105 sequences)

SEQ ID NO: 1 - DNA, 43 bp, synthetic construct
  source 1..43: mol_type = other DNA
cggcaaatagcgaagacagcnnnnnnnngtctctatgagttgt

SEQ ID NO: 2 - DNA, 43 bp, synthetic construct
  source 1..43: mol_type = other DNA
gtgaggcgctagagcataggnnnnnnnnacttgcgccaacaac

SEQ ID NO: 3 - DNA, 20 bp, synthetic construct
  source 1..20: mol_type = other DNA
gctgtcttcgctatttgccg

SEQ ID NO: 4 - DNA, 20 bp, synthetic construct
  source 1..20: mol_type = other DNA
cctatgctctagcgcctcac

SEQ ID NO: 5 - DNA, 43 bp, synthetic construct
  source 1..43: mol_type = other DNA
tggtcctacgagacatcggtnnnnnnnngtatggcgtcacaac

SEQ ID NO: 6 - DNA, 43 bp, synthetic construct
  source 1..43: mol_type = other DNA
gtagcgccttgcatgttagcnnnnnnnncaggtcggaaacaac

SEQ ID NO: 7 - DNA, 43 bp, synthetic construct
  source 1..43: mol_type = other DNA
gctgccggcatcctatagttnnnnnnnnggactgcgatacaac

SEQ ID NO: 8 - DNA, 43 bp, synthetic construct
  source 1..43: mol_type = other DNA
caggtataccatcgccgcttnnnnnnnngcgacttcagacaac

SEQ ID NO: 9 - DNA, 43 bp, synthetic construct
  source 1..43: mol_type = other DNA
ccgaccagtaaacacgtggannnnnnnngtcaggtgaaacaac

SEQ ID NO: 10 - DNA, 43 bp, synthetic construct
  source 1..43: mol_type = other DNA
cgctagagctgggtgatctgnnnnnnnntgtatggtacacaac

SEQ ID NO: 11 - DNA, 43 bp, synthetic construct
  source 1..43: mol_type = other DNA
catgctatcaccggcaggatnnnnnnnntcggtgagaagttgt

SEQ ID NO: 12 - DNA, 43 bp, synthetic construct
  source 1..43: mol_type = other DNA
agctaactgaaccagggcacnnnnnnnngtgtgcgtctgttgt

SEQ ID NO: 13 - DNA, 43 bp, synthetic construct
  source 1..43: mol_type = other DNA
ccgtcgtcatgcgacaattcnnnnnnnntgatgcgttggttgt

SEQ ID NO: 14 - DNA, 43 bp, synthetic construct
  source 1..43: mol_type = other DNA
gcgctgccgtgcattaaatannnnnnnnggcgcaacaagttgt

SEQ ID NO: 15 - DNA, 43 bp, synthetic construct
  source 1..43: mol_type = other DNA
aggtgcggtaacaacgcttannnnnnnngtacttgtccgttgt

SEQ ID NO: 16 - DNA, 43 bp, synthetic construct
  source 1..43: mol_type = other DNA
ggttggcgagtccattggtannnnnnnntcttgtagcagttgt

SEQ ID NO: 17 - DNA, 44 bp, synthetic construct
  source 1..44: mol_type = other DNA
cggcaaatagcgaagacagcnnnnnnnntcacatacagctcgcg

SEQ ID NO: 18 - DNA, 44 bp, synthetic construct
  source 1..44: mol_type = other DNA
gtgaggcgctagagcataggnnnnnnnntgagtaacgacgcgag

SEQ ID NO: 19 - DNA, 44 bp, synthetic construct
  source 1..44: mol_type = other DNA
cggcaaatagcgaagacagcnnnnnnnntagacagaagccaatt

SEQ ID NO: 20 - DNA, 44 bp, synthetic construct
  source 1..44: mol_type = other DNA
gtgaggcgctagagcataggnnnnnnnnaccatatcgaaattgg

SEQ ID NO: 21 - DNA, 37 bp, synthetic construct
  source 1..37: mol_type = other DNA
gtgaggcgctagagcataggctgcatcgaggtcataa

SEQ ID NO: 22 - DNA, 37 bp, synthetic construct
  source 1..37: mol_type = other DNA
cggcaaatagcgaagacagcttcgcacctattatgac

SEQ ID NO: 23 - DNA, 52 bp, synthetic construct
  source 1..52: mol_type = other DNA
gtgaggcgctagagcataggtgacatgagtctgcatcgagccagggtcataa

SEQ ID NO: 24 - DNA, 20 bp, synthetic construct
  source 1..20: mol_type = other DNA
  misc_feature 20: note = 3' phosphate, or dideoxynucleotide, or other 3' chain terminator
ctcgatgcagactcatgtca

SEQ ID NO: 25 - DNA, 12 bp, synthetic construct
  source 1..12: mol_type = other DNA
ttatgaccctgg

SEQ ID NO: 26 - DNA, 52 bp, synthetic construct
  source 1..52: mol_type = other DNA
cggcaaatagcgaagacagcctaagtgtacttcgcacctataccgttatgac

SEQ ID NO: 27 - DNA, 20 bp, synthetic construct
  source 1..20: mol_type = other DNA
  misc_feature 20: note = 3' phosphate, or dideoxynucleotide, or other 3' chain terminator
taggtgcgaagtacacttag

SEQ ID NO: 28 - DNA, 12 bp, synthetic construct
  source 1..12: mol_type = other DNA
gtcataacggta

SEQ ID NO: 29 - DNA, 28 bp, synthetic construct
  source 1..28: mol_type = other DNA
ctcgatgcagactcatgtcaatcgtttg

SEQ ID NO: 30 - DNA, 28 bp, synthetic construct
  source 1..28: mol_type = other DNA
taggtgcgaagtacacttaggttcacgt

SEQ ID NO: 31 - DNA, 39 bp, synthetic construct
  source 1..39: mol_type = other DNA
gtgaggcgctagagcataggatctgcatcgaggtcataa

SEQ ID NO: 32 - DNA, 41 bp, synthetic construct
  source 1..41: mol_type = other DNA
cggcaaatagcgaagacagctgacttcgcacctattatgac

SEQ ID NO: 33 - DNA, 24 bp, synthetic construct
  source 1..24: mol_type = other DNA
gtcagctgtcttcgctatttgccg

SEQ ID NO: 34 - DNA, 39 bp, synthetic construct
  source 1..39: mol_type = other DNA
gtgaggcgctagagcataggcgctgcatcgaggtcataa

SEQ ID NO: 35 - DNA, 41 bp, synthetic construct
  source 1..41: mol_type = other DNA
cggcaaatagcgaagacagcagtcttcgcacctattatgac

SEQ ID NO: 36 - DNA, 24 bp, synthetic construct
  source 1..24: mol_type = other DNA
gactgctgtcttcgctatttgccg

SEQ ID NO: 37 - DNA, 39 bp, synthetic construct
  source 1..39: mol_type = other DNA
gtgaggcgctagagcataggaactgcatcgaggtcataa

SEQ ID NO: 38 - DNA, 41 bp, synthetic construct
  source 1..41: mol_type = other DNA
cggcaaatagcgaagacagcttcattcgcacctattatgac

SEQ ID NO: 39 - DNA, 24 bp, synthetic construct
  source 1..24: mol_type = other DNA
tgaagctgtcttcgctatttgccg

SEQ ID NO: 40 - DNA, 39 bp, synthetic construct
  source 1..39: mol_type = other DNA
gtgaggcgctagagcataggtgctgcatcgaggtcataa

SEQ ID NO: 41 - DNA, 41 bp, synthetic construct
  source 1..41: mol_type = other DNA
cggcaaatagcgaagacagcaaggttcgcacctattatgac

SEQ ID NO: 42 - DNA, 24 bp, synthetic construct
  source 1..24: mol_type = other DNA
ccttgctgtcttcgctatttgccg

SEQ ID NO: 43 - DNA, 41 bp, synthetic construct
  source 1..41: mol_type = other DNA
gtgaggcgctagagcataggccggctgcatcgaggtcataa

SEQ ID NO: 44 - DNA, 14 bp, synthetic construct
  source 1..14: mol_type = other DNA
ctcgatgcagccgg

SEQ ID NO: 45 - DNA, 14 bp, synthetic construct
  source 1..14: mol_type = other DNA
taggtgcgaagtca

SEQ ID NO: 46 - DNA, 41 bp, synthetic construct
  source 1..41: mol_type = other DNA
gtgaggcgctagagcataggtgcgctgcatcgaggtcataa

SEQ ID NO: 47 - DNA, 14 bp, synthetic construct
  source 1..14: mol_type = other DNA
ctcgatgcagcgca

SEQ ID NO: 48 - DNA, 14 bp, synthetic construct
  source 1..14: mol_type = other DNA
taggtgcgaagact

SEQ ID NO: 49 - DNA, 41 bp, synthetic construct
  source 1..41: mol_type = other DNA
gtgaggcgctagagcataggagccctgcatcgaggtcataa

SEQ ID NO: 50 - DNA, 14 bp, synthetic construct
  source 1..14: mol_type = other DNA
ctcgatgcagggct

SEQ ID NO: 51 - DNA, 14 bp, synthetic construct
  source 1..14: mol_type = other DNA
taggtgcgaatgaa

SEQ ID NO: 52 - DNA, 41 bp, synthetic construct
  source 1..41: mol_type = other DNA
gtgaggcgctagagcataggtaatctgcatcgaggtcataa

SEQ ID NO: 53 - DNA, 14 bp, synthetic construct
  source 1..14: mol_type = other DNA
ctcgatgcagatta

SEQ ID NO: 54 - DNA, 14 bp, synthetic construct
  source 1..14: mol_type = other DNA
taggtgcgaacctt

SEQ ID NO: 55 - DNA, 14 bp, synthetic construct
  source 1..14: mol_type = other DNA
agagatgtcggtgt

SEQ ID NO: 56 - DNA, 12 bp, synthetic construct
  source 1..12: mol_type = other DNA
aacttacaccga

SEQ ID NO: 57 - DNA, 13 bp, synthetic construct
  source 1..13: mol_type = other DNA
agagatgtcggtg

SEQ ID NO: 58 - DNA, 44 bp, synthetic construct
  source 1..44: mol_type = other DNA
cggcaaatagcgaagacagcnnnnnnnngcggagatagtcgaac

SEQ ID NO: 59 - DNA, 50 bp, synthetic construct
  source 1..50: mol_type = other DNA
gtgaggcgctagagcataggnnnnnnnnaacacgaggagttcgactatct

SEQ ID NO: 60 - DNA, 49 bp, synthetic construct
  source 1..49: mol_type = other DNA
gtgaggcgctagagcataggnnnnnnnnaacacgaggagttcgactatc

SEQ ID NO: 61 - DNA, 48 bp, synthetic construct
  source 1..48: mol_type = other DNA
gtgaggcgctagagcataggnnnnnnnnaacacgaggagttcgactat

SEQ ID NO: 62 - DNA, 47 bp, synthetic construct
  source 1..47: mol_type = other DNA
gtgaggcgctagagcataggnnnnnnnnaacacgaggagttcgacta

SEQ ID NO: 63 - DNA, 46 bp, synthetic construct
  source 1..46: mol_type = other DNA
gtgaggcgctagagcataggnnnnnnnnaacacgaggagttcgact

SEQ ID NO: 64 - DNA, 45 bp, synthetic construct
  source 1..45: mol_type = other DNA
gtgaggcgctagagcataggnnnnnnnnaacacgaggagttcgac

SEQ ID NO: 65 - DNA, 44 bp, synthetic construct
  source 1..44: mol_type = other DNA
gtgaggcgctagagcataggnnnnnnnnaacacgaggagttcga

SEQ ID NO: 66 - DNA, 43 bp, synthetic construct
  source 1..43: mol_type = other DNA
gtgaggcgctagagcataggnnnnnnnnaacacgaggagttcg

SEQ ID NO: 67 - DNA, 44 bp, synthetic construct
  source 1..44: mol_type = other DNA
cggcaaatagcgaagacagcnnnnnnnntaatcgaaggcttcgc

SEQ ID NO: 68 - DNA, 50 bp, synthetic construct
  source 1..50: mol_type = other DNA
gtgaggcgctagagcataggnnnnnnnngccaagaagggcgaagccttcg

SEQ ID NO: 69 - DNA, 49 bp, synthetic construct
  source 1..49: mol_type = other DNA
gtgaggcgctagagcataggnnnnnnnngccaagaagggcgaagccttc

SEQ ID NO: 70 - DNA, 48 bp, synthetic construct
  source 1..48: mol_type = other DNA
gtgaggcgctagagcataggnnnnnnnngccaagaagggcgaagcctt

SEQ ID NO: 71 - DNA, 47 bp, synthetic construct
  source 1..47: mol_type = other DNA
gtgaggcgctagagcataggnnnnnnnngccaagaagggcgaagcct

SEQ ID NO: 72 - DNA, 46 bp, synthetic construct
  source 1..46: mol_type = other DNA
gtgaggcgctagagcataggnnnnnnnngccaagaagggcgaagcc

SEQ ID NO: 73 - DNA, 45 bp, synthetic construct
  source 1..45: mol_type = other DNA
gtgaggcgctagagcataggnnnnnnnngccaagaagggcgaagc

SEQ ID NO: 74 - DNA, 44 bp, synthetic construct
  source 1..44: mol_type = other DNA
gtgaggcgctagagcataggnnnnnnnngccaagaagggcgaag

SEQ ID NO: 75 - DNA, 43 bp, synthetic construct
  source 1..43: mol_type = other DNA
gtgaggcgctagagcataggnnnnnnnngccaagaagggcgaa

SEQ ID NO: 76 - DNA, 44 bp, synthetic construct
  source 1..44: mol_type = other DNA
cggcaaatagcgaagacagcnnnnnnnnaacgaacaaggttcct

SEQ ID NO: 77 - DNA, 50 bp, synthetic construct
  source 1..50: mol_type = other DNA
gtgaggcgctagagcataggnnnnnnnnctcaatctgaaggaaccttgtt

SEQ ID NO: 78 - DNA, 49 bp, synthetic construct
  source 1..49: mol_type = other DNA
gtgaggcgctagagcataggnnnnnnnnctcaatctgaaggaaccttgt

SEQ ID NO: 79 - DNA, 48 bp, synthetic construct
  source 1..48: mol_type = other DNA
gtgaggcgctagagcataggnnnnnnnnctcaatctgaaggaaccttg

SEQ ID NO: 80 - DNA, 47 bp, synthetic construct
  source 1..47: mol_type = other DNA
gtgaggcgctagagcataggnnnnnnnnctcaatctgaaggaacctt

SEQ ID NO: 81 - DNA, 46 bp, synthetic construct
  source 1..46: mol_type = other DNA
gtgaggcgctagagcataggnnnnnnnnctcaatctgaaggaacct

SEQ ID NO: 82 - DNA, 45 bp, synthetic construct
  source 1..45: mol_type = other DNA
gtgaggcgctagagcataggnnnnnnnnctcaatctgaaggaacc

SEQ ID NO: 83 - DNA, 44 bp, synthetic construct
  source 1..44: mol_type = other DNA
gtgaggcgctagagcataggnnnnnnnnctcaatctgaaggaac

SEQ ID NO: 84 - DNA, 43 bp, synthetic construct
  source 1..43: mol_type = other DNA
gtgaggcgctagagcataggnnnnnnnnctcaatctgaaggaa

SEQ ID NO: 85 - DNA, 47 bp, synthetic construct
  source 1..47: mol_type = other DNA
gtatgcgatgccaggtttgannnnnnngggctaaattgtcttacaac

SEQ ID NO: 86 - DNA, 47 bp, synthetic construct
  source 1..47: mol_type = other DNA
agacaacggcagcattctagnnnnnnntatgaccagcgtttccttgt

SEQ ID NO: 87 - DNA, 30 bp, synthetic construct
  source 1..30: mol_type = other DNA
ctagaatgctgccgttgtctgttgtaagac

SEQ ID NO: 88 - DNA, 29 bp, synthetic construct
  source 1..29: mol_type = other DNA
ctagaatgctgccgttgtctgttgtaaga

SEQ ID NO: 89 - DNA, 28 bp, synthetic construct
  source 1..28: mol_type = other DNA
ctagaatgctgccgttgtctgttgtaag

SEQ ID NO: 90 - DNA, 27 bp, synthetic construct
  source 1..27: mol_type = other DNA
ctagaatgctgccgttgtctgttgtaa

SEQ ID NO: 91 - DNA, 26 bp, synthetic construct
  source 1..26: mol_type = other DNA
ctagaatgctgccgttgtctgttgta

SEQ ID NO: 92 - DNA, 25 bp, synthetic construct
  source 1..25: mol_type = other DNA
ctagaatgctgccgttgtctgttgt

SEQ ID NO: 93 - DNA, 24 bp, synthetic construct
  source 1..24: mol_type = other DNA
ctagaatgctgccgttgtctgttg

SEQ ID NO: 94 - DNA, 23 bp, synthetic construct
  source 1..23: mol_type = other DNA
ctagaatgctgccgttgtctgtt

SEQ ID NO: 95 - DNA, 10 bp, synthetic construct
  source 1..10: mol_type = other DNA
gtatggcgtc

SEQ ID NO: 96 - DNA, 25 bp, synthetic construct
  source 1..25: mol_type = other DNA
gccgttgtctgacgccatacgttgt

SEQ ID NO: 97 - DNA, 30 bp, synthetic construct
  source 1..30: mol_type = other DNA
tggtcctacgagacatcggtacgataaaaa

SEQ ID NO: 98 - DNA, 69 bp, synthetic construct
  source 1..69: mol_type = other DNA
gcaggtataccatcgccgcttatctaatactggaaatcctgccggtgatagcatggaattctgattctg

SEQ ID NO: 99 - DNA, 34 bp, synthetic construct
  source 1..34: mol_type = other DNA
cagaatcagaattcgatgctatcaccggcaggat

SEQ ID NO: 100 - DNA, 26 bp, synthetic construct
  source 1..26: mol_type = other DNA
aagcggcgatggtatacctgcttttt

SEQ ID NO: 101 - DNA, 25 bp, synthetic construct
  source 1..25: mol_type = other DNA
accgatgtctcgtaggaccagttat

SEQ ID NO: 102 - DNA, 20 bp, synthetic construct
  source 1..20: mol_type = other DNA
catgctatcaccggcaggat

SEQ ID NO: 103 - DNA, 104 bp, synthetic construct
  source 1..104: mol_type = other DNA
ataactggtcctacgagacatcggtacgataaaaagcaggtataccatcgccgcttatctaatactggaaatcctgccggtgatagcatggaattctgattctg

SEQ ID NO: 104 - DNA, 104 bp, synthetic construct
  source 1..104: mol_type = other DNA
cagaatcagaattccatgctatcaccggcaggatttccagtattagataagcggcgatggtatacctgctttttatcgtaccgatgtctcgtaggaccagttat

SEQ ID NO: 105 - DNA, 20 bp, synthetic construct
  source 1..20: mol_type = other DNA
tggtcctacgagacatcggt

(19) United States
(12) Patent Application Publication
(10) Pub. No.: US 2024/0409983 A1
(43) Pub. Date: Dec. 12, 2024

(54) METHODS FOR ANALYZING A SAMPLE

(71) Applicant: MESO SCALE TECHNOLOGIES, LLC., Rockville, MD (US)

(72) Inventors: David ROUTENBERG, Rockville, MD (US); Evan GIZZIE, Rockville, MD (US); Alexander K. TUCKER-SCHWARTZ, Rockville, MD (US); George SIGAL, Rockville, MD (US); Khondaker RAHMAN, Rockville, MD (US); Deepak NANJAPPA, Rockville, MD (US)

(73) Assignee: MESO SCALE TECHNOLOGIES, LLC., Rockville, MD (US)

(21) Appl. No.: 18/734,455

(22) Filed: Jun. 5, 2024

Related U.S. Application Data
(60) Provisional application No. 63/506,573, filed on Jun. 6, 2023.
(60) Provisional application No. 63/595,551, filed on Nov. 2, 2023.
(60) Provisional application No. 63/644,393, filed on May 8, 2024.

(51) Int. Cl. C12Q 1/6823; C12Q 1/6806; C12Q 1/6834; C12Q 1/6851

(52) U.S. Cl. CPC C12Q 1/6823; C12Q 1/6806; C12Q 1/6834; C12Q 1/6851

(57) ABSTRACT

Provided herein are methods for analyzing a sample for an analyte using proximity-based extension, such as proximity extension strand displacement. Also provided are multiplexed methods for analyzing a sample using proximity-based extension. Provided herein are methods for screening for pairwise combination of binding moieties, for use in a sandwich-type assay. Also provided are compositions that find use in the present methods.

### REFERENCE TO RELATED APPLICATIONS

The present application claims the priority benefit of U.S. provisional application No. 63/644,393, filed May 8, 2024; U.S. provisional application No. 63/595,551, filed Nov. 2, 2023; and U.S. provisional application No. 63/506,573, filed Jun. 6, 2023, the entire contents of each of which are incorporated herein by reference.

### REFERENCE TO A SEQUENCE LISTING

The present application is being filed along with a Sequence Listing in electronic format. The Sequence Listing is provided as a file entitled MESOP0002_ST26.xml, created and last saved on Jun. 4, 2024, which is 96,042 bytes in size. The information in the electronic format of the Sequence Listing is incorporated herein by reference in its entirety.

### BACKGROUND

The present disclosure generally relates to in vitro assays for detecting analytes in a sample.

Highly multiplex protein assays can be used in, for example, biomarker discovery and/or drug screening. Such highly multiplex assays could employ PCR and/or immunosequencing. Methods that can simultaneously quantify the concentration of multiple protein biomarkers, and that are also calibrated and quantitative are needed.

### SUMMARY

Provided herein is a method of analyzing a sample for an analyte, comprising: a) providing a solid support comprising: a capture moiety attached to the solid support, wherein the capture moiety binds an analyte; and a capture oligonucleotide attached to the solid support, wherein the capture oligonucleotide comprises a 3′ hybridizing region; b) providing a detection conjugate comprising: a detection moiety that binds the analyte; and a detection oligonucleotide attached to the detection moiety, wherein the detection oligonucleotide comprises a 3′ hybridizing region complementary to the 3′ hybridizing region of the capture oligonucleotide; c) preparing a complexing solution by: i) combining in a solution the solid support provided in a) and the detection conjugate provided in b) with a sample, thereby allowing the capture moiety of the solid support and the detection moiety of the detection conjugate to be bound to the analyte if present in the sample; ii) contacting the solid support provided in a) with a sample, thereby allowing the capture moiety of the solid support to be bound to the analyte if present in the sample, and combining in a solution the sample-contacted solid support and the detection conjugate provided in b); or iii) contacting the detection conjugate provided in b) with a sample, thereby allowing the detection moiety to be bound to the analyte if present in the sample, and combining in a solution the sample-contacted detection conjugate with the solid support provided in a), thereby allowing the capture moiety and the detection moiety in the complexing solution to both be bound (or to be simultaneously bound) to the analyte if present such that the capture oligonucleotide and detection oligonucleotide are in proximity if the analyte is present in the sample; d) permitting the 3′ hybridizing region of the capture oligonucleotide and the 3′ hybridizing region of the detection oligonucleotide that are in proximity to hybridize to each other; e) extending the hybridized capture oligonucleotide and/or the hybridized detection oligonucleotide to generate an on-target extension product that comprises the extended capture oligonucleotide and/or the extended detection oligonucleotide; f) releasing the on-target extension product from the solid support and, optionally, from the detection moiety; and g) determining the presence and/or amount, or the absence of the released on-target extension product to thereby determine the presence and/or amount, or the absence, of the analyte in the sample.

Also provided is a composition comprising a plurality of (e.g., at least 30, or 30-200, or 100-500, etc.) pairs of oligonucleotides, each pair comprising: a capture oligonucleotide comprising: a 3′ hybridizing region and a 5′ tethering region; and a detection oligonucleotide comprising: a 3′ hybridizing region complementary to the 3′ hybridizing region of the capture oligonucleotide, and a 5′ tethering region, wherein the 3′ hybridizing region of the capture oligonucleotide and detection oligonucleotide of each pair of oligonucleotides is not complementary to the 3′ hybridizing region of the detection oligonucleotide and the capture oligonucleotide, respectively, of any other pair of the plurality of (e.g., at least 30, or 30-200, or 100-500, etc.) pairs of oligonucleotides, wherein each of the plurality of (e.g., at least 30, or 30-200, etc.) pairs of oligonucleotides has a mis-pairing rate of at most about 1% in the presence of the other plurality of (e.g., at least 30, or 30-200, or 100-500, etc.) pairs of oligonucleotides in a proximity-based extension assay. In certain embodiments, the capture oligonucleotide is at least 25 nucleotides long and its 3′ hybridizing region is at most 10 nucleotides long, and its 5′ tethering region is at least 15 nucleotides long; and the detection oligonucleotide is at least 25 nucleotides long and its 3′ hybridizing region is at most 10 nucleotides long and complementary to the 3′ hybridizing region of the capture oligonucleotide, and its 5′ tethering region is at least 15 nucleotides long, wherein the 3′ hybridizing region of the capture oligonucleotide and detection oligonucleotide of each pair of oligonucleotides is not complementary to the 3′ hybridizing region of the detection oligonucleotide and the capture oligonucleotide, respectively, of any other pair of the plurality of (e.g., at least 30, or 30-200, or 100-500, etc.) pairs of oligonucleotides, wherein each of the plurality of (e.g., at least 30, or 30-200, or 100-500, etc.) pairs of oligonucleotides has a mis-pairing rate of at most about 1% in the presence of the other plurality of (e.g., at least 30, or 30-200, or 100-500, etc.) pairs of oligonucleotides in a proximity-based extension assay.

Further provided is a composition comprising: a first pool of a plurality of (e.g., at least 30, or 30-200, or 100-500, etc.) solid supports, each solid support comprising: a capture moiety attached to the solid support, wherein the capture moiety binds an analyte; and a capture oligonucleotide attached to the solid support, wherein the capture oligonucleotide comprises a 3′ hybridizing region; and a second pool of a plurality of (e.g., at least 30, or 30-200, or 100-500, etc.) detection conjugates, each detection conjugate comprising: a detection moiety that binds the analyte; and a detection oligonucleotide attached to the detection moiety, wherein the detection oligonucleotide comprises a 3′ hybridizing region complementary to the 3′ hybridizing region of the capture oligonucleotide, wherein each of the plurality of (e.g., at least 30, or 30-200, or 100-500, etc.) solid supports forms a paired combination with a corresponding detection conjugate of the plurality of (e.g., at least 30, or 30-200, or 100-500, etc.) detection conjugates, wherein a binding target of the capture moiety and detection moiety of each paired combination is the same, and wherein different paired combinations have different binding targets, wherein the 3′ hybridizing region of the capture oligonucleotide and detection oligonucleotide of each paired combination is not complementary to the 3′ hybridizing region of the detection oligonucleotide and the capture oligonucleotide, respectively, of any other paired combination. In some embodiments, each of the paired combinations has a mis-hybridization rate of at most about 1% in the presence of the other paired combinations in a proximity-based extension assay.

Also provided is a composition comprising: a solid support comprising: a capture moiety attached to the solid support, wherein the capture moiety binds an analyte; and a capture oligonucleotide attached to the solid support, wherein the capture oligonucleotide comprises a 3′ hybridizing region; a detection conjugate comprising: a detection moiety that binds the analyte; and a detection oligonucleotide attached to the detection moiety, wherein the detection oligonucleotide comprises a 3′ hybridizing region complementary to the 3′ hybridizing region of the capture oligonucleotide, wherein the capture moiety and the detection moiety are both bound (or are simultaneously bound) to the analyte such that the 3′ hybridizing region of the capture oligonucleotide and the 3′ hybridizing region of the detection oligonucleotide are in proximity to allow hybridization to each other.

Provided herein is a composition comprising a plurality of partially double-stranded nucleic acids, each partially double-stranded nucleic acid comprising: a capture oligonucleotide hybridized at the 5′ end with a first tether oligonucleotide and the capture oligonucleotide comprising a 3′ hybridizing region of at most 10 nucleotides; and a detection oligonucleotide hybridized at the 5′ end with a second tether oligonucleotide and the detection oligonucleotide comprising a 3′ hybridizing region of at most 10 nucleotides, wherein the 3′ hybridizing region of the capture oligonucleotide is hybridized to the 3′ hybridizing region of the detection oligonucleotide. In certain embodiments, each partially double-stranded nucleic acid comprises: a capture oligonucleotide hybridized at the 5′ end with a first tether oligonucleotide of 15-25 or 15-30 nucleotides in length and the capture oligonucleotide comprising a 3′ hybridizing region of at most 10 nucleotides; and a detection oligonucleotide hybridized at the 5′ end with a second tether oligonucleotide of 15-25 or 15-30 nucleotides in length and the detection oligonucleotide comprising a 3′ hybridizing region of at most 10 nucleotides, wherein the 3′ hybridizing region of the capture oligonucleotide is hybridized to the 3′ hybridizing region of the detection oligonucleotide.

Provided herein is a method of analyzing a sample for an analyte, comprising: a) providing a first conjugate comprising: a first moiety that binds an analyte; and a first splint oligonucleotide attached to the first moiety, wherein the first splint oligonucleotide comprises a 3′ hybridizing region; b) providing a second conjugate comprising: a second moiety that binds the analyte; and a second splint oligonucleotide attached to the second moiety, wherein the second splint oligonucleotide comprises a 3′ hybridizing region complementary to the 3′ hybridizing region of the first splint oligonucleotide; wherein the first splint oligonucleotide is attached to the first moiety via hybridization to a first tether oligonucleotide attached to the first moiety, and/or the second splint oligonucleotide is attached to the second moiety via hybridization to a second tether oligonucleotide attached to the second moiety; wherein the first and/or the second splint oligonucleotide comprises a barcode sequence that identifies the moiety to which the splint oligonucleotide is attached and/or a binding target thereof; c) preparing a complexing solution by: i) combining in a solution the first conjugate provided in a) and the second conjugate provided in b) with a sample, thereby allowing the first conjugate and the second conjugate to be bound to the analyte if present in the sample; or ii) contacting the first conjugate provided in a) with a sample, thereby allowing the first moiety of the first conjugate to be bound to the analyte if present in the sample, and combining in a solution the sample-contacted first conjugate and the second conjugate provided in b); thereby allowing the first moiety and the second moiety in the complexing solution to both be bound to the analyte if present such that the first splint oligonucleotide and second splint oligonucleotide are in proximity if the analyte is present in the sample; d) permitting the 3′ hybridizing region of the first splint oligonucleotide and the 3′ hybridizing region of the second splint oligonucleotide that are in proximity to hybridize to each other; e) extending the hybridized first splint oligonucleotide and/or the hybridized second splint oligonucleotide to generate an on-target extension product that comprises the extended first splint oligonucleotide and/or the extended second splint oligonucleotide; f) releasing the on-target extension product from the first moiety and/or the second moiety; and g) determining the presence and/or amount, or the absence of the on-target extension product to thereby determine the presence and/or amount, or the absence, of the analyte in the sample.

Provided herein is a composition comprising: a first pool of a plurality of first conjugates, each first conjugate comprising: a first moiety that binds an analyte; and a first splint oligonucleotide attached to the first moiety, wherein the first splint oligonucleotide comprises a 3′ hybridizing region; and a second pool of a plurality of second conjugates, each second conjugate comprising: a second moiety that binds the analyte; and a second splint oligonucleotide attached to the second moiety, wherein the second splint oligonucleotide comprises a 3′ hybridizing region complementary to the 3′ hybridizing region of the capture oligonucleotide, wherein each of the plurality of first conjugates forms a paired combination with a corresponding second conjugate of the plurality of second conjugates, wherein a binding target of the first moiety and second moiety of each paired combination is the same, and wherein different paired combinations have different binding targets (or wherein different paired combinations have different first and/or second moieties), wherein for each paired combination, the first splint oligonucleotide is attached to the first moiety via hybridization to a first tether oligonucleotide attached to the first moiety, and/or the second splint oligonucleotide is attached to the second moiety via hybridization to a second tether oligonucleotide attached to the second moiety, wherein for each paired combination, the first and/or the second splint oligonucleotide comprises a barcode sequence that identifies the moiety to which the splint oligonucleotide is attached and/or a binding target thereof, wherein the 3′ hybridizing region of the first splint oligonucleotide and second splint oligonucleotide of each paired combination is not complementary to the 3′ hybridizing region of the second splint oligonucleotide and the first splint oligonucleotide, respectively, of any other paired combination.

Also provided is a method of identifying a pairwise combination of binding moieties that can both be bound (or can be simultaneously bound) to a binding target, comprising: a) providing a plurality of solid supports, each solid support comprising: a first binding moiety attached to the solid support, wherein the binding moiety binds a binding target; and a capture oligonucleotide attached to the solid support, wherein the capture oligonucleotide comprises: a 3′ hybridizing region; and a capture barcode region, wherein the plurality of solid supports comprises a first plurality of different binding moieties that bind the same binding target, wherein the capture barcode region of each solid support of the plurality of solid supports identifies one of the first plurality of different binding moieties attached to the respective solid support, and wherein the 3′ hybridizing regions of the capture oligonucleotides attached to the plurality of solid supports are the same; b) providing a plurality of detection conjugates comprising: a second binding moiety; and a detection oligonucleotide attached to the binding moiety, wherein the detection oligonucleotide comprises: a 3′ hybridizing region complementary to the 3′ hybridizing region of the capture oligonucleotide; and a detector barcode region, wherein the plurality of detection conjugates comprises a second plurality of different binding moieties, wherein the detector barcode region of each detection conjugate of the plurality of detection conjugates identifies one of the second plurality of different binding moieties attached to the detection oligonucleotide, and wherein the 3′ hybridizing regions of the detection oligonucleotides of the plurality of detection conjugates are the same; c) preparing a complexing solution by: i) combining in a solution the plurality of solid supports provided in a) and the plurality of detection conjugates provided in b) with a sample comprising a plurality of molecules of the binding target, thereby allowing one or more of the first plurality of different binding moieties of the plurality of solid supports and one or more of the second plurality of different binding moieties of the plurality of detection conjugates to be bound to one or more molecules of the plurality of molecules of the binding target, ii) contacting the plurality of solid supports provided in a) with the sample, thereby allowing one or more of the first plurality of different binding moieties of the plurality of solid supports to be bound to one or more molecules of the plurality of molecules of the binding target, and combining in a solution the sample-contacted plurality of solid supports and the plurality of detection conjugates provided in b), or iii) contacting the plurality of detection conjugates provided in b) with the sample, thereby allowing one or more of the second plurality of different binding moieties of the plurality of detection conjugates to be bound to one or more molecules of the plurality of molecules of the binding target, and combining in a solution the sample-contacted plurality of detection conjugates and the plurality of solid supports provided in a), wherein the 3′ hybridizing region of a capture oligonucleotide attached to a first solid support of the plurality of solid supports and the 3′ hybridizing region of a detection oligonucleotide of a first detection conjugate of the plurality of detection conjugates are in proximity when the first binding moiety attached to the first solid support and the second binding moiety of the first detection conjugate are both bound (or are simultaneously bound) to the same molecule of the plurality of molecules of the binding target; d) permitting the 3′ hybridizing region of the capture oligonucleotides attached to the plurality of solid supports and the 3′ hybridizing region of the detection oligonucleotides of the plurality of detection conjugates that are in proximity to hybridize to each other; e) extending hybridized capture oligonucleotides and/or hybridized detection oligonucleotides to generate extension products that each comprise an extended capture oligonucleotide and/or an extended detection oligonucleotide; f) releasing each of the extension products from the respective solid supports and, optionally, from the respective second binding moieties; and g) determining: the presence and/or amount, or the absence of the released extension products; and the respective capture barcode region and the detector barcode region in each of the released extension products, to thereby determine the suitability of a combination of binding moieties identified by the capture barcode region and the detector barcode region in the released extension products for use in a sandwich-type assay.

Also provided is a method of analyzing a sample for an analyte, comprising: a) providing a first construct comprising: a first moiety that binds an analyte; and a first splint oligonucleotide attached to the first moiety, wherein the first splint oligonucleotide comprises a 3′ hybridizing region; b) providing a second construct comprising: a second moiety that binds the analyte; and a second splint oligonucleotide attached to the second moiety, wherein the second splint oligonucleotide comprises a 3′ hybridizing region complementary to the 3′ hybridizing region of the first splint oligonucleotide; c) preparing a complexing solution by: i) combining in a solution the first construct provided in a) and the second construct provided in b) with a sample, thereby allowing the first moiety and the second moiety to be bound to the analyte if present in the sample; ii) contacting the first construct provided in a) with a sample, thereby allowing the first moiety to be bound to the analyte if present in the sample, and combining in a solution the sample-contacted construct and the second construct provided in b); or iii) contacting the second construct provided in b) with a sample, thereby allowing the second moiety to be bound to the analyte if present in the sample, and combining in a solution the sample-contacted construct with the first construct provided in a), thereby allowing the first moiety and the second moiety in the complexing solution to both be bound to the analyte if present such that the first splint oligonucleotide and second splint oligonucleotide are in proximity if the analyte is present in the sample; d) permitting the 3′ hybridizing region of the first splint oligonucleotide and the 3′ hybridizing region of the second splint oligonucleotide that are in proximity to hybridize to each other; e) extending the hybridized first splint oligonucleotide and/or the hybridized second splint oligonucleotide to generate an on-target extension product that comprises the extended first splint oligonucleotide and/or the extended second splint oligonucleotide; f) optionally, releasing the on-target extension product from the first construct and/or the second construct; and g) determining the presence and/or amount, or the absence of the on-target extension product to thereby determine the presence and/or amount, or the absence, of the analyte in the sample, wherein the method comprises one or more of the following: (I) the complexing solution comprises one or more blocker oligonucleotides, wherein each blocker oligonucleotide hybridizes to a subpart of the first splint oligonucleotide and/or of the second splint oligonucleotide; (II) the method comprises analyzing the sample for a first analyte and a second analyte, wherein preparing the complexing solution in c) comprises: preparing the complexing solution in a plurality of subpools comprising a first subpool and a second subpool, wherein the first moiety and second moiety in the prepared complexing solution of the first subpool bind the first analyte, and the first moiety and second moiety in the prepared complexing solution of the second subpool bind the second analyte; and combining the plurality of subpools before determining the presence and/or amount, or the absence of the on-target extension product in g); (III) providing a plurality of paired combinations of the first construct and second construct, wherein the plurality of paired combinations comprises one or more trimmed paired combinations comprising splint oligonucleotides having a 3′ hybridizing region that is 1, 2, 3 or more nucleotides shorter than the 3′ hybridizing region of the splint oligonucleotides of at least one other paired combination of the plurality of paired combinations, wherein the 3′ hybridizing regions of the splint oligonucleotides of the at least one other paired combination of the plurality of paired combinations is different from and is not complementary to any contiguous stretch of the 3′ hybridizing region of the splint oligonucleotides of the one or more trimmed paired combinations, optionally wherein the on-target extension products generated from substantially all (e.g., at least 95%) of the plurality of paired combinations of the first construct and second construct have the same length; or (IV) attenuating an amount of amplification products by reducing or interfering with a binding interaction between the analyte and the first moiety or the second moiety, and/or suppressing on-target interactions between the conjugate splint oligonucleotide and the first splint oligonucleotide when the first moiety and the second moiety are both bound to the analyte.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a flow diagram showing a non-limiting embodiment of a method of analyzing a sample.

FIG. 2 is a flow diagram showing a non-limiting embodiment of a method of identifying a pairwise combination of binding moieties that can both be bound (or can be simultaneously bound) to a binding target.

FIGS. 3A, 3B, 3C, and 3D are a collection of schematic diagrams showing non-limiting embodiments of the components of methods of the present disclosure.

FIG. 4 is a schematic diagram showing non-limiting embodiments of a method of analyzing a sample for an analyte.

FIGS. 5A, 5B, 5C, 5D, and 5E are a collection of schematic diagrams showing non-limiting embodiments of the components of methods of the present disclosure.

FIG. 6 is a schematic diagram showing non-limiting embodiments of a method of analyzing a sample for an analyte.

FIGS. 7A and 7B are a collection of schematic diagrams showing non-limiting embodiments of the components of methods of the present disclosure.

FIG. 8 is a schematic diagram showing non-limiting embodiments of a method of analyzing a sample for an analyte.

FIG. 9 is a schematic diagram showing non-limiting embodiments of methods of analyzing a sample for an analyte.

FIG. 10 is a schematic diagram showing non-limiting embodiments of a capture and detection oligonucleotide.

FIG. 11 is a collection of a data table and a plot showing results of a singleplex PESD assay, according to non-limiting embodiments of the present disclosure.

FIGS. 12A and 12B are a collection of a data table and a plot showing results of a multiplex PESD assay, according to non-limiting embodiments of the present disclosure.

FIG. 13 is a graph showing results of a multiplex PESD assay, according to non-limiting embodiments of the present disclosure.

FIG. 14 is a data table showing results of a multiplex PESD assay, according to non-limiting embodiments of the present disclosure.

FIG. 15 is a schematic diagram showing a non-limiting embodiment of an experimental design for screening oligonucleotides with unique hybridization overlaps.

FIG. 16 is a schematic diagram showing a non-limiting embodiment of a method of screening oligonucleotides with unique hybridization overlaps.

FIG. 17 is a heatmap plot showing the results of screening oligonucleotides with unique hybridization overlaps, according to non-limiting embodiments of the present disclosure.

FIG. 18 is a plot showing the results of screening oligonucleotides with unique hybridization overlaps, according to non-limiting embodiments of the present disclosure.

FIG. 19 is a schematic diagram showing a non-limiting embodiment of a method of identifying a pairwise combination of binding moieties that can both be bound (or can be simultaneously bound) to a binding target.

FIG. 20 is a schematic diagram showing a non-limiting embodiment of a method of designing barcode regions.

FIG. 21 is a heatmap plot showing the results of an assay for designing barcode regions, according to non-limiting embodiments of the present disclosure.

FIG. 22 is a schematic diagram showing non-limiting embodiments of detection conjugates and solid supports.

FIG. 23 is a schematic diagram showing non-limiting embodiments of a method of analyzing a sample for an analyte.

FIG. 24 is a schematic diagram showing non-limiting embodiments of a method of analyzing a sample for an analyte.

FIG. 25 is a collection of data tables showing results of a method of analyzing a sample for an analyte, according to non-limiting embodiments of the present disclosure.

FIG. 26 is a collection of plots showing results of a method of analyzing a sample for an analyte, according to non-limiting embodiments of the present disclosure.

FIG. 27 is a data table showing results of a method of analyzing a sample for an analyte, according to non-limiting embodiments of the present disclosure.

FIG. 28 is a flow diagram showing a non-limiting embodiment of a method of analyzing a sample.

FIGS. 29A and 29B are a collection of schematic diagrams showing a non-limiting embodiment of a method of analyzing a sample for an analyte.

FIGS. 30A and 30B are a flow diagram showing a non-limiting embodiment of a method of analyzing a sample.

FIG. 31A is a schematic diagram showing a non-limiting example of a pair of splint oligonucleotides (e.g., a pair of capture and detection oligonucleotides) that hybridize to each other, where one side is provided attached to a solid support (e.g., a bead) via a tether oligonucleotide, and the other side is provided attached to a moiety (e.g., antibody) via a tether oligonucleotide.

FIG. 31B is a schematic diagram showing non-limiting examples of blocker oligonucleotides that hybridize to a portion of a splint oligonucleotide (e.g., a capture oligonucleotide or a detection oligonucleotide).

FIG. 31C is a schematic diagram showing non-limiting examples of blocker oligonucleotides that hybridize to a portion of a splint oligonucleotide (e.g., a capture oligonucleotide or a detection oligonucleotide).

FIG. 31D is a schematic diagram showing non-limiting examples of blocker oligonucleotides, a splint oligonucleotide, and a tether oligonucleotide.

FIG. 32A is a schematic diagram showing non-limiting examples of pairs of splint oligonucleotides (e.g., pairs of capture and detection oligonucleotides that hybridize to each other) that have a subpool barcode.

FIG. 32B is a schematic diagram showing non-limiting examples of pairs of splint oligonucleotides (e.g., pairs of capture and detection oligonucleotides that hybridize to each other) that have a subpool barcode, and non-limiting examples of blocker oligonucleotides that hybridize a portion of the splint oligonucleotides.

FIG. 33 is a schematic diagram showing trimming of a splint oligonucleotide (e.g., detection oligonucleotide), according to some non-limiting embodiments of the present disclosure.

FIGS. 34A, 34B, and 34C are a collection of scatter plots showing qPCR Ct values with and without IL-13 analyte in a PESD assay, according to some non-limiting embodiments of the present disclosure.

FIG. 35 is a tabular plot showing Ct and delta Ct values for detecting IL-13 analyte using beads prepared with the indicated amounts of capture oligonucleotides (as measured by the amount of tether (“anchor”) oligonucleotides), according to some non-limiting embodiments of the present disclosure.

FIGS. 36A and 36B are graphs showing Ct and delta Ct values for detecting IL-13 analyte using beads with the indicated amounts of tether oligonucleotides and the indicated percentage stoichiometry of capture to detector oligonucleotide, according to some non-limiting embodiments of the present disclosure.

FIGS. 37A and 37B are graphs showing Ct and delta Ct values for detecting IL-13 analyte using beads with the indicated amounts of tether oligonucleotides and the indicated percentage stoichiometry of capture to detector oligonucleotide, according to some non-limiting embodiments of the present disclosure.

FIG. 38 is a tabular plot showing Ct values (top panel) for detecting IL-13 analyte using a competitor oligonucleotide that binds to the 3′ hybridization region of the detector or capture oligonucleotides, and delta Ct values between specific (“TOC”) and non-specific (“NSB”) product CT values, according to some non-limiting embodiments of the present disclosure.

FIGS. 39A, 39B, and 39C show the effects of changing the length of the 3′ hybridization sequence in a PESD assay. FIG. 39A is a schematic diagram showing non-limiting examples of tether oligonucleotides that have different 3′ hybridization sequence lengths. FIG. 39B is a collection of a table and graphs showing specific signal and non-specific background in a singleplex PESD IL-13 assay using capture and detector oligonucleotides that have different 3′ hybridization sequence lengths. FIG. 39C is a collection of a table and a graph showing the signal-to-background ratio and dynamic range of a singleplex PESD IL-13 assay using capture and detector oligonucleotides that have different 3′ hybridization sequence lengths.

FIGS. 40A and 40B show attenuation of signal by cold-capture antibody in a PESD assay. FIG. 40A is a tabular representation of signal and background noise of a singleplex PESD IL-13 assay at different concentrations of analyte and cold capture antibody. FIG. 40B is a graph showing Ct value as a function of the analyte concentration, at different cold capture antibody concentrations.

FIGS. 41A, 41B, and 41C show normalization of multiplexed amplicons during library preparation for next generation sequencing analysis by depletion of adapter primers. FIG. 41A is a schematic showing a non-limiting theoretical example of primer depletion normalization for reducing calibrator NGS burden. FIG. 41B is an experimental example of primer depletion normalization for reducing calibrator NGS burden. FIG. 41C is an experimental example of primer depletion normalization for sample balancing.

FIG. 42 shows the analytical quantitation after primer depletion normalization.

FIG. 43 is a schematic diagram showing non-limiting embodiments of a proximity ligation assay (PLA) employing asymmetrical splints.

FIGS. 44A and 44B is a schematic diagram showing non-limiting embodiments of a proximity ligation ligation assay (PLLA).

FIG. 45 is a schematic diagram showing non-limiting embodiments of a proximity ligation strand displacement (PLSD) assay.

FIGS. 46A and 46B show Ct values from qPCR analysis of ligation products from a proximity ligation assay (PLA).

FIG. 47 shows Ct values from qPCR analysis of ligation products from a proximity ligation ligation assay (PLLA).

FIG. 48 shows Ct values from qPCR analysis of ligation products from a PLSD (Proximity Ligation Strand Displacement) assay.

FIG. 49 shows the signal versus calibrator concentration and a 4PL fit for ten capture and detection antibody pairs.

FIG. 50 shows signal versus capture antibody concentration for an antibody pair using a capture antibody with affinity expected to be approximately 1 nM. Kd was calculated by fitting data to single-site binding model.

FIG. 51 shows calculated affinities for two antibodies, one with relatively poor affinity and one with relatively good affinity.

### DETAILED DESCRIPTION

Proximity extension assay (PEA) technology that combines binding assay methodologies for measuring biomarkers, such as antibody-based immunoassays, and DNA-based methodologies (PCR and readout using either quantitative real-time PCR or next generation sequencing (NGS)) can be used in a multiplex format to simultaneously quantify the concentration of multiple protein biomarkers.

Provided herein are methods for analyzing a sample for an analyte, multiplexed methods for analyzing a sample, methods for identifying a pairwise combination of binding moieties that can both be bound (or be simultaneously bound) to a binding target, for use in sandwich-type assays, and compositions that find use in the present methods.

### I. TERMS

Unless defined otherwise, technical and scientific terms used herein have the same meaning as commonly understood by one of ordinary skill in the art to which the present disclosure belongs.

A “moiety” or “binding moiety” as used herein with reference to a capture moiety or detection moiety denotes a molecule (e.g., protein, nucleic acid, etc.) that can bind another molecule. Non-limiting examples of a moiety include an antibody, a receptor, a lectin, an enzyme, or an aptamer.

As used herein, “binding target” denotes a molecule, or a portion (e.g., epitope) thereof, to which a binding moiety (e.g., capture moiety, detection moiety) binds.

A “binding pair” as used herein denotes a pair of molecules (“members”) that bind to each other. A binding pair can mediate attachment of two or more molecules with each other through attachment (e.g., covalent attachment) of one member of the binding pair to one molecule, and attachment (e.g., covalent attachment) of the other member of the binding pair to another molecule. The binding affinity between members of the binding pair can be 10⁻⁹ M or less, e.g., 10⁻¹⁰ M or less, 10⁻¹¹ M or less, 10⁻¹² M or less, 10⁻¹³ M or less, 10⁻¹⁴ M or less, 10⁻¹⁵ M or less, 10⁻¹⁶ M or less. Non-limiting examples of a binding pair include biotin and streptavidin/avidin, an IgG and protein A or protein G, a drug and drug receptor, a toxin and toxin receptor, a carbohydrate and lectin or carbohydrate receptor, a peptide or protein and peptide or protein receptor, etc.

As used herein, an antibody can be a full-length (e.g., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. In some embodiments, an antibody is a functional antibody fragment. For example, an antibody fragment can be a portion of an antibody such as F(ab′)2, Fab′, Fab, Fv, sFv and the like. An antibody fragment can bind with the same antigen that is recognized by the full-length antibody. An antibody fragment can include isolated fragments consisting of the variable regions of antibodies, such as the “Fv” fragments consisting of the variable regions of the heavy and light chains and recombinant single chain polypeptide molecules in which light and heavy variable regions are connected by a peptide linker (“scFv proteins”). Exemplary antibodies can include, but are not limited to, antibodies for cancer cells, antibodies for viruses, antibodies that bind to cell surface receptors (for example, CD8, CD34, and CD45), and therapeutic antibodies.

As used herein, the term “complementary” can refer to the capacity for hybridization between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule it may be the complement of the molecule that is hybridizing.

As used herein, the term “nucleic acid” refers to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide,” “oligonucleotide” can be used interchangeably.

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid may also include nucleobase (often referred to simply as “base”) modifications or substitutions. As used herein, “unmodified” or “natural” nucleobases can include the purine bases, (e.g., adenine (A) and guanine (G)), and the pyrimidine bases, (e.g., thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benothiazin-2(3H)-one), G-clamps such as a substituted phenoxazine cytidine (e.g., 9-(2-aminoethoxy)-H-pyrimido(5,4-(b) (1,4) benzoxazine-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazine-2(3H)-one), carbazole cytidine (2H-pyrimido(4,5-b)indol-2-one), pyridoindole cytidine (H-pyrido(3′,2′:4,5)pyrrolo[2,3-d]pyrimidin-2-one).

As used herein, the term “solid support” can refer to discrete solid or semi-solid surfaces. A solid support may encompass any type of solid, porous, or hollow sphere, ball, bearing, slide, microwell, cylinder, or other similar configuration composed of plastic, ceramic, metal, glass, or polymeric material (e.g., hydrogel) onto which a nucleic acid and a binding moiety may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. A bead can be non-spherical in shape. A solid support may comprise a magnetic or paramagnetic particle, a magnetic or paramagnetic microparticle, or a magnetic or paramagnetic bead. In some embodiments, a solid support may be used interchangeably with the term “bead.”

As used herein, a “splint oligonucleotide” denotes a nucleic acid molecule that can bridge two or more distinct molecular entities based on hybridization of at least a portion of the nucleotide sequence in the nucleic acid molecule to a complementary sequence in another nucleic acid molecule. In some embodiments, the splint oligonucleotide is conjugated to one of the two distinct molecular entities. In some embodiments, a splint oligonucleotide bridges two or more distinct nucleic acid molecules (e.g., two other distinct oligonucleotides) based on hybridization of at least a first portion of the nucleotide sequence in the nucleic acid molecule to a complementary sequence in a nucleic acid molecule of one of the molecular entities, and hybridization of at least a second portion of the nucleic acid molecule to a complementary sequence in a nucleic acid molecule of another of the molecular entities. In some embodiments, a splint oligonucleotide includes two (or more) portions that are complementary to a sequence in two (or more) other nucleic acid molecules. A capture oligonucleotide or a detection oligonucleotide described herein are non-limiting examples of splint oligonucleotides. For example, a capture oligonucleotide and a detection oligonucleotide, when held in proximity due to the capture moiety and detection moiety both being bound to an analyte, can bridge a solid support to a detection conjugate through hybridization of the 3′ hybridization regions. In some embodiments, the 3′ hybridization regions of the capture oligonucleotide (or a first splint oligonucleotide) and the detection oligonucleotide (or a second splint oligonucleotide) hybridize to complementary portions of a third splint oligonucleotide, which third splint oligonucleotide can bridge a capture moiety associated with the capture oligonucleotide and a detection moiety associated with the detection oligonucleotide through hybridization with the 3′ hybridization regions of the capture oligonucleotide (or a first splint oligonucleotide) and the detection oligonucleotide (or a second splint oligonucleotide).

The singular terms “a,” “an,” and “the” include plural referents unless context clearly indicates otherwise. Similarly, the word “or” is intended to include “and” unless the context clearly indicates otherwise. The abbreviation, “e.g.” is used herein to indicate a non-limiting example. Thus, the abbreviation “e.g.” is synonymous with the term “for example.” The term “about” as used herein to, for example, define the values and ranges of molecular weights means that the indicated values and/or range limits can vary within ±20%, e.g., within ±10%, including within ±5%. The use of “about” before a number includes the number itself. For example, “about 5” provides express support for “5.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### II. METHODS

### A. Methods of Analyzing a Sample

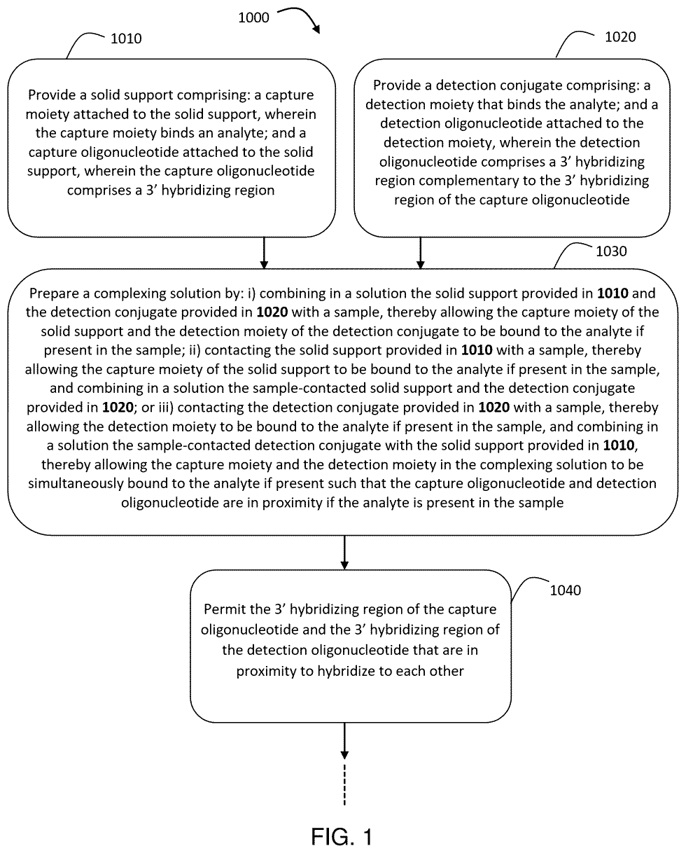

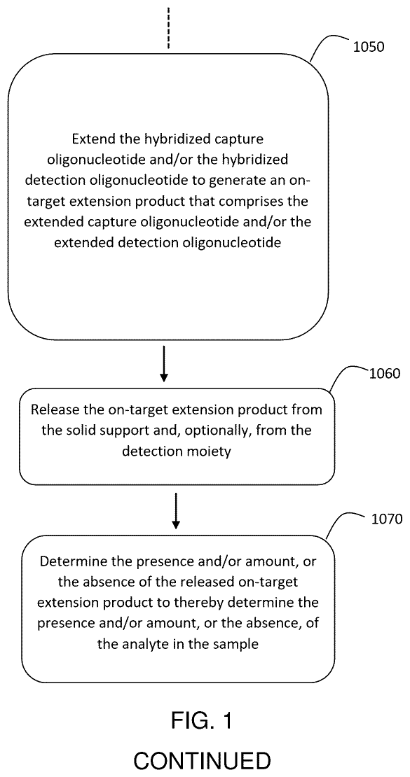

With reference to FIG. 1, a non-limiting example of a method 1000 of analyzing a sample for an analyte is provided (which for convenience may be referred to herein as an “analyte detection method”). The method can include, at block 1010, providing a solid support that includes: a capture moiety attached to the solid support, wherein the capture moiety binds an analyte; and a capture oligonucleotide attached to the solid support, wherein the capture oligonucleotide includes a 3′ hybridizing region. The method can further include, at block 1020, providing a detection conjugate including: a detection moiety that binds the analyte; and a detection oligonucleotide attached to the detection moiety, wherein the detection oligonucleotide includes a 3′ hybridizing region complementary to the 3′ hybridizing region of the capture oligonucleotide. The method can also include, at block 1030, preparing a complexing solution by: i) combining in a solution the solid support provided at block 1010 and the detection conjugate provided at block 1020 with a sample, thereby allowing the capture moiety of the solid support and the detection moiety of the detection conjugate to be bound to the analyte if present in the sample; ii) contacting the solid support provided at block 1010 with a sample, thereby allowing the capture moiety of the solid support to be bound to the analyte if present in the sample, and combining in a solution the sample-contacted solid support and the detection conjugate provided at block 1020; or iii) contacting the detection conjugate provided at block 1020 with a sample, thereby allowing the detection moiety to be bound to the analyte if present in the sample, and combining in a solution the sample-contacted detection conjugate with the solid support provided at block 1010, thereby allowing the capture moiety and the detection moiety in the complexing solution to both be bound (or be simultaneously bound) to the analyte if present such that the capture oligonucleotide and detection oligonucleotide are in proximity if the analyte is present in the sample. The method can include, at block 1040, permitting the 3′ hybridizing region of the capture oligonucleotide and the 3′ hybridizing region of the detection oligonucleotide that are in proximity to hybridize to each other. The method can also include, at block 1050, extending the hybridized capture oligonucleotide and/or the hybridized detection oligonucleotide to generate an on-target extension product that includes the extended capture oligonucleotide and/or the extended detection oligonucleotide. The method can further include, at block 1060, releasing the on-target extension product from the solid support and, optionally, from the detection moiety. The method can also include, at block 1070, determining the presence and/or amount, or the absence of the released on-target extension product to thereby determine the presence and/or amount, or the absence, of the analyte in the sample. In some embodiments, the on-target extension product is released into a supernatant fraction of a reaction (e.g., primer extension reaction) in which the hybridized capture oligonucleotide and/or the hybridized detection oligonucleotide are extended. In some embodiments, releasing the on-target extension product at block 1060 involves releasing from the solid support and the detection moiety.

Providing the solid support at block 1010 and providing the detection conjugate at block 1020 can be performed in any suitable order. In some embodiments, the solid support is provided before providing the detection conjugate. In some embodiments, detection conjugate is provided before providing the solid support. In some embodiments, providing the solid support is done concurrently to providing the detection conjugate.

In some embodiments, the analyte detection method can be used to determine whether an analyte of interest is present in a sample. In some embodiments, the sample includes the analyte (e.g., a detectable amount of the analyte). In some embodiments, the sample does not include the analyte (e.g., does not include the analyte in a detectable amount). In some embodiments, when the analyte is not present in the sample, or is not present in a sufficient amount to allow the analyte to be bound (or to be simultaneously bound) to the detection moiety and the capture moiety within a relevant volume so as to maintain proximity of the capture oligonucleotide and detection oligonucleotide, then hybridization between the 3′ hybridizing region of the capture oligonucleotide and the 3′ hybridizing region of the detection oligonucleotide that may occur (e.g., when the capture oligonucleotide and detection oligonucleotide come into proximity of each other without the corresponding capture and detection moieties both being bound (or being simultaneously bound) to the analyte) is not stable enough to allow extension to occur across the 3′ hybridizing region (either from the capture oligonucleotide side to the detection oligonucleotide side or vice versa) to a sufficient extent to generate a detectable or significant amount of extension product.

The analyte, if present, can be present in the sample at any suitable amount. In some embodiments, the analyte is present in the sample at a concentration of, of about, or of at least, or on the order of, 10⁻¹⁵, 10⁻¹⁴, 10⁻¹³, 10⁻¹², 10⁻¹¹, 10⁻¹⁰, 10⁻⁹, 10⁻¹, 10⁻⁷, or 10⁻⁶ M, or optionally at a concentration in a range defined by any two of the preceding values (e.g., 10⁻¹⁵-10⁻⁶ M, 10⁻¹⁴-10⁻⁷ M, 10⁻¹⁴-10⁻⁹ M, 10⁻¹³-10⁻⁸ M, etc.). In some embodiments, the analyte is (or is expected to be) present in the sample at a concentration of, of about, or of at least, or on the order of, 10⁻¹⁵, 10⁻¹⁴, 10⁻¹³, 10⁻¹², 10⁻¹¹, 10⁻¹⁰, 10⁻⁹, 10⁻¹, 10⁻⁷, or 10⁻⁶ g/mL, or optionally at a concentration in a range defined by any two of the preceding values (e.g., 10⁻¹⁵-10⁻⁶ g/mL, 10⁻¹⁴-10⁻⁷ g/mL, 10⁻¹⁴-10⁻⁹ g/mL, 10⁻¹³-10⁻⁸ g/mL, etc.). In some embodiments, the sample is a diluted fraction of an original sample containing the analyte at a higher concentration. In some embodiments, the method includes diluting at least a portion of the original sample containing the analyte at a higher concentration to obtain the sample to be analyzed by the present method. Any suitable solution can be used to prepare the sample (e.g., dilute that sample). In some embodiments, the sample is prepared in a solution comprising bovine serum albumin, fetal bovine serum, potassium phosphate dibasic, potassium phosphate monobasic, kathon CG/ICP II, sucrose, and/or Triton™ X-100. In some embodiments, the sample is prepared in a solution comprising about 2.0% bovine serum albumin, about 2.1% potassium phosphate dibasic, about 0.5% potassium phosphate monobasic, about 0.04% kathon CG/ICP II, about 2.0% sucrose, and about 0.022% Triton™ X-100. In some embodiments, the sample is prepared in a solution comprising about 2.0% bovine serum albumin, about 2.1% potassium phosphate dibasic, about 0.5% potassium phosphate monobasic, about 0.04% kathon CG/ICP II, about 2.0% sucrose, about 0.022% Triton™ X-100, about 0.3% IgG, about 500 mM NaCl, and fetal bovine serum.

The complexing solution can be prepared using any suitable option. In some embodiments, preparing a complexing solution includes: contacting the solid support provided at block 1010 with a sample, thereby allowing the capture moiety of the solid support to be bound to the analyte if present in the sample, and combining in a solution the sample-contacted solid support and the detection conjugate provided at block 1020. Contacting the solid support provided at block 1010 with the sample can be performed in any suitable manner. In some embodiments, contacting includes incubating the solid support with the sample. In some embodiments, contacting includes adding the solid support to the sample. In some embodiments, contacting includes adding the sample to a partition (e.g., a microwell) containing the solid support.

Contacting (or incubating) the solid support provided at block 1010 with the sample can be performed under any suitable condition. In some embodiments, the contacting (or incubating) is performed at room temperature. In some embodiments, the contacting (or incubating) is performed at a temperature of, or of about 4° C. or higher, e.g., about 8° C. or higher, about 12° C. or higher, about 15° C. or higher, about 18° C. or higher, about 20° C. or higher, about 22° C. or higher, about 25° C. or higher, about 27° C. or higher, about 30° C. or higher, about 35° C. or higher, or about 50° C. or lower, e.g., about 45° C. or lower, about 40° C. or lower, about 37° C. or lower, about 35° C. or lower, about 32° C. or lower, about 30° C. or lower, about 28° C. or lower, about 26° C. or lower, about 23° C. or lower, about 20° C. or lower, about 15° C. or lower, or at a temperature in range defined by any two of the preceding values (e.g., 4-50° C., 15-35° C., 20-25° C., 12-20° C., 20-45° C., 15-30° C., etc.). In some embodiments, the contacting (or incubating) is performed at a temperature of 12-28° C. In some embodiments, the contacting (or incubating) is performed at a temperature of 15-25° C.

Contacting (or incubating) the solid support provided at block 1010 with the sample can be performed in any suitable volume (e.g., volume of the sample). In some embodiments, the contacting is performed in a volume of, of about, or of at least 1, 2, 5, 10, 15, 20, 25, 30, 35, 40, 45, 50, 60, 70, 80, 90, 100 μL, or of, or about, or of at most 1,000, 900, 800, 700, 600, 500, 400, 300, 200, 150, 100, or 50 μL, or a volume in range defined by any two of the preceding values (e.g., about 1-1,000 μL, about 2-500 μL, about 5-100 μL, about 10⁻²⁰⁰ μL, about 30-150 μL, about 50-150 μL, etc.). In some embodiments, the contacting is performed in a volume of about 5-100 L. In some embodiments, the contacting is performed in a volume of about 50-150 μL.

Contacting (or incubating) the solid support provided at block 1010 with the sample can be (e.g., the solid support can be incubated with the sample) for any suitable length of time. In some embodiments, the contacting (or incubating) is for, for about, or for at least, 10, 30, 45, or 60 minutes, 1.25, 1.5, 2, or 3 hours, or the contacting (or incubating) is for, for about, or for not more than, 12, 9, 6, 3, 2.5, or 2 hours, or for a length of time in a range defined by any two of the preceding values (e.g., 10 minutes to 12 hours, 10 minutes to 6 hours, 30 minutes to 3 hours, 1 hours to 3 hours, 1 hour to 9 hours, 10 minutes to 1 hour, etc.). In some embodiments, the solid support is contacted (or incubated) with the sample for about 30 minutes to about 6 hours. In some embodiments, the solid support is contacted (or incubated) with the sample for about 1 hour to about 4 hours. In some embodiments, the solid support is contacted (or incubated) with the sample for about 2 hours.

Contacting the solid support provided at block 1010 with the sample can be performed in any suitable solution. In some embodiments, contacting the solid support with the sample is performed in a solution comprising a carrier protein, a surfactant, a buffer, a salt, and/or other additives, to inhibit nonspecific binding of sample to the solid support. In some embodiments, contacting the solid support with the sample is performed in a solution comprising one or more blockers. Suitable blockers include, without limitation, mouse IgG, BSA, and casein. In some embodiments, contacting the solid support with the sample is performed in a solution comprising bovine serum albumin, fetal bovine serum, potassium phosphate dibasic, potassium phosphate monobasic, kathon CG/ICP II, sucrose, and/or Triton™ X-100. In some embodiments, contacting the solid support with the sample is performed in a solution comprising about 2.0% bovine serum albumin, about 2.1% potassium phosphate dibasic, about 0.5% potassium phosphate monobasic, about 0.04% kathon CG/ICP II, about 2.0% sucrose, and about 0.022% Triton™ X-100. In some embodiments, contacting the solid support with the sample is performed in a solution comprising about 2.0% bovine serum albumin, about 2.1% potassium phosphate dibasic, about 0.5% potassium phosphate monobasic, about 0.04% kathon CG/ICP II, about 2.0% sucrose, about 0.022% Triton™ X-100, about 0.3% IgG, about 500 mM NaCl, and fetal bovine serum.

Any suitable amount of the solid support can be contacted with the sample. In some embodiments, the solid support is a bead (e.g., a magnetic or paramagnetic bead) as provided herein, and contacting with sample includes contacting the sample with the solid support at a final concentration of solid support (e.g., beads) per sample volume of, or of about 0.001 μg/mL or more, e.g., about 0.005 μg/mL or more, about 0.01 μg/mL or more, about 0.02 μg/mL or more, about 0.05 μg/mL or more, about 0.1 μg/mL or more, about 0.15 μg/mL or more, about 0.2 μg/mL or more, about 0.25 μg/mL or more, about 0.3 μg/mL or more, about 0.35 μg/mL or more, about 0.4 μg/mL or more, about 0.45 μg/mL or more, about 0.5 μg/mL or more, about 0.55 μg/mL or more, about 0.6 μg/mL or more, about 0.65 μg/mL or more, about 0.7 μg/mL or more, about 0.75 g/mL or more, about 0.8 μg/mL or more, about 0.85 μg/mL or more, about 0.9 μg/mL or more, about 0.95 μg/mL or more, about 1 μg/mL or more, or about 5 μg/mL or less, about 4 μg/mL or less, about 3.5 μg/mL or less, about 3 μg/mL or less, about 2.5 μg/mL or less, about 2 μg/mL or less, about 1.8 μg/mL or less, about 1.6 μg/mL or less, about 1.5 μg/mL or less, about 1.4 μg/mL or less, about 1.3 μg/mL or less, about 1.2 μg/mL or less, about 1.1 μg/mL or less, about 1.0 μg/mL or less, about 0.9 μg/mL or less, about 0.8 μg/mL or less, about 0.7 μg/mL or less, about 0.6 μg/mL or less, about 0.5 μg/mL or less, or a concentration in a range defined by any two of the preceding values (e.g., 0.001-5 μg/mL, 0.01-2 μg/mL, 0.05-1.5 μg/mL, 0.08-1.5 μg/mL, 0.1-1 μg/mL, 0.3-0.7 μg/mL, 0.1-0.5 μg/mL, 0.5-1 μg/mL, etc.) of the solid support with the sample. In some embodiments, where the solid support is a bead, the solid support is contacted with the sample at a concentration of, or of about 0.005-3 μg/mL. In some embodiments, where the solid support is a bead, the solid support is contacted with the sample at a concentration of, or of about 0.005-2 g/mL. In some embodiments, where the solid support is a bead, the solid support is contacted with the sample at a concentration of, or of about 0.01-1 μg/mL.

The capture moiety on the solid support can be contacted with the sample at any suitable amount. In some embodiments, the capture moiety (as provided on the solid support) is at a concentration of, of about, or of at least 1, 2, 5, 10, 15, 20, 25, 30, 35, 40, 50, 60, 70, 80, 90, 100 ng/100 μL, or a concentration in a range defined by any two of the preceding values (e.g., about 1-100 ng/100 μL, about 5-75 ng/100 μL, about 25-55 ng/100 μL, about 30-50 ng/100 μL, etc.) when contacted with the sample (e.g., based on a sample volume of about 100 μL). In some embodiments, the capture moiety (as provided on the solid support) is at a concentration of about 25-55 ng/100 μL of the sample volume.

The capture oligonucleotide on the solid support can be combined with the sample at any suitable concentration. In some embodiments, the capture oligonucleotide provided on the solid support is at a concentration of, or of about, or of at least 0.001, 0.002, 0.005, 0.01, 0.02, 0.03, 0.04, 0.05, 0.06, 0.07, 0.08, 0.09, or 0.1 μmol, or a concentration in a range defined by any two of the preceding values (e.g., about 0.001-0.1 μmol, about 0.002-0.08 μmol, about 0.005-0.08 μmol, about 0.01-0.06 μmol, etc.) when contacted with the sample (e.g., in a sample volume of about 100 μL). In some embodiments, the capture oligonucleotide provided on the solid support is at a concentration of about 0.001-0.1 pmol when contacted with the sample.

In some embodiments, where preparing the complexing solution includes contacting the solid support provided at block 1010 with the sample, the method further includes removing the sample before combining in the solution the sample-contacted solid support and the detection conjugate provided at block 1020, thereby removing analyte if present that is not bound to the capture moiety. The sample can be removed from the solid support using any suitable option. In some embodiments, removing the sample includes washing the solid support. In some embodiments, removing the sample includes washing the solid support by transferring the solid support to a wash solution (e.g., a buffer solution) that does not include any analyte. In some embodiments, removing the sample includes replacing the sample with a wash solution (e.g., a buffer solution) that does not include any analyte. Washing the solid support can be done any suitable number of times. In some embodiments, the solid support is washed 1, 2, 3, 4, 5 or more times. In some embodiments, the solid support is washed 2-4 times. In some embodiments, the solid support is washed 3 times. In some embodiments, the washing involves an equivalent of about 1, 2, 3, 4, 5, or more volume exchanges with a wash solution. Any suitable wash solution can be used to wash the solid support after contacting with the sample. In some embodiments, the wash solution comprises phosphate buffered saline (PBS). In some embodiments, the wash solution comprises up to, or up to about 5% (e.g., up to, or up to about 1%, 2%, 3%, 4%, or about 5%) detergent. In some embodiments, the wash solution comprises PBS plus polysorbate 20. In some embodiments, the wash solution comprises about 0.01 to 0.1% polysorbate 20. In some embodiments, the wash solution comprises PBS plus 0.05% polysorbate 20.

In some embodiments, preparing the complexing solution includes, following contacting the solid support with the sample, combining in a solution the sample-contacted solid support and the detection conjugate provided at block 1020, using any suitable option. In some embodiments, the combining includes adding the sample-contacted solid support to a solution comprising the detection conjugate. The sample-contacted solid support and the detection conjugate can be combined under any suitable condition to allow the capture moiety and the detection moiety to both be bound (or to be simultaneously bound) to the analyte if present in the sample. In some embodiments, combining the sample-contacted solid support and the detection conjugate includes incubating the solution under a suitable condition to allow the capture moiety and the detection moiety to both be bound (or to be simultaneously bound) to the analyte if present in the sample.

The sample-contacted solid support and the detection conjugate provided at block 1020 can be combined in any suitable solution. In some embodiments, the sample-contacted solid support and the detection conjugate are combined in a solution comprising a carrier protein, a surfactant, a buffer, a salt, and/or other additives to inhibit nonspecific binding of detection conjugates to the sample-contacted solid support. In some embodiments, the sample-contacted solid support and the detection conjugate are combined in a solution comprising one or more blockers. Suitable blockers include, without limitation, mouse IgG, BSA, casein, and salmon sperm DNA. In some embodiments, the sample-contacted solid support and the detection conjugate are combined in a solution comprising BSA, potassium phosphate dibasic, potassium phosphate monobasic, sucrose, Kathon CG/CP II and/or Triton™ X-100. In some embodiments, the sample-contacted solid support and the detection conjugate are combined in a solution comprising BSA, potassium phosphate dibasic, potassium phosphate monobasic, sucrose, Kathon CG/CP II and/or Triton™ X-100. In some embodiments, the sample-contacted solid support and the detection conjugate are combined in a solution comprising IgG, e.g., mouse IgG. In some embodiments, the sample-contacted solid support and the detection conjugate are combined in a solution comprising 2.0% sucrose, 2.0% BSA, 2.1% potassium phosphate dibasic, 0.5% potassium phosphate monobasic, 0.04% Kathon CG/ICP II, 0.022% Triton™ X-100, 0.1% mouse IgG, and 0.5% goat IgG.

The sample-contacted solid support can be combined (or incubated) with the detection conjugate provided at block 1020 in any suitable volume of the solution. In some embodiments, the contacting is performed in a volume of, of about, or of at least 1, 2, 5, 10, 15, 20, 25, 30, 35, 40, 45, 50, 60, 70, 80, 90, 100 μL, or of, or about, or of at most 1,000, 900, 800, 700, 600, 500, 400, 300, 200, 150, 100, or 50 μL, or a volume in range defined by any two of the preceding values (e.g., about 1-1,000 μL, about 2-500 μL, about 5-100 μL, about 10⁻²⁰⁰ μL, about 30-150 μL, about 50-150 μL, etc.). In some embodiments, the contacting is performed in a volume of about 5-100 μL.

Any suitable amount of the sample-contacted solid support can be combined with the detection conjugate provided at block 1020. In some embodiments, substantially all (e.g., at least 80%, 85%, 90%, 95%, 97%, 98%, or 99%) of the solid support that is contacted with the sample is then combined with the detection conjugate. In some embodiments, substantially all (e.g., at least 80%, 85%, 90%, 95%, 97%, 98%, or 99%) of the solid support that is contacted with the sample, after taking into account any washing steps after contacting with the sample, is then combined with the detection conjugate. In some embodiments, a portion (e.g., about 95%, 90%, 85%, 80%, 70%, 60%, 50%, 40%, 30% or less, or a percentage in a range defined by any two of the preceding values, such as 30-95%, 50-90%, 80-95%, 75-85%, etc.) of the solid support that is contacted with the sample is then combined with the detection conjugate. In some embodiments, a portion (e.g., about 95%, 90%, 85%, 80%, 70%, 60%, 50%, 40%, 30% or less, or a percentage in a range defined by any two of the preceding values, such as 30-95%, 50-90%, 80-95%, 75-85%, etc.) of the solid support that is contacted with the sample, after taking into account any washing steps after contacting with the sample, is then combined with the detection conjugate.

Any suitable amount of the capture moiety (e.g., as provided on the sample-contacted solid support) can be combined with the detection conjugate. In some embodiments, the sample-contacted solid support provides, provides about, or provides at least 0.5, 1, 2, 5, 10, 15, 20, 25, 30, 35, 40, 50, 60, 70, 80, 90, 100 ng, or an amount in a range defined by any two of the preceding values (e.g., about 1-100 ng/100 μL, about 5-75 ng/100 μL, about 25-55 ng/100 μL, about 30-50 ng/100 μL, etc.) of the capture moiety that is combined with the detection conjugate. In some embodiments, the sample-contacted solid support provides about 25-55 ng/100 μL of sample volume of the capture moiety that is combined with the detection conjugate.

Any suitable amount of the capture oligonucleotide (e.g., as provided on the sample-contacted solid support) can be combined with the detection conjugate. In some embodiments, the sample-contacted solid support provides, provides about, or provides at least 0.001, 0.002, 0.005, 0.01, 0.02, 0.03, 0.04, 0.05, 0.06, 0.07, 0.08, 0.09, or 0.1 μmol, or a concentration in a range defined by any two of the preceding values (e.g., about 0.001-0.1 μmol, about 0.002-0.08 μmol, about 0.005-0.08 μmol, about 0.01-0.06 μmol, etc.) of the capture oligonucleotide that is combined with the detection conjugate. In some embodiments, the sample-contacted solid support provides about 0.001-0.1 pmol of the capture oligonucleotide that is combined with the detection conjugate.

Any suitable amount of the detection conjugate can be combined with the sample-contacted solid support. In some embodiments, the detection conjugate (e.g., an antibody conjugate) is combined with the complexing solution at a concentration of, or of about 5 pM or more, e.g., about 10 pM or more, about 20 pM or more, about 30 pM or more, about 40 pM or more, about 50 pM or more, about 75 pM or more, about 100 pM or more, about 150 pM or more, about 200 pM or more, about 250 pM or more, about 300 pM or more, about 400 pM or more, about 500 pM or more, about 600 pM or more, about 700 pM or more, about 800 pM or more, about 900 pM or more, about 1,000 pM or more, about 2,000 pM or more, about 3,000 pM or more, about 4,000 pM or more, about 5,000 pM or more, 6,000 pM or more, about 7,000 pM or more, about 8,000 pM or more, about 9,000 pM or more, about 10,000 pM or more, about 20,000 pM or more, about 50,000 pM or more, about 10⁵ pM or more or more, or optionally a concentration in a range defined by any two of the preceding values (e.g., about 5-50,000 pM, about 10-20,000 pM, about 50-10,000 pM, about 20-8,000 pM, about 500-10,000 pM, etc.). In some embodiments, the detection conjugate (e.g., antibody conjugate) is combined at about 50-10,000 pM. In some embodiments, the detection conjugate (e.g., antibody conjugate) is combined at about 500-10,000 pM. In some embodiments, the detection conjugate (e.g., antibody conjugate) is combined at about 1,000-3,000 pM.

In some embodiments, the detection conjugate (e.g., an antibody conjugate) is combined with the complexing solution at a concentration of, or of about 0.001 μg/mL or more, e.g., about 0.005 μg/mL or more, about 0.01 μg/mL or more, about 0.02 μg/mL or more, about 0.05 μg/mL or more, about 0.1 μg/mL or more, about 0.15 μg/mL or more, about 0.2 μg/mL or more, about 0.25 μg/mL or more, about 0.3 μg/mL or more, about 0.35 μg/mL or more, about 0.4 g/mL or more, about 0.45 μg/mL or more, about 0.5 μg/mL or more, about 0.55 μg/mL or more, about 0.6 μg/mL or more, about 0.65 μg/mL or more, about 0.7 μg/mL or more, about 0.75 μg/mL or more, about 0.8 μg/mL or more, about 0.85 μg/mL or more, about 0.9 μg/mL or more, about 0.95 μg/mL or more, about 1 μg/mL or more, or about 2 μg/mL or less, about 1.8 μg/mL or less, about 1.6 μg/mL or less, about 1.5 μg/mL or less, about 1.4 μg/mL or less, about 1.3 μg/mL or less, about 1.2 μg/mL or less, about 1.1 μg/mL or less, about 1.0 μg/mL or less, about 0.9 μg/mL or less, about 0.8 μg/mL or less, about 0.7 μg/mL or less, about 0.6 μg/mL or less, about 0.5 μg/mL or less, or a concentration in a range defined by any two of the preceding values (e.g., 0.001-2 g/mL, 0.01-2 μg/mL, 0.05-1.5 μg/mL, 0.08-1.5 μg/mL, 0.1-1 μg/mL, 0.3-0.7 μg/mL, 0.1-0.5 g/mL, 0.5-1 μg/mL, etc.). In some embodiments, the detection conjugate is an antibody conjugate (e.g., a full-length antibody conjugate), and is present at about 0.01-1 μg/mL. In some embodiments, the detection conjugate is an antibody conjugate (e.g., a full-length antibody conjugate), and is present at about 0.1-1 μg/mL. In some embodiments, the concentration is based on the concentration of the detection moiety portion of the detection conjugate (e.g., excluding the contribution of the mass of the detection oligonucleotide).

In some embodiments, preparing the complexing solution at block 1030 includes contacting the detection conjugate provided at block 1020 with a sample, thereby allowing the detection moiety to be bound to the analyte if present in the sample, and combining in a solution the sample-contacted detection conjugate with the solid support provided at block 1010. Contacting the detection conjugate provided at block 1020 with the sample can be performed in any suitable manner. In some embodiments, contacting includes incubating the detection conjugate with the sample. In some embodiments, contacting includes adding the detection conjugate (e.g., a solution containing the detection conjugate) to the sample. In some embodiments, contacting includes adding the sample to a partition (e.g., a microwell) containing the detection conjugate.

Contacting (or incubating) the detection conjugate provided at block 1020 with the sample can be performed under any suitable condition. In some embodiments, the contacting (or incubating) is performed at room temperature. In some embodiments, the contacting (or incubating) is performed at a temperature of about 4° C. or higher, e.g., about 8° C. or higher, about 12° C. or higher, about 15° C. or higher, about 18° C. or higher, about 20° C. or higher, about 22° C. or higher, about 25° C. or higher, about 27° C. or higher, about 30° C. or higher, about 35° C. or higher, or about 50° C. or lower, e.g., about 45° C. or lower, about 40° C. or lower, about 37° C. or lower, about 35° C. or lower, about 32° C. or lower, about 30° C. or lower, about 28° C. or lower, about 26° C. or lower, about 23° C. or lower, about 20° C. or lower, about 15° C. or lower, or at a temperature in range defined by any two of the preceding values (e.g., 4-50° C., 15-35° C., 20-25° C., 12-20° C., 20-45° C., 15-30° C., etc.). In some embodiments, the contacting (or incubating) is performed at a temperature of 12-28° C. In some embodiments, the contacting (or incubating) is performed at a temperature of 15-25° C.

Contacting (or incubating) the detection conjugate provided at block 1020 with the sample can be performed in any suitable volume (e.g., volume of the sample). In some embodiments, the contacting is performed in a volume of, of about, or of at least 1, 2, 5, 10, 15, 20, 25, 30, 35, 40, 45, 50, 60, 70, 80, 90, 100 μL, or of, or about, or of at most 1,000, 900, 800, 700, 600, 500, 400, 300, 200, 150, 100, or 50 μL, or a volume in range defined by any two of the preceding values (e.g., about 1-1,000 μL, about 2-500 μL, about 5-100 μL, about 10⁻²⁰⁰ μL, about 30-150 μL, about 50-150 μL, etc.). In some embodiments, the contacting is performed in a volume of about 5-100 μL. In some embodiments, the contacting is performed in a volume of about 50-150 μL.

Contacting (or incubating) the detection conjugate provided at block 1020 with the sample can be (e.g., the detection conjugate can be incubated with the sample) for any suitable length of time. In some embodiments, the contacting (or incubating) is for, for about, or for at least, 10, 30, 45, or 60 minutes, 1.25, 1.5, 2, or 3 hours or more, or the contacting (or incubating) is for, for about, for not more than 12, 9, 6, 3, 2.5, or 2 hours, or for a length of time in a range defined by any two of the preceding values (e.g., 10 minutes to 12 hours, 10 minutes to 6 hours, 30 minutes to 3 hours, 1 hours to 3 hours, 1 hour to 9 hours, 10 minutes to 1 hour, etc.). In some embodiments, the detection conjugate is contacted (or incubated) with the sample for about 30 minutes to about 6 hours. In some embodiments, the detection conjugate is contacted (or incubated) with the sample for about 30 minutes to about 2 hours. In some embodiments, the detection conjugate is contacted (or incubated) with the sample for about 1 hour.

Contacting the detection conjugate provided at block 1020 with the sample can be performed in any suitable solution. In some embodiments, contacting the detection conjugate with the sample is performed in a solution comprising a carrier protein, a surfactant, a buffer, a salt, and/or other additives, to inhibit nonspecific binding of sample to the solid support. In some embodiments, contacting the detection conjugate with the sample is performed in a solution comprising one or more blockers. Suitable blockers include, without limitation, mouse IgG, BSA, and casein. In some embodiments, contacting the detection conjugate with the sample is performed in a solution comprising bovine serum albumin, fetal bovine serum, potassium phosphate dibasic, potassium phosphate monobasic, kathon CG/ICP II, sucrose, and/or Triton™ X-100. In some embodiments, contacting the detection conjugate with the sample is performed in a solution comprising about 2.0% bovine serum albumin, about 2.1% potassium phosphate dibasic, about 0.5% potassium phosphate monobasic, about 0.04% kathon CG/ICP II, about 2.0% sucrose, and about 0.022% Triton™ X-100. In some embodiments, contacting the detection conjugate with the sample is performed in a solution comprising about 2.0% bovine serum albumin, about 2.1% potassium phosphate dibasic, about 0.5% potassium phosphate monobasic, about 0.04% kathon CG/ICP II, about 2.0% sucrose, about 0.022% Triton™ X-100, about 0.3% IgG, about 500 mM NaCl, and fetal bovine serum.

Any suitable amount of the detection conjugate can be contacted with the sample. In some embodiments, the detection conjugate (e.g., antibody conjugate) is contacted with the sample at a final concentration of detection conjugate (e.g., antibody conjugate) per sample volume of, or of about 5 pM or more, e.g., about 10 pM or more, about 20 pM or more, about 30 pM or more, about 40 pM or more, about 50 pM or more, about 75 pM or more, about 100 pM or more, about 150 pM or more, about 200 pM or more, about 250 pM or more, about 300 pM or more, about 400 pM or more, about 500 pM or more, about 600 pM or more, about 700 pM or more, about 800 pM or more, about 900 pM or more, about 1,000 pM or more, about 2,000 pM or more, about 3,000 pM or more, about 4,000 pM or more, about 5,000 pM or more, 6,000 pM or more, about 7,000 pM or more, about 8,000 pM or more, about 9,000 pM or more, about 10,000 pM or more, about 20,000 pM or more, about 50,000 pM or more, or a concentration in a range defined by any two of the preceding values (e.g., about 5-50,000 pM, about 10-20,000 pM, about 50-10,000 pM, about 20-8,000 pM, about 500-10,000 pM, etc.). In some embodiments, the detection conjugate (e.g., antibody conjugate) is contacted with the sample at a concentration of, or of about 50-10,000 pM.

Any suitable amount of the detection moiety and/or detection oligonucleotide (e.g., as provided by the detection conjugate) can be contacted with the sample. In some embodiments, the detection moiety and/or detection oligonucleotide (e.g., as provided by the detection conjugate) is contacted with the sample (e.g., contacted in a sample volume) at a concentration of, of about 5 pM or more, e.g., about 10 pM or more, about 20 pM or more, about 30 pM or more, about 40 pM or more, about 50 pM or more, about 75 pM or more, about 100 pM or more, about 150 pM or more, about 200 pM or more, about 250 pM or more, about 300 pM or more, about 400 pM or more, about 500 pM or more, about 600 pM or more, about 700 pM or more, about 800 pM or more, about 900 pM or more, about 1,000 pM or more, about 2,000 pM or more, about 3,000 pM or more, about 4,000 pM or more, about 5,000 pM or more, 6,000 pM or more, about 7,000 pM or more, about 8,000 pM or more, about 9,000 pM or more, about 10,000 pM or more, about 20,000 pM or more, about 50,000 pM or more, or a concentration in a range defined by any two of the preceding values (e.g., about 5-50,000 pM, about 10-20,000 pM, about 50-10,000 pM, about 20-8,000 pM, about 500-10,000 pM, etc.). In some embodiments, the detection conjugate (e.g., antibody conjugate) is combined at about 50-10,000 pM. In some embodiments, the detection conjugate (e.g., antibody conjugate) is combined at about 500-10,000 pM. In some embodiments, the detection conjugate (e.g., antibody conjugate) is combined at about 1,000-3,000 pM.

In some embodiments, the detection conjugate (e.g., an antibody conjugate) is contacted with the sample (e.g., contacted in a sample volume) at a concentration of, or of about 0.001 μg/mL or more, e.g., about 0.005 μg/mL or more, about 0.01 μg/mL or more, about 0.02 g/mL or more, about 0.05 μg/mL or more, about 0.1 μg/mL or more, about 0.15 μg/mL or more, about 0.2 μg/mL or more, about 0.25 μg/mL or more, about 0.3 μg/mL or more, about 0.35 μg/mL or more, about 0.4 μg/mL or more, about 0.45 μg/mL or more, about 0.5 μg/mL or more, about 0.55 μg/mL or more, about 0.6 μg/mL or more, about 0.65 μg/mL or more, about 0.7 μg/mL or more, about 0.75 μg/mL or more, about 0.8 μg/mL or more, about 0.85 μg/mL or more, about 0.9 g/mL or more, about 0.95 μg/mL or more, about 1 μg/mL or more, or about 2 μg/mL or less, about 1.8 μg/mL or less, about 1.6 μg/mL or less, about 1.5 μg/mL or less, about 1.4 μg/mL or less, about 1.3 μg/mL or less, about 1.2 μg/mL or less, about 1.1 μg/mL or less, about 1.0 μg/mL or less, about 0.9 μg/mL or less, about 0.8 μg/mL or less, about 0.7 μg/mL or less, about 0.6 μg/mL or less, about 0.5 μg/mL or less, or a concentration in a range defined by any two of the preceding values (e.g., 0.001-2 μg/mL, 0.01-2 μg/mL, 0.05-1.5 μg/mL, 0.08-1.5 μg/mL, 0.1-1 μg/mL, 0.3-0.7 μg/mL, 0.1-0.5 μg/mL, 0.5-1 μg/mL, etc.). In some embodiments, the detection conjugate is an antibody conjugate (e.g., a full-length antibody conjugate), and is present at about 0.01-1 μg/mL in the sample volume when contacted with the sample. In some embodiments, the detection conjugate is an antibody conjugate (e.g., a full-length antibody conjugate), and is present at about 0.1-1 μg/mL in the sample volume when contacted with the sample. In some embodiments, the concentration is based on the concentration of the detection moiety portion of the detection conjugate (e.g., excluding the contribution of the mass of the detection oligonucleotide).

In some embodiments, where preparing the complexing solution includes contacting the detection conjugate provided at block 1020 with the sample, the method further includes removing the sample before combining in the solution the sample-contacted detection conjugate and the solid support provided at block 1010, thereby removing any analyte if present that is not bound to the detection moiety. The sample can be removed from the detection conjugate using any suitable option. In some embodiments, removing the sample includes using a chromatographic separation technique (e.g., affinity chromatography, size exclusion chromatography, etc.).

In some embodiments, preparing the complexing solution includes, following contacting the detection conjugate with the sample, combining in a solution the sample-contacted detection conjugate and the solid support provided at block 1010, using any suitable option. In some embodiments, the combining includes adding the sample-contacted detection conjugate to a solution comprising the solid support. In some embodiments, the combining includes adding the solid support to a solution comprising the sample-contacted detection conjugate. The sample-contacted detection conjugate and the solid support can be combined under any suitable condition to allow the capture moiety and the detection moiety to both be bound (or to be simultaneously bound) to the analyte if present in the sample. In some embodiments, combining the sample-contacted detection conjugate and the solid support includes incubating the solution to allow the capture moiety and the detection moiety to both be bound (or to be simultaneously bound) to the analyte if present in the sample.

The sample-contacted detection conjugate and the solid support provided at block 1010 can be combined in any suitable solution. In some embodiments, the sample-contacted detection conjugate and the solid support are combined in a solution comprising a carrier protein, a surfactant, a buffer, a salt, and/or other additives to inhibit nonspecific binding of detection conjugates to the sample-contacted detection conjugate. In some embodiments, the sample-contacted detection conjugate and the solid support are combined in a solution comprising one or more blockers. Suitable blockers include, without limitation, mouse IgG, BSA, casein, and salmon sperm DNA. In some embodiments, the sample-contacted detection conjugate and the solid support are combined in a solution comprising BSA, potassium phosphate dibasic, potassium phosphate monobasic, sucrose, Kathon CG/CP II and/or Triton™ X-100. In some embodiments, the sample-contacted detection conjugate and the solid support are combined in a solution comprising BSA, potassium phosphate dibasic, potassium phosphate monobasic, sucrose, Kathon CG/CP II and/or Triton™ X-100. In some embodiments, the sample-contacted detection conjugate and the solid support are combined in a solution comprising IgG, e.g., mouse IgG. In some embodiments, the sample-contacted detection conjugate and the solid support are combined in a solution comprising 2.0% sucrose, 2.0% BSA, 2.1% potassium phosphate dibasic, 0.5% potassium phosphate monobasic, 0.04% Kathon CG/ICP II, 0.022% Triton™ X-100, 0.1% mouse IgG, and 0.5% goat IgG.

The sample-contacted detection conjugate can be combined (or incubated) with the solid support provided at block 1010 in any suitable volume of the solution. In some embodiments, the sample-contacted detection conjugate and the solid support are combined in a volume of, of about, or of at least 1, 2, 5, 10, 15, 20, 25, 30, 35, 40, 45, 50, 60, 70, 80, 90, 100 μL, or of, or about, or of at most 1,000, 900, 800, 700, 600, 500, 400, 300, 200, 150, 100, or 50 μL, or a volume in range defined by any two of the preceding values (e.g., about 1-1,000 μL, about 2-500 μL, about 5-100 μL, about 10⁻²⁰⁰ μL, about 30-150 μL, about 50-150 μL, etc.). In some embodiments, the sample-contacted detection conjugate and the solid support are combined in a volume of about 5-100 μL.

Any suitable amount of the sample-contacted detection conjugate can be combined with the solid support provided at block 1010. In some embodiments, substantially all (e.g., at least 80%, 85%, 90%, 95%, 97%, 98%, or 99%, optionally at least 95%) of the detection conjugate that is contacted with the sample is then combined with the solid support. In some embodiments, substantially all (e.g., at least 80%, 85%, 90%, 95%, 97%, 98%, or 99%, optionally at least 95%) of the detection conjugate that is contacted with the sample, after taking into account any washing steps after contacting with the sample, is then combined with the solid support. In some embodiments, a portion (e.g., about 95%, 90%, 85%, 80%, 70%, 60%, 50%, 40%, 30% or less, or optionally a percentage in a range defined by any two of the preceding values, such as 30-95%, 50-90%, 80-95%, 75-85%, etc., or optionally at least 95%) of the detection conjugate that is contacted with the sample is then combined with the solid support. In some embodiments, a portion (e.g., about 95%, 90%, 85%, 80%, 70%, 60%, 50%, 40%, 30% or less, or optionally a percentage in a range defined by any two of the preceding values, such as 30-95%, 50-90%, 80-95%, 75-85%, etc., or optionally about 95%) of the detection conjugate that is contacted with the sample, after taking into account any washing steps after contacting with the sample, is then combined with the solid support.

Any suitable amount of the detection moiety and/or detection oligonucleotide (e.g., as provided by the sample-contacted detection conjugate) can be combined with the solid support. In some embodiments, the detection moiety and/or detection oligonucleotide is present (e.g., as provided by the sample-contacted detection conjugate) in the solution at a concentration of, or of about 5 pM or more, e.g., about 10 pM or more, about 20 pM or more, about 30 pM or more, about 40 pM or more, about 50 pM or more, about 75 pM or more, about 100 pM or more, about 150 pM or more, about 200 pM or more, about 250 pM or more, about 300 pM or more, about 400 pM or more, about 500 pM or more, about 600 pM or more, about 700 pM or more, about 800 pM or more, about 900 pM or more, about 1,000 pM or more, about 2,000 pM or more, about 3,000 pM or more, about 4,000 pM or more, about 5,000 pM or more, 6,000 pM or more, about 7,000 pM or more, about 8,000 pM or more, about 9,000 pM or more, about 10,000 pM or more, about 20,000 pM or more, about 50,000 pM or more, or optionally at a concentration in a range defined by any two of the preceding values (e.g., about 5-50,000 pM, about 10-20,000 pM, about 50-10,000 pM, about 20-8,000 pM, about 500-10,000 pM, etc.). In some embodiments, the detection conjugate (e.g., antibody conjugate) is present in the solution at a concentration of about 50 to about 10,000 pM. In some embodiments, the detection conjugate (e.g., antibody conjugate) is present in the solution at a concentration of about 500 to about 10,000 pM. In some embodiments, the detection conjugate (e.g., antibody conjugate) is present in the solution at a concentration of about 1,000 to about 3,000 pM.

In some embodiments, the detection conjugate (e.g., an antibody conjugate) is present in the solution at a concentration of, or of about 0.001 μg/mL or more, e.g., about 0.005 g/mL or more, about 0.01 μg/mL or more, about 0.02 μg/mL or more, about 0.05 μg/mL or more, about 0.1 μg/mL or more, about 0.15 μg/mL or more, about 0.2 μg/mL or more, about 0.25 μg/mL or more, about 0.3 μg/mL or more, about 0.35 μg/mL or more, about 0.4 μg/mL or more, about 0.45 μg/mL or more, about 0.5 μg/mL or more, about 0.55 μg/mL or more, about 0.6 μg/mL or more, about 0.65 μg/mL or more, about 0.7 μg/mL or more, about 0.75 μg/mL or more, about 0.8 g/mL or more, about 0.85 μg/mL or more, about 0.9 μg/mL or more, about 0.95 μg/mL or more, about 1 μg/mL or more, or about 2 μg/mL or less, about 1.8 μg/mL or less, about 1.6 μg/mL or less, about 1.5 μg/mL or less, about 1.4 μg/mL or less, about 1.3 μg/mL or less, about 1.2 μg/mL or less, about 1.1 μg/mL or less, about 1.0 μg/mL or less, about 0.9 μg/mL or less, about 0.8 μg/mL or less, about 0.7 μg/mL or less, about 0.6 μg/mL or less, about 0.5 μg/mL or less, or a concentration in a range defined by any two of the preceding values (e.g., 0.001-2 μg/mL, 0.01-2 g/mL, 0.05-1.5 μg/mL, 0.08-1.5 μg/mL, 0.1-1 μg/mL, 0.3-0.7 μg/mL, 0.1-0.5 μg/mL, 0.5-1 g/mL, etc.). In some embodiments, the detection conjugate is an antibody conjugate (e.g., a full-length antibody conjugate), and is present in the solution at a concentration of about 0.01 to about 1 g/mL. In some embodiments, the detection conjugate is an antibody conjugate (e.g., a full-length antibody conjugate), and is present in the solution at a concentration of about 0.1 to about 1 μg/mL. In some embodiments, the concentration is based on the concentration of the detection moiety portion of the detection conjugate (e.g., excluding the contribution of the mass of the detection oligonucleotide).

Any suitable amount of the solid support can be combined with the sample-contacted detection conjugate. In some embodiments, the solid support is a bead (e.g., a magnetic or paramagnetic bead) as provided herein, and about 0.001 μg/mL or more, e.g., about 0.005 g/mL or more, about 0.01 μg/mL or more, about 0.02 μg/mL or more, about 0.05 μg/mL or more, about 0.1 μg/mL or more, about 0.15 μg/mL or more, about 0.2 μg/mL or more, about 0.25 μg/mL or more, about 0.3 μg/mL or more, about 0.35 μg/mL or more, about 0.4 μg/mL or more, about 0.45 μg/mL or more, about 0.5 μg/mL or more, about 0.55 μg/mL or more, about 0.6 μg/mL or more, about 0.65 μg/mL or more, about 0.7 μg/mL or more, about 0.75 μg/mL or more, about 0.8 g/mL or more, about 0.85 μg/mL or more, about 0.9 μg/mL or more, about 0.95 μg/mL or more, about 1 μg/mL or more, or about 5 μg/mL or less, about 4 μg/mL or less, about 3.5 μg/mL or less, about 3 μg/mL or less, about 2.5 μg/mL or less, about 2 μg/mL or less, about 1.8 μg/mL or less, about 1.6 μg/mL or less, about 1.5 μg/mL or less, about 1.4 μg/mL or less, about 1.3 μg/mL or less, about 1.2 μg/mL or less, about 1.1 μg/mL or less, about 1.0 μg/mL or less, about 0.9 μg/mL or less, about 0.8 μg/mL or less, about 0.7 μg/mL or less, about 0.6 μg/mL or less, about 0.5 μg/mL or less, or a concentration in a range defined by any two of the preceding values (e.g., 0.001-5 g/mL, 0.01-2 μg/mL, 0.05-1.5 μg/mL, 0.08-1.5 μg/mL, 0.1-1 μg/mL, 0.3-0.7 μg/mL, 0.1-0.5 g/mL, 0.5-1 μg/mL, etc.) of the solid support is combined with the sample-contacted detection conjugate. In some embodiments, where the solid support is a bead, about 0.005 to about 3 μg/mL of the solid support is combined with the sample-contacted detection conjugate. In some embodiments, where the solid support is a bead, about 0.005 to about 2 μg/mL of the solid support is combined with the sample-contacted detection conjugate.

Any suitable amount of the capture moiety (e.g., as provided on the solid support) can be combined with the sample-contacted detection conjugate. In some embodiments, the capture moiety provided on the solid support is at a concentration of, of about, or of at least 1, 2, 5, 10, 15, 20, 25, 30, 35, 40, 50, 60, 70, 80, 90, 100 ng/100 μL, or a concentration in a range defined by any two of the preceding values (e.g., about 1-100 ng/100 μL, about 5-75 ng/100 μL, about 25-55 ng/100 μL, about 30-50 ng/100 μL, etc.) in the solution (e.g., in a solution of about 100 μL) when combined with the sample-contacted detection conjugate. In some embodiments, the capture moiety (as provided on the solid support) is at a concentration of about 25 to about 55 ng/100 μL of the solution volume.

Any suitable amount of the capture oligonucleotide (e.g., as provided by the detection conjugate) can be combined with the sample-contacted detection conjugate. In some embodiments, the capture oligonucleotide (e.g., as provided by the sample-contacted solid support) is combined with the sample-contacted detection conjugate at a concentration of, or of about, or of at least 0.001, 0.002, 0.005, 0.01, 0.02, 0.03, 0.04, 0.05, 0.06, 0.07, 0.08, 0.09, or 0.1 μmol, or a concentration in a range defined by any two of the preceding values (e.g., about 0.001-0.1 μmol, about 0.002-0.08 μmol, about 0.005-0.08 μmol, about 0.01-0.06 μmol, etc.) in the solution (e.g., in a solution of about 100 μL). In some embodiments, the capture oligonucleotide (e.g., as provided by the sample-contacted solid support) is at a concentration of about 0.001 to about 0.1 pmol when combined with the sample-contacted detection conjugate.

In some embodiments, preparing the complexing solution includes: combining in a solution the solid support provided at block 1010 and the detection conjugate provided at block 1020 with a sample, thereby allowing the capture moiety of the solid support and the detection moiety of the detection conjugate to be bound to the analyte if present in the sample. Combining the solid support provided at block 1010 and the detection conjugate provided at block 1020 with the sample can be performed in any suitable manner. In some embodiments, combining is performed sequentially (e.g., contacting the solid support provided at block 1010 with the sample, and then combining the sample-contacted solid support with the detection conjugate provided at block 1020, as provided above; or contacting the detection conjugate provided at block 1020 with the sample, and then combining the sample-contacted detection conjugate with the solid support provided at block 1010, as provided above). In some embodiments, combining is performed concurrently (e.g., the solid support provided at block 1010 and the detection conjugate provided at block 1020 are combined with the sample before incubation with either is carried out for a substantial amount of time (e.g., not more than 30%, 25%, 20%, 15%, 10%, 5%, 4%, 3%, 2%, or 1% of the total amount of time for incubation)). In some embodiments, the solid support and the detection conjugate are combined, and then the combination is combined with the sample. In some embodiments, the solid support is contacted with the sample, and then the detection conjugate is combined with the combination of the solid support and the sample. In some embodiments, the detection conjugate is contacted with the sample, and then the solid support is combined with the combination of the detection conjugate and the sample.

The detection conjugate, the solid support, and the sample can be combined in any suitable solution. In some embodiments, the detection conjugate, solid support, and the sample are combined in a solution comprising a carrier protein, a surfactant, a buffer, a salt, and/or other additives, to inhibit nonspecific binding of sample to the solid support. In some embodiments, the detection conjugate, solid support, and the sample are combined in a solution comprising one or more blockers. Suitable blockers include, without limitation, mouse IgG, BSA, casein, and salmon sperm DNA. In some embodiments, the detection conjugate, solid support, and the sample are combined in a solution comprising bovine serum albumin, fetal bovine serum, potassium phosphate dibasic, potassium phosphate monobasic, kathon CG/ICP II, sucrose, and/or Triton™ X-100. In some embodiments, the detection conjugate, solid support, and the sample are combined in a solution comprising about 2.0% bovine serum albumin, about 2.1% potassium phosphate dibasic, about 0.5% potassium phosphate monobasic, about 0.04% kathon CG/ICP II, about 2.0% sucrose, and about 0.022% Triton™ X-100. In some embodiments, the detection conjugate, solid support, and the sample are combined in a solution comprising about 2.0% bovine serum albumin, about 2.1% potassium phosphate dibasic, about 0.5% potassium phosphate monobasic, about 0.04% kathon CG/ICP II, about 2.0% sucrose, about 0.022% Triton™ X-100, about 0.3% IgG, about 500 mM NaCl, and fetal bovine serum.

In the prepared complexing solution, the capture moiety on the solid support and the detection moiety of the detection conjugate can be allowed to both be bound (or to be simultaneously bound) to the analyte, if present, such that the capture oligonucleotide and detection oligonucleotide are in proximity if the analyte is present in the sample. In some embodiments, where the analyte is present (e.g. present in a detectable amount), an analyte molecule can be bound to a capture moiety (or a detection moiety) for a sufficiently long time to allow a detection moiety (or a capture moiety) to also become bound to the same analyte molecule.

Following preparing the complexing solution, permitting the 3′ hybridizing region of the capture oligonucleotide and the 3′ hybridizing region of the detection oligonucleotide that are in proximity to hybridize to each other or to a splint oligonucleotide at block 1040 can be carried out under any suitable condition. In some embodiments, the complexing solution is incubated at room temperature. In some embodiments, the complexing solution is incubated at a temperature of about 4° C. or higher, e.g., about 8° C. or higher, about 12° C. or higher, about 15° C. or higher, about 18° C. or higher, about 20° C. or higher, about 22° C. or higher, about 25° C. or higher, about 27° C. or higher, about 30° C. or higher, about 35° C. or higher, or about 50° C. or lower, e.g., about 45° C. or lower, about 40° C. or lower, about 37° C. or lower, about 35° C. or lower, about 32° C. or lower, about 30° C. or lower, about 28° C. or lower, about 26° C. or lower, about 23° C. or lower, about 20° C. or lower, about 15° C. or lower, or at a temperature in range defined by any two of the preceding values (e.g., 4-50° C., 15-35° C., 20-25° C., 12-20° C., 20-45° C., 15-30° C., etc.). In some embodiments, the complexing solution is incubated at a temperature of 12-28° C. In some embodiments, the complexing solution is incubated at a temperature of 15-25° C.

The complexing solution can be incubated for any suitable length of time. In some embodiments, the complexing solution is incubated for, for about, or for at least 10, 30, 45, or 60 minutes, 1.25, 1.5, 2, or 3 hours, or is incubated for, for about, or for not more than 12, 9, 6, 3, 2.5, or 2 hours, or for a length of time in a range defined by any two of the preceding values (e.g., 10 minutes to 12 hours, 10 minutes to 6 hours, 30 minutes to 3 hours, 1 hours to 3 hours, 1 hour to 9 hours, 10 minutes to 1 hour, etc.). In some embodiments, the complexing solution is incubated for about 30 minutes to about 6 hours. In some embodiments, the complexing solution is incubated for about 30 minutes to about 2 hours. In some embodiments, the complexing solution is incubated for about 1 hour.

In some embodiments, after preparing a complexing solution at block 1030 and permitting hybridization of the 3′ hybridizing regions to each other or the splint oligonucleotide at block 1040, the method can include removing the solution from the solid support, thereby removing a detection conjugate whose detection moiety is not bound (or is not simultaneously bound) with the capture moiety of the solid support to the analyte if present. In some embodiments, the solid support comprises a plurality of the capture moieties and a plurality of the capture oligonucleotides attached to the solid support, wherein providing the detection conjugate comprises providing a plurality of the detection conjugates, and wherein the method further comprises following preparing the complexing solution at block 1030 and prior to the extending or ligating at block 1050, removing any detection conjugate that is not bound to an analyte that is bound (or is simultaneously bound) to a capture moiety. In some embodiments, each solid support comprises a plurality of copies of the same capture moieties and a plurality of copies of the same capture oligonucleotides attached to the solid support, and a plurality of copies of the same detection conjugates are provided, and wherein the method further comprises following preparing the complexing solution at block 1030 and prior to the extending or ligating at block 1050, removing any copies of the detection conjugate that is not bound to an analyte that is bound to a copy of the capture moiety.

The solution containing any unbound detection conjugate can be removed from the solid support using any suitable option. In some embodiments, removing the solution includes washing the solid support. In some embodiments, removing the solution includes washing the solid support by transferring the solid support to a wash solution (e.g., a buffer solution) that does not include any detection conjugate. In some embodiments, removing the sample includes replacing the solution component (e.g., of the complexing solution or a subsequent washed solution) with a wash solution (e.g., a buffer solution) that does not include any detection conjugate. Washing the solid support can be done any suitable number of times. In some embodiments, the solid support is washed 1, 2, 3, 4, 5 or more times. In some embodiments, the solid support is washed 2-4 times. In some embodiments, the solid support is washed 3 times. In some embodiments, the washing involves an equivalent of about 1, 2, 3, 4, 5, or more volume exchanges with a wash solution.

Any suitable wash solution can be used to wash the solid support after the complexing solution is prepared (and after incubating as described herein to allow the capture moiety and the detection moiety in the complexing solution to both be bound (or to be simultaneously bound) to the analyte if present). In some embodiments, the wash solution includes a non-stringent wash buffer. In some embodiments, the non-stringent wash buffer includes phosphate-buffered saline (PBS) or PBS with polysorbate 20 (PBST). In some embodiments, removing unbound detection conjugate comprises washing the solid support, e.g., under high stringency conditions, for example, prior to block 1050. In some embodiments, the wash solution includes a stringent wash buffer (e.g., a low-salt buffer). In some embodiments, the stringent wash buffer includes a phosphate buffer. In some embodiments, the stringent wash buffer includes a polysorbate, e.g., polysorbate 20. In some embodiments, the stringent wash buffer includes polysorbate, e.g., polysorbate 20, at about 0.01% to about 0.5%. In some embodiments, the stringent wash buffer includes less than 10 mM phosphate. In some embodiments, the stringent wash buffer includes about 5 mM phosphate. In some embodiments, the stringent wash buffer includes about 5 mM phosphate and about 0.05% polysorbate 20. In some embodiments, the stringent wash buffer has a total sodium concentration of, of about, or of at most 20, 10, 9, 8, 7, 6, 5, 4, 3, 2, or 1 mM, or a concentration in a range defined by any two of the preceding values (e.g., about 1-20 mM, about 3-10 mM, about 5-10 mM, etc.). In some embodiments, the stringent wash buffer has a total sodium concentration of about 5 mM to about 10 mM. In some embodiments, washing the solid support includes washing one or more times (e.g., 1, 2, 3, 4, 5, or more times) with a non-stringent wash buffer, followed by washing one or more times (e.g., 1, 2, 3, 4, 5, or more times) with a stringent wash buffer. In some embodiments, washing the solid support includes washing once with a non-stringent wash buffer, followed by washing twice with a stringent wash buffer.

Where the 3′ hybridizing region of the capture oligonucleotide that is associated with a solid support to which a capture moiety bound to an analyte molecule is hybridized to the 3′ hybridizing region of the detection oligonucleotide that is attached to a detection moiety that is also bound to the same analyte molecule, extending the detection oligonucleotide (using the capture oligonucleotide as template strand), or extending the capture oligonucleotide (using the detection oligonucleotide as template strand) at block 1050 can generate an on-target extension product. In some embodiments, both the detection oligonucleotide and the capture oligonucleotide are extended to generate the on-target extension product (e.g., a double-stranded extension product).

As used herein, “on-target” denotes an arrangement of the capture oligonucleotide associated with the capture moiety and the detection oligonucleotide associated with the detection moiety being in proximity to each other due to the capture moiety and detection moiety both being bound (or simultaneously bound) to the analyte molecule to which both the capture moiety and detection moiety bind, and the 3′ hybridizing regions of the capture oligonucleotide and the detection oligonucleotide hybridizing to each other due to complementarity of the 3′ hybridizing regions to each other also referred to herein as an “on-target arrangement”). In some embodiments, an on-target interaction can render the hybridization between 3′ hybridizing regions to be stable enough (due to the proximity) to allow extension (e.g., allow a polymerase to use the hybridized region as substrate), even if the hybridization between the 3′ hybridizing regions are transient. An on-target extension product can be generated upon extension of the capture oligonucleotide or the detection oligonucleotide, or both, in an on-target arrangement. An on-target extension product can include a nucleic acid that includes a nucleotide sequence derived from the capture oligonucleotide and the detection oligonucleotide. For example, an on-target extension product generated by extension of the detection oligonucleotide using the capture oligonucleotide with which it is in an on-target arrangement as template strand can include the nucleotide sequence of the detection oligonucleotide and the reverse complement of the nucleotide sequence of the capture oligonucleotide (less the 3′ hybridizing region of the capture oligonucleotide, which overlaps with that of the detection oligonucleotide). A combination of solid support and detection conjugate that are capable of generating an on-target extension product can be said to be a “paired combination.” As used herein, “off-target” denotes an arrangement of a capture oligonucleotide associated with a capture moiety and a detection oligonucleotide associated with a detection moiety other than an on-target arrangement. In some embodiments, an off-target arrangement may be due to non-specific pairing of a capture oligonucleotide associated with a capture moiety and a detection oligonucleotide associated with the detection moiety (e.g., the 3′ hybridizing region of a capture oligonucleotide associated with a capture moiety hybridizing to a 3′ hybridizing region of a detection oligonucleotide associated with a detection moiety where the capture moiety and the detection moiety are not able to both be bound (or not able to be simultaneously bound) to the same analyte), or due to mis-priming of the capture oligonucleotide or the detection oligonucleotide of a correctly paired capture moiety and detection moiety, where the 3′ hybridizing region of the capture oligonucleotide (or detection oligonucleotide) hybridizes at a position other than the 3′ hybridizing region of the detection oligonucleotide (or capture oligonucleotide).

Extending the hybridized capture oligonucleotide and/or the hybridized detection oligonucleotide at block 1050 can be carried out in any suitable manner. In some embodiments, extending includes treating the solid support (to which the detection conjugate is attached via the detection moiety that is bound to the analyte, to which the capture moiety attached to the solid support is also bound) with a polymerase (e.g., a template-directed polymerase). Any suitable polymerase can be used. In some embodiments, the polymerase is a DNA polymerase, RNA polymerase, reverse transcriptase, etc. In some embodiments, the polymerase is, without limitation, Taq polymerase, DNA polymerase I, Klenow fragment, T4 DNA polymerase, T7 RNA polymerase. In some embodiments, the polymerase is a strand-displacing polymerase. In some embodiments, the strand-displacing polymerase is a strand-displacing DNA polymerase. In some embodiments, the strand-displacing polymerase is a 3′→5′ exo-polymerase. In some embodiments, the strand-displacing polymerase is a Klenow fragment. In some embodiments, the strand-displacing polymerase is an exo-Klenow fragment.

The solid support can be treated with the polymerase (e.g., a strand-displacing polymerase, such as an exo-Klenow fragment) for any suitable amount of time. In some embodiments, the solid support is treated with the polymerase for, for about, or for at least 15, 30, 45, 60, 75, or 90 minutes, 2 or 3 hours, or a length of time in a range defined by any two of the preceding values (e.g., about 15 minutes to 3 hours, about 30 minutes to 90 minutes, about 45 minutes to 1 hour, etc.). In some embodiments, the solid support is treated with the polymerase for about 30 minutes to about 90 minutes. In some embodiments, the solid support is treated with the polymerase for about 60 minutes.

The solid support can be treated with the polymerase (e.g., a strand-displacing polymerase, such as an exo-Klenow fragment) at any suitable temperature. In some embodiments, the solid support is treated with the polymerase at room temperature. In some embodiments, the solid support is treated with the polymerase at a temperature of about 12° C. or higher, e.g., about 15° C. or higher, about 18° C. or higher, about 20° C. or higher, about 22° C. or higher, about 25° C. or higher, about 27° C. or higher, about 30° C. or higher, about 35° C. or higher, about 40° C. or higher, about 45° C. or higher, about 50° C. or higher, or about 60° C. or lower, e.g., about 55° C. or lower, about 50° C. or lower, about 45° C. or lower, about 40° C. or lower, about 35° C. or lower, about 30° C. or lower, about 28° C. or lower, about 26° C. or lower, about 23° C. or lower, about 20° C. or lower, about 18° C. or lower, or at a temperature in range defined by any two of the preceding values (e.g., about 12-60° C., about 15-55° C., about 15-28° C., about 30-50° C., about 20-45° C., about 18-30° C., about 15-25° C., etc.). In some embodiments, the solid support is treated with the polymerase at a temperature of about 15-28° C. In some embodiments, the solid support is treated with the polymerase at a temperature of about 15-25° C.

The solid support can be treated with any suitable amount of the polymerase (e.g., a strand-displacing polymerase, such as an exo-Klenow fragment). In some embodiments, the polymerase is treated with about 1 U/mL or more, e.g., about 2 U/mL, about 5 U/mL, about 10 U/mL, about 15 U/mL, about 20 U/mL, about 25 U/mL, or an amount in a range defined by any two of the preceding values (e.g., about 1-25 U/mL, about 2-20 U/mL, about 5-15 U/mL, about, etc.) of the polymerase. In some embodiments, the polymerase is treated with about 5 to about 15 U/mL of the polymerase. In some embodiments, the polymerase is treated with about 10 U/mL of the polymerase.

Releasing the on-target extension product from the solid support at block 1060 can be done using any suitable option. In some embodiments, releasing the on-target extension product includes releasing from the solid support only. In some embodiments, releasing the on-target extension product includes releasing from the solid support and from the detection conjugate. In some embodiments, the on-target extension product is released into the supernatant fraction. In some embodiments, releasing the on-target extension product includes treating the solid support with a strand-displacing polymerase, a restriction enzyme, a protease, and/or a high-stringency wash. In some embodiments, the option selected from releasing the on-target extension product depends on the manner by which the capture oligonucleotide is attached to the solid support and/or the manner by which the detection oligonucleotide is attached to the detection moiety. In some embodiments, the on-target extension product is released from the solid support using an option that is different from the option used to release the on-target extension product from the detection conjugate. In some embodiments, the on-target extension product is released from the solid support using the same option as that used to release the on-target extension product from the detection conjugate.

The releasing can be done under any suitable temperature. In some embodiments, the releasing at block 1060 is performed at room temperature. In some embodiments, the releasing at block 1060 is performed at a temperature between, or in the range of, 10⁻³⁷° C. In some embodiments, the releasing is performed at a temperature of about 10° C. or higher, e.g., about 15° C. or higher, about 18° C. or higher, about 20° C. or higher, about 22° C. or higher, about 25° C. or higher, about 27° C. or higher, about 30° C. or higher, about 35° C. or higher, about 40° C. or higher, about 45° C. or higher, about 50° C. or higher, or about 75° C. or lower, e.g., about 70° C. or lower, about 65° C. or lower, about 60° C. or lower, 55° C. or lower, about 50° C. or lower, about 45° C. or lower, about 40° C. or lower, about 35° C. or lower, about 30° C. or lower, about 28° C. or lower, about 26° C. or lower, about 23° C. or lower, about 20° C. or lower, about 18° C. or lower, or at a temperature in range defined by any two of the preceding values (e.g., about 10⁻⁷⁵° C., about 15-70° C., about 15-28° C., about 50-65° C., about 20-45° C., about 18-30° C., about 15-25° C., etc.).

In some embodiments, extending and releasing are performed by a single enzyme. In some embodiments, extending and releasing are performed by the same enzyme. In some embodiments, the releasing does not require using a protease or restriction enzyme. In some embodiments, by designing the assay such that a single enzyme performs the extending and releasing aspects, there is no need for a separate step for releasing the extension products after extending and the assay time may be reduced compared to another assay that requires separate steps for extending and releasing the extension products (e.g., by using an enzyme for extension and a separate process for releasing the extension products). In some embodiments, the single enzyme includes a strand-displacing polymerase, e.g., an exo-Klenow fragment. In some embodiments, releasing the on-target extension product includes treating the solid support with a strand-displacing polymerase (e.g., an exo-Klenow fragment), where the capture oligonucleotide is attached to the solid support via hybridization to a first tether oligonucleotide attached (e.g., via a biotin-streptavidin binding interaction) to the solid support, as described herein. In some embodiments, releasing the on-target extension product includes treating the solid support with a strand-displacing polymerase (e.g., an exo-Klenow fragment), where the capture oligonucleotide is attached to the solid support via hybridization to a first tether oligonucleotide attached (e.g., via a biotin-streptavidin binding interaction) to the solid support and the detection oligonucleotide is attached to the detection moiety via hybridization to a second tether oligonucleotide attached (e.g., covalently attached) to the detection moiety, as described herein. In some embodiments, extending the hybridized capture oligonucleotide and/or the hybridized detection oligonucleotide at block 1050 includes treating the solid support with a strand-displacing polymerase under conditions sufficient to extend the hybridized capture oligonucleotide and/or the hybridized detection oligonucleotide, and releasing the on-target extension product at block 1060 includes allowing the strand-displacing polymerase (e.g., exo-Klenow fragment) to displace at least the first tether oligonucleotide hybridized to the capture oligonucleotide during extension, and optionally to displace the second tether oligonucleotide hybridized to the detection oligonucleotide during extension. In some embodiments, the on-target extension product is released from both the capture solid support and the detection conjugate through displacement of the first and second tether oligonucleotides, respectively, from the on-target extension product upon extension. In some embodiments, releasing does not include the use of a protease.

In some embodiments, releasing the on-target extension product at block 1060 includes treating the solid support with a protease (e.g., proteinase K) where the capture oligonucleotide is attached to the solid support via a bonding interaction that is independent of the nucleotide sequence of the capture oligonucleotide (e.g., does not involve hybridization of the capture oligonucleotide to a tether oligonucleotide that is attached to the solid support), as provided herein. In some embodiments, the on-target extension product is covalently attached to a first member (e.g., biotin) of a binding pair bound to a second member (e.g., streptavidin) of the binding pair, wherein the second member is attached (e.g., covalently attached) to the solid support, and releasing the on-target extension product from the solid support at block 1060 includes cleaving the second member of the binding pair (e.g., proteolysis). In some embodiments, releasing the on-target extension product from the solid support at block 1060 includes cleaving the first member of the binding pair. In some embodiments, releasing the on-target extension product includes treating the solid support with a protease (e.g., proteinase K). In some embodiments, treating the solid support with a protease cleaves a second member (e.g., streptavidin) of a binding pair bound to a first member (e.g., biotin), where the second member is attached to the solid support and the first member is covalently attached to the on-target extension product. Any suitable protease can be used to release the on-target extension product. In some embodiments, the protease is proteinase K, trypsin, or LysC. In some embodiments, the protease is proteinase K.

In some embodiments, the on-target extension product is covalently attached to the detection moiety, and releasing the on-target extension product at block 1060 includes cleaving the covalent attachment of the on-target extension product from the detection moiety. In some embodiments, releasing the on-target extension product at block 1060 includes treating the solid support with a protease (e.g., proteinase K). In some embodiments, treating the solid support with a protease cleaves the detection moiety to which the on-target extension product (formed by on-target extension of the detection oligonucleotide) is conjugated.

In some embodiments, releasing the on-target extension product at block 1060 includes treating the solid support with a high-stringency wash, where the capture oligonucleotide is attached to the solid support via hybridization to a first tether oligonucleotide attached (e.g., via a biotin-streptavidin binding interaction) to the solid support, as described herein, where the region of hybridization between the capture oligonucleotide and the first tether oligonucleotide is sufficiently short such that hybridization is disrupted by the high-stringency wash. In some embodiments, releasing the on-target extension product includes treating the solid support with a high-stringency wash, where the capture oligonucleotide is attached to the solid support via hybridization to a first tether oligonucleotide attached (e.g., via a biotin-streptavidin binding interaction) to the solid support, as described herein, where the region of hybridization between the capture oligonucleotide and the first tether oligonucleotide is sufficiently short such that hybridization is disrupted by the high-stringency wash, and the detection oligonucleotide is attached to the detection moiety via hybridization to a second tether oligonucleotide attached (e.g., covalently attached) to the detection moiety, as described herein, where the region of hybridization between the detection oligonucleotide and the second tether oligonucleotide is sufficiently short such that hybridization is disrupted by the high-stringency wash.

In some embodiments, releasing the on-target extension product at block 1060 includes treating the solid support with a restriction enzyme. In some embodiments, where the capture oligonucleotide and/or the detection oligonucleotide includes a restriction enzyme cleavage site, releasing the on-target extension product includes treating the solid support with the restriction enzyme that cleaves at the cleavage site. Any suitable restriction enzyme can be used. In some embodiments, the restriction enzyme has a recognition site that is 4, 5, 6, 7, 8, 9, 10 nucleotides long or longer. In some embodiments, the restriction enzyme has a recognition site that is 6 nucleotides long. Suitable restriction enzymes include, without limitation, EcoRI, EcoRV, HindIII, XbaI, NotI, SpeI, SacI, BamHI, etc.

Determining the presence and/or amount, or the absence of the released on-target extension product at block 1070 can be done using any suitable option. In some embodiments, determining the presence and/or amount, or the absence of the released on-target extension product includes detecting a level of the on-target extension product in the supernatant of the extension reaction, or the releasing reaction. In some embodiments, the presence or absence of the product, and/or the amount of the extension product (e.g., on-target extension product) is determined using a suitable option for nucleic acid analysis, including, without limitation, PCR, qPCR, sequencing, hybridization, microarray, etc. As used herein, determining the presence or absence of the product, and/or the amount of the extension product contemplates detecting either or both the extension product itself, or an amplification product thereof (e.g., a library of amplified nucleic acids prepared from the extension products). In some embodiments, the product of the extension reaction (as carried out in block 1050) is released (at block 1060) into the supernatant of the extension reaction (or the release reaction), and the supernatant is assayed for the presence or absence, and/or amount of the on-target extension product. In some embodiments, the method includes extending at least one of the hybridized capture oligonucleotide and the hybridized detection oligonucleotide via a primer extension reaction; isolating a supernatant fraction of the primer extension reaction, the supernatant fraction comprising the one or both strands of the on-target extension product released from at least the solid support; and determining the presence and/or amount, or the absence of the released on-target extension product in the supernatant fraction. In some embodiments, the detected level of the on-target extension product is used to determine the amount of the analyte in the sample by comparing the detected level to a reference level or a standard curve.

In some embodiments, the method includes generating a calibration curve of the analyte by: generating a plurality of serially diluted calibrator samples, each calibrator sample comprising a known amount of the analyte in a dilution series; and determining the presence and/or amount, or the absence of the released on-target extension product in each of the plurality of serially diluted calibrator samples. Any suitable dilution series of the analyte can be used as the serially diluted calibrator samples. In some embodiments, the analyte is serially diluted at 2 fold, 3 fold, 5 fold, 10 fold, 20 fold, 30 fold, 50 fold, 100 fold or more intervals, or optionally the analyte is serially diluted at intervals of a fold amount in a range defined by any two of the preceding values (e.g., 2-100 fold, 2-20 fold, 3-10 fold, 3-50 fold, etc.). In some embodiments, the analyte is serially diluted at a constant interval (e.g., at an interval of about 1 μg/mL, about 5 μg/mL, about 10 μg/mL, about 20 μg/mL, about 50 μg/mL, about 100 μg/mL, about 1,000 μg/mL, or optionally the analyte is serially diluted at a constant interval in a range defined by any two of the preceding values (e.g., about 1-1,000 μg/mL, about 5-100 μg/mL, about 10⁻⁵⁰ pg/mL, etc.)). In some embodiments, the analyte is present in a calibrator sample at about 0.01 pg/mL, about 0.02 pg/mL, about 0.05 pg/mL, about 0.1 pg/mL, 0.2 pg/mL, about 0.5 pg/mL, about 1 μg/mL, about 2 μg/mL, about 5 μg/mL, about 10 μg/mL, about 15 μg/mL, about 20 μg/mL, about 25 μg/mL, about 50 μg/mL, about 75 μg/mL, about 100 μg/mL, about 150 μg/mL, about 200 μg/mL, about 250 μg/mL, about 300 μg/mL, about 400 μg/mL, about 500 μg/mL, about 600 μg/mL, about 700 μg/mL, about 800 μg/mL, about 900 μg/mL, about 1,000 μg/mL, about 2,000 μg/mL, about 5,000 μg/mL, about 10,000 μg/mL, about 50,000 μg/mL, about 100,000 μg/mL, about 500,000 μg/mL, about 1,000,000 μg/mL, or more, or optionally the analyte is present in a calibrator sample at a concentration in a range defined by any two of the preceding values (e.g., about 0.01-1,000,000 μg/mL, about 0.02-100,000 μg/mL, about 0.1-1,000 μg/mL, about 0.1-500 μg/mL, about 1-50,000 μg/mL, etc.).

In some embodiments, the sample includes the analyte (e.g., a detectable level of the analyte), or is known to include the analyte. In some embodiments, where the sample comprises the analyte, the detection moiety and capture moiety are both bound (or are simultaneously bound) to the analyte such that the capture oligonucleotide and detection oligonucleotide are in proximity after block 1030, the 3′ hybridizing region of the capture oligonucleotide and the 3′ hybridizing region of the detection oligonucleotide are hybridized to each other at block 1040; the hybridized capture oligonucleotide and the hybridized detection oligonucleotide are extended to generate on-target extension product that comprises the extended capture oligonucleotide and the extended detection oligonucleotide at block 1050, and one or both strands of the on-target extension product is released from the solid support and/or the detection moiety at block 1060, and wherein the method comprises at block 1070, determining the presence and/or amount of the released extension product to determine the presence and/or amount of the analyte in the sample.

### B. Solid Support and Detection Conjugate

Solid supports and detection conjugates that find use in the methods herein are provided. A solid support can include a capture moiety attached to the solid support, and a capture oligonucleotide attached to the solid support. In some embodiments, the solid support includes a plurality of capture moieties and a plurality of capture oligonucleotides. In some embodiments, the number and/or density of capture moieties on the solid support is greater than the number and/or density of capture oligonucleotides on the solid support. In some embodiments, a higher number and/or density of the capture moiety compared to the number and/or density of the capture oligonucleotide on the solid support can reduce non-specific interaction of the capture oligonucleotide with the detection oligonucleotide in the detection conjugate. In some embodiments, the solid support comprises a ratio of the number and/or density of the capture moiety to the capture oligonucleotide of, of about, or of at least, 2:1, 2.5:1, 3:1, 3.5:1, 4:1, 4.5:1, 5:1, 6:1, 7:1, 8:1, 9:1, 10:1, 15:1, 20:1, 25:1, 30:1, 40:1, 45:1, 50:1, 55:1, 60:1, 70:1, 80:1, 90:1, 100:1, 150:1, 200:1, 250:1, 300:1, 400:1, 500:1, 1,000:1, 2,000:1, 5,000:1, 10,000:1, 50,000:1, 100,000:1, or optionally wherein the solid support comprises a ratio of the number and/or density of the capture moiety to the capture oligonucleotide in a range defined by any two of the preceding values (e.g., 2:1-100,000:1, 5:1-5,000:1, 10:1-1,000:1, 5:1-500:1, etc.), optionally about 2:1 to about 50:1, about 2:1 to about 10:1, about 3:1 to about 7:1, or about 5:1, optionally about 50:1. In some embodiments, the solid support comprises a ratio of the capture moiety to the capture oligonucleotide of about 5:1. In some embodiments, the solid support comprises a ratio of the capture moiety to the capture oligonucleotide of about 50:1.

In some embodiments, the solid support comprises, comprises about, or comprises at least 0.03, 0.05, 0.1, 0.2, 0.3, 0.4, 0.5, 0.6, 0.7, 0.8, 0.9, 1, 1.2, 1.4, 1.6, 1.8, 2 μmol, or comprises, comprises about, or comprises at most 3, 2.5, 2, 1.5, 1.2, 1.0, 0.8, 0.7, 0.6, or 0.5 μmol, or an amount in a range defined by any two of the preceding values (e.g., about 0.03-3 μmol, about 0.05-2 μmol, about 0.1-1.5 μmol, about 0.1-1 μmol, about 0.2-0.6 μmol, etc.) of the capture moiety per g of solid support, where the solid support includes a bead (e.g., a SA-coated bead that having a diameter of about 1 m). In some embodiments, the solid support comprises about 0.03-3 pmol of the capture moiety per g of solid support, where the solid support includes a bead (e.g., a SA-coated bead having a diameter of about 1 m). In some embodiments, the solid support comprises about 0.1-0.5 pmol of the capture moiety per g of solid support, where the solid support includes a bead (e.g., a SA-coated bead having a diameter of about 1 m). In some embodiments, the solid support comprises about 0.3 pmol of the capture moiety per g of solid support, where the solid support includes a bead (e.g., a SA-coated bead having a diameter of about 1 m).

Any suitable solid support can be used. In some embodiments, the solid support includes, without limitation, a bead, a microparticle, a resin, a gel, a slide, chip, or a microwell. In some embodiments, the solid support includes a polymeric solid support. In some embodiments, the solid support includes polymer selected from, without limitation, polystyrene, polypropylene, polyethylene, polydimethylsiloxane (PDMS), silicone, agarose, gelatin. In some embodiments, the solid support includes a magnetic, paramagnetic or superparamagnetic bead.

In some embodiments, the solid support is a bead (e.g., magnetic or paramagnetic bead). In some embodiments, the bead is a polymeric bead with a magnetic core. In some embodiments, the bead includes a hydrophilic outer layer. In some embodiments, the bead is a streptavidin-coated bead. In some embodiments, the bead has a diameter of about 0.1 m to about m. In some embodiments, the bead has a diameter of about 0.5 m to about 3 m. In some embodiments, the bead has a diameter of about 0.5 m to about 2 m. In some embodiments, the bead has an average diameter of about 1 m. In some embodiments, the solid support has a binding capacity of, or equivalent to about 55 μg of a biotinylated IgG per mg of a bead having an average diameter of about 1 m. In some embodiments, the solid support has a binding capacity of, or equivalent to about 10 μg or more, e.g., about 15 μg or more, about 20 μg or more, about 25 μg or more, about 30 μg or more, about 35 μg or more, about 40 μg or more, about 45 μg or more, about 50 μg or more, about 55 μg or more, about 60 μg or more, about 65 μg or more, about 70 μg or more, about 80 μg or more, about 90 μg or more, about 100 μg or more, or an amount in a range defined by any two of the preceding values (e.g., about 10⁻¹⁰⁰ μg, about 20-80 μg, about 30-70 g, about 40-90 μg, etc.) of a biotinylated IgG per mg of a bead having an average diameter of about 1 m.

The capture moiety can be attached to the solid support through any suitable option. In some embodiments, capture moiety is indirectly attached to the solid support (e.g., attachment is mediated by one or more other molecules). In some embodiments, the capture moiety is covalently attached to a first member of a binding pair that binds to a second member of the binding pair, wherein the second member is attached to the solid support (e.g., via a covalent interaction between the second member and the solid support). Any suitable binding pair can be used. In some embodiments, the binding pair is a biotin-streptavidin binding pair. In some embodiments, the capture moiety is covalently attached to biotin (or is biotinylated), and the streptavidin is directly attached to the solid support (e.g., a streptavidin-coated bead). In general, attachment to the solid support does not interfere with the binding of the capture moiety to the analyte. In some embodiments, attachment to the solid support is at a site on the capture moiety distal to the analyte binding region. In some embodiments, where the capture moiety includes an antibody, attachment to the solid support is at the C-terminus of the antibody, at a C-terminal end of the antibody molecule, or in a constant region of the antibody.

In some embodiments, the capture moiety is directly attached to the solid support. In some embodiments, the capture moiety is non-specifically adsorbed onto the solid support. In some embodiments, the capture moiety is covalently attached to the solid support. The capture moiety may be covalently attached to the solid support using any suitable option. In some embodiments, the covalent attachment includes, without limitation, amine-thiol crosslinking, maleimide crosslinking, N-hydroxysuccinimide or N-hydroxysulfosuccinimide. In some embodiments, the capture moiety is covalently attached to the solid support via one or more linkers.

The capture oligonucleotide can be attached to the solid support through any suitable option. In some embodiments, the capture oligonucleotide is attached (directly or indirectly) to the solid support at the 5′ end or closer to the 5′ end than to the 3′ end. In some embodiments, the capture oligonucleotide is attached to the solid support via a bonding interaction that is independent of the nucleotide sequence in the capture oligonucleotide. In some embodiments, a bonding interaction that is independent of the nucleotide sequence in the capture oligonucleotide is not disrupted by a strand displacing polymerase, e.g., upon extension.

In some embodiments, the capture oligonucleotide is directly attached to the solid support. In some embodiments, the capture oligonucleotide is non-specifically adsorbed onto the solid support. In some embodiments, the capture oligonucleotide is covalently attached to the solid support. In some embodiments, the covalent attachment includes, without limitation, amine-thiol crosslinking, maleimide crosslinking, N-hydroxysuccinimide or N-hydroxysulfosuccinimide. In some embodiments, the capture oligonucleotide is covalently attached to the solid support via one or more linkers.

In some embodiments, the capture oligonucleotide is indirectly attached to the solid support (e.g., attachment is mediated by one or more other molecules). In some embodiments, the capture oligonucleotide is attached to the solid support independently of the capture moiety. Binding independently of the capture moiety denotes that binding of the capture oligonucleotide to the solid support does not require that the capture moiety is also attached to the solid support. In some embodiments, the capture oligonucleotide is not directly or covalently attached to the capture moiety.

In some embodiments, the capture oligonucleotide is attached to the solid support via a binding pair. In some embodiments, the capture oligonucleotide is covalently attached to a first member of a binding pair bound to a second member of the binding pair, wherein the second member is attached to the solid support (e.g., via a covalent interaction between the second member and the solid support). Any suitable binding pair can be used. In some embodiments, the binding pair is a biotin-streptavidin binding pair. In some embodiments, the capture oligonucleotide is covalently attached to biotin, and the streptavidin is directly attached to the solid support (e.g., a streptavidin-coated bead).

In some embodiments, the capture oligonucleotide is attached to the solid support via an oligonucleotide tether (also referred to as a capture tether). In some embodiments, the capture oligonucleotide is attached to the solid support via hybridization to a tether oligonucleotide attached to the solid support. In some embodiments, an attachment of the capture oligonucleotide through hybridization to a tether oligonucleotide attached to the solid support can be disrupted by a strand displacing polymerase. The tether oligonucleotide can be attached to the solid support though any suitable option. In some embodiments, the tether oligonucleotide is covalently attached to the solid support, or is adsorbed onto the solid support. In some embodiments, the tether oligonucleotide is attached (directly or indirectly) to the solid support at the 3′ end or closer to the 3′ end than to the 5′ end. In some embodiments, the tether oligonucleotide is indirectly attached to the solid support (e.g., attachment is mediated by one or more other molecules).

In some embodiments, the tether oligonucleotide is attached to the solid support via a binding pair. In some embodiments, the tether oligonucleotide is covalently attached to a first member of a binding pair bound to a second member of the binding pair, wherein the second member is attached to the solid support (e.g., via a covalent interaction between the second member and the solid support). Any suitable binding pair can be used. In some embodiments, the binding pair is a biotin-streptavidin binding pair. In some embodiments, the tether oligonucleotide is covalently attached to biotin, and the streptavidin is directly attached to the solid support (e.g., a streptavidin-coated bead).

In some embodiments, the tether oligonucleotide is directly attached to the solid support. In some embodiments, the tether oligonucleotide is non-specifically adsorbed onto the solid support. In some embodiments, the tether oligonucleotide is covalently attached to the solid support. In some embodiments, the covalent attachment includes, without limitation, amine-thiol crosslinking, maleimide crosslinking, N-hydroxysuccinimide or N-hydroxysulfosuccinimide. In some embodiments, the tether oligonucleotide is covalently attached to the solid support via one or more linkers.

The tether oligonucleotide can be any suitable length. In some embodiments, the tether oligonucleotide is 10, 11, 12, 13, 14, 15, 16, 17, 18, 19, 20, 21, 22, 23, 24, 25, 26, 27, 28, 29, or 30 nucleotides long, or longer. In some embodiments, the tether oligonucleotide is about 15 to about 25 nucleotides long. In some embodiments, the tether oligonucleotide is about 15 to about 30 nucleotides long. In some embodiments, the tether oligonucleotide is, or is about 20 nucleotides long. The tether oligonucleotide can include a nucleotide sequence that is complementary to a sequence in the capture oligonucleotide to which the tether oligonucleotide hybridizes. In some embodiments, the nucleotide sequence that is complementary to a sequence in the capture oligonucleotide is 10, 11, 12, 13, 14, 15, 16, 17, 18, 19, 20, 21, 22, 23, 24, 25, 26, 27, 28, 29, or 30 nucleotides long, or longer. In some embodiments, the nucleotide sequence that is complementary to a sequence in the capture oligonucleotide is about 15 to about 25 nucleotides long. In some embodiments, the nucleotide sequence that is complementary to a sequence in the capture oligonucleotide is about 15 to about 30 nucleotides long. In some embodiments, the nucleotide sequence that is complementary to a sequence in the capture oligonucleotide is, or is about, 20 nucleotides long. In some embodiments, the nucleotide sequence of the tether oligonucleotide consists of or consists essentially of the sequence that is complementary to a sequence in the capture oligonucleotide. In some embodiments, the tether oligonucleotide includes a sequence complementary to a sequence in the capture oligonucleotide that is longer than the 3′ hybridizing region of the capture oligonucleotide.

A detection conjugate can include a detection moiety and a detection oligonucleotide attached to the detection moiety. The detection oligonucleotide can be attached to the detection moiety through any suitable option. In some embodiments, the detection oligonucleotide is attached (directly or indirectly) to the detection moiety at the 5′ end or closer to the 5′ end than to the 3′ end. In some embodiments, the detection oligonucleotide is attached to the detection moiety via a bonding interaction that is independent of the nucleotide sequence in the detection oligonucleotide. In some embodiments, a bonding interaction that is independent of the nucleotide sequence in the detection oligonucleotide is not disrupted by a strand displacing polymerase, e.g., upon extension. In some embodiments, an oligonucleotide (e.g., detection oligonucleotide or a tether oligonucleotide) is attached to the detection moiety (e.g., antibody) with a degree of labeling (DOL) of about 1 or greater, e.g., about 2 or greater, about 2.5 or greater, about 3 or greater, about 3.5 or greater, about 4 or greater, about 4.5 or greater, about 5 or greater, or with an amount in a range defined by any two of the preceding values (e.g., about 1-5, about 2-4.5, about 3-4.5, etc.). In some embodiments, an oligonucleotide (e.g., detection oligonucleotide or a tether nucleotide) is attached to the detection moiety (e.g., antibody) with a DOL of about 3-4.5, or about 3.5-4, e.g., about 3.7.

In some embodiments, the detection oligonucleotide is directly attached to the detection moiety. In some embodiments, the detection oligonucleotide is covalently attached to the detection moiety. In some embodiments, the covalent attachment includes, without limitation, amine-thiol crosslinking, maleimide crosslinking, N-hydroxysuccinimide (NHS) or N-hydroxysulfosuccinimide. In some embodiments, the detection oligonucleotide is covalently attached to the detection moiety via one or more linkers. In some embodiments, the detection oligonucleotide is covalently attached via a NHS ester to a lysine residue in the detection moiety. In some embodiments, the detection moiety is an antibody, and the detection oligonucleotide is covalently attached to a Fc domain of the antibody.

In some embodiments, the detection oligonucleotide is indirectly attached to the detection moiety (e.g., attachment is mediated by one or more other molecules). In some embodiments, the detection oligonucleotide is attached to the detection moiety via an oligonucleotide tether (also referred to as a detection tether). In some embodiments, the detection oligonucleotide is attached to the detection moiety via hybridization to a tether oligonucleotide attached to the detection moiety. In some embodiments, an attachment of the detection oligonucleotide through hybridization to a tether oligonucleotide attached to the detection moiety can be disrupted by a strand displacing polymerase. The tether oligonucleotide can be attached to the detection moiety though any suitable option. In some embodiments, tether oligonucleotide is covalently attached to the detection moiety. In some embodiments, the tether oligonucleotide is attached (directly or indirectly) to the detection moiety at the 3′ end or closer to the 3′ end than to the 5′ end. In some embodiments, the tether oligonucleotide is attached (directly or indirectly) to the detection moiety such that the 3′ end is proximal to the detection moiety.

In some embodiments, the tether oligonucleotide is directly attached to the detection moiety. In some embodiments, the tether oligonucleotide is covalently attached to the detection moiety. In some embodiments, the covalent attachment includes, without limitation, amine-thiol crosslinking, maleimide crosslinking, N-hydroxysuccinimide or N-hydroxysulfosuccinimide. In some embodiments, the tether oligonucleotide is covalently attached to the detection moiety via one or more linkers. In some embodiments, the tether oligonucleotide is covalently attached via a NHS ester to a lysine residue in the detection moiety. In some embodiments, the detection moiety is an antibody, and the tether oligonucleotide is covalently attached to a Fc domain of the antibody.

In some embodiments, the detection oligonucleotide is attached to the detection moiety in a deterministic manner in order to provide a uniform distribution of detection oligonucleotide on each detection moiety. For example, detection conjugates may be produced by labeling detection antibodies with exactly 2 capture oligonucleotides. By way of further example, detection conjugates may be produced by labeling detection antigen binding fragments of antibodies (Fabs) with exactly one detection oligonucleotide. Several methods for site-specific modification are known including, for example—Genovis GlyClick, which allows exactly 2 labels per antibody.

The tether oligonucleotide can be any suitable length. In some embodiments, the tether oligonucleotide is 10, 11, 12, 13, 14, 15, 16, 17, 18, 19, 20, 21, 22, 23, 24, 25, 26, 27, 28, 29, or 30 nucleotides long, or longer. In some embodiments, the tether oligonucleotide is about 15 to about 25 nucleotides long. In some embodiments, the tether oligonucleotide is about 15 to about 30 nucleotides long. In some embodiments, the tether oligonucleotide is, or is about 20 nucleotides long. The tether oligonucleotide can include a nucleotide sequence that is complementary to a sequence in the detection oligonucleotide to which the tether oligonucleotide hybridizes. In some embodiments, the nucleotide sequence that is complementary to a sequence in the capture oligonucleotide is 10, 11, 12, 13, 14, 15, 16, 17, 18, 19, 20, 21, 22, 23, 24, 25, 26, 27, 28, 29, or 30 nucleotides long, or longer. In some embodiments, the nucleotide sequence that is complementary to a sequence in the detection oligonucleotide is about 15 to about 25 nucleotides long. In some embodiments, the nucleotide sequence that is complementary to a sequence in the detection oligonucleotide is about 15 to about 30 nucleotides long. In some embodiments, the nucleotide sequence that is complementary to a sequence in the detection oligonucleotide is, or is about, 20 nucleotides long. In some embodiments, the nucleotide sequence of the tether oligonucleotide consists of or consists essentially of the sequence that is complementary to a sequence in the detection oligonucleotide. In some embodiments, the tether oligonucleotide includes a sequence complementary to a sequence in the detection oligonucleotide that is longer than the 3′ hybridizing region of the detection oligonucleotide.

The tether oligonucleotide attached to the solid support (before the solid support and detection conjugate are combined) and the tether oligonucleotide attached to the detection moiety (before the solid support and detection conjugate are combined) can be the same or can be different (e.g., can have the same or different nucleotide sequence, can have the same or different length).

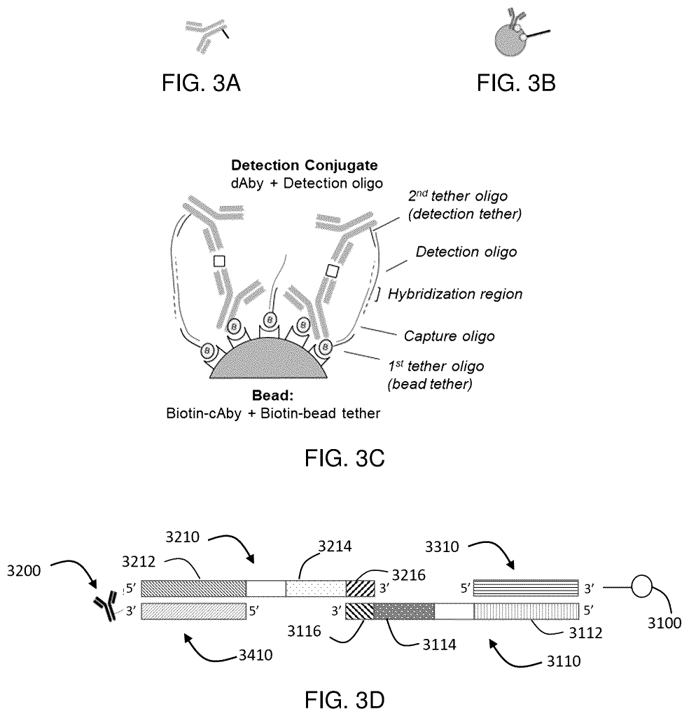

With reference to FIG. 3D, non-limiting embodiments of a detection oligonucleotide and a capture oligonucleotide are provided. The capture oligonucleotide and detection oligonucleotide can each independently be any suitable length. In some embodiments, the capture oligonucleotide and detection oligonucleotide are each independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 nucleotides long, or longer, or a length in a range defined by any two of the preceding values (e.g., 15-100 nucleotides, 20-90 nucleotides, 30-50 nucleotides, 40-80 nucleotides, 50-100 nucleotides, etc.). In some embodiments, the capture oligonucleotide and detection oligonucleotide are each independently about 30-50 nucleotides long.

A capture oligonucleotide 3110 may be attached to a capture moiety 3100 and include a 3′ hybridizing region 3116. A detection oligonucleotide 3210 may be attached to a detection moiety 3200 and include a 3′ hybridizing region 3216. In some embodiments, the 3′ hybridizing region of the capture oligonucleotide and/or the detection oligonucleotide is sufficiently short (or has a sufficiently low number of complementary nucleotides) such that in the absence of an analyte bound to both (or simultaneously bound to) the corresponding capture and detection moieties, no extension product (e.g., no detectable amount of extension product) is generated upon carrying out the extension reaction with the solid support. In some embodiments, the 3′ hybridizing region of the capture oligonucleotide and/or the detection oligonucleotide is long enough to allow hybridization to each other that is stable enough to generate detectable amount of extension product upon extension only when an analyte is bound to both (or simultaneously bound to) the corresponding capture and detection moieties. In some embodiments, the 3′ hybridizing region of the capture oligonucleotide and/or the detection oligonucleotide is long enough to provide sufficient sequence diversity in a multiplex format (e.g., to prevent mis-pairing). In some embodiments, the 3′ hybridizing region of the capture oligonucleotide and/or the detection oligonucleotide is at most 10, 9, 8, 7, 6, 5, or 4 nucleotides long, or has a length in a range defined by any two of the preceding values (e.g., 4-10 nucleotides long, 5-7 nucleotides long, 6-9 nucleotides long, etc.). In some embodiments, the 3′ hybridizing region of the capture oligonucleotide and/or the detection oligonucleotide is 5-7 nucleotides long. In some embodiments, the 3′ hybridizing region of the capture oligonucleotide and/or the detection oligonucleotide is 6 or 7 nucleotides long.

In some embodiments, the capture oligonucleotide 3110 is indirectly attached to a solid support 3100, e.g., via a tether oligonucleotide 3310. In some embodiments, the capture oligonucleotide includes a 5′ tethering region 3112 that hybridizes to a tether oligonucleotide 3310 attached to the solid support. In some embodiments, the capture oligonucleotide includes a 5′ tethering region 3112 that includes a nucleotide sequence that is complementary to at least a portion (or substantially all (e.g., at least 80%, 85%, 90%, 95%, 97%, 98%, or 99%, or optionally 100%)) of the tether oligonucleotide 3310 attached to the solid support. In some embodiments, the nucleotide sequence of the 5′ tethering region that is complementary to the tether oligonucleotide attached to the solid support is 10, 11, 12, 13, 14, 15, 16, 17, 18, 19, 20, 21, 22, 23, 24, 25, 26, 27, 28, 29, 30, 35, 40, 45, 50, 55, 60 nucleotides, or longer, or optionally a length in a range defined by any two of the preceding values (e.g., 10-60, 20-55, 15-50, 30-40 nucleotides long, etc.). In some embodiments, the nucleotide sequence of the 5′ tethering region that is complementary to the tether oligonucleotide attached to the solid support is about 15-50 nucleotides long. In some embodiments, the nucleotide sequence of the 5′ tethering region that is complementary to the tether oligonucleotide attached to the solid support is about 15-30 nucleotides long. In some embodiments, the nucleotide sequence of the 5′ tethering region that is complementary to the tether oligonucleotide attached to the solid support is about 20 nucleotides long.

In some embodiments, the detection oligonucleotide 3210 is indirectly attached to a detection moiety 3200, e.g., via a tether oligonucleotide 3410. In some embodiments, the detection oligonucleotide includes a 5′ tethering region 3212 that hybridizes to a tether oligonucleotide 3410 attached to the detection moiety. In some embodiments, the detection oligonucleotide includes a 5′ tethering region 3212 that includes a nucleotide sequence that is complementary to at least a portion (or substantially all (e.g., at least 80%, 85%, 90%, 95%, 97%, 98%, or 99% or more, or optionally 100%)) of the tether oligonucleotide 3410 attached to the detection moiety. In some embodiments, the nucleotide sequence of the 5′ tethering region that is complementary to the tether oligonucleotide attached to the detection moiety is 10, 11, 12, 13, 14, 15, 16, 17, 18, 19, 20, 21, 22, 23, 24, 25, 26, 27, 28, 29, 30, 35, 40, 45, 50, 55, 60 nucleotides long, or longer, or a length in a range defined by any two of the preceding values (e.g., 10-60, 20-55, 15-50, 30-40 nucleotides long, etc.). In some embodiments, the nucleotide sequence of the 5′ tethering region that is complementary to the tether oligonucleotide attached to the detection moiety is about 15-50 nucleotides long. In some embodiments, the nucleotide sequence of the 5′ tethering region that is complementary to the tether oligonucleotide attached to the detection moiety is about 15-30 nucleotides long. In some embodiments, the nucleotide sequence of the 5′ tethering region that is complementary to the tether oligonucleotide attached to the detection moiety is about 20 nucleotides long.

In some embodiments, the orientation of either the capture oligonucleotide or the detection oligonucleotide is reversed to permit the ligation of the capture oligonucleotide and the detection oligonucleotide in proximity ligation assays as described herein. For example, the capture oligonucleotide may comprises the 3′ hybridization region and a 5′ tethering region and the detection oligonucleotide may comprise a 5′ hybridization region and a 3′ tethering region such that when the capture oligonucleotide and detection oligonucleotide are brought into proximity and hybridize to a splint oligonucleotide, the 3′ end of the capture oligonucleotide can be ligated to the 5′ end of the detection oligonucleotide (either directly or via a second splint oligonucleotide as described herein). As a further example, the capture oligonucleotide may comprise a 5′ hybridization region and a 3′ tethering region and the detection oligonucleotide may comprises a 3′ hybridization region and a 5′ tethering region such that when the capture oligonucleotide and detection oligonucleotide are brought into proximity and hybridize to a splint oligonucleotide, the 5′ end of the capture oligonucleotide can be ligated to the 3′ end of the detection oligonucleotide (either directly or via a second splint oligonucleotide as described herein).

In some embodiments, one or more of the capture oligonucleotide and the detection oligonucleotide comprises a primer binding region configured to bind a primer pair for amplifying the released on-target extension product. In some embodiments, where one or more of the capture oligonucleotide and the detection oligonucleotide comprises a 5′ tethering region, the 5′ tethering region includes the primer binding region or a portion thereof. In some embodiments, the primer binding region is partially in the 5′ tethering region. In some embodiments, the primer binding region is not in the 5′ tethering region.

In some embodiments, a splint oligonucleotide (e.g., the capture oligonucleotide, the detection oligonucleotide, the first splint oligonucleotide, and/or the second splint oligonucleotide) includes a unique molecular identifier (UMI). In some embodiments, the capture oligonucleotide and/or the detection oligonucleotide includes a unique molecular identifier (UMI). In some embodiments, the capture oligonucleotide and/or the detection oligonucleotide does not include a UMI. In some embodiments, the first splint oligonucleotide and/or the second splint oligonucleotide includes a unique molecular identifier (UMI). In some embodiments, the first splint oligonucleotide and/or the second splint oligonucleotide does not include a UMI. In some embodiments, omitting the UMI from the capture oligonucleotide and/or the detection oligonucleotide, or the first and/or second splint oligonucleotide, can reduce the frequency of mis-priming of the 3′ hybridizing region.

In some embodiments, determining the presence and/or amount, or the absence of the released on-target extension product comprises determining the number of UMI with distinct sequences associated with the splint oligonucleotide (e.g., the capture oligonucleotide and/or detection oligonucleotide, or the first splint oligonucleotide and/or second splint oligonucleotide). In some embodiments, where the capture oligonucleotide and/or the detection oligonucleotide includes a unique molecular identifier (UMI), determining the presence and/or amount, or the absence of the released on-target extension product comprises determining the number of UMI with distinct sequences associated with the capture oligonucleotide and/or detection oligonucleotide. In some embodiments, determining the presence and/or amount, or the absence of the released on-target extension product comprises determining the number of UMI with distinct sequences associated with the first splint oligonucleotide and/or second splint oligonucleotide. Any suitable UMI sequence can be used in the splint oligonucleotide (e.g., capture oligonucleotide and/or detection oligonucleotide, or the first splint oligonucleotide and/or second splint oligonucleotide). In some embodiments, the UMI is at least 5, at least 6, at least 7, at least 8, at least 9, at least 10, at least 11, at least 12, at least 13, at least 14, at least 15, at least 16, at least 17, or at least 18, nucleotides long. In some embodiments, the UMI is about 8-16 nucleotides long. In some embodiments, a collection of splint oligonucleotides (e.g., capture oligonucleotides and/or detection oligonucleotides, or the first splint oligonucleotides and/or second splint oligonucleotides) having the UMI includes a diverse collection of UMI nucleotide sequences such that no two molecules of the extension products (e.g., on-target extension products) generated by extension of the splint oligonucleotides (e.g., capture oligonucleotides and/or detection oligonucleotides, or the first splint oligonucleotides and/or second splint oligonucleotides) have the same UMI sequence (or such that the likelihood that any two molecules of the extension products (e.g., on-target extension products) have the same sequence is low enough to uniquely label the extension products (e.g., on-target extension products) that are sequenced). In some embodiments, a collection of splint oligonucleotides (e.g., capture oligonucleotides and/or detection oligonucleotides, or the first splint oligonucleotides and/or second splint oligonucleotides) having the UMI includes a random sequence of nucleotides in each UMI.

The oligonucleotides (e.g., capture oligonucleotide, detection oligonucleotide, tether oligonucleotide, splint oligonucleotide) can include any suitable nucleotides. In some embodiments, the oligonucleotides include DNA, RNA, and analogues and derivatives thereof. In some embodiments, the DNA or RNA includes a modified backbone or sugar. In some embodiments, the oligonucleotides include DNA or RNA comprising one or more locked nucleic acids (LNA) or peptide nucleic acids (PNA).

In some embodiments, the sequencing analysis of the extension products involves determining the sequencing depth (or average sequencing depth). In some embodiments, where the splint oligonucleotides (e.g., capture oligonucleotides and/or detection oligonucleotides, or the first splint oligonucleotides and/or second splint oligonucleotides) include the UMI, sequencing depth is determined based on an analysis of the UMI in the extension products. In some embodiments, where the capture oligonucleotide and/or detection oligonucleotide includes the UMI, sequencing depth is determined based on an analysis of the UMI in the extension product.

In some embodiments, the capture oligonucleotide and/or the detection oligonucleotide includes a barcode sequence. In some embodiments, the capture oligonucleotide includes, from 5′ to 3′: a tethering region, a barcode sequence, and the 3′ hybridizing region. In some embodiments, the detection oligonucleotide includes, from 5′ to 3′: a tethering region, a barcode sequence, and the 3′ hybridizing region. In some embodiments, the barcode sequence is 4, 5, 6, 7, 8, 9, 10, 11, 12, 13, 14, 15, 16, 17, 18 nucleotides long or longer. In some embodiments, the barcode sequence is about 5-15 nucleotides long.

In some embodiments, the capture oligonucleotide 3110 includes a barcode sequence 3114 that identifies a binding target of the capture moiety that is attached to the solid support 3100 to which the capture oligonucleotide is attached. In some embodiments, the barcode sequence 3114 identifies the capture moiety that is attached to the solid support 3100 to which the capture oligonucleotide is attached. In some embodiments, the detection oligonucleotide 3210 includes a barcode sequence 3214 that identifies a binding target of the detection moiety 3200 to which the detection oligonucleotide is attached. In some embodiments, the barcode sequence 3214 identifies the detection moiety 3200 to which the detection oligonucleotide is attached. In some embodiments, during analysis of an extension product sequence in the sequencing data, comparing the barcode sequence from the capture oligonucleotide with the barcode sequence from the detection oligonucleotide indicates whether the extension product was generated due to an on-target arrangement of the capture oligonucleotide and the detection oligonucleotide. In some embodiments, the 3′ hybridizing region identifies the binding target of the capture moiety associated with the capture oligonucleotide and with the detection moiety associated with the detection oligonucleotide.

Providing the solid support at block 1010 can be done using any suitable option. In some embodiments, providing the solid support comprises attaching the capture moiety and/or the capture oligonucleotide to the solid support. In some embodiments, providing the solid support comprises concurrently attaching the capture moiety and the capture oligonucleotide to the solid support. In some embodiments, providing the solid support includes attaching the capture moiety to the solid support first, then attaching the capture oligonucleotide to the solid support. In some embodiments, providing the solid support includes attaching the capture oligonucleotide to the solid support first, then attaching the capture moiety to the solid support.

In some embodiments, where the capture oligonucleotide is attached to the solid support via a tether oligonucleotide, providing the solid support comprises attaching the tether oligonucleotide to the solid support. In some embodiments, where the capture oligonucleotide is attached to the solid support via a tether oligonucleotide, the method includes providing the solid support by hybridizing the capture oligonucleotide to the tether oligonucleotide. In some embodiments, the tether oligonucleotide is attached to a member of a binding pair (e.g., is biotinylated) before hybridizing the capture oligonucleotide to the tether oligonucleotide. Hybridizing the capture oligonucleotide to the tether oligonucleotide and attaching the tether oligonucleotide to the solid support can be performed in any suitable order. In some embodiments, the tether oligonucleotide is attached to the solid support before hybridizing the capture oligonucleotide to the first tether oligonucleotide. In some embodiments, the tether oligonucleotide is attached to the solid support after hybridizing the capture oligonucleotide to the tether oligonucleotide.

In some embodiments, attaching the capture moiety to the solid support comprises combining the capture moiety configured to attach to the solid support (e.g., biotinylated capture moiety) with the solid support (e.g., streptavidin-coated solid support) in a coating solution. In some embodiments, attaching the capture oligonucleotide to the solid support comprises combining the capture oligonucleotide configured to attach to the solid support (e.g., biotinylated capture oligonucleotide) with the solid support (e.g., streptavidin-coated solid support) in a coating solution. In some embodiments, the capture moiety and the capture oligonucleotide are combined with the solid support in a coating solution.

In some embodiments, the capture moiety configured to attach to the solid support (e.g., biotinylated antibody) is combined in the coating solution at about 5 pM or more, e.g., about 10 pM or more, about 20 pM or more, about 30 pM or more, about 40 pM or more, about 50 pM or more, about 75 pM or more, about 100 pM or more, about 150 pM or more, about 200 pM or more, about 250 pM or more, about 300 pM or more, about 400 pM or more, about 500 pM or more, about 600 pM or more, about 700 pM or more, about 800 pM or more, about 900 pM or more, about 1,000 pM or more, about 2,000 pM or more, about 3,000 pM or more, about 4,000 pM or more, about 5,000 pM or more, 6,000 pM or more, about 7,000 pM or more, about 8,000 pM or more, about 9,000 pM or more, about 10,000 pM or more, about 20,000 pM or more, about 50,000 pM or more, or a concentration in a range defined by any two of the preceding values (e.g., about 5-50,000 pM, about 10-20,000 pM, about 50-10,000 pM, about 20-8,000 pM, about 500-10,000 pM, etc.). In some embodiments, the capture moiety (e.g., biotinylated antibody) is combined at about 50-10,000 pM. In some embodiments, the capture moiety (e.g., biotinylated antibody) is combined at about 500-10,000 pM.

In some embodiments, the capture moiety is an antibody (e.g., a full-length, biotinylated antibody), and the capture moiety configured to attach to the solid support (e.g., biotinylated antibody) is combined in the coating solution at about 0.001 μg/mL or more, e.g., about 0.005 μg/mL or more, about 0.01 μg/mL or more, about 0.02 μg/mL or more, about 0.05 g/mL or more, about 0.1 μg/mL or more, about 0.15 μg/mL or more, about 0.2 μg/mL or more, about 0.25 μg/mL or more, about 0.3 μg/mL or more, about 0.35 μg/mL or more, about 0.4 μg/mL or more, about 0.45 μg/mL or more, about 0.5 μg/mL or more, about 0.55 μg/mL or more, about 0.6 μg/mL or more, about 0.65 μg/mL or more, about 0.7 μg/mL or more, about 0.75 μg/mL or more, about 0.8 μg/mL or more, about 0.85 μg/mL or more, about 0.9 μg/mL or more, about 0.95 g/mL or more, about 1 μg/mL or more, or about 2 μg/mL or less, about 1.8 μg/mL or less, about 1.6 μg/mL or less, about 1.5 μg/mL or less, about 1.4 μg/mL or less, about 1.3 μg/mL or less, about 1.2 μg/mL or less, about 1.1 μg/mL or less, about 1.0 μg/mL or less, about 0.9 μg/mL or less, about 0.8 μg/mL or less, about 0.7 μg/mL or less, about 0.6 μg/mL or less, about 0.5 μg/mL or less, or a concentration in a range defined by any two of the preceding values (e.g., 0.001-2 μg/mL, 0.01-2 g/mL, 0.05-1.5 μg/mL, 0.08-1.5 μg/mL, 0.1-1 μg/mL, 0.3-0.7 μg/mL, 0.1-0.5 μg/mL, 0.5-1 g/mL, etc.). In some embodiments, the capture moiety is an antibody (e.g., a full-length, biotinylated antibody), and is combined at about 0.01-1 μg/mL. In some embodiments, the capture moiety is an antibody (e.g., a full-length, biotinylated antibody), and is combined at about 0.1-1 g/mL.

In some embodiments, the capture oligonucleotide is configured to attach to the solid support (e.g., a biotinylated capture oligonucleotide, or a capture oligonucleotide hybridized to a biotinylated tether oligonucleotide), and is combined in the coating solution at about 0.01 nM or more, e.g., about 0.05 nM or more, about 0.1 nM or more, about 0.2 nM or more, about 0.5 nM or more, about 0.75 nM or more, about 1 nM or more, about 1.5 nM or more, about 2 nM or more, about 2.5 nM or more, about 3 nM or more, about 4 nM or more, about 5 nM or more, about 10 nM or more, or at a concentration in a range defined by any two of the preceding values (e.g., about 0.01-10 nM, about 0.05-3 nM, about 0.1-1.5 nM, about 0.5-1 nM, about 0.5-2 nM, etc.). In some embodiments, the capture oligonucleotide is combined in the coating solution at about 0.1-2 nM.

In some embodiments, the solid support includes a bead (e.g., a streptavidin-coated magnetic or paramagnetic bead) as provided herein, and is combined in the coating solution at about 0.001 μg/mL or more, e.g., about 0.005 μg/mL or more, about 0.01 μg/mL or more, about 0.02 μg/mL or more, about 0.05 μg/mL or more, about 0.1 μg/mL or more, about 0.15 μg/mL or more, about 0.2 μg/mL or more, about 0.25 μg/mL or more, about 0.3 μg/mL or more, about 0.35 g/mL or more, about 0.4 μg/mL or more, about 0.45 μg/mL or more, about 0.5 μg/mL or more, about 0.55 μg/mL or more, about 0.6 μg/mL or more, about 0.65 μg/mL or more, about 0.7 μg/mL or more, about 0.75 μg/mL or more, about 0.8 μg/mL or more, about 0.85 μg/mL or more, about 0.9 μg/mL or more, about 0.95 μg/mL or more, about 1 μg/mL or more, or about 5 μg/mL or less, about 4 μg/mL or less, about 3.5 μg/mL or less, about 3 μg/mL or less, about 2.5 μg/mL or less, about 2 μg/mL or less, about 1.8 μg/mL or less, about 1.6 μg/mL or less, about 1.5 μg/mL or less, about 1.4 μg/mL or less, about 1.3 μg/mL or less, about 1.2 μg/mL or less, about 1.1 μg/mL or less, about 1.0 μg/mL or less, about 0.9 μg/mL or less, about 0.8 μg/mL or less, about 0.7 μg/mL or less, about 0.6 μg/mL or less, about 0.5 μg/mL or less, or a concentration in a range defined by any two of the preceding values (e.g., 0.001-5 μg/mL, 0.01-2 μg/mL, 0.05-1.5 μg/mL, 0.08-1.5 g/mL, 0.1-3 μg/mL, 0.3-0.7 μg/mL, 0.1-0.5 μg/mL, 0.5-1 μg/mL, etc.). In some embodiments, where the solid support is a bead, about 0.1-3 μg/mL of the solid support is combined.

Combining the capture moiety and/or the capture oligonucleotide with the solid support is carried out under any suitable condition. In some embodiments, combining the capture moiety and the capture oligonucleotide with the solid support is carried out in a coating solution that includes sodium chloride. In some embodiments, the coating solution includes about 100-1,000 mM sodium chloride. In some embodiments, the coating solution includes about 200-800 mM sodium chloride. In some embodiments, the coating solution includes about 250-750 mM sodium chloride. In some embodiments, the coating solution includes about 500 mM sodium chloride. In some embodiments, combining the capture moiety and the capture oligonucleotide with the solid support is carried out in a coating solution comprising bovine serum albumin (BSA), potassium phosphate dibasic, potassium phosphate monobasic, sodium chloride, and/or a detergent or surfactant. In some embodiments, combining the capture moiety and the capture oligonucleotide with the solid support is carried out in a coating solution comprising about 2.0% sucrose, about 2.0% BSA, about 2.1% potassium phosphate dibasic, about 0.5% potassium phosphate monobasic, about 0.04% kathon CG/ICP II, and about 0.022% Triton™ X-100 with about 500 mM NaCl.

In some embodiments, the capture moiety and/or the capture oligonucleotide is incubated with the solid support in the coating solution for, for about, or for at least, 10, 30, 45, or 60 minutes, 1.25, 1.5, 2, or 3 hours or more, or the contacting (or incubating) is for, for about, for not more than 12, 9, 6, 3, 2.5, or 2 hours, or for a length of time in a range defined by any two of the preceding values (e.g., 10 minutes to 12 hours, 10 minutes to 6 hours, 30 minutes to 3 hours, 1 hours to 3 hours, 1 hour to 9 hours, 10 minutes to 1 hour, etc.). In some embodiments, the complexing solution is incubated for about 30 minutes to about 6 hours. In some embodiments, the capture moiety and/or the capture oligonucleotide is incubated with the solid support for about 30 minutes to about 2 hours. In some embodiments, the capture moiety and/or the capture oligonucleotide is incubated with the solid support for about 1 hour.

In some embodiments, the capture moiety and/or the capture oligonucleotide is incubated with the solid support in the coating solution at a temperature of about 4° C. or higher, e.g., about 8° C. or higher, about 12° C. or higher, about 15° C. or higher, about 18° C. or higher, about 20° C. or higher, about 22° C. or higher, about 25° C. or higher, about 27° C. or higher, about 30° C. or higher, about 35° C. or higher, or about 50° C. or lower, e.g., about 45° C. or lower, about 40° C. or lower, about 37° C. or lower, about 35° C. or lower, about 32° C. or lower, about 30° C. or lower, about 28° C. or lower, about 26° C. or lower, about 23° C. or lower, about 20° C. or lower, about 15° C. or lower, or at a temperature in range defined by any two of the preceding values (e.g., 4-50° C., 15-35° C., 20-25° C., 12-20° C., 20-45° C., 15-30° C., etc.). In some embodiments, the capture moiety and/or the capture oligonucleotide is incubated with the solid support in the coating solution at a temperature of 12-28° C. In some embodiments, the capture moiety and/or the capture oligonucleotide is incubated with the solid support in the coating solution at a temperature of 15-25° C.

In some embodiments, after attaching the capture moiety and the capture oligonucleotide to the solid support (e.g., hybridizing the capture oligonucleotide to the tether oligonucleotide attached to the solid support), the method includes removing excess unattached capture oligonucleotide and/or unattached capture moiety. In some embodiments, removing excess unattached capture oligonucleotide and/or unattached capture moiety includes washing the solid support with a wash solution (e.g., a buffer solution). Washing the solid support can be done any suitable number of times. In some embodiments, the solid support is washed 1, 2, 3, 4, 5 or more times. In some embodiments, the solid support is washed 2-4 times. In some embodiments, the solid support is washed 3 times. In some embodiments, the washing involves an equivalent of about 1, 2, 3, 4, 5, or more volume exchanges with a wash solution. Any suitable wash solution can be used to wash the solid support after contacting with the sample. In some embodiments, the wash buffer includes phosphate-buffered saline (PBS) or PBS with polysorbate 20 (PBST).

Providing the detection conjugate at block 1020 can be done using any suitable option. In some embodiments, providing the detection conjugate comprises attaching the tether oligonucleotide to the detection moiety. In some embodiments, where the detection oligonucleotide is attached to the detection moiety via a tether oligonucleotide, providing the detection conjugate comprises hybridizing the detection oligonucleotide to the tether oligonucleotide. Hybridizing the detection oligonucleotide to the tether oligonucleotide and attaching the tether oligonucleotide to the detection moiety can be performed in any suitable order. In some embodiments, the tether oligonucleotide is attached to the detection moiety before hybridizing the detection oligonucleotide to the tether oligonucleotide. In some embodiments, the tether oligonucleotide is attached to the detection moiety after hybridizing the detection oligonucleotide to the tether oligonucleotide.

In some embodiments, hybridizing the detection oligonucleotide to the tether oligonucleotide comprises combining in a solution the detection oligonucleotide with the tether oligonucleotide at a molar ratio of at least about 1:1. In some embodiments, hybridizing the detection oligonucleotide to the tether oligonucleotide comprises combining in a solution the detection oligonucleotide with the tether oligonucleotide at a molar ratio of at least about 1.2:1, at least about 1.4:1, at least about 1.6:1, at least about 1.8:1, at least about 2:1, at least about 2.2:1, at least about 2.4:1, at least about 2.6:1, at least about 2.8:1, at least about 3:1, at least about 3.5:1, or at least about 4:1, or a ratio in a range defined by any two of the preceding values (e.g., about 1:1-4:1, about 1.6:1-3:1, about 1.8:2.2, about 1.2:1-3:1, etc.). In some embodiments, hybridizing the detection oligonucleotide to the tether oligonucleotide comprises combining in a solution the detection oligonucleotide with the tether oligonucleotide at a molar ratio of about 1:1-2:1.

In some embodiments, hybridizing the detection oligonucleotide to the tether oligonucleotide comprises combining in a solution the detection moiety comprising the tether oligonucleotide with the detection oligonucleotide, wherein the detection moiety is at a concentration in a range of about 5 nM to about 10 M. In some embodiments, hybridizing the detection oligonucleotide to the tether oligonucleotide comprises combining in a solution the detection moiety comprising the tether oligonucleotide with the detection oligonucleotide, wherein the detection moiety is at a concentration in a range of about 5 nM or more, e.g., about 10 nM or more, about 20 nM or more, about 30 nM or more, about 40 nM or more, about 50 nM or more, about 75 nM or more, about 100 nM or more, about 150 nM or more, about 200 nM or more, about 250 nM or more, about 300 nM or more, about 400 nM or more, about 500 nM or more, about 600 nM or more, about 700 nM or more, about 800 nM or more, about 900 nM or more, about 1,000 nM or more, about 2,000 nM or more, about 3,000 nM or more, about 4,000 nM or more, about 5,000 nM or more, 6,000 nM or more, about 7,000 nM or more, about 8,000 nM or more, about 9,000 nM or more, about 10,000 nM or more, or a concentration in a range defined by any two of the preceding values (e.g., about 5-10,000 nM, about 10-5,000 nM, about 50-3,000 nM, about 20-8,000 nM, 50-500 nM, etc.). In some embodiments, the detection moiety is combined at about 50-500 nM. In some embodiments, the detection moiety is combined at about 100-1000 nM.

The analyte-binding moiety (e.g., capture moiety and detection moiety, or the first and second moiety, as described herein) can be any suitable moiety that can bind to an analyte, and can both be bound (or can be bound simultaneously) to the analyte. In some embodiments, the analyte-binding moiety (e.g., capture moiety and/or the detection moiety, or the first and/or second moiety) binds specifically to an analyte (e.g., with an affinity (KD) of at least about 10⁻⁵, 10⁻⁶, 10⁻⁷, 10⁻⁸, 10⁻⁹, 10⁻¹⁰, or 10⁻¹¹ M, or lower KD value). In some embodiments, the analyte-binding moiety (e.g., capture moiety and/or the detection moiety, or the first and/or second moiety) is or includes, without limitation, an antibody or binding fragment thereof, a lectin, a receptor, a cofactor, a polynucleotide, an aptamer, a single chain protein binder, a peptide, a modified enzyme substrate, or a suicide inhibitor. In some embodiments, the capture moiety and/or the detection moiety is or includes an antibody or binding fragment thereof (e.g., scFv, Fab, F(ab′)2, etc.). In some embodiments, the analyte-binding moiety (e.g., capture moiety and/or the detection moiety, or the first and/or second moiety) is or includes a full-length antibody. In some embodiments, the analyte-binding moieties (e.g., capture moiety and detection moiety, or the first and second moiety) are each an antibody or binding fragment thereof, where both antibodies or binding fragment thereof can both be bound (or can be simultaneously bound) to an analyte. In some embodiments, the analyte-binding moiety is biotinylated. In some embodiments, the capture moiety is biotinylated.

The capture moiety and detection moiety can bind to any suitable analyte. In some embodiments, the analyte is a protein, a polypeptide, or a small molecule. In some embodiments, the analyte is a carbohydrate, a glycoprotein, or a glycan.

In some embodiments, the analyte is a cytokine. In some embodiments, the analyte is a pro-inflammatory cytokine. In some embodiments, the analyte is selected from: IFN-γ, Eotaxin-3, IL-15, IL-2Rα, IL-1β, TARC, IL-31, IL-33, IL-2, IP-10, IL-17a, Tie-2, IL-4, MIP-1α, TNF-β, VEGF-D, IL-6, MCP-1, VEGF-A, VEGF-C, IL-8, MDC, FLT-1/VEGFR-1, FGF (basic), IL-10, MCP-4, Granzyme A, IL-22, IL-12p70, GM-CSF, IL-27, IL-23, IL-13, IL-1α, IL-18, MIP-3α, IL-5, IL-21, Eotaxin, IL-7, PIGF, MIP-1β, IL-12/IL-23p40, IL-29/IFN-λ1.

In some embodiments, the analyte is an antibody. In some embodiments, the analyte is an antibody that is an IgA, IgE, IgD, IgG, or IgM.

### C. Multiplexing

In some embodiments, an analyte detection method of the present disclosure is a multiplexed method. As used herein, “multiplex” denotes parallel (or pooled) processing of two or more different assays (e.g., involving two or more different paired combinations of solid support and detection conjugate, or two or more different paired combinations of the first conjugate and second conjugate) in the same reaction during at least some portion of the method. In some embodiments, multiplexing involves analyzing in a parallel manner two or more different analytes in a sample. In some embodiments, multiplexing involves analyzing a parallel manner two or more different samples. In some embodiments, the method includes multiplex detection of the presence and/or amount, or the absence of, of about, or of at least 2, 3, 4, 5, 6, 7, 8, 9, 10, 12, 14, 16, 18, 20, 25, 30, 40, 50, 60, 70, 80, 90, 100, 120, 150, 200, 300, 400, 500, 1000, 1500, 2000, 5000, 10000 different analytes, or of, of about, or of at most 10000, 5000, 2000, 1500, 1000, 500, 400, 300, 200, 190, 180, 160, 140, 120, 100, 90, 80, 70, 60, 50, 40 different analytes, or a number of different analytes in a range defined by any two of the preceding values (e.g., about 2-500, about 2-400, about 2-200, about 5-150, about 10⁻¹⁰⁰, about 10⁻⁵⁰, about 30-50, about 300-1500, about 5000-10000, etc.). In some embodiments, different analytes are present in the sample at different concentrations. In some embodiments, a first analyte in the sample is (or is expected to be) present at a concentration that is, is about, or is at least 1.2, 1.5, 2, 2.5, 3, 4, 5, 6, 7, 8, 9, 10, 15, 20, 25, 30, 40, 50, 100, 150, 200, 250, 300, 400, 500, 600, 700, 800, 900, 1,000, 2,000, 5,000, 10,000, 20,000, 50,000, 100,000, 10⁶, 10⁷, 10⁸, 10⁹, fold or more higher than the concentration of a second analyte in the sample, or optionally at a concentration that is higher by a fold amount in a range defined by any two of the preceding values (e.g., 1.2-5 fold, 5-10 fold, 10-20 fold, 20-50 fold, 50-100 fold, 100-500 fold, 500-1,000 fold, 1,000-10,000 fold, 10,000-100,000 fold, 10⁵-10⁶ fold, 10⁶-10⁷ fold, 10⁷-10¹ fold, 10⁸-10⁹ fold, etc.). In some embodiments, a first analyte in the sample is (or is expected to be) present at a concentration that is 5-10,000 fold higher than the concentration of a second analyte in the sample.

In some embodiments, the method (e.g., a multiplexed analyte detection method) includes, providing: a plurality of paired combinations of the solid supports and the detection conjugates, wherein a binding target of the capture moiety and detection moiety of each paired combination is the same, and wherein different paired combinations of the plurality of paired combinations have different binding targets. As used herein, “different binding targets” includes structurally different molecules or structurally different portions of the same molecule. In some embodiments, different binding targets are different from each other due to a difference in structure of the molecule that effectively prevents moieties that do not constitute a paired combination from providing an on-target interaction in methods of the present disclosure. For example, in some embodiments, one paired combination whose moieties can be simultaneously bound to a protein, and another paired combination whose moieties can be simultaneously bound to the same protein (e.g., having the same amino acid sequence) having a different post-translational modification, have different binding targets, where neither of the moieties from one paired combination can be bound to the same molecule simultaneously, or otherwise can provide an on-target interaction with either of the moieties of the other paired combination. In some embodiments, different binding targets are different from each other due to moieties from each paired combination binding to different epitopes on the same molecule. For example, in some embodiments, one paired combination whose moieties can be simultaneously bound to an epitope on a molecule, and another paired combination whose moieties can be simultaneously bound to a different epitope on the same molecule, have different binding targets, where neither of the moieties from one paired combination can provide an on-target interaction with either of the moieties of the other paired combination. In some embodiments, the method (e.g., a multiplexed analyte detection method) includes, providing: a plurality of paired combinations of the solid supports and the detection conjugates, wherein a binding target of the capture moiety and detection moiety of each paired combination is the same, and wherein different paired combinations of the plurality of paired combinations have different first and/or second moieties.

In some embodiments, the method (e.g., a multiplexed analyte detection method) includes, providing: a plurality of paired combinations of the first conjugates and the second conjugates, wherein a binding target of the first moiety and second moiety of each paired combination is the same, and wherein different paired combinations of the plurality of paired combinations have different binding targets. Any suitable number of paired combinations of the solid supports and the detection conjugates (or of the first conjugate and second conjugate) can be provided. In some embodiments, the plurality of paired combinations includes about 2 or more, about 5 or more, about 10 or more, about 15 or more, about 20 or more, about 25 or more, about 30 or more, about 40 or more, about 50 or more, about 60 or more, about 70 or more, about 80 or more, about 90 or more, about 100 or more, about 150 or more, about 200 or more, about 300 or more, about 400 or more, about 500 or more, or a number in a range defined by any two of the preceding values (e.g., 2-500, 2-400, 2-200, 5-100, 10-50, 20-100, 30-50, etc.) paired combinations (e.g., different paired combinations having different binding targets). In some embodiments, the plurality of paired combinations includes 10⁻⁵⁰ paired combinations (e.g., different paired combinations having different binding targets). In some embodiments, the plurality of paired combinations includes 50-400 paired combinations (e.g., different paired combinations having different binding targets). In some embodiments, the plurality of paired combinations includes at least 5 paired combinations (e.g., different paired combinations having different binding targets).

In some embodiments, the 3′ hybridizing region of the splint oligonucleotides (e.g., capture oligonucleotide and detection oligonucleotide, or first splint oligonucleotide and second splint oligonucleotide) of each of the plurality of paired combinations have the same sequence (i.e., common hybridization regions). In some embodiments, the 3′ hybridizing region of the capture oligonucleotide and detection oligonucleotide of each of the plurality of paired combinations of the solid supports and detection conjugates have the same sequence (i.e., common hybridization regions).

In some embodiments, the 3′ hybridizing region of the splint oligonucleotides of at least 2, 3, 4, 5, 6, 7, 8, 9, 10, 11, 12, 13, 14, 15, 16, 17, 18, 19, 20, 50, 100, 200, 300, 400, 500 or more (or all) of the plurality of paired combinations, or optionally a number of the plurality of paired combinations in a range defined by any two of the preceding values (e.g., 2-500, 2-400, 50-400, 10-50, 20-100, 30-50, etc.), have different sequences (e.g., do not cross-hybridize, or are orthogonal to each other). In some embodiments, the 3′ hybridizing region of the capture oligonucleotide and detection oligonucleotide of at least 2, 3, 4, 5, 6, 7, 8, 9, 10, 11, 12, 13, 14, 15, 16, 17, 18, 19, 20, 50, 100, 200, 300, 400, 500 or more (or all) of the plurality of paired combinations, or optionally a number of the plurality of paired combinations in a range defined by any two of the preceding values (e.g., 2-500, 2-400, 50-400, 10-50, 20-100, 30-50, etc.), of the solid supports and detection conjugates have different sequences (e.g., do not cross-hybridize, or are orthogonal to each other). In some embodiments, the 3′ hybridizing region of the capture oligonucleotide and detection oligonucleotide of each of the plurality of paired combinations of the solid supports and detection conjugates have different sequences from the 3′ hybridizing region of the other plurality of paired combinations (e.g., the 3′ hybridizing regions do not cross-hybridize between different paired combinations; the paired combinations have unique 3′ hybridization regions).

In some embodiments, the 3′ hybridizing region of the capture oligonucleotide and detection oligonucleotide of a first paired combination of the plurality of paired combinations is not complementary to the 3′ hybridizing region of the detection oligonucleotide and capture oligonucleotide, respectively, of at least one other paired combination of the plurality of paired combinations. In some embodiments, the 3′ hybridizing region of the capture oligonucleotide and detection oligonucleotide of at most two, at most 3, at most 4, at most 5, at most 6, at most 7, at most 8, at most 9, at most 10, or more of the plurality of paired combinations of the solid supports and detection conjugates have the same sequence (e.g., can hybridize to each other but for the different binding targets of the capture and detection moieties of the different paired combinations). In some embodiments, the 3′ hybridizing region of the first splint oligonucleotide and second splint oligonucleotide of a first paired combination of the plurality of paired combinations is not complementary to the 3′ hybridizing region of the second splint oligonucleotide and first splint oligonucleotide, respectively, of at least one other paired combination of the plurality of paired combinations.

In some embodiments, the 3′ hybridizing region of the splint oligonucleotides (e.g., capture oligonucleotide and detection oligonucleotide, or first splint oligonucleotide and second splint oligonucleotide) of a paired combination of the plurality of paired combinations identifies a binding target that is different from a binding target identified by the 3′ hybridizing region of the splint oligonucleotides of at least one (e.g., at least 2, 3, 4, 5, 6, 7, 8, 9, 10, 11, 12, 13, 14, 15, 16, 17, 18, 19, 20, 50, 100, 200, 300, 400, 500, or all) other paired combination(s) of the plurality of paired combinations. In some embodiments, the 3′ hybridizing region of the capture oligonucleotide and detection oligonucleotide of a paired combination of the plurality of paired combinations of the solid supports and detection conjugates identifies a binding target that is different from a binding target identified by the 3′ hybridizing region of the capture oligonucleotide and detection oligonucleotide of at least one (e.g., at least 2, 3, 4, 5, 6, 7, 8, 9, 10, 11, 12, 13, 14, 15, 16, 17, 18, 19, 20, 50, 100, 200, 300, 400, 500, or all) other paired combination(s) of the plurality of paired combinations.

In some embodiments, the 3′ hybridizing region of the capture oligonucleotide and detection oligonucleotide of at least 2, 3, 4, 5, 6, 7, 8, 9, 10, 11, 12, 13, 14, 15, 16, 17, 18, 19, 20, 50, 100, 200, 300, 400, 500, or more of the plurality of paired combinations of the solid supports and detection conjugates identify different binding targets of each of the at least 2, 3, 4, 5, 6, 7, 8, 9, 10, 11, 12, 13, 14, 15, 16, 17, 18, 19, 20, 50, 100, 200, 300, 400, 500, or more paired combinations. In some embodiments, the 3′ hybridizing region of the capture oligonucleotide and detection oligonucleotide of each of the plurality of paired combinations of the solid supports and detection conjugates identifies the binding target of the corresponding paired combination.

In some embodiments, the 3′ hybridizing region of the splint oligonucleotides (e.g., capture oligonucleotide and detection oligonucleotide, or first splint oligonucleotide and second splint oligonucleotide) of a first paired combination has a Hamming distance of at least 2 relative to the 3′ hybridizing region of the splint oligonucleotides of at least one other paired combination of the plurality of paired combinations. In some embodiments, the 3′ hybridizing region of the capture oligonucleotide and/or detection oligonucleotide of the first paired combination has a Hamming distance of at least 2, at least 3, at least 4, at least 5, or more relative to the 3′ hybridizing region of the capture oligonucleotide and/or detection oligonucleotide, respectively, of at least one other paired combination of the plurality of paired combinations. In some embodiments, the 3′ hybridizing region of the first splint oligonucleotide and/or second splint oligonucleotide of the first paired combination has a Hamming distance of at least 2 relative to the 3′ hybridizing region of the first splint oligonucleotide and/or second splint oligonucleotide, respectively, of at least one other paired combination of the plurality of paired combinations.

In some embodiments, a first calculated ΔG of hybridization between the 3′ hybridizing regions of the splint oligonucleotides (e.g., capture oligonucleotide and detection oligonucleotide, or first splint oligonucleotide and second splint oligonucleotide) of the first paired combination is, or is about −4 kcal/mol or more negative than a second calculated ΔG of hybridization between the 3′ hybridizing region of one of the splint oligonucleotides of the first paired combination and the 3′ hybridizing region of one of the splint oligonucleotides of any of the other paired combination (or each of the other paired combinations) of the plurality of paired combinations. In some embodiments, a first calculated ΔG of hybridization between the 3′ hybridizing regions of the capture oligonucleotide and detection oligonucleotide of the first paired combination is, or is about −4 kcal/mol or more negative, e.g., about −5 kcal/mol, about −6 kcal/mol, or more negative than a second calculated ΔG of hybridization between: (1) the 3′ hybridizing region of the capture oligonucleotide of a paired combination and the 3′ hybridizing region of the detection oligonucleotide of any (or each) of the at least one other paired combination of the plurality of paired combinations; and/or (2) the 3′ hybridizing region of the detection oligonucleotide of the paired combination and the 3′ hybridizing region of the capture oligonucleotide of any (or each) of the at least one other paired combination of the plurality of paired combinations. In some embodiments, a first calculated ΔG of hybridization between the 3′ hybridizing regions of the capture oligonucleotide and detection oligonucleotide of the first paired combination is less than a second calculated ΔG of hybridization between each of: (1) the 3′ hybridizing region of the capture oligonucleotide of a paired combination and the 3′ hybridizing region of the detection oligonucleotide of each of the at least one other paired combination of the plurality of paired combinations; and (2) the 3′ hybridizing region of the detection oligonucleotide of the paired combination and the 3′ hybridizing region of the capture oligonucleotide of each of the at least one other paired combination of the plurality of paired combinations, by at least 4 kcal/mol. In some embodiments, a first calculated ΔG of hybridization between the 3′ hybridizing regions of the first splint oligonucleotide and second splint oligonucleotide of the first paired combination is or is about −4 kcal/mol or more negative, e.g., about −5 kcal/mol, about −6 kcal/mol, or more negative than a second calculated ΔG of hybridization between: (1) the 3′ hybridizing region of the first splint oligonucleotide of the first paired combination and the 3′ hybridizing region of the second splint oligonucleotide of any (or each) of the at least one other paired combination of the plurality of paired combinations; and/or (2) the 3′ hybridizing region of the second splint oligonucleotide of the first paired combination and the 3′ hybridizing region of the first splint oligonucleotide of any (or each) of the at least one other paired combination of the plurality of paired combinations. In some embodiments, a first calculated ΔG of hybridization between the 3′ hybridizing regions of the first splint oligonucleotide and second splint oligonucleotide of the first paired combination is less than a second calculated ΔG of hybridization between each of: (1) the 3′ hybridizing region of the first splint oligonucleotide of the first paired combination and the 3′ hybridizing region of the second splint oligonucleotide of each of the at least one other paired combination of the plurality of paired combinations; and (2) the 3′ hybridizing region of the second splint oligonucleotide of the first paired combination and the 3′ hybridizing region of the first splint oligonucleotide of each of the at least one other paired combination of the plurality of paired combinations, by at least 4 kcal/mol. The ΔG of hybridization can be determined using any suitable option, e.g., as set forth in Wang et al., Nucleic Acids Research, Volume 47, Issue W1, 2 Jul. 2019, Pages W610-W613. In some embodiments, a first calculated ΔG of hybridization between the 3′ hybridizing regions of the splint oligonucleotides (e.g., capture oligonucleotide and detection oligonucleotide, or first splint oligonucleotide and second splint oligonucleotide) of the first paired combination is, is about, or is at least −4 kcal/mol or more negative than a second calculated ΔG of hybridization between the 3′ hybridizing region of one of the splint oligonucleotides of the first paired combination and the 3′ hybridizing region of one of the splint oligonucleotides of at least one, e.g., at least 1, 2, 3, 4, 5, 6, 7, 8, 9, 10, 15, 20, 25, 30, 40, 50, 100, 200, 300, 400, 500, or more, other paired combinations, or a number in a range defined by any two of the preceding values (e.g., 1-500, 1-400, 1-200, 10-100, 5-50, 5-40, 50-400, etc.), or of each of the other paired combinations, of the plurality of paired combinations.

The 3′ hybridizing regions of the capture oligonucleotide and the detection oligonucleotide can be designed using any suitable option. In some embodiments, the 3′ hybridizing regions of the capture oligonucleotide and the detection oligonucleotide are designed in silico. In some embodiments, designing the 3′ hybridizing regions of the capture oligonucleotide and the detection oligonucleotide takes into account the calculated hybridization energy. In some embodiments, 3′ hybridizing regions that can be paired for use in a multiplex assay format of the present methods (e.g., analyte detection methods) are designed using any suitable option. In some embodiments, designing 3′ hybridizing regions suitable for use in a multiplex assay format of the present methods (e.g., analyte detection methods) includes screening for hybridization specificity (or “orthogonality”) of candidate 3′ hybridizing regions. In some embodiments, screening for hybridization specificity of candidate 3′ hybridizing regions includes using a hybridization specificity method as provided herein.

In some embodiments, preparing the complexing solution in block 1030 (with reference to FIG. 1) comprises: (i) combining in a solution the solid supports and the detection conjugates of the plurality of paired combinations with the sample; or (ii) contacting the solid supports of the plurality of paired combinations with the sample, and then combining in a solution the sample-contacted solid supports and the detection conjugates of the plurality of paired combinations; or (iii) contacting the detection conjugates of the plurality of paired combinations with the sample, and then combining in a solution the sample-contacted detection conjugates and the solid supports of the plurality of paired combinations.

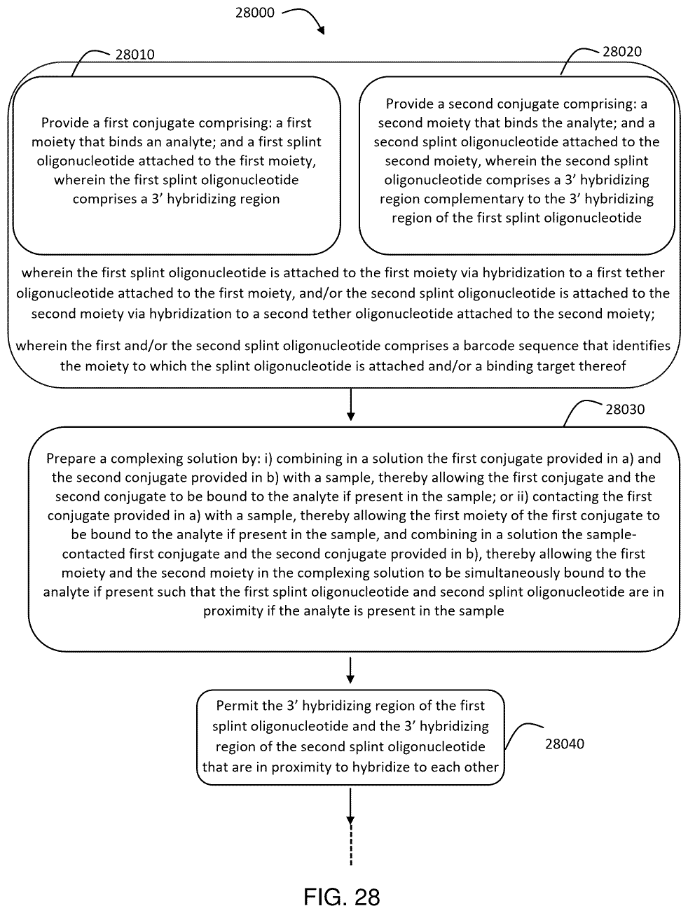

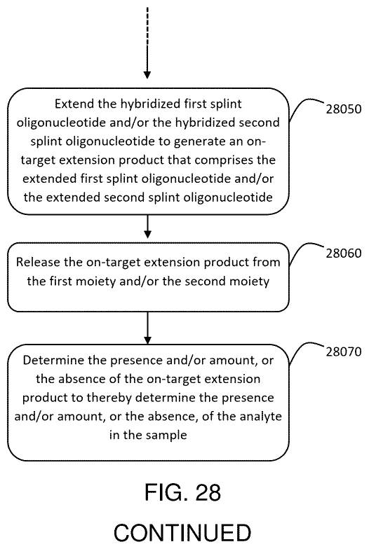

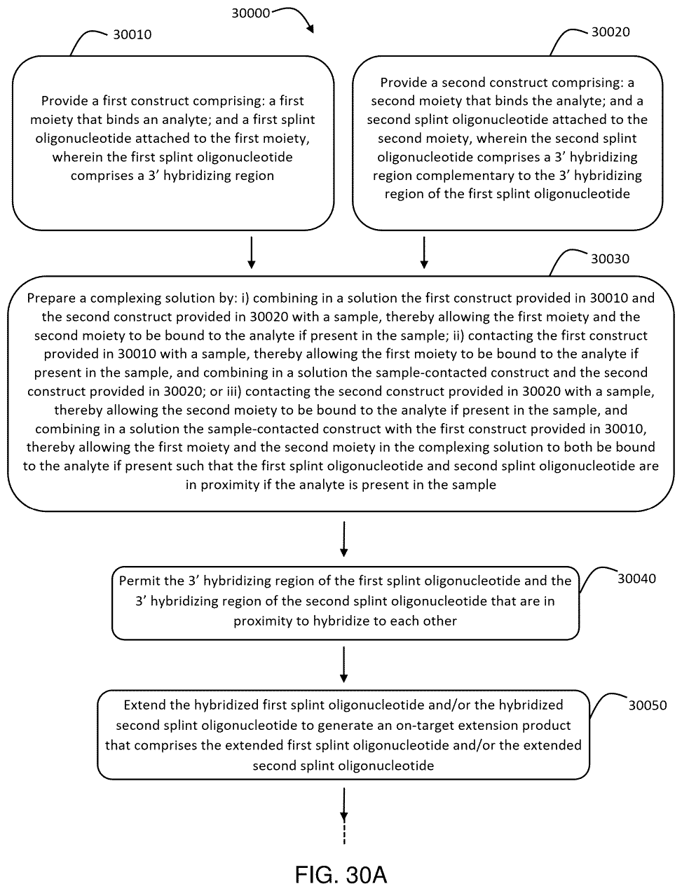

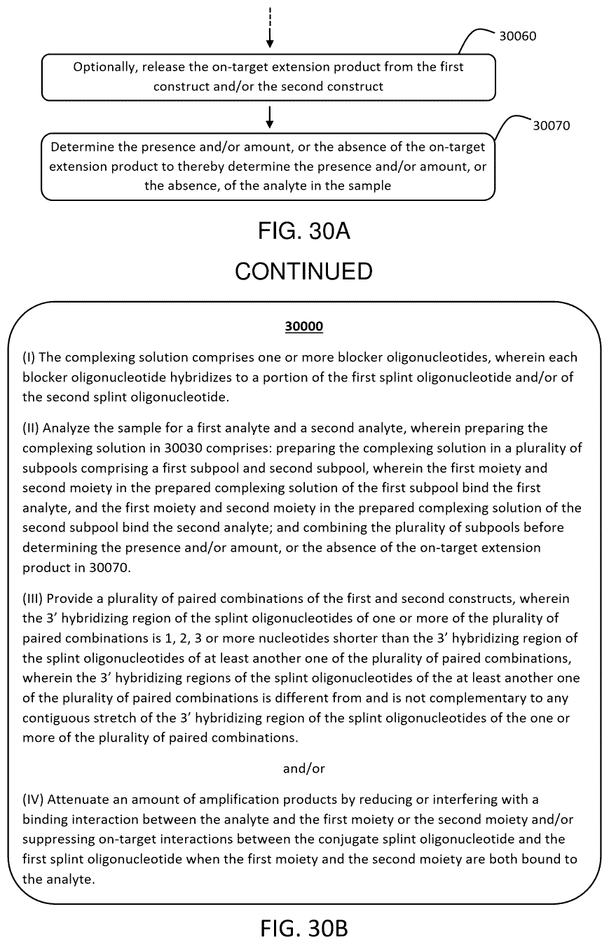

In some embodiments, clonally distinct conjugates (e.g., first or second conjugates, or detection conjugates, or first and/or second constructs that are a conjugate) are provided in spatially distinct partitions (e.g., separate wells of a multi-well plate) before preparing the complexing solution in block 1030 or block 28030 or block 30030 (with reference to FIG. 1 or 28 or 30, respectively). In some embodiments, the clonally distinct conjugates in the spatially distinct partitions are pooled before preparing the complexing solution in block 1030 or block 28030 or block 30030 (with reference to FIG. 1 or 28 or 30, respectively). In some embodiments, clonally distinct detection conjugates are initially provided in spatially distinct partitions (e.g., separate wells of a multi-well plate), and then pooled before adding to the sample-contacted solid support (or to the sample). In some embodiments, the method includes providing a plurality of spatially distinct partitions (e.g., separate wells of a multi-well plate), each comprising at least one detection conjugate of the plurality of paired combinations of the solid supports and the detection conjugates, wherein the detection moiety of the at least one detection conjugate in a partition of the plurality of spatially distinct partitions has a different binding target from the detection moiety of the at least one detection conjugate in a different partition of the plurality of spatially distinct partitions. In some embodiments, the method includes pooling the detection conjugates in the plurality of spatially distinct partitions before preparing the complexing solution in block 1030 (with reference to FIG. 1) or in block 30030 (with reference to FIG. 30). In some embodiments, the method includes providing a plurality of spatially distinct partitions, each comprising at least one first or second conjugate of the plurality of paired combinations of the first conjugates and the second conjugates, wherein the first or second moiety of the at least one first or second conjugate in a partition of the plurality of spatially distinct partitions has a different binding target from the first or second moiety of the at least one first or second conjugate in a different partition of the plurality of spatially distinct partitions. In some embodiments, preparing the complexing solution (for example, at block 28030, with reference to FIG. 28, or at block 30030, with reference to FIG. 30) includes contacting portions of the sample with each of the first or second conjugates in the plurality of spatially distinct partitions, then pooling the first or second conjugates in the plurality of spatially distinct partitions, and then contacting the pooled first conjugates with the second conjugates of the plurality of paired combinations or contacting the pooled second conjugates with the first conjugates of the plurality of paired combinations.

In some embodiments, clonally distinct solid supports are initially provided in spatially distinct partitions (e.g., separate wells of a multi-well plate), and then pooled before adding to the sample (or the sample-contacted detection conjugate). In some embodiments, the method includes providing a plurality of spatially distinct partitions, each comprising at least one solid support of the plurality of paired combinations of the solid supports and the detection conjugates, wherein the capture moiety attached to a solid support in a partition of the plurality of spatially distinct partitions has a different binding target from the capture moiety attached to a solid support in a different partition of the plurality of spatially distinct partitions. In some embodiments, the method includes pooling the solid supports in the plurality of spatially distinct partitions before preparing the complexing solution in block 1030 (with reference to FIG. 1) or in block 30030 (with reference to FIG. 30). In some embodiments, the sample can be contacted with the solid supports in the spatially distinct partitions. In some embodiments, preparing the complexing solution in block 1030 (or block 30030) comprises: contacting portions of the sample (e.g., portions evenly divided across the plurality of spatially distinct partitions) with each of the solid supports in the plurality of spatially distinct partitions, then pooling the solid supports in the plurality of spatially distinct partitions, and then contacting the pooled solid supports with the detection conjugates of the plurality of paired combinations.

Any suitable spatially distinct partitions can be used. In some embodiments, the plurality of spatially distinct partitions comprises a plurality of microtubes, microwells, and/or microfluidic chambers.

In some embodiments, a barcode that provides identification of the analyte-binding moiety (e.g., the capture moiety or detection moiety, or the first or second moieties) associated with the barcode, or of the binding target thereof, can increase specificity of the assay in multiplex format. In some embodiments, each splint oligonucleotide (e.g., capture oligonucleotide and detection oligonucleotide, or first splint oligonucleotide and second splint oligonucleotide) comprises a barcode sequence that identifies a binding target of the respective analyte-binding moiety with which the splint oligonucleotide is associated.

In some embodiments, each capture oligonucleotide attached to a solid support of the plurality of the solid supports comprises a barcode sequence that identifies a binding target of the capture moiety attached to the respective solid support. In some embodiments, barcode sequences of capture oligonucleotides that are attached to solid supports attached to capture moieties having the same binding target have the same barcode sequence. In some embodiments, each detection oligonucleotide attached to a detection moiety of the plurality of the detection conjugates comprises a barcode sequence that identifies a binding target of the respective detection moiety. In some embodiments, barcode sequences of splint oligonucleotides (e.g., detection oligonucleotides, first splint oligonucleotides or second splint oligonucleotides) that are attached to analyte-binding moieties having the same binding target have the same barcode sequence. In some embodiments, barcode sequences of detection oligonucleotides that are attached to detection moieties having the same binding target have the same barcode sequence. In some embodiments, both the capture oligonucleotide and the detection oligonucleotide include a barcode sequence. In some embodiments, the capture oligonucleotide includes a capture barcode sequence that identifies the binding target of the capture moiety that is attached to the solid support to which the capture oligonucleotide is also attached, and the detection oligonucleotide includes a detection barcode sequence that identifies the binding target of the detection moiety to which the detection oligonucleotide is attached.

In some embodiments, barcode sequences associated with different paired combinations (e.g., combinations of solid supports and detection conjugates having the same binding target, combinations of first conjugate and second conjugate having the same binding target) are provided in a lookup table that lists the binding target associated with each barcode sequence. In some embodiments, a barcode sequence of a splint oligonucleotide associated with an analyte-binding moiety can be used to identify in the lookup table the barcode sequence of a splint oligonucleotide associated with another analyte-binding moiety having the same binding target. For example, a sequenced extension product may include a barcode sequence for a splint oligonucleotide associated with an analyte-binding moiety, and the lookup table may be used to determine the corresponding analyte-binding moiety of the paired combination that includes the analyte-binding moiety associated with the identified barcode sequence. This information can then be used to determine if the sequenced extension product includes the correct barcode sequence of a splint oligonucleotide associated with the corresponding analyte-binding moiety of the paired combination and if so, determine that the sequence is from an on-target extension product. If the sequence extension product does not include the correct barcode sequence of the associated splint oligonucleotide, then it is determined that the sequence is from an off-target extension product. In some embodiments, a barcode sequence of a detection oligonucleotide attached to a detection moiety that has the same binding target as a capture moiety is identifiable based on the barcode sequence of a capture oligonucleotide attached to a solid support to which the capture moiety is attached. In some embodiments, a barcode sequence of a capture oligonucleotide attached to a solid support to which a capture moiety having the same binding target as a detection moiety is identifiable based on the barcode sequence of a detection oligonucleotide attached to the detection moiety.

In some embodiments, barcode sequences of two different splint oligonucleotides (e.g., two different capture oligonucleotides, two different detection oligonucleotides, two different first splint oligonucleotides, or two different second splint oligonucleotides) associated with analyte-binding moieties having different binding targets have a Hamming distance of at least 3, at least 4, at least 5, or at least 6. In some embodiments, two different barcode sequences of capture oligonucleotides that are attached to solid supports that are attached to capture moieties having different binding targets have a Hamming distance of 3 or 4. In some embodiments, two different barcode sequences of capture oligonucleotides (e.g., barcode sequences of two different capture oligonucleotides) that are attached to solid supports that are attached to capture moieties having different binding targets have a Hamming distance of at least 3, at least 4, at least 5, or at least 6. In some embodiments, two different barcode sequences of capture oligonucleotides (e.g., barcode sequences of two different capture oligonucleotides) that are attached to solid supports that are attached to capture moieties having different binding targets have a Hamming distance of 3 or 4. In some embodiments, a barcode sequence of a capture oligonucleotide that is attached to a solid support attached to a capture moiety has a Hamming distance of at least 3, at least 4, at least 5, or at least 6 with a barcode sequence of a capture oligonucleotide that is attached to any other solid support that is attached to a capture moiety having a different binding target. In some embodiments, a barcode sequence of a capture oligonucleotide that is attached to a solid support attached to a capture moiety has a Hamming distance of 3 or 4 with a barcode sequence of a capture oligonucleotide that is attached to any other solid support that is attached to a capture moiety having a different binding target. In some embodiments, two different barcode sequences of detection oligonucleotides (e.g., barcode sequences of two different detection oligonucleotides) attached to detection moieties having different binding targets have a Hamming distance of at least 3, at least 4, at least 5, or at least 6. In some embodiments, two different barcode sequences of detection oligonucleotides (e.g., barcode sequences of two different detection oligonucleotides) attached to detection moieties having different binding targets have a Hamming distance of 3 or 4. In some embodiments, a barcode sequence of a detection oligonucleotide attached to a detection moiety has a Hamming distance of at least 3, at least 4, at least 5, or at least 6 with a barcode sequence of a detection oligonucleotide attached to any other detection moiety having a different binding target. In some embodiments, a barcode sequence of a detection oligonucleotide attached to a detection moiety has a Hamming distance of 3 or 4 with a barcode sequence of a detection oligonucleotide attached to any other detection moiety having a different binding target.

In general, extension of capture or detection oligonucleotides is more efficient from on-target arrangements than from off-target arrangements. In some embodiments, the on-target extension product from each paired combination of the plurality of paired combinations is at least about 10 times more abundant than off-target extension products (e.g., extension products that do not include a paired combination of the capture and detection oligonucleotides, or have the length that deviates from the expected length). In some embodiments, the presence of the analyte in the sample is determined with a specificity of about 99 parts in 100 or higher (e.g., mis-pairing between capture and detection oligonucleotides from different paired combinations occur at 1 part in 100 or less). As used herein, “specificity” denotes the frequency with which the on-target extension product is produced relative to any off-target extension products (due to mis-pairing) in the presence of one or more other analytes. In some embodiments, the presence of the analyte in the sample is determined with a specificity of about 99 parts in 100 or higher, e.g., about 995 parts in 1000, about 999 parts in 1,000, about 9,999 parts in 10,000, about 99,999 parts in 100,000, or about 999,999 parts in 1,000,000, or higher, or with a specificity in a range defined by any two of the preceding values (e.g., about 99 parts in 100 to 999,999 parts in 1,000,000, 999 parts in 1000 to 999,999 parts in 1,000,000, about 9,999 parts in 10,000 to 999,999 parts in 1,000,000, etc.).

Any suitable sample can be used. In some embodiments, the sample includes without limitation a clinical, environmental, or industrial sample. In some embodiments, the sample includes plasma, serum, blood, stool, urine, saliva, cerebral spinal fluid, or amniotic fluid. In some embodiments, the sample includes a tissue homogenate. In some embodiments, a sample has been processed from an original source. In some embodiments, a sample includes a sample that has been processed, e.g., purified, concentrated, etc. For example, a blood sample can be processed to be a plasma sample, which can be a sample suitable for use in the methods herein.

### D. Additional Analyte Detection Method Embodiments

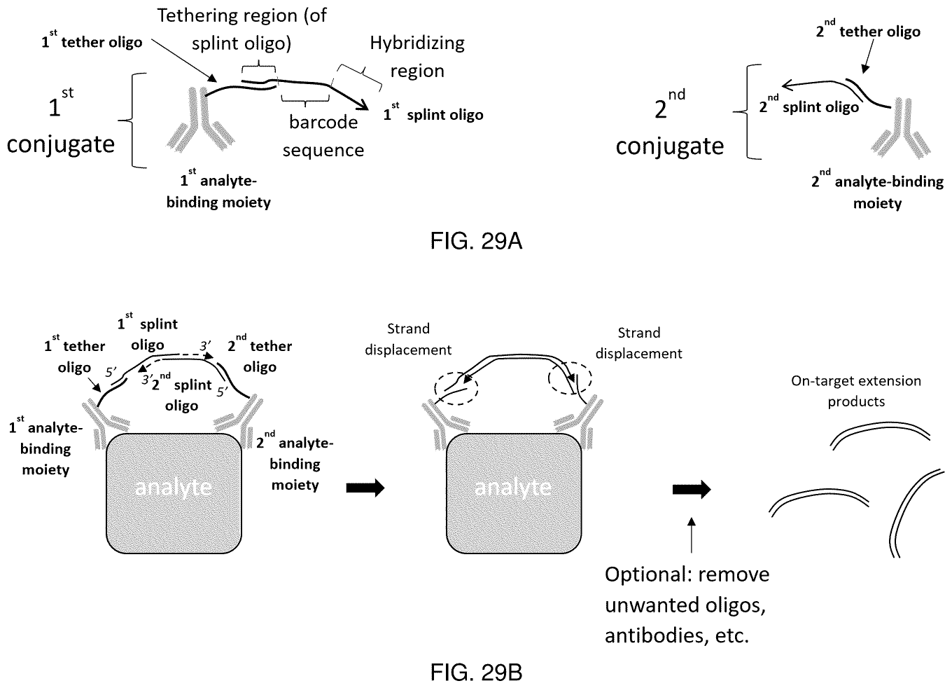

With reference to FIG. 28, a non-limiting example of a method 28000 of analyzing a sample for an analyte is provided. The method can include, at block 28010, providing a first conjugate comprising: a first moiety that binds an analyte; and a first splint oligonucleotide attached to the first moiety, wherein the first splint oligonucleotide comprises a 3′ hybridizing region. The method can further include, at block 28020, providing a second conjugate comprising: a second moiety that binds the analyte; and a second splint oligonucleotide attached to the second moiety, wherein the second splint oligonucleotide comprises a 3′ hybridizing region complementary to the 3′ hybridizing region of the first splint oligonucleotide. The first splint oligonucleotide can be attached to the first moiety via hybridization to a first tether oligonucleotide attached to the first moiety, and/or the second splint oligonucleotide can be attached to the second moiety via hybridization to a second tether oligonucleotide attached to the second moiety; wherein the first and/or the second splint oligonucleotide can include a barcode sequence that identifies the moiety to which the splint oligonucleotide is attached and/or a binding target thereof. The method can also include, at block 28030, preparing a complexing solution by: i) combining in a solution the first conjugate provided at block 28010 and the second conjugate provided at block 28020 with a sample, thereby allowing the first conjugate and the second conjugate to be bound to the analyte if present in the sample; or ii) contacting the first conjugate provided at block 28010 with a sample, thereby allowing the first moiety of the first conjugate to be bound to the analyte if present in the sample, and combining in a solution the sample-contacted first conjugate and the second conjugate provided at block 28020; thereby allowing the first moiety and the second moiety in the complexing solution to both be bound to the analyte if present such that the first splint oligonucleotide and second splint oligonucleotide are in proximity if the analyte is present in the sample. The method can include, at block 28040, permitting the 3′ hybridizing region of the first splint oligonucleotide and the 3′ hybridizing region of the second splint oligonucleotide that are in proximity to hybridize to each other. The method can also include, at block 28050, extending the hybridized first splint oligonucleotide and/or the hybridized second splint oligonucleotide to generate an on-target extension product that comprises the extended first splint oligonucleotide and/or the extended second splint oligonucleotide. The method can further include, at block 28060, releasing the on-target extension product from the first moiety and/or the second moiety. The method can also include, at block 28070, determining the presence and/or amount, or the absence of the on-target extension product to thereby determine the presence and/or amount, or the absence, of the analyte in the sample. In some embodiments, releasing the on-target extension product at block 28070 includes releasing from the first moiety and the second moiety. In some embodiments, the method of analyzing a sample for an analyte as provided in FIG. 28 can be carried out using any suitable options as described herein for the method of FIG. 1, taking into account any differences between the two non-limiting embodiments. For example, in the embodiment of FIG. 1, the capture oligonucleotide is attached to the solid support to which the capture moiety is also attached (and independently of the capture moiety), while in the embodiment of FIG. 28, the first splint oligonucleotide is attached to the first moiety without an intervening solid support, and the second splint oligonucleotide is attached to the second moiety without an intervening solid support (see, e.g., FIGS. 29A and 29B).

Providing the first conjugate at block 28010 and providing the second conjugate at block 28020 can be performed in any suitable order. In some embodiments, the first conjugate is provided before providing the second conjugate. In some embodiments, the second conjugate is provided before providing the first conjugate. In some embodiments, providing the first conjugate is done concurrently to providing the second conjugate.

The sample can include any suitable amount of the analyte of interest (including no detectable amount) as described herein. The sample can be prepared in any suitable solution, as described herein. Suitable options for providing the first conjugate, providing the second conjugate, and preparing a complexing solution, as described herein.

The complexing solution can be prepared using any suitable option. In some embodiments, preparing a complexing solution includes: contacting the first conjugate provided at block 28010 with a sample, thereby allowing the first moiety of the first conjugate to be bound to the analyte if present in the sample, and combining in a solution the sample-contacted first conjugate and the second conjugate provided at block 28020. Contacting the first conjugate provided at block 28010 with the sample can be performed in any suitable manner. In some embodiments, contacting includes incubating the first conjugate with the sample. In some embodiments, contacting includes adding the first conjugate to the sample. In some embodiments, contacting includes adding the sample to a partition (e.g., a microwell) containing the first conjugate. In some embodiments, the first conjugate is attached to a solid support (e.g., a bead, a microwell, etc.).

Any suitable amount of the first conjugate (or the first moiety and/or first splint oligonucleotide (e.g., as provided by the first conjugate)) can be contacted with the sample. In some embodiments, the first conjugate (e.g., antibody conjugate) is contacted with the sample at a final concentration per sample volume of, of about, or of at least 5, 10, 20, 30, 40, 50, 75, 100, 150, 200, 250, 300, 400, 500, 600, 700, 800, 900, 1,000, 2,000, 3,000, 4,000, 5,000 pM or more, 6,000, 7,000, 8,000, 9,000, 10,000, 20,000, or 50,000 pM, or a concentration in a range defined by any two of the preceding values (e.g., about 5-50,000 pM, about 10-20,000 pM, about 50-10,000 pM, about 20-8,000 pM, about 500-10,000 pM, etc.). In some embodiments, the first conjugate (e.g., antibody conjugate) is contacted with the sample at a concentration in a range of 50-10,000 pM. In some embodiments, the first conjugate (e.g., antibody conjugate) is contacted with the sample at a concentration in a range of 500-10,000 pM. In some embodiments, the first conjugate (e.g., antibody conjugate) is contacted with the sample at a concentration in a range of 1,000-3,000 pM.

In some embodiments, the first conjugate (e.g., an antibody conjugate) is contacted with the sample (e.g., contacted in a sample volume) at a concentration of, of about, or of at least 0.001, 0.005, 0.01, 0.02, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 μg/mL or more, or of, of about, or of at most 2, 1.8, 1.6, 1.5, 1.4, 1.3, 1.2, 1.1, 1.0, 0.9, 0.8, 0.7, 0.6, or 0.5 μg/mL or less, or a concentration in a range defined by any two of the preceding values (e.g., 0.001-2 μg/mL, 0.01-2 μg/mL, 0.05-1.5 μg/mL, 0.08-1.5 g/mL, 0.1-1 μg/mL, 0.3-0.7 μg/mL, 0.1-0.5 μg/mL, 0.5-1 μg/mL, etc.). In some embodiments, the first conjugate is an antibody conjugate (e.g., a full-length antibody conjugate), and is present at about 0.01-1 μg/mL in the sample volume when contacted with the sample. In some embodiments, the first conjugate is an antibody conjugate (e.g., a full-length antibody conjugate), and is present at about 0.1-1 μg/mL in the sample volume when contacted with the sample. In some embodiments, the concentration is based on the concentration of the first moiety portion of the first conjugate (e.g., excluding the contribution of the mass of the splint oligonucleotide).

In some embodiments, the method further includes removing the sample before combining in the solution the sample-contacted first conjugate and the second conjugate provided at block 28020. Removing the sample can be done using any suitable option, as described herein. In some embodiments, the first conjugate is attached to a solid support, and removing the sample includes washing the solid support, as described herein.

In some embodiments, preparing the complexing solution includes, following contacting the first conjugate with the sample, combining in a solution the sample-contacted first conjugate and the second conjugate provided at block 28020, using any suitable option. In some embodiments, the combining includes adding the sample-contacted first conjugate to a solution comprising the second conjugate. The sample-contacted first conjugate and the second conjugate can be combined under any suitable condition to allow the first moiety and the second moiety to both be bound to the analyte if present in the sample. In some embodiments, combining the sample-contacted first conjugate and the second conjugate includes incubating the solution under a suitable condition to allow the first moiety and the second moiety to both be bound to the analyte if present in the sample.

The sample-contacted first conjugate and the second conjugate provided at block 28020 can be combined in any suitable solution, as described herein. In some embodiments, the sample-contacted first conjugate and the second conjugate are combined in a solution comprising a carrier protein, a surfactant, a buffer, a salt, and/or other additives to inhibit nonspecific binding of second conjugates to the sample-contacted first conjugate. In some embodiments, the sample-contacted first conjugate and the second conjugate are combined in a solution comprising one or more blockers. Suitable blockers include, without limitation, mouse IgG, BSA, casein, and salmon sperm DNA.

Any suitable amount of the second conjugate can be combined with the sample-contacted first conjugate. In some embodiments, the second conjugate (e.g., an antibody conjugate) is combined with the complexing solution at a concentration of, of about, or of at least 5, 10, 20, 30, 40, 50, 75, 100, 150, 200, 250, 300, 400, 500, 600, 700, 800, 900, 1,000, 2,000, 3,000, 4,000, 5,000 pM or more, 6,000, 7,000, 8,000, 9,000, 10,000, 20,000, or 50,000 pM, or a concentration in a range defined by any two of the preceding values (e.g., about 5-50,000 pM, about 10-20,000 pM, about 50-10,000 pM, about 20-8,000 pM, about 500-10,000 pM, etc.). In some embodiments, the second conjugate (e.g., antibody conjugate) is combined with the complexing solution at a concentration in a range of 50-10,000 pM. In some embodiments, the second conjugate (e.g., antibody conjugate) is combined with the complexing solution a concentration in a range of 500-10,000 pM. In some embodiments, the second conjugate (e.g., antibody conjugate) is combined with the complexing solution at a concentration in a range of 1,000-3,000 pM.

In some embodiments, the second conjugate (e.g., an antibody conjugate) is combined with the complexing solution at a concentration of, of about, or of at least 0.001, 0.005, 0.01, 0.02, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 μg/mL or more, or of, of about, or of at most 2, 1.8, 1.6, 1.5, 1.4, 1.3, 1.2, 1.1, 1.0, 0.9, 0.8, 0.7, 0.6, or 0.5 μg/mL or less, or a concentration in a range defined by any two of the preceding values (e.g., 0.001-2 μg/mL, 0.01-2 μg/mL, 0.05-1.5 μg/mL, 0.08-1.5 μg/mL, 0.1-1 g/mL, 0.3-0.7 μg/mL, 0.1-0.5 μg/mL, 0.5-1 μg/mL, etc.). In some embodiments, the second conjugate is an antibody conjugate (e.g., a full-length antibody conjugate), and is present at about 0.01-1 μg/mL. In some embodiments, the second conjugate is an antibody conjugate (e.g., a full-length antibody conjugate), and is present at about 0.1-1 μg/mL. In some embodiments, the concentration is based on the concentration of the second moiety portion of the second conjugate (e.g., excluding the contribution of the mass of the splint oligonucleotide).

In some embodiments, preparing the complexing solution comprises combining in a solution the first conjugate provided at block 28010 and the second conjugate provided at block 28020 with the sample, thereby allowing the first conjugate and the second conjugate to be bound to the analyte if present in the sample. Combining the first conjugate provided at block 28010 and the second conjugate provided at block 28020 with the sample can be performed in any suitable manner. In some embodiments, combining is performed sequentially (e.g., contacting the first conjugate provided at block 28010 with the sample, and then combining the sample-contacted first conjugate with the second conjugate provided at block 28020, as provided above). In some embodiments, combining is performed concurrently (e.g., the first conjugate provided at block 28010 and the second conjugate provided at block 28020 are combined with the sample before incubation with either is carried out for a substantial amount of time (for example, not more than 30%, 25%, 20%, 15%, 10%, 5%, 4%, 3%, 2%, or 1% of the total amount of time for incubation)). In some embodiments, the first conjugate and the second conjugate are combined, and then the combination is combined with the sample. In some embodiments, the first conjugate is contacted with the sample, and then the second conjugate is combined with the combination of the first conjugate and the sample.

Extending the hybridized first splint oligonucleotide and/or the hybridized second splint oligonucleotide at block 28050 can be carried out in any suitable manner. In some embodiments, the extending at block 28050 is performed by a polymerase. As discussed herein, extending a splint oligonucleotide that is hybridized to a paired counterpart splint oligonucleotide can generate an on-target extension product when the corresponding analyte-binding moieties are both bound to the analyte. In some embodiments, the extending at block 28050 includes treating the on-target extension product with a polymerase. Any suitable polymerase can be used, as described herein. In some embodiments, the extending at block 28050 is performed by a strand-displacing polymerase. Any suitable strand-displacing polymerase can be used. In some embodiments, the strand-displacing polymerase is a 3-5′ exo-polymerase. In some embodiments, the strand-displacing polymerase is a Klenow fragment. In some embodiments, the strand-displacing polymerase is an exo-Klenow fragment.

In some embodiments, releasing the on-target extension product comprises treating the on-target extension product with a restriction enzyme, a protease, and/or a high-stringency wash, as described herein.

In some embodiments, at least one or both of the first and second splint oligonucleotides are attached to their respective moiety via hybridization to a tether oligonucleotide attached to the moiety. In some embodiments, both of the first and second splint oligonucleotides are attached to their respective moiety via hybridization to a tether oligonucleotide attached to the moiety. In some embodiments, the first splint oligonucleotide is attached to the first moiety via hybridization to a first tether oligonucleotide attached to the first moiety, and the second splint oligonucleotide is attached to the second moiety via hybridization to a second tether oligonucleotide attached to the second moiety. In some embodiments, the extending and releasing are performed by a single enzyme. In some embodiments, the releasing does not require using a protease or restriction enzyme. In some embodiments, extending and releasing are performed by the same enzyme. In some embodiments, the single enzyme comprises a strand-displacing polymerase, as described herein. In some embodiments, the releasing at block 28060 is performed at a temperature in a range of 10⁻³⁷° C. In some embodiments, the extending at block 28050 comprises contacting the hybridized first splint oligonucleotide and/or the hybridized second splint oligonucleotide with a strand-displacing polymerase under conditions sufficient to extend the hybridized first splint oligonucleotide and/or the hybridized second splint oligonucleotide, and wherein the releasing at block 28060 comprises allowing the strand-displacing polymerase to displace the first tether oligonucleotide hybridized to the first splint oligonucleotide during extension, and/or to displace the second tether oligonucleotide hybridized to the second splint oligonucleotide during extension. In some embodiments, the extending at block 28050 comprises contacting the hybridized first splint oligonucleotide and the hybridized second splint oligonucleotide with a strand-displacing polymerase under conditions sufficient to extend the hybridized first splint oligonucleotide and the hybridized second splint oligonucleotide, and wherein the releasing at block 28060 comprises allowing the strand-displacing polymerase to displace the first tether oligonucleotide hybridized to the first splint oligonucleotide during extension, and to displace the second tether oligonucleotide hybridized to the second splint oligonucleotide during extension. In some embodiments, the strand-displacing polymerase is allowed to displace the first tether oligonucleotide hybridized to the first splint oligonucleotide during extension, and to displace the second tether oligonucleotide hybridized to the second splint oligonucleotide during extension.

In some embodiments, the tether oligonucleotide is covalently attached to a first member of a binding pair that binds to a second member of the binding pair, wherein the second member is attached to the corresponding moiety (e.g., via a covalent interaction between the second member and the corresponding moiety). In some embodiments, the tether oligonucleotide is covalently attached to the corresponding moiety. Any suitable binding pair can be used, as described herein (e.g., biotin/streptavidin, etc.). In some embodiments, the first member of the binding pair comprises biotin, and the second member of the binding pair comprises streptavidin.

In some embodiments, the splint oligonucleotide (e.g., the first and/or second splint oligonucleotide) is attached to the corresponding moiety (e.g., the first and/or second moiety) via a bonding interaction that is independent of the nucleotide sequence in the splint oligonucleotide. In some embodiments, a bonding interaction that is independent of the nucleotide sequence in the splint oligonucleotide is not disrupted by a strand displacing polymerase, e.g., upon extension. In some embodiments, the splint oligonucleotide (e.g., the first and/or second splint oligonucleotide) is covalently attached to the corresponding moiety (e.g., the first and/or second moiety). Any suitable option can be used to covalently attach the splint oligonucleotide to the corresponding moiety, as described herein (e.g., via amine-thiol crosslinking, maleimide crosslinking, N-hydroxysuccinimide or N-hydroxysulfosuccinimide, etc.). In some embodiments, either the first splint oligonucleotide is covalently attached to the first moiety, or the second splint oligonucleotide is covalently attached to the second moiety. In some embodiments, releasing at block 28060 comprises cleaving a covalent attachment of the on-target extension product to the first or second moiety. In some embodiments, releasing at block 28060 comprises contacting the on-target extension product with a protease. In some embodiments, either of the splint oligonucleotides (e.g., the first or second splint oligonucleotide) is covalently attached to a first member of a binding pair that binds to a second member of the binding pair, where the second member is attached to the corresponding moiety (e.g., via a covalent interaction between the second member and the corresponding moiety). Any suitable binding pair can be used, as described herein (e.g., biotin/streptavidin, etc.). In some embodiments, the first member of the binding pair comprises biotin, and the second member of the binding pair comprises streptavidin. In some embodiments, either of the splint oligonucleotides (e.g., the first or second splint oligonucleotide) is covalently attached to a first member of a binding pair that binds to a second member of the binding pair, and the corresponding moiety is attached to another first member of the binding pair, and the splint oligonucleotide is attached to the corresponding moiety via binding of the first member and the other first member to the second member of the binding pair.

In some embodiments, the method includes separating any unreleased and/or unextended splint oligonucleotides that remain attached to the first or second moieties from the released, on-target extension products. Without being bound by theory, it is believed that in some embodiments the unextended conjugates can interfere with the downstream PCR reactions as they can contain the primer sequences in some embodiments. In some embodiments, releasing the on-target extension products allows effective separation of product from unreacted conjugates. In some embodiments, the method includes providing a plurality of the first and second conjugates, wherein the method further comprises, following releasing the on-target extension product from the first moiety and/or the second moiety at block 28060 and before determining the presence and/or amount, or the absence of the on-target extension product at block 28070, separating the released on-target extension product from first conjugates of the plurality of first conjugates comprising the first splint oligonucleotide and from second conjugates of the plurality of second conjugates comprising the second splint oligonucleotide. In some embodiments, the first conjugates of the plurality of first conjugates includes an unextended first splint oligonucleotide. In some embodiments, the second conjugates of the plurality of second conjugates includes an unextended second splint oligonucleotide. In some embodiments, the method includes, following releasing the on-target extension product from the first moiety and/or the second moiety at block 28060 and before determining the presence and/or amount, or the absence of the on-target extension product at block 28070, separating the released on-target extension product from the first conjugates of the plurality of first conjugates comprising an unextended first splint oligonucleotide and from second conjugates of the plurality of second conjugates comprising an unextended second splint oligonucleotide. Any suitable option can be used to separate the released on-target extension product from the unreleased and/or unextended splint oligonucleotides. In some embodiments, the separating comprises size-exclusion chromatography, affinity chromatography, ion-exchange chromatography, and/or solid-phase reversible immobilization (SPRI). In some embodiments, the separating involves a size-based or filtration-based separation option. In some embodiments, the released product is smaller than the antibody conjugates. In some embodiments, the separating includes an affinity-based separation option, including, without limitation, protein G, protein A, or anti-species antibodies, to deplete the antibody conjugates without depleting the on-target extension product. In some embodiments, the separating includes a charge-based separation option, such as, without limitation, SPRI beads or ion-exchange membranes.

In some embodiments, either the first conjugate is attached to a solid support, or the second conjugate is attached to a solid support. In some embodiments, the first conjugate is attached to a first member of a binding pair that binds to a second member of the binding pair, wherein the second member is attached to the solid support, or wherein the second conjugate is attached to a first member of a binding pair that binds to a second member of the binding pair, wherein the second member is attached to the solid support. Any suitable binding pair can be used, as described herein (e.g., biotin/streptavidin, etc.). In some embodiments, the first member of the binding pair comprises biotin, and the second member of the binding pair comprises streptavidin. In some embodiments, the first conjugate is covalently attached to or is adsorbed onto the solid support, or wherein the second conjugate is covalently attached to or is adsorbed onto the solid support. Any suitable solid support can be used, as described herein. In some embodiments, where a conjugate (e.g., antibody conjugate) is attached to a solid support, the solid support does not include a splint oligonucleotide that is attached to the solid support independently of the analyte-binding moiety, or an analyte-binding moiety that is not conjugated to an oligonucleotide (e.g., a splint oligonucleotide, or a tether oligonucleotide).

In some embodiments, where at least one of the first and second conjugates is attached to a solid support, the method can include, following preparing the complexing solution in block 28030 and prior to the extending at block 28050, removing any first conjugate that is not bound to an analyte bound to a second conjugate attached to the solid support, or removing any second conjugate that is not bound to an analyte bound to a first conjugate attached to the solid support. In some embodiments, the removing comprises washing the solid support, optionally under high stringency conditions. In some embodiments, the method includes removing the sample before combining in the solution the sample-contacted first conjugate and the second conjugate provided at block 28020, thereby removing analyte if present that is not bound to the first moiety.

In some embodiments, the splint oligonucleotide (e.g., the first splint oligonucleotide and/or second splint oligonucleotide) includes one or more of: a barcode sequence, a tethering region, and a primer binding region. In some embodiments, the splint oligonucleotide includes from 5′ to 3′: a tethering region, the barcode sequence, and the 3′ hybridizing region.

In some embodiments, the 3′ hybridizing region of the first splint oligonucleotide and second splint oligonucleotide of each of the plurality of paired combinations of the first conjugates and the second conjugates identifies the binding target of the corresponding paired combination. In some embodiments, the splint oligonucleotide (e.g., the first splint oligonucleotide and/or second splint oligonucleotide) includes a barcode sequence that identifies the moiety to which the splint oligonucleotide is attached and/or a binding target thereof. Any suitable barcode sequence can be used, as described herein, e.g., for the capture oligonucleotide and/or the detection oligonucleotide. In some embodiments, the barcode sequence is 4, 5, 6, 7, 8, 9, 10, 11, 12, 13, 14, 15, 16, 17, 18 nucleotides long or longer, or a length in a range defined by any two of the preceding values (e.g., 4-18, 5-17, 6-15, 10-18 nucleotides long, etc.). In some embodiments, the barcode sequence is about 5-15 nucleotides long.

In some embodiments, the tethering region includes a sequence that hybridizes to a tether oligonucleotide attached to the analyte-binding moiety (e.g., the first moiety and/or the second moiety). In some embodiments, the tethering region includes a sequence that is complementary to a tether oligonucleotide attached to the analyte-binding moiety (e.g., the first moiety and/or the second moiety). In some embodiments, the tethering region includes the barcode sequence. Any suitable tethering region can be used, as described herein for the capture oligonucleotide and/or the detection oligonucleotide.

In some embodiments, the splint oligonucleotide (e.g., the first splint oligonucleotide and/or second splint oligonucleotide) includes a primer binding region configured to bind a primer pair for amplifying the released on-target extension product. In some embodiments, the splint oligonucleotide includes a 5′ tethering region that contains the primer binding region or a portion thereof. In some embodiments, the primer binding region is partially in the 5′ tethering region. In some embodiments, the primer binding region is not in the 5′ tethering region.

Determining the presence and/or amount, or the absence of the on-target extension product at block 28070 can be done using any suitable option, as described herein. In some embodiments, determining the presence and/or amount, or the absence of the on-target extension product comprises performing qPCR on one or more extension products generated at block 28050 and released at block 28060. In some embodiments, determining the presence and/or amount, or the absence of the on-target extension product comprises obtaining sequencing data (e.g., by sequencing) of one or more extension products generated at block 28050 and released at block 28060. Any suitable option for performing qPCR or obtaining sequencing data (e.g., sequencing) can be used, as described herein.

Also provided are methods of analyzing a sample having one or more non-limiting features that enhance performance of proximity-based assays and immunosequencing assays (e.g., multiplexed assays), including PESD. With reference to FIG. 30A, a method 30000 of analyzing a sample for an analyte can include, at block 30010, providing a first construct that includes a first moiety that binds an analyte; and a first splint oligonucleotide attached to the first moiety, wherein the first splint oligonucleotide comprises a 3′ hybridizing region. The method can include, at block 30020, providing a second construct that includes: a second moiety that binds the analyte; and a second splint oligonucleotide attached to the second moiety, wherein the second splint oligonucleotide comprises a 3′ hybridizing region complementary to the 3′ hybridizing region of the first splint oligonucleotide. The method can also include, at block 30030, preparing a complexing solution by: i) combining in a solution the first construct provided in 30010 and the second construct provided in 30020 with a sample, thereby allowing the first moiety and the second moiety to be bound to the analyte if present in the sample; ii) contacting the first construct provided in 30010 with a sample, thereby allowing the first moiety to be bound to the analyte if present in the sample, and combining in a solution the sample-contacted construct and the second construct provided in 30020; or iii) contacting the second construct provided in 30020 with a sample, thereby allowing the second moiety to be bound to the analyte if present in the sample, and combining in a solution the sample-contacted construct with the first construct provided in 30010, thereby allowing the first moiety and the second moiety in the complexing solution to both be bound to the analyte if present such that the first splint oligonucleotide and second splint oligonucleotide are in proximity if the analyte is present in the sample. The method can include, at block 30040, permitting the 3′ hybridizing region of the first splint oligonucleotide and the 3′ hybridizing region of the second splint oligonucleotide that are in proximity to hybridize to each other. The method can also include, at block 30050, extending the hybridized first splint oligonucleotide and/or the hybridized second splint oligonucleotide to generate an on-target extension product that comprises the extended first splint oligonucleotide and/or the extended second splint oligonucleotide. The method can also include, at block 30070, determining the presence and/or amount, or the absence of the on-target extension product to thereby determine the presence and/or amount, or the absence, of the analyte in the sample.

In any method of analyzing a sample for an analyte herein, in some embodiments, the first construct and the second construct are a first conjugate and second conjugate, respectively, as described herein. In any method of analyzing a sample for an analyte herein, in some embodiments, the first construct is a solid support comprising a capture moiety and a capture oligonucleotide attached thereto, and the second construct is a detection conjugate, as described herein. In some embodiments, the first and/or the second construct includes a nano-particle attached to the first and/or second moiety, respectively, and to the first and/or second splint oligonucleotide, respectively. In some embodiments, the first and/or the second construct includes a moiety attached to a first member (e.g., streptavidin) of a binding pair (e.g., biotin-streptavidin) that binds to a second member (e.g., biotin) of the binding pair, wherein the second member is attached to the first and/or second splint oligonucleotide, respectively. In some embodiments, the first and/or the second construct and the first and/or second splint oligonucleotide are attached to biotin, and respectively attached to each other via streptavidin.

In some embodiments, the method includes, at block 30060, releasing the on-target extension product from the first construct and/or the second construct. Any suitable option can be used to release the on-target extension product, as described herein. In some embodiments, the method does not include the releasing at block 30060.

The method 30000 can further include one or more of the following: (I) the complexing solution includes one or more blocker oligonucleotides (e.g., single-stranded oligonucleotides), wherein each blocker oligonucleotide hybridizes to a subpart of the first splint oligonucleotide and/or of the second splint oligonucleotide; (II) preparing the complexing solution in 30030 includes: preparing the complexing solution in a plurality of subpools comprising a first subpool and a second subpool, wherein the first moiety and second moiety in the prepared complexing solution of the first subpool bind the first analyte, and the first moiety and second moiety in the prepared complexing solution of the second subpool bind the second analyte; and combining the plurality of subpools before determining the presence and/or amount, or the absence of the on-target extension product in 30070; (III) providing a plurality of paired combinations of the first construct and second construct, wherein the plurality of paired combinations comprises one or more trimmed paired combinations comprising splint oligonucleotides having a 3′ hybridizing region that is 1, 2, 3 or more nucleotides shorter than the 3′ hybridizing region of the splint oligonucleotides of at least one other paired combination of the plurality of paired combinations, wherein the 3′ hybridizing regions of the splint oligonucleotides of the at least one other paired combination of the plurality of paired combinations is different from and is not complementary to any contiguous stretch of the 3′ hybridizing region of the splint oligonucleotides of the one or more trimmed paired combinations; and/or (IV) attenuating an amount of amplification products by reducing or interfering with a binding interaction between the analyte and the first moiety or the second moiety, and/or suppressing on-target interactions between the conjugate splint oligonucleotide and the first splint oligonucleotide when the first moiety and the second moiety are both bound to the analyte (e.g., such that the 3′ hybridizing region of the first splint oligonucleotide and the 3′ hybridizing region of the second splint oligonucleotide are in proximity) (FIG. 30B).

### E. Blocker Oligonucleotides

Without being bound by theory, there are two highly consequential forms of off-target interaction that occur in highly multiplexed immunosequencing assays: 1) pulldown of detector (in the absence of analyte) through complementary “paired” hybridization overlaps due to relatively strong oligo-oligo interactions in a defined oligonucleotide set (or pair), and 2) mispriming of the 3′ terminus of forward (e.g., detection) or reverse (e.g., capture) oligonucleotides with the barcode regions of other oligonucleotides during extension. Pulldown of detector in the absence of analyte increases the assay background, cannot be removed by demultiplexing, and cannot be minimized by additional sequencing depth. The phenomenon of mispriming of the 3′ termini can be informatically removed by demultiplexing. However, there are practical limitations to this as there is a finite number of NGS reads produced by any NGS run and the higher number of misprimed (or illegitimate) reads will come at the expense of productive, correctly matched reads.

In some embodiments, these two phenomena are driven by different oligonucleotide:oligonucleotide interactions, and require different mitigation strategies. First, in some embodiments, the pulldown of detector in the absence of analyte is eliminated by adding a short (e.g., 10-14 nt) blocker that binds to the hybridization region stably under normal salt conditions (e.g., 137 mM NaCl), but denatures at low salt conditions (e.g., <10 mM NaCl). Second, in some embodiments, 3′ mispriming is minimized by adding a non-labile, salt-stable long (e.g., 18-24 nt) blocker that binds to the barcode region. When used in tandem, in some embodiments, both of these oligonucleotide blockers minimize the two types of oligo-oligo interactions, and improve the quality of PESD assays.

In some embodiments, in a method of analyzing a sample for an analyte, the complexing solution includes one or more blocker oligonucleotides, wherein each blocker oligonucleotide hybridizes to a subpart of the first splint oligonucleotide and/or of the second splint oligonucleotide (FIG. 30B, option (I)). In some embodiments, the complexing solution includes one or more blocker oligonucleotides that hybridize to a subpart of the first splint oligonucleotide. In some embodiments, the complexing solution includes one or more blocker oligonucleotides that hybridize to a subpart of the second splint oligonucleotide. In some embodiments, the complexing solution includes one or more blocker oligonucleotides that hybridize to a subpart of the first splint oligonucleotide, and one or more blocker oligonucleotides that hybridize to a subpart of the second splint oligonucleotide. In some embodiments, the blocker oligonucleotide is pre-annealed to the first splint oligonucleotide and/or the second splint oligonucleotide when preparing the complexing solution.

In some embodiments, the one or more blocker oligonucleotides reduce an analyte-independent interaction between the first splint oligonucleotide and the second splint oligonucleotide, and/or reduce an off-target interaction between the first splint oligonucleotide and the second splint oligonucleotide. In some embodiments, the blocker oligonucleotide reduces an analyte-independent interaction between the first splint oligonucleotide and the second splint oligonucleotide. In some embodiments, an interaction between the first splint oligonucleotide and the second splint oligonucleotide having complementary 3′ hybridization regions is an analyte-independent interaction when the first moiety and second moiety associated with the first splint oligonucleotide and second splint oligonucleotide, respectively, are not bound to the analyte (e.g., not bound to the same analyte molecule such that the 3′ hybridizing region of the first splint oligonucleotide and the 3′ hybridizing region of the second splint oligonucleotide are in proximity). In some embodiments, an analyte-independent interaction between the first splint oligonucleotide and the second splint oligonucleotide is observed when the assay is performed in the absence of the analyte. In some embodiments, the amount of on-target extension product determined in the absence of the analyte is due to an analyte-independent interaction between the first splint oligonucleotide and the second splint oligonucleotide, and is characterized as noise or background.

The blocker oligonucleotides, if used, can reduce or effectively eliminate regions of the splint oligonucleotides that are single stranded during the capture reaction (e.g., preparing the complexing solution at block 30040), thereby reducing or preventing non-specific interaction between the oligonucleotides in the solution. In some embodiments, the first splint oligonucleotide and/or second splint oligonucleotide in the complexing solution is, is about, or is at most 40, 35, 30, 25, 20, 15, 10, 5, 1%, or 0% single stranded, or in some embodiments, the first splint oligonucleotide and/or second splint oligonucleotide in the complexing solution is a percentage in a range defined by any two of the preceding values (e.g., 1-40%, 5-35%, 10-25%, 1-10%, etc.) single-stranded along its length upon hybridization of the one or more blocker oligonucleotides to the one or more subparts of the splint oligonucleotides. In some embodiments, the first splint oligonucleotide and/or second splint oligonucleotide in the complexing solution is about 0% single-stranded along its length upon hybridization of the one or more blocker oligonucleotides to the one or more subparts of the splint oligonucleotides. In some embodiments, the splint oligonucleotide has substantially no single-stranded region (e.g., not more than 20%, 15%, 10%, 5%, 4%, 3%, 2%, or 1% is single-stranded) when the blocker oligonucleotides are hybridized thereto (e.g., in the complexing solution). For example, the splint oligonucleotide may be double-stranded due to hybridization with a tether oligonucleotide and one or more blocker oligonucleotides, and optionally with the 3′ hybridization region of the corresponding paired splint oligonucleotide. In some embodiments, 1, 2, or 3 nucleotides adjacent and 5′ to the barcode region remains single-stranded when the blocker oligonucleotides are hybridized to the splint oligonucleotides (e.g., in the complexing solution). In some embodiments, the method is a multiplexed method that involves the use of different splint oligonucleotide pairs associated with moieties that bind different analytes in the sample. In some embodiments, the method (e.g., multiplexed method) includes providing a plurality of the first constructs comprising a plurality of first splint oligonucleotides; and providing a plurality of the second constructs comprising a plurality of second splint oligonucleotides, wherein substantially all (e.g., at least 80%, 85%, 90%, 95%, 97%, 98%, or 99%) of the plurality of first splint oligonucleotides and/or second splint oligonucleotides are each, each about, or each at most 40, 35, 30, 25, 20, 15, 10, 5, 1%, or 0% single-stranded, or optionally wherein each is a percentage in a range defined by any two of the preceding values (e.g., 1-40%, 5-35%, 10-25%, 1-10%, etc.) single-stranded along its length when the one or more blocker oligonucleotides are hybridized to one or more subparts of the substantially all (e.g., at least 80%, 85%, 90%, 95%, 97%, 98%, or 99%) of the plurality of first splint oligonucleotides and/or second splint oligonucleotides (e.g., in the complexing solution). In some embodiments, at least or about 10, 20, 30, 40, 50, 60, 70, 80, 85, 90, 95, 97, 98, 99% or about 100%, or a percentage in a range defined by any two of the preceding values (e.g., 10-100%, 20-98%, 30-90%, 50-97%, etc.) of the plurality of first splint oligonucleotide are each, each about, or each at most 40, 35, 30, 25, 20, 15, 10, 5, 1%, or a percentage in a range defined by any two of the preceding values (e.g., 1-40%, 5-35%, 10-25%, 1-10%, etc.), single-stranded along its length when the one or more blocker oligonucleotides are hybridized to one or more subparts of the plurality of first splint oligonucleotides (e.g., in the complexing solution). In some embodiments, at least or about 10, 20, 30, 40, 50, 60, 70, 80, 85, 90, 95, 97, 98, 99% or about 100% of the plurality of first splint oligonucleotide are each or are each substantially double stranded (e.g., at least 80%, 85%, 90%, 95%, 97%, 98%, or 99% double stranded) along its length (for example, in the complexing solution), or optionally wherein a percentage in a range defined by any two of the preceding values (e.g., 10-100%, 20-98%, 30-90%, 50-97%, etc.) of the plurality of first splint oligonucleotide are each or are each substantially double stranded (e.g., at least 80%, 85%, 90%, 95%, 97%, 98%, or 99% double stranded) along its length (for example, in the complexing solution). In some embodiments, at least or about 10, 20, 30, 40, 50, 60, 70, 80, 85, 90, 95, 97, 98% or about 100% are each, each about, or each at most 40, 35, 30, 25, 20, 15, 10, 5, 1%, or a percentage in a range defined by any two of the preceding values (e.g., 1-40%, 5-35%, 10-25%, 1-10%, etc.), single-stranded along its length when the one or more blocker oligonucleotides are hybridized to one or more subparts of the plurality of second splint oligonucleotides (e.g., in the complexing solution), or optionally wherein a percentage in a range defined by any two of the preceding values (e.g., 10-100%, 20-98%, 30-90%, 50-97%, etc.) of the plurality of second splint oligonucleotide are each, each about, or each at most 40, 35, 30, 25, 20, 15, 10, 5, 1%, or a percentage in a range defined by any two of the preceding values (e.g., 1-40%, 5-35%, 10-25%, 1-10%, etc.), single-stranded along its length when the one or more blocker oligonucleotides are hybridized to one or more subparts of the plurality of second splint oligonucleotides (e.g., in the complexing solution). In some embodiments, at least or about 10, 20, 30, 40, 50, 60, 70, 80, 85, 90, 95, 97, 98, 99% or about 100%, of the plurality of second splint oligonucleotide are each or are each substantially double-stranded (e.g., at least 80%, 85%, 90%, 95%, 97%, 98%, or 99% double stranded) along its length (for example, in the complexing solution), or optionally wherein a percentage in a range defined by any two of the preceding values (e.g., 10-100%, 20-98%, 30-90%, 50-97%, etc.) of the plurality of second splint oligonucleotide are each or are each substantially double-stranded (e.g., at least 80%, 85%, 90%, 95%, 97%, 98%, or 99% double stranded) along its length (for example, in the complexing solution).

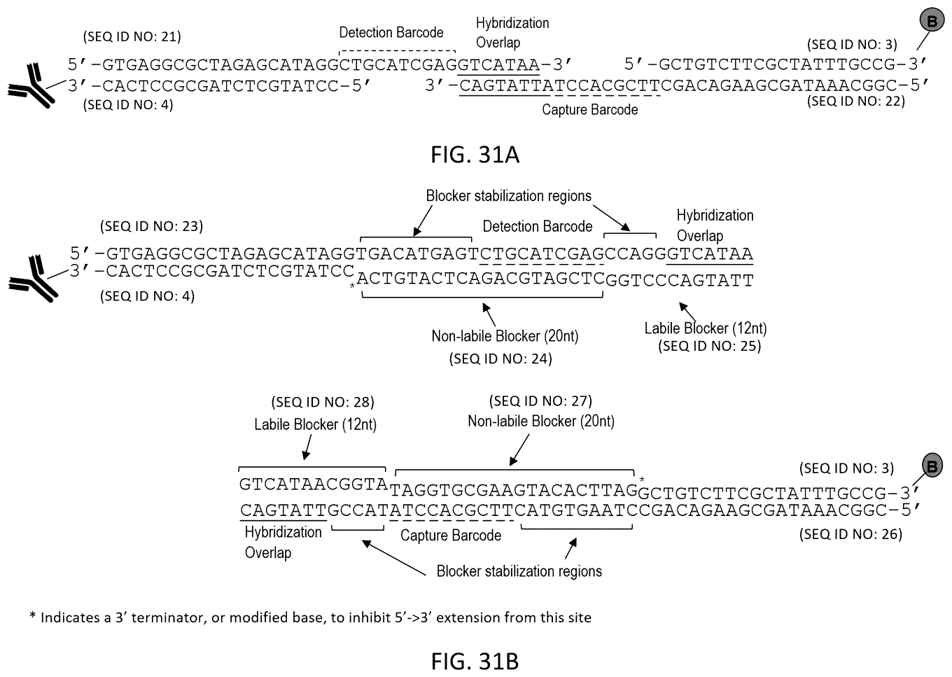

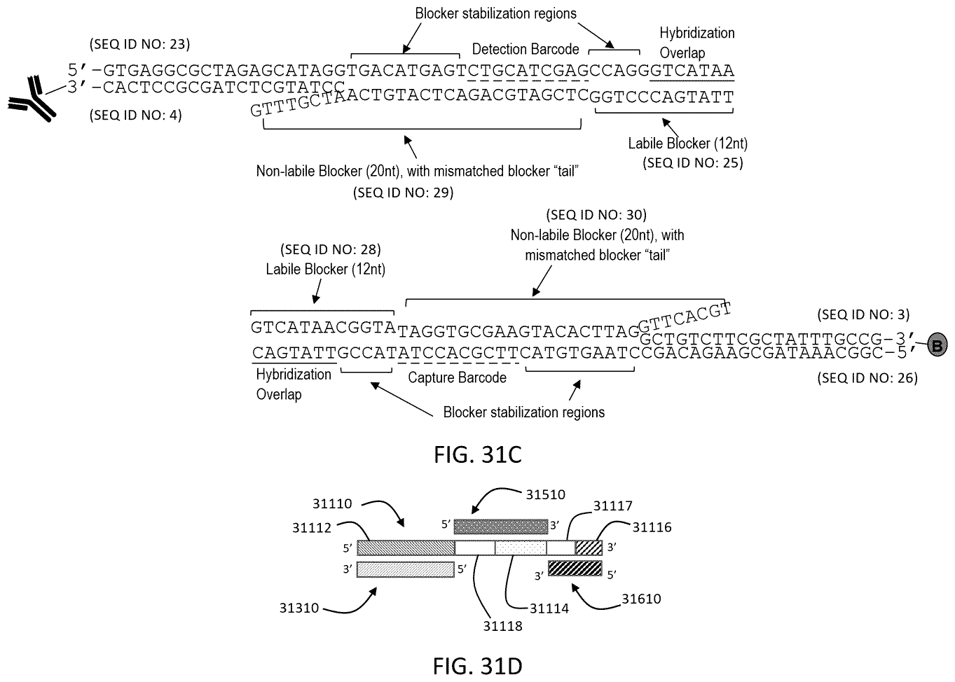

With reference to FIG. 31D, non-limiting schematic examples of blocker oligonucleotides of the present disclosure are provided. In some embodiments, a splint oligonucleotide 31110 (e.g., a first or second splint oligonucleotide, a capture or detection oligonucleotide, etc.) includes a 3′ hybridization region 31116, configured to hybridize to a 3′ hybridization region of a corresponding paired splint oligonucleotide, as described herein. In some embodiments, a hybridization blocker oligonucleotide 31610 (“hyb blocker”) hybridizes to the 3′ hybridization region. The hybridization blocker oligonucleotide can reduce or prevent non-specific interaction of the 3′ hybridization region with oligonucleotides (e.g., single-stranded oligonucleotides or a single-stranded part thereof) in the reaction other than the 3′ hybridization region of a corresponding paired splint oligonucleotide that are in an on-target arrangement (e.g., where the associated moieties are bound to the same analyte), for example, the 3′ hybridization region of a splint oligonucleotide from a different paired combination. In some embodiments, the splint oligonucleotide includes a stabilization region 31117 immediately 5′ of the 3′ hybridization region, and the hybridization blocker oligonucleotide hybridizes along both the 3′ hybridization region and the stabilization region that is contiguous to the 3′ hybridization region. In some embodiments, the stabilization region increases the hybridization energy of the hybridization blocker oligonucleotide and makes the interaction more stable. In some embodiments, this further reduces the background compared to the reduction in background that may be achieved by a shorter hybridization blocker oligonucleotide. In some embodiments, the hybridization blocker oligonucleotide is labile (e.g., can be removed by stringent wash), as described herein. In some embodiments, the splint oligonucleotide is hybridized to a barcode blocker oligonucleotide and/or hybridization blocker oligonucleotide, and optionally to the tether oligonucleotide, at least in the complexing solution (e.g., before extending). In some embodiments, the hybridization blocker oligonucleotide is removed from the splint oligonucleotide before the extending. In some embodiments, the splint oligonucleotide is hybridized to a barcode blocker oligonucleotide, and optionally to the tether oligonucleotide, in the extension reaction, and the barcode blocker oligonucleotide is removed from the splint oligonucleotide if the splint oligonucleotide participates in the extension (e.g., the splint oligonucleotide is hybridized to its paired counterpart splint oligonucleotide through the 3′ hybridization region to allow extension from the 3′ end of the counterpart splint oligonucleotide).

In some embodiments, the subpart to which a first blocker oligonucleotide (e.g., a hybridization blocker oligonucleotide) of the one or more blocker oligonucleotides hybridizes includes the 3′ hybridizing region or a portion thereof of the first splint oligonucleotide or second splint oligonucleotide, wherein the first blocker oligonucleotide competes with: the 3′ hybridizing region of the first splint oligonucleotide for binding to the 3′ hybridizing region of the second splint oligonucleotide; or the 3′ hybridizing region of the second splint oligonucleotide for binding to the 3′ hybridizing region of the first splint oligonucleotide. In some embodiments, the subpart to which the first blocker oligonucleotide (e.g., a hybridization blocker oligonucleotide) hybridizes includes the 3′ hybridizing region or a portion thereof of the first splint oligonucleotide, wherein the first blocker oligonucleotide competes with the 3′ hybridizing region of the second splint oligonucleotide for binding to the 3′ hybridizing region of the first splint oligonucleotide. In some embodiments, the subpart to which a first blocker oligonucleotide (e.g., a hybridization blocker oligonucleotide) hybridizes includes the 3′ hybridizing region or a portion thereof of the second splint oligonucleotide, wherein the first blocker oligonucleotide competes with the 3′ hybridizing region of the first splint oligonucleotide for binding to the 3′ hybridizing region of the second splint oligonucleotide. In some embodiments, the first blocker oligonucleotide comprises a sequence that is at least partially complementary (e.g., about or at least 50, 60, 70, 75, 80, 85, 90, 95, 97, 98, 99%, or about 100%, or a percentage in a range defined by any two of the preceding values (e.g., 50-100%, 60-99%, 70-98%, 75-95%, 80-99%, etc.) complementary) to the 3′ hybridizing region of the first or second splint oligonucleotide. In some embodiments, the first blocker oligonucleotide comprises a sequence that is complementary to the 3′ hybridizing region of the first or second splint oligonucleotide.

In some embodiments, the hybridization blocker targets the first splint oligonucleotide (e.g., capture side). In some embodiments, the first construct comprises a solid support comprising: the first moiety attached to the solid support; and the first splint oligonucleotide attached to the solid support, wherein the first splint oligonucleotide is attached to the first moiety via the solid support, wherein the subpart to which a first blocker oligonucleotide of the one or more blocker oligonucleotides hybridizes comprises the 3′ hybridizing region or a portion thereof of the first splint oligonucleotide, wherein the blocker oligonucleotide competes with the 3′ hybridizing region of the second splint oligonucleotide for binding to the 3′ hybridizing region of the first splint oligonucleotide.

The first blocker oligonucleotide (e.g., hybridization blocker oligonucleotide) that is bound to the 3′ hybridizing region of the first splint oligonucleotide or second splint oligonucleotide can be removed from the splint oligonucleotides using any suitable option. In some embodiments, the method includes removing the first blocker oligonucleotide (e.g., hybridization blocker oligonucleotide) bound to the 3′ hybridizing region of the first splint oligonucleotide or second splint oligonucleotide after preparing the complexing solution and before the extending. In some embodiments, removing the first blocker oligonucleotide (e.g., hybridization blocker oligonucleotide) bound to the 3′ hybridizing region of the splint oligonucleotides does not remove a second blocker oligonucleotide (e.g., barcode blocker oligonucleotide) that is also bound to the splint oligonucleotides. In some embodiments, removing the first blocker oligonucleotide includes washing the first construct comprising the first moiety bound to the analyte and/or the second construct comprising the second moiety bound to the analyte (e.g., washing the complex of the first construct and second construct bound to the analyte). In some embodiments, removing the first blocker oligonucleotide includes contacting the first construct comprising the first moiety bound to the analyte and/or the second construct comprising the second moiety bound to the analyte with a nuclease specific to the first blocker oligonucleotide bound to the 3′ hybridizing region of the first splint oligonucleotide and/or second splint oligonucleotide. In some embodiments, the first construct includes a solid support that includes the first moiety attached to the solid support; and the first splint oligonucleotide attached to the solid support, wherein the first splint oligonucleotide is attached to the first moiety via the solid support, and washing includes washing the solid support (e.g., washing the solid support that includes the first moiety bound to the analyte, to which the second moiety of the second construct is also bound), and/or contacting the solid support with the nuclease specific to the first blocker oligonucleotide bound to the 3′ hybridizing region of the first splint oligonucleotide and/or second splint oligonucleotide. Any suitable nuclease can be used to remove the first blocker oligonucleotide. In some embodiments, the nuclease is an endonuclease or an exonuclease. In some embodiments, the nuclease is a restriction endonuclease or enzyme. Suitable restriction enzymes include, without limitation, EcoRI, EcoRV, HindIII, XbaI, NotI, SpeI, SacI, BamHI, etc.

The first blocker oligonucleotide (e.g., hybridization blocker oligonucleotide) can include a nucleotide sequence of any suitable length that hybridizes to the splint oligonucleotide. In some embodiments, the first blocker oligonucleotide includes a nucleotide sequence of, of about, or of at least 4, 5, 6, 7, 8, 9, 10, 11, 12, 13, 14, 15 nucleotides in length that hybridizes to the subpart of the first splint oligonucleotide and/or of the second splint oligonucleotide. In some embodiments, an at least 5 nucleotide sequence (e.g., a 5-13 nucleotide or 7-13 nucleotide or 10-14 nucleotide sequence) of the first blocker oligonucleotide hybridizes to the subpart of the first splint oligonucleotide and/or of the second splint oligonucleotide. In some embodiments, the first blocker oligonucleotide includes a nucleotide sequence of 12 nucleotides in length that hybridizes to the subpart of the first splint oligonucleotide and/or of the second splint oligonucleotide. In some embodiments, the first blocker oligonucleotide has a length that renders it labile, such that the hybridized first blocker oligonucleotide can be effectively removed by a stringent wash.

In some embodiments, the first and/or second splint oligonucleotide includes a stabilization region immediately 5′ of the 3′ hybridization region. In some embodiments, the first blocker oligonucleotide (e.g., hybridization blocker oligonucleotide) hybridizes to at least part of the stabilization region. In some embodiments, the subpart of the first splint oligonucleotide or second splint oligonucleotide to which the first blocker oligonucleotide of the one or more blocker oligonucleotides hybridizes comprises one or more 5′ residues (e.g., a stabilization region) adjacent the 3′ hybridizing region of the first splint oligonucleotide or second splint oligonucleotide. The stabilization region can be any suitable length. In some embodiments, the one or more 5′ residues (e.g., a stabilization region) adjacent the 3′ hybridizing region of the first splint oligonucleotide or second splint oligonucleotide includes a sequence of 1, 2, 3, 4, 5, 6, 7, 8, 9, 10 or more nucleotides. In some embodiments, the 3′ hybridizing region is 7 nucleotides long, and the stabilization region is 5 nucleotides long. In some embodiments, the one or more 5′ residues (e.g., a stabilization region) adjacent the 3′ hybridizing region does not comprise a barcode region of the first splint oligonucleotide or second splint oligonucleotide or a part thereof. In some embodiments, the first and/or second splint oligonucleotide includes a stabilization region between a barcode region and a 3′ hybridizing region. In some embodiments, the stabilization region includes a nucleotide sequence that is unique to each paired combination of splint oligonucleotides. In some embodiments, the stabilization region includes a nucleotide sequence that is common between two or more different paired combination of splint oligonucleotides.

The first blocker oligonucleotide (e.g., hybridization blocker oligonucleotide) can be any suitable length. In some embodiments, the first blocker oligonucleotide is, is about, or is at least 4, 5, 6, 7, 8, 9, 10, 11, 12, 13, 14, 15 nucleotides long. In some embodiments, the first blocker oligonucleotide is about 5 to about 13 nucleotides long, about 7 to about 13 nucleotides long, or about 4 to about 15 nucleotides long. In some embodiments, the first blocker oligonucleotide is, or is about 12 nucleotides long. In some embodiments, a multiplex assay includes first blocker oligonucleotides of different lengths. For example, a longer first blocker oligonucleotide can bind a splint oligonucleotide of a paired combination of splint oligonucleotides that generates higher background, compared to a shorter first blocker oligonucleotide that binds a splint oligonucleotide of a paired combination of splint oligonucleotides that generates lower background. In some embodiments, a multiplex assay includes first blocker oligonucleotides of the same length (e.g., regardless of the splint oligonucleotides or expected amount of analyte).

The first blocker oligonucleotide (e.g., hybridization blocker oligonucleotide) can be present in the complexing solution at any suitable concentration. In some embodiments, the first blocker oligonucleotide is present in the complexing solution at a concentration of, of about, or of at least 1, 10, 20, 50, 100, 200, 500, 1,000, 2,000, 5,000, 10,000, 50,000, 100,000 nM, or in a range defined by any two of the preceding values (e.g., about 1-100,000 nM, about 10-10,000 nM, 50-5,000 nM, 100-10,000 nM, etc.). In some embodiments, the first blocker oligonucleotide is present in the complexing solution at a concentration that is at about the same or greater than the concentration of the first splint oligonucleotide and/or second splint oligonucleotide to which the first blocker oligonucleotide hybridizes. In some embodiments, the first blocker oligonucleotide is present in the complexing solution at a concentration that is greater than the concentration of the first splint oligonucleotide and/or second splint oligonucleotide to which the first blocker oligonucleotide hybridizes by, by about, or by at least 1.1, 1.2, 1.5, 2, 2.5, 3, 4, 5, 10, 20, 50, or 100 fold, or a fold amount in a range defined by any two of the preceding values (e.g., 1.1-100 fold, 1.2-50 fold, 1.5-50 fold, etc.).

In some embodiments, the method is multiplexed method, as described herein. In some embodiments, the method includes providing a plurality of the first constructs comprising a plurality of first splint oligonucleotides; and providing a plurality of the second constructs comprising a plurality of second splint oligonucleotides, wherein the plurality of first splint oligonucleotides comprises two or more different first splint oligonucleotides and/or the plurality of second splint oligonucleotides comprises two or more different second splint oligonucleotides, wherein the one or more blocker oligonucleotides comprises at least one first blocker oligonucleotide that hybridizes to the 3′ hybridizing region of at least one of the two or more different first splint oligonucleotides or of at least one of the two or more different second splint oligonucleotides. In some embodiments, the method includes providing a plurality of paired combinations of the first construct and second construct, wherein a binding target of the first moiety and second moiety of each paired combination is the same, and wherein different paired combinations of the plurality of paired combinations have different binding targets, wherein the 3′ hybridizing region of the first splint oligonucleotide and second splint oligonucleotide of a paired combination of the plurality of paired combinations is different from and is not complementary to at least one other paired combination of the plurality of paired combinations having a different binding target. As used herein, a “plurality of paired combinations of the first construct and second construct” indicates that each paired combination of the plurality of paired combinations includes the first construct and the second construct. In some embodiments, the 3′ hybridizing regions of each of the paired combinations are different from all other paired combinations (e.g., the 3′ hybridizing regions are orthogonal to each other). In some embodiments, the 3′ hybridizing regions of all paired combinations of the plurality of paired combinations have a common 3′ hybridizing region.

In some embodiments, a splint oligonucleotide 31110 (e.g., a first or second splint oligonucleotide, a capture or detection oligonucleotide, etc.) includes a barcode region 31114, as described herein. In some embodiments, a barcode blocker oligonucleotide 31510 (“barcode blocker”) hybridizes to the barcode region. The barcode blocker oligonucleotide can reduce or prevent non-specific interaction of the barcode with oligonucleotides (e.g., single-stranded oligonucleotides or a single-stranded part thereof) in the reaction, for example, the 3′ hybridization region of another splint oligonucleotide. In some embodiments, the splint oligonucleotide includes a stabilization region 31118 immediately 5′ (and/or 3′) of the barcode region, and the barcode blocker oligonucleotide hybridizes along both the barcode region and the stabilization region that is 5′ of the barcode region. In some embodiments, the splint oligonucleotide includes a stabilization region immediately 3′ of the barcode region, and the barcode blocker oligonucleotide hybridizes along both the barcode region and the stabilization region that is 3′ of the barcode region. In some embodiments, the splint oligonucleotide includes a stabilization region flanking the 5′ and 3′ sides of the barcode region, and the barcode blocker oligonucleotide hybridizes along both the barcode region and the stabilization regions flanking the barcode region. In some embodiments, the stabilization region increases the hybridization energy of the barcode blocker oligonucleotide and makes the interaction more stable. In some embodiments, this further reduces the background compared to the reduction in background that may be achieved by a shorter barcode blocker oligonucleotide. In some embodiments, the barcode blocker oligonucleotide is non-labile (e.g., cannot be removed by stringent wash), as described herein.

In some embodiments, the first splint oligonucleotide and/or second splint oligonucleotide comprises a barcode sequence, and wherein the subpart to which a second blocker oligonucleotide (e.g., a barcode blocker oligonucleotide) of the one or more blocker oligonucleotides hybridizes includes the barcode sequence, or a portion thereof. In some embodiments, the first splint oligonucleotide comprises a barcode sequence, and wherein the subpart to which a second blocker oligonucleotide (e.g., a barcode blocker oligonucleotide) of the one or more blocker oligonucleotides hybridizes includes the barcode sequence, or a portion thereof. In some embodiments, the second splint oligonucleotide comprises a barcode sequence, and wherein the subpart to which a second blocker oligonucleotide (e.g., a barcode blocker oligonucleotide) of the one or more blocker oligonucleotides hybridizes includes the barcode sequence, or a portion thereof. In some embodiments, the second blocker oligonucleotide comprises a sequence that is at least partially complementary (e.g., about or at least 50, 60, 70, 75, 80, 85, 90, 95, 97, 98, 99%, or about 100%, or a percentage in a range defined by any two of the preceding values (e.g., 50-100%, 60-99%, 70-98%, 75-95%, 80-99%, etc.) complementary) to the barcode region of the first or second splint oligonucleotide. In some embodiments, the second blocker oligonucleotide comprises a sequence that is complementary to the barcode region of the first or second splint oligonucleotide.

The second blocker oligonucleotide (e.g., barcode blocker oligonucleotide) can include a nucleotide sequence of any suitable length that hybridizes to the splint oligonucleotide. In some embodiments, the second blocker oligonucleotide includes a nucleotide sequence of, of about, or of at least 12, 13, 14, 15, 16, 17, 18, 19, 20, 21, 22, 23, 24 nucleotides or more in length that hybridizes to the subpart of the first splint oligonucleotide and/or of the second splint oligonucleotide. In some embodiments, an at least 12 nucleotide sequence (e.g., a 12-24 nucleotide or 15-22 nucleotide or 18-24 nucleotide sequence) of the second blocker oligonucleotide hybridizes to the subpart of the first splint oligonucleotide and/or of the second splint oligonucleotide. In some embodiments, the second blocker oligonucleotide includes a nucleotide sequence of 20 nucleotides in length that hybridizes to the subpart of the first splint oligonucleotide and/or of the second splint oligonucleotide. In some embodiments, the second blocker oligonucleotide has a sufficient length to render it non-labile, such that the hybridized second blocker oligonucleotide is not removed by a stringent wash. In some embodiments, the second blocker oligonucleotide is not labile under conditions in which the first blocker oligonucleotide is labile.

In some embodiments, the first and/or second splint oligonucleotide includes a stabilization region immediately 5′ and/or 3′ of the barcode region. In some embodiments, the second blocker oligonucleotide (e.g., barcode blocker oligonucleotide) hybridizes to at least part of the stabilization region(s). In some embodiments, the subpart of the first splint oligonucleotide or second splint oligonucleotide to which the second blocker oligonucleotide of the one or more blocker oligonucleotides hybridizes comprises one or more 5′ and/or 3′ residues (e.g., stabilization region(s)) adjacent the barcode region of the first splint oligonucleotide or second splint oligonucleotide. The stabilization region(s) can be any suitable length. In some embodiments, the one or more 5′ and/or 3′ residues (e.g., stabilization region(s)) adjacent the barcode region of the first splint oligonucleotide or second splint oligonucleotide includes a sequence of 1, 2, 3, 4, 5, 6, 7, 8, 9, 10, 11, 12, 13, 14, 15 or more nucleotides. In some embodiments, the barcode region is 10 nucleotides long, and the stabilization region(s) is 10 nucleotides long. In some embodiments, the one or more 5′ residues (e.g., a stabilization region) adjacent the barcode region does not comprise a tethering region of the first splint oligonucleotide or second splint oligonucleotide or a part thereof. In some embodiments, the first and/or second splint oligonucleotide includes a stabilization region between a tethering region and a barcode region. In some embodiments, the one or more 3′ residues (e.g., a stabilization region) adjacent the barcode region does not comprise a 3′ hybridization region or a stabilization region for a first blocker oligonucleotide that hybridizes to the 3′ hybridization region. In some embodiments, the first and/or second splint oligonucleotide includes a stabilization region between a barcode region and a 3′ hybridization region or a stabilization region for a first blocker oligonucleotide that hybridizes to the 3′ hybridization region.

The second blocker oligonucleotide (e.g., barcode blocker oligonucleotide) can be any suitable length. In some embodiments, the second blocker oligonucleotide is, is about, or is at least 12, 13, 14, 15, 16, 17, 18, 19, 20, 21, 22, 23, 24, 25, 26, 27, 28, 29, or 30 nucleotides long. In some embodiments, the second blocker oligonucleotide is about 12 to about 30 nucleotides long, about 15 to about 25 nucleotides long, or about 18 to about 24 nucleotides long. In some embodiments, the second blocker oligonucleotide is, or is about 20 nucleotides long. In some embodiments, a multiplex assay includes second blocker oligonucleotides of different lengths. For example, a longer second blocker oligonucleotide can bind a splint oligonucleotide of a paired combination of splint oligonucleotides that generates higher background, compared to a shorter second blocker oligonucleotide that binds a splint oligonucleotide of a paired combination of splint oligonucleotides that generates lower background. In some embodiments, a multiplex assay includes second blocker oligonucleotides of the same length (e.g., regardless of the splint oligonucleotides or expected amount of analyte).

In some embodiments, hybridizing of the second blocker oligonucleotide to the first splint oligonucleotide and/or second splint oligonucleotide renders the first splint oligonucleotide and/or second splint oligonucleotide, respectively, double-stranded substantially (e.g., at least 80%, 85%, 90%, 95%, 97%, 98%, or 99% double stranded) along its length other than the 3′ hybridization region. In some embodiments, at most 1 or 2 nucleotides of the first splint oligonucleotide and/or second splint oligonucleotide is single-stranded 5′ (e.g., immediately 5′) of the 3′ hybridization region upon hybridizing of the second blocker oligonucleotide to the first splint oligonucleotide and/or second splint oligonucleotide.

The second blocker oligonucleotide (e.g., barcode blocker oligonucleotide) that is bound to the barcode region of the first splint oligonucleotide and/or second splint oligonucleotide can be removed from the splint oligonucleotides using any suitable option. In some embodiments, the method includes removing the second blocker oligonucleotide (e.g., barcode blocker oligonucleotide) from the first splint oligonucleotide and/or second splint oligonucleotide by contacting the first splint oligonucleotide and/or second splint oligonucleotide with a 5′→3′ exonuclease. In some embodiments, the second blocker oligonucleotide is removed using a strand-displacing polymerase. In some embodiments, extending the hybridized first splint oligonucleotide and/or the hybridized second splint oligonucleotide includes contacting the hybridized first splint oligonucleotide and/or the hybridized second splint oligonucleotide with a strand-displacing polymerase under conditions sufficient to extend the hybridized first splint oligonucleotide and/or the hybridized second splint oligonucleotide, whereby the strand-displacing polymerase displaces the second blocker oligonucleotide from the first splint oligonucleotide and/or second splint oligonucleotide.

In some embodiments, the one or more blocker oligonucleotides comprise one or more chemically modified nucleotides (e.g., to prevent extension from the blocker oligonucleotide, or to increase hybridization energy). In some embodiments, the blocker oligonucleotides are modified to prevent the blocker oligonucleotides from functioning as an extension primer. In some embodiments, the blocker oligonucleotides are modified to increase hybridization energy and stabilize the duplex of the blocker oligonucleotides bound to the splint oligonucleotides. Any suitable option can be used to prevent extension from the blocker oligonucleotide, and/or to increase hybridization energy. In some embodiments, the one or more chemically modified nucleotides includes a 3′ phosphate or inverted dT, and/or a backbone modification. In some embodiments, the backbone modification includes a locked nucleic acid (LNA).

In some embodiments, the second blocker oligonucleotide includes one or more 3′ overhang nucleotides. In some embodiments, the overhang nucleotides include a sequence that does not hybridize to (e.g., is not complementary to) a region of the splint oligonucleotide immediately 5′ of the barcode region or the stabilization region 5′ of the barcode region. The 3′ overhang nucleotides can include any suitable number of nucleotides. In some embodiments, the 3′ overhang nucleotides includes 1, 2, 3, 4, 5, 6, 7, 8, 9, 10 or more nucleotides.

In some embodiments, the method includes providing the first construct by annealing the one or more blocker oligonucleotides to the first splint oligonucleotide, whereby the first construct comprises the one or more blocker oligonucleotides. In some embodiments, the method includes providing the second construct by annealing the one or more blocker oligonucleotides to the second splint oligonucleotide, whereby the second construct comprises the one or more blocker oligonucleotides. The blocker oligonucleotides can be hybridized or annealed to the splint oligonucleotides using any suitable option. In some embodiments, the blocker oligonucleotides are hybridized to splint oligonucleotides at the clonal preparation stage (e.g., when providing the first or second construct and before preparing the complexing solution). In some embodiments, the blocker oligonucleotides that are hybridized to splint oligonucleotides at the clonal preparation stage are non-labile. In some embodiments, the blocker oligonucleotides are combined with the splint oligonucleotides when the splint oligonucleotides are being hybridized to the tether oligonucleotides (e.g., biotinylated tether oligonucleotides for attaching to a solid support, tether oligonucleotides attached to a moiety that binds the analyte). In some embodiments, the blocker oligonucleotides are hybridized to splint oligonucleotides for hybridization after pooling (e.g., after pooling clonally prepared barcoded solid supports, after pooling barcoded detection moieties/antibodies, etc.). In some embodiments, the blocker oligonucleotides are combined with the first construct (e.g., a solid support having a capture moiety attached to the solid support and a capture oligonucleotide attached to the solid support), clonally or in a pooled population. In some embodiments, the blocker oligonucleotides are combined with the second construct (e.g., a detection moiety attached to a detection oligonucleotide), clonally or in a pooled population. In some embodiments, the blocker oligonucleotides are not pre-annealed to the first splint oligonucleotide and/or the second splint oligonucleotide, and the blocker oligonucleotides (e.g., un-annealed blocker oligonucleotides) are added to the complexing solution (e.g., before extending).

In some embodiments, the splint oligonucleotide 31110 (e.g., a first or second splint oligonucleotide, a capture or detection oligonucleotide, etc.) includes a 5′ tethering region 31112. In some embodiments, the splint oligonucleotide is hybridized to a tether oligonucleotide 31310 (“anchor”) via the 5′ tethering region. In some embodiments, the tether oligonucleotide is attached to a moiety (e.g., antibody as a conjugate) or a solid support (e.g., a bead), as described herein. In some embodiments, the splint oligonucleotide is double stranded substantially (e.g., at least 80%, 85%, 90%, 95%, 97%, 98%, or 99% double stranded) along its length, for example, through hybridization with a tether oligonucleotide, a barcode oligonucleotide, and a hybridization oligonucleotide, e.g., in a complexing solution, as described herein.

### F. Subpooling and Subpool Barcodes

In some embodiments, developing high multiplex immunosequencing based assays (e.g., number of analytes, N>100 plex) takes into account the concentration of analytes in relevant samples that can span many orders of magnitude. For example, in human plasma some cytokines may be present at only a few fg/mL, whereas some acute phase proteins may be present at several g/mL, a billion-fold disparity. For example, a small number of high abundance analytes in a patient sample may create a disproportionate number of amplicons in the sequencing library and may account for a disproportionate number of reads in any NGS run with a finite number of reads. In some embodiments, multiple strategies are used to measure analytes present at concentrations that span many orders of magnitude in multiplexing assays.

In some embodiments, in the context of immunosequencing based assays, for example, multiplex PESD, two factors can be addressed to allow each assay to retain a linear response over a sufficient dynamic range and to allow accurate quantification of each analyte in unknown samples.

A. Provide sufficient analyte capture capacity (number of capture antibodies and physical space on solid support) to avoid saturation by the high-abundance analytes. Saturation may lead to non-linear signal generation with respect to analyte concentration. Multiple strategies may be used to ensure sufficient capture capacity for high abundance analytes.

B. Prevent the total amplicon concentration generated by the high abundance analyte from being many-fold higher than that of the low abundance analytes. In NGS-based assays there can be a finite number of sequencing reads available, and the assay can be configured to produce roughly the same amplicon concentration for all analytes. Large disparities may cause a loss in sensitivity for low abundance analytes. Multiple strategies may be used to attenuate the amplicon generation for high abundance analytes and to produce a more uniform distribution of amplicons across analytes.

In some embodiments, these two factors are addressed together to improve the assay. For example, in some embodiments, increasing the number of capture beads to accommodate the higher level of some analytes, may solve the problem of capture saturation, but may also cause the assays for high abundance analytes to produce much more amplicon, leading to a higher disparity of amplicons in the sequencing pool, and causing loss of sensitivity for the assays measuring low abundance analytes.

In some embodiments, the method includes separating the multiplex assay panel into multiple sub-panels (or subpools) (e.g. those that can be run on undiluted sample, those that require a 20-fold dilution to bring the analytes into the linear range of the assays, those that require a 400-fold dilution and those that require an 8,000 fold dilution). Having multiple subpanels (or subpools) ensures that assays are only grouped with other assays that produce similar levels of amplicon (within 20-fold). In some embodiments, there is up to a 20-fold disparity between the lowest and highest analytes in a sample being analyzed based on the number of sequencing reads available, and this subpanel arrangement allows for a single concentration of beads, capture antibody, and reverse oligonucleotide to be used for all analytes. In some embodiments, the method includes running large multiplexes split across a small number of ‘subpools’ or ‘subpanels’ to analyze a wide range of endogenous analyte concentrations; to avoid known biological cross reactivity of antibodies/analytes; and/or to take into account compatibility/incompatibility of some analytes with certain diluents, especially those containing animal serum.

In some embodiments, in a method for analyte detection, the method includes analyzing the sample for a first analyte and a second analyte, wherein preparing the complexing solution includes: preparing the complexing solution in a plurality of subpools comprising a first and a second subpool, wherein the first moiety and second moiety in the prepared complexing solution of the first subpool bind the first analyte, and the first moiety and second moiety in the prepared complexing solution of the second subpool bind the second analyte; and combining the plurality of subpools before determining the presence and/or amount, or the absence of the on-target extension product (FIG. 30B, option (II)).

Combining the plurality of subpools can be done at any suitable stage of the assay before determining the presence and/or amount, or the absence of the on-target extension product. In some embodiments, the plurality of subpools are combined after contacting the first or second construct with the sample, and before contacting the sample-contacted sample with the other construct (e.g., at block 30030 in FIG. 30A: in ii), after contacting the first construct provided in 30010 with a sample, and before combining in a solution the sample-contacted construct and the second construct provided in 30020; or in iii), after contacting the second construct provided in 30020 with a sample, and before combining in a solution the sample-contacted construct with the first construct provided in 30010). For example, the subpools can be processed separately to capture an analyte with a corresponding capture moiety (e.g., on a solid support or a bead) in each subpool, and then the subpools can be pooled and the detection conjugates added to the pooled reaction. In some embodiments, the plurality of subpools are combined after preparing the complexing solution (e.g., having at least the first and second constructs and the sample), but before extension (e.g., after 30030 and before 30050 in FIG. 30A). For example, the subpools can be processed separately to capture an analyte with a corresponding capture moiety (e.g., on a solid support or a bead) in each subpool, the corresponding detection conjugates can then be added to each subpool, and then the subpools can be pooled to carry out the extension reaction in a pooled reaction. In some embodiments, the plurality of subpools are combined after the extension and before determining the presence and/or amount, or the absence of the on-target extension product (e.g., after 30050 and before 30070 in FIG. 30A). For example, the subpools can be processed separately to capture an analyte with a corresponding capture moiety (e.g., on a solid support or a bead) in each subpool, the corresponding detection conjugates can then be added to each subpool, extension can be performed in each subpool separately, and then the subpools can be pooled for sequencing library preparation and/or qPCR.

In some embodiments, the use of subpools, as described herein, is useful when different analytes of interest in a sample (e.g., a sample from a patient) are expected to be present at concentrations that span many orders of magnitude; when the sensitivity for generating on-target extension product from different analytes is different; or when binding of different analytes by the first moiety and second moiety is performed under different conditions (e.g., in different diluents). In some embodiments, the concentration of the first analyte in the sample is expected to be higher than the concentration of the second analyte in the sample. In some embodiments, the concentration of the first analyte in the sample is expected to be higher than a threshold concentration and the concentration of the second analyte in the sample is expected to be lower than the threshold concentration. In some embodiments, the first construct and second construct of the first subpool have a first sensitivity for generating on-target extension product from the first analyte, and the first construct and second construct of the second subpool have a second sensitivity for generating on-target extension product from the second analyte, wherein the first sensitivity is higher than the second sensitivity. In some embodiments, the first sensitivity is higher than a threshold sensitivity and the second sensitivity is lower than the threshold sensitivity. In some embodiments, the complexing solution in the first subpool does not include a paired combination of the first construct and second construct that includes a first moiety and second moiety that bind (e.g., effectively bind) to the first analyte. In some embodiments, an analyte (e.g., first analyte) that is (or is expected to be) present at high abundance in a sample has a concentration of, of about, or of at least, or on the order of 10⁻¹¹, 10⁻¹⁰, 10⁻¹, 10⁻⁸, 10⁻⁷, or 10⁻⁶ M or higher, or optionally at a concentration in a range defined by any two of the preceding values (e.g., 10⁻¹¹-10⁻⁶ M, 10⁻¹⁰-10⁻⁶ M, 10⁻⁹-10⁻⁶ M, 10⁻⁸-10⁻⁶ M, 10⁻⁸-10⁻⁷ M, or 10⁻⁷-10⁻⁶ M). In some embodiments, the analyte (e.g., first analyte) that is (or is expected to be) present at high abundance in the sample has a concentration of, of about, or of at least, or on the order of, 10⁻⁸, 10⁻⁷, 10⁻⁶, 10⁻⁵, 10⁻⁴, or 10⁻³ g/mL or higher, or optionally at a concentration in a range defined by any two of the preceding values (e.g., 10⁻⁸-10⁻³ g/mL, 10⁻⁸-10⁻⁵ g/mL, 10⁻⁸-10⁻⁴ g/mL, 10⁻⁸-10⁻⁶ g/mL, 10⁻⁸-10⁻⁷ g/mL, or 10⁻⁷-10⁻⁶ g/mL). In some embodiments, an analyte (e.g., second analyte) that is (or is expected to be) present at low abundance in a sample has a concentration of, of about, or of at least, or no the order of 10⁻¹⁸ 10⁻¹⁷, 10⁻¹⁶, 10¹⁵, 10⁻¹⁴, 10⁻¹³, or 10⁻¹² M, or optionally at a concentration in a range defined by any two of the preceding values (e.g., 10⁻¹⁸-10⁻¹² M, 10⁻¹⁷-10⁻¹² M, 10⁻¹⁶-10⁻¹² M, 10⁻¹-10⁻¹² M, 10⁻¹⁴-10⁻¹² M, 10⁻¹-10⁻¹² M, etc.). In some embodiments, the analyte (e.g., second analyte) that is (or is expected to be) present at low abundance in the sample has a concentration of, of about, or of at least, or on the order of, 10⁻¹⁵, 10⁻¹⁴, 10⁻¹³, 10⁻¹², 10⁻¹¹, 10⁻¹⁰, or 10⁻⁹ g/mL, or optionally at a concentration in a range defined by any two of the preceding values (e.g., 10⁻¹-10⁻⁹ g/mL, 10⁻¹⁵ 10⁻¹⁰ g/mL, 10⁻¹⁵-10⁻¹¹ g/mL, 10⁻¹⁵-10⁻¹² g/mL, 10⁻¹⁴-10⁻¹² g/mL, 10⁻¹³-10⁻¹² g/mL, etc.).

In some embodiments, the first subpool and the second subpool comprise different amounts of the sample. As used herein, the “amount of sample” in a subpool denotes a quantity (e.g., by weight mass), fraction, or concentration of the sample in the subpool, or dilution of the sample into the subpool. In some embodiments, the first subpool and the second subpool comprise different concentrations of the sample. In some embodiments, the first subpool and the second subpool comprise different dilutions of the sample. In some embodiments, the method includes providing a plurality of paired combinations of the first construct and second construct, wherein a first paired combination of the plurality of paired combinations comprises a first moiety and second moiety that bind to the first analyte, and a second paired combination of the plurality of paired combinations comprises a first moiety and second moiety that bind to the second analyte; and preparing the complexing solution in the plurality of subpools, wherein the amount of sample in the first subpool is higher than the amount of sample in the second subpool, wherein the complexing solution in the first subpool comprises the second paired combination of the plurality of paired combinations, and wherein the complexing solution in the second subpool comprises the first paired combination of the plurality of paired combinations.

Any suitable relative amount of the sample can be provided between the first or the second subpools. In some embodiments, the amount of sample in the first subpool is, is about, or is at least 2, 3, 4, 5, 6, 7, 8, 9, 10, 15, 20, 30, 40, 50, 75, 100, 150, 200, 300, 400, 500, 750, 1,000, 2,000, 4,000, 8,000, 10,000, 20,000, 50,000, 100,000, 200,000, 500,000, 10⁶ fold or more higher, or optionally the amount of sample in the first subpool is higher by a fold amount in a range defined by any two of the preceding values (e.g., about 2-10⁶ fold, about 10-100,000 fold, about 20-8,000 fold, about 4-400 fold, etc.). In some embodiments, the amount of sample in the first subpool is higher than the amount of sample in the second subpool by a fold amount in the range of about 5 to about 100,000.

In some embodiments, the method includes generating a dilution series of the sample before preparing the complexing solution in a plurality of subpools, wherein the complexing solution in the first subpool comprises a different dilution of the sample than the second subpool. As used herein, a “dilution” of the sample can include an undiluted (“neat”) sample or portion thereof in the subpool. In some embodiments, the dilution series of the sample comprises a, an about, or an at least 10, 20, 50, 100, 200, 400, 1,000, 2,000, 4,000, 8,000, 20,000, 50,000, 100,000, 10⁶, 10⁷, or 10⁸-fold or greater dilution, or optionally a dilution by a fold amount in a range defined by any two of the preceding values (e.g., 10-100,000 fold, 20-8,000 fold, 40-400 fold, 10⁵-10⁶ fold, 10⁶-10⁷ fold, 10⁷-10⁸ fold, etc.), of the sample between the first subpool and second subpool. In some embodiments, a concentration of the first analyte in the complexing solution of the second subpool is, or is expected to be, within, within about, or within at most 100, 90, 80, 60, 50, 40, 30, 20, 10, 5, 2-fold the concentration of the second analyte, or optionally the concentration of the first analyte is, or is expected to be, a fold amount of the concentration of the second analyte in a range defined by any two of the preceding values (e.g., about 2-100 fold, about 10-90 fold, 20-50 fold, etc.), in the sample (e.g., the sample before dilution, or a portion of the sample that has not been diluted) or in the first subpool. In some embodiments, a concentration of the first analyte in the complexing solution of the second subpool is, or is expected to be, at most within 20 fold the concentration of the second analyte.

In some embodiments, the detected amount of extension product for the first analyte is, or is expected to be, within, within about, or within at most 100, 90, 80, 60, 50, 40, 30, 20, 10, 5, or 2-fold the detected amount of extension product for the second analyte, or optionally the detected amount of extension product for the first analyte is, or is expected to be, a fold amount of the detected amount of extension product for the second analyte in a range defined by any two of the preceding values (e.g., about 2-100 fold, about 10⁻⁹⁰ fold, 20-50 fold, etc.). In some embodiments, the detected amount of extension product for the first analyte is, or is expected to be, within or within about 20-fold the detected amount of extension product for the second analyte. In some embodiments, the analytes comprising a subpool are selected so that the amount of extension product is expected to be within 20-fold between different subpools. In some embodiments, this accounts for not only differences in analyte concentration but also assay efficiency (e.g., due to differences in capture and detection moiety binding and also differences in oligo hybridization or extension efficiency). For example, the amount of extension product generated for analyte A assayed in subpool A (e.g., and later combined with other subpools before detecting extension products) is within 20 fold of the amount of extension product generated for analyte B assayed in subpool B (e.g., and also later combined with other subpools before detecting extension products), where subpool A and subpool B include different dilutions of the sample (or different reaction conditions), and where there would be a greater than 20 fold difference in the amount of extension product for analyte A compared to analyte B if both analytes were assayed without subpooling.

In some embodiments, different subpools are provided to allow binding of analytes in a sample under different conditions. In some embodiments, the complexing solution in the first subpool comprises a first diluent and wherein the complexing solution in the second subpool comprises a second diluent that is different from the first diluent. In some embodiments, the first diluent comprises one or more components to which the first moiety and/or second moiety in the prepared complexing solution of the second subpool bind. For example, serum-containing diluents or high salt are used in the first subpool, and first and/or second moieties that bind an analyte but also cross react with components in serum or due to high salt are used in a second subpool that does not use a diluent that contains serum or high salt.

In some embodiments, the use of subpools, as described herein, is useful for analyzing the same analyte, first moiety, and/or second moiety under different conditions or across a range of conditions (e.g., different buffers, different incubation periods, incubation for different amounts of time and or at different temperatures). For example, binding kinetics of the analyte, first moiety, and/or second moiety can be determined by combining the analyte with the first moiety and/or second moiety in the different subpools for different amounts of time. The subpools can be combined prior to determining the presence or the absence of any on-target extension product as described herein.

Any suitable number of subpools can be used in the present methods. In some embodiments, the plurality of subpools comprises 2, 3, 4, 5, 6, 7, 8, 9, 10, 15, 20, 50, 100 or more subpools, or optionally the plurality of subpools comprises a number of subpools in a range defined by any two of the preceding values (e.g., 4-100 subpools, 4-20 subpools, 5-10 subpools, etc.).

In some embodiments, one or more of the splint oligonucleotides of the plurality of paired combinations of the first and second constructs each comprises a subpool barcode, or a part thereof, that identifies a subpool of the plurality of subpools. In some embodiments, the first splint oligonucleotide of a paired combination of the plurality of paired combinations comprises a first subpool barcode, or a part thereof, that identifies the subpool of the plurality of subpools, and the second splint oligonucleotide of the paired combination of the plurality of paired combinations comprises a second subpool barcode, or a part thereof, that identifies the subpool of the plurality of subpools. In some embodiments, the combination of the first subpool barcode and the second subpool barcode identifies the subpool. The subpool barcode can be positioned at any suitable region of the splint oligonucleotide. In some embodiments, the subpool barcode is 5′ of the 3′ hybridization region (e.g., between the 3′ hybridization region and the barcode region), or 5′ of the barcode region (e.g., between the barcode region and a 5′ tethering region). In some embodiments, the subpool barcode is part of the 5′ tethering region.

In some embodiments, the set of splint oligonucleotides used in one subpool and the set of splint oligonucleotides used in another subpool includes the same sequences except for the barcode region(s) that is unique to each subpool. In some embodiments, the first splint oligonucleotide of a first subpool of the plurality of subpools is the same as the first splint oligonucleotide of a second subpool of the plurality of subpools except for the subpool barcode. In some embodiments, the first splint oligonucleotides of a plurality of the first splint oligonucleotides of a first subpool of the plurality of subpools is the same as the first splint oligonucleotides of a plurality of the first splint oligonucleotides of a second subpool of the plurality of subpools, except for the subpool barcode. In some embodiments, subpooling increases the number of analytes that can be assayed than may be possible without subpooling. For example, without limitation, a paired combination of 500 splint oligonucleotides, where each pair is orthogonal to every pair, can be used to assay for 2000 analytes using 4 subpools, where the splint oligonucleotides in each subpool have subpool barcodes by way of which the subpool from which on-target extension products generated from each pair of splint oligonucleotides originated can be identified after the subpools are combined (e.g., before sequencing). In some embodiments, the first splint oligonucleotide is attached to the first construct via hybridization to a first tether oligonucleotide attached to the first construct, and/or the second splint oligonucleotide is attached to the second moiety via hybridization to a second tether oligonucleotide attached to the second moiety, wherein the first splint oligonucleotide and/or second splint oligonucleotide comprises a 5′ tethering region that hybridizes to the first tether oligonucleotide and/or second tether oligonucleotide, wherein the 5′ tethering region comprises the subpool barcode.

The subpool barcode can be any suitable length. In some embodiments, the first splint oligonucleotide comprises a first subpool barcode that is 1, 2, 3, 4, 5, 6, 7, or 8 nucleotides long. In some embodiments, the first splint oligonucleotide comprises a first subpool barcode that is 2-4 nucleotides long. In some embodiments, the second splint oligonucleotide includes a second subpool barcode that is 1, 2, 3, 4, 5, 6, 7, or 8 nucleotides long. In some embodiments, the second subpool barcode is 2-4 nucleotides long. In some embodiments, the first splint oligonucleotide and second splint oligonucleotide each includes a subpool barcode. In some embodiments, the first splint oligonucleotide and second splint oligonucleotide each includes a subpool barcode of, or of at least 1, 2, 3, 4, 5, 6, 7, or 8 nucleotides in length. In some embodiments, the first splint oligonucleotide and second splint oligonucleotide each includes a subpool barcode of 2-4 nucleotides in length. In some embodiments, the length of the subpool barcodes in the first splint oligonucleotide and second splint oligonucleotide is the same (e.g., where the first construct includes a solid support to which the first splint oligonucleotide is attached). For example, without limitation, the first splint oligonucleotide can include a subpool barcode of 4 nt, and the second splint oligonucleotide can include a subpool barcode of 2 nt. In some embodiments, the length of the subpool barcodes in the first splint oligonucleotide and second splint oligonucleotide is different. For example, without limitation, the first splint oligonucleotide can include a subpool barcode of 4 nt, and the second splint oligonucleotide can include a subpool barcode of 4 nt.

### G. Oligonucleotide Trimming

In some embodiments, hybridization lengths in proximity extension assays can affect the performance of the assay. Increased hybridization region (“hyb”) length can increase the efficiency of extension reaction but can reach a tipping point where proximity effects are diminished and the hybridization energy is too strong leading to analyte-independent pulldown of detector and huge increases in assay background. Furthermore, a highly multiplexed proximity extension-type assays can have unique hybridization overlaps for each assay's assigned oligo pair. In some embodiments, proximity-type assays are sensitive to sequence specific differences in hybridization region. In some embodiments, ‘trimming’ refines and balances the variance of efficiencies across different unique hybridization lengths in a large multiplex. For example, 7 nucleotide (nt) hybs may have one base trimmed off the 3′ end of either capture-side or detect-side oligonucleotide (but not both), and still produce a 6 hyb length, and full-length amplicon that is identical to the non-trimmed version.

In some embodiments, the method includes providing a plurality of paired combinations of the first construct and second construct, wherein the 3′ hybridizing region of the splint oligonucleotides of one or more trimmed paired combinations is 1, 2, 3 or more nucleotides shorter than the 3′ hybridizing region of the splint oligonucleotides of at least one other paired combination of the plurality of paired combinations, wherein the 3′ hybridizing regions of the splint oligonucleotides of the at least one other paired combination of the plurality of paired combinations is different from and is not complementary to any contiguous stretch of the 3′ hybridizing region of the splint oligonucleotides of the one or more trimmed paired combinations. In some embodiments, a difference in the length of the 3′ hybridization regions among all of the splint oligonucleotides of the plurality of paired combinations is no more than 1, 2, or 3 nucleotides. In some embodiments, the length of the 3′ hybridization regions of the splint oligonucleotides of the plurality of paired combinations (e.g., including trimmed and non-trimmed splint oligonucleotides) is in a range of 4-9 nt (e.g., 4-7 nt, 5-7 nt, 6-7 nt, 6-8 nt, 7-8 nt, 7-9 nt, etc.). In some embodiments, the length of the 3′ hybridization regions of the splint oligonucleotides of the plurality of paired combinations is 6 or 7 nt.

The plurality of paired combinations can be obtained using any suitable option. In some embodiments, an initial plurality of paired combinations of the first construct and second construct, wherein the 3′ hybridizing regions of the splint oligonucleotides of each paired combination of the initial plurality of paired combinations have the same length as each other. In some embodiments, the 3′ hybridizing regions of the splint oligonucleotides of each paired combination of the initial plurality of paired combinations are orthogonal to each other. Then, 1, 2, or 3 nucleotides of the 3′ end of the first or second splint oligonucleotide (but not both) are removed (“trimmed”) to generate the plurality of paired combinations, wherein the 3′ hybridizing region of the splint oligonucleotides of one or more trimmed paired combinations is 1, 2, 3 or more nucleotides shorter than the 3′ hybridizing region of the splint oligonucleotides of at least one other paired combination of the plurality of paired combinations. In some embodiments, the 3′ hybridizing regions of the splint oligonucleotides of each paired combination of the plurality of paired combinations remain orthogonal to each other, after trimming. In some embodiments, the capture side (e.g., the splint oligonucleotide attached to a solid support) is trimmed. In some embodiments, the detection side (e.g., the splint oligonucleotide conjugated to the moiety) is trimmed.

Any suitable proportion of the paired combinations in the assay can include a trimmed splint oligonucleotide. In some embodiments, the one or more paired combinations (e.g., trimmed paired combinations having a trimmed splint oligonucleotide) comprises, comprises about, or comprises at most 10, 20, 30, 40, 50, 60, 70, 80, 90% or more of the plurality of paired combinations, or optionally wherein the one or more trimmed paired combinations comprises a percentage in a range defined by any two of the preceding values (e.g., about 10-90%, about 20-80%, about 40-60%, about 20-40%, etc.) of the plurality of paired combinations. In some embodiments, the one or more paired combinations (e.g., trimmed paired combinations having a trimmed splint oligonucleotide) comprises about one third of the plurality of paired combinations.

In some embodiments, trimming the splint oligonucleotides reduces non-specific background signal compared to the untrimmed version. In some embodiments, a frequency of analyte-independent on-target interactions (e.g., as observed in the absence of the analyte) between the splint oligonucleotides of the plurality of paired combinations is reduced compared to when the 3′ hybridizing region of the splint oligonucleotides of the one or more trimmed paired combinations is the same length as the 3′ hybridizing region of the splint oligonucleotides of the at least one other paired combination of the plurality of paired combinations. In some embodiments, a total variation in a calculated ΔG of hybridization between the 3′ hybridizing regions among the plurality of paired combinations is reduced, compared to when the 3′ hybridizing region of the splint oligonucleotides of the one or more trimmed paired combinations is the same length as the 3′ hybridizing region of the splint oligonucleotides of the at least one other paired combination of the plurality of paired combinations. In some embodiments, a calculated ΔG of hybridization between the 3′ hybridizing region of each of the plurality of paired combinations of the first splint oligonucleotide and second splint oligonucleotide is in a range of about −3 to −4 kcal/mol. In some embodiments, the on-target extension products generated from, from at least, or from about 10, 20, 30, 40, 50, 55, 60, 65, 70, 75, 80, 85, 90, 95, 96, 97, 98, 99%, or about 100% of the plurality of paired combinations of the first construct and second construct have the same length (e.g., where the 3′ hybridizing region of at least one of the plurality of paired combinations has a different length from the other paired combinations), optionally where the percentage is in a range defined by any two of the preceding values (e.g., 10-100%, 20-90%, 50-99%, 70-98%, 75-95%, etc.). In some embodiments, the on-target extension products generated from all, or substantially all (e.g., at least 95%), of the plurality of paired combinations of the first construct and second construct have the same length (e.g., where the 3′ hybridizing region of at least one of the plurality of paired combinations has a different length from the other paired combinations). As used herein, the length of the on-target extension product denotes the sum of the lengths of the first and second splint oligonucleotides, minus the length of the 3′ hybridizing region (to account for the overlap due to hybridization between the splint oligonucleotides).

### H. Attenuation

In some embodiments, different oligonucleotide sets (e.g., splint oligonucleotide sets) can have different efficiencies of producing amplicon. In some embodiments, the assignment of high efficiency sets to low abundance analytes and vice versa could be used to improve the uniformity of the distribution of amplicons within a pool. In some embodiments, lower efficiency oligonucleotide sets or other options to attenuate the production of on-target amplicon from the most abundant analytes can be used, such that the disparity in abundance that each subpanel can accommodate is expanded. In some embodiments, this range is increased from 20-fold to 400-fold using one or more attenuation options described herein. In some embodiments, use of one or more attenuation options described herein allows running the assay in fewer number of subpanels/subpools (e.g., two with attenuation, instead of four without attenuation), which produces a significant improvement in throughput. In some embodiments, all analytes are assayed in one panel/pool, when using one or more attenuation options described herein. In some embodiments, use of one or more attenuation options described herein reduces the variance in signal (e.g., amount of on-target extension product detected) across different analytes in a reaction or subpool (e.g., by lowering the signal for high abundance analytes).

In some embodiments, the method includes attenuating an amount of amplification products by reducing or interfering with a binding interaction between the analyte and the first moiety or the second moiety, and/or suppressing on-target interactions between the conjugate splint oligonucleotide and the first splint oligonucleotide when the first moiety and the second moiety are both bound to the analyte (e.g., bound to the same analyte molecule such that the 3′ hybridizing region of the first splint oligonucleotide and the 3′ hybridizing region of the second splint oligonucleotide are in proximity). Any suitable option for attenuating signal can be used.

In some embodiments, reducing or interfering with a binding interaction between the analyte and the first moiety or second moiety comprises: preparing the complexing solution in the presence of an attenuating moiety (e.g., a “cold” antibody) that competes for binding to the analyte with the first moiety and/or second moiety, wherein the attenuating moiety is not attached to a splint oligonucleotide comprising a 3′ hybridization region complementary to the 3′ hybridizing region of the first splint oligonucleotide or second splint oligonucleotide. In some embodiments, the attenuating moiety competes for binding to the analyte with the first moiety and is not attached to a first splint oligonucleotide comprising a 3′ hybridization region complementary to the 3′ hybridizing region of the second splint oligonucleotide. In some embodiments, the attenuating moiety competes for binding to the analyte with the first moiety and is not attached to a solid support to which the first moiety and the first splint oligonucleotide is attached (e.g., is not attached to a barcoded bead). In some embodiments, the attenuating moiety competes for binding to the analyte with the first moiety and is not biotinylated (e.g., is not a biotinylated antibody).

Any suitable amount of the attenuating moiety can be present in the complexing solution to reduce or interfere with a binding interaction between the analyte and the first moiety or second moiety. In some embodiments, the attenuating moiety is present in the complexing solution at a concentration of, of about, or of at least 5, 10, 20, 30, 40, 50, 75, 100, 150, 200, 250, 300, 400, 500, 600, 700, 800, 900, 1,000, 2,000, 3,000, 4,000, 5,000, 6,000, 7,000, 8,000, 9,000, 10,000, 20,000, 50,000, 10⁵ pM or more, or optionally at a concentration in a range defined by any two of the preceding values (e.g., about 5-50,000 pM, about 10-20,000 pM, about 50-10,000 pM, about 20-8,000 pM, about 500-10,000 pM, etc.). In some embodiments, the attenuating moiety is present in the complexing solution at stoichiometric concentrations to the first moiety and/or second moiety. In some embodiments, the attenuating moiety is present in the complexing solution at a concentration in excess of the first moiety and/or second moiety. In some embodiments, the attenuating moiety is present in the complexing solution at a concentration of, of about, or of at least 2, 3, 4, 5, 6, 7, 8, 9, 10, 20, 30, 40, 50, 60, 70, 80, 90, 100, 1000, 5000, 10,000 fold or more higher than the concentration of the first moiety and/or second moiety, or optionally is present in the complexing solution at a concentration that is higher than the concentration of the first moiety and/or second moiety by a fold amount in a range defined by any two of the preceding values (e.g., 2-10 fold, 10-50 fold, 50-100 fold, 100-1000 fold, 1000-10,000 fold, etc.). In some embodiments, the attenuating moiety is present in the complexing solution at a concentration of, of about, or of at least 2, 3, 4, 5, 6, 7, 8, 9, 10, 20, 30, 40, 50, 60, 70, 80, 90, 100, 1000, 5000, 10,000 fold or more lower than the concentration of the first moiety and/or second moiety, or optionally is present in the complexing solution at a concentration that is lower than the concentration of the first moiety and/or second moiety by a fold amount in a range defined by any two of the preceding values (e.g., 2-10 fold, 10-50 fold, 50-100 fold, 100-1000 fold, 1000-10,000 fold, etc.).

In some embodiments, reducing or interfering with a binding interaction between the analyte and the first moiety or second moiety comprises: preparing the complexing solution by: i) combining in the solution the first construct and the second construct with the sample in the presence of an attenuating moiety that binds the analyte and is not attached to a splint oligonucleotide comprising a 3′ hybridization region complementary to the 3′ hybridizing region of the first splint oligonucleotide or second splint oligonucleotide, wherein the attenuating moiety competes for binding to the analyte with the first moiety and/or second moiety; ii) contacting the first construct with a sample in the presence of an attenuating moiety that binds the analyte and is not attached to a splint oligonucleotide comprising a 3′ hybridization region complementary to the 3′ hybridizing region of the second splint oligonucleotide, wherein the attenuating moiety competes for binding to the analyte with the first moiety; and combining in a solution the sample-contacted construct and the second construct; or iii) contacting the second construct with a sample in the presence of an attenuating moiety that binds the analyte and is not attached to a splint oligonucleotide comprising a 3′ hybridization region complementary to the 3′ hybridizing region of the second splint oligonucleotide, wherein the attenuating moiety competes for binding to the analyte with the first moiety; and combining in a solution the sample-contacted second construct with the first construct. In some embodiments, the attenuating moiety is attached to a solid support (e.g., a bead), wherein the solid support is not attached to the splint oligonucleotide comprising the 3′ hybridization region complementary to the 3′ hybridizing region of the first splint oligonucleotide or second splint oligonucleotide.

In some embodiments, reducing or interfering with a binding interaction between the analyte and the first moiety or second moiety comprises: preparing the complexing solution in the presence of a pseudo-analyte that can be bound by either one of the first moiety or second moiety but not by the other. In some embodiments, the pseudo-analyte competes with the target analyte for binding to the first or second moiety, but cannot be bound by both the first moiety and second moiety. In some embodiments, the pseudo-analyte can be bound by the first moiety (e.g., the capture moiety bound to a solid support) but cannot be bound by the second moiety (e.g., the detection moiety of the detection conjugate). In some embodiments, the determined amount of the on-target extension product represents the ratio of the amount of target analyte and the amount of pseudo-analyte.

Any suitable amount of the pseudo-analyte can be present in the complexing solution to reducing or interfering with a binding interaction between the analyte and the first moiety or second moiety. In some embodiments, the pseudo-analyte is present in the complexing solution at a concentration of, of about, or of at least 5, 10, 20, 30, 40, 50, 75, 100, 150, 200, 250, 300, 400, 500, 600, 700, 800, 900, 1,000, 2,000, 3,000, 4,000, 5,000, 6,000, 7,000, 8,000, 9,000, 10,000, 20,000, 50,000, 10⁵, 10⁶ pM or more, or optionally at a concentration in a range defined by any two of the preceding values (e.g., about 5-10⁶ pM, about 5-50,000 pM, about 10-20,000 pM, about 50-10,000 pM, about 20-8,000 pM, about 500-10,000 pM, etc.). In some embodiments, the pseudo-analyte is present in the complexing solution at stoichiometric concentrations to the first moiety and/or second moiety. In some embodiments, the pseudo-analyte is present in the complexing solution at a concentration in excess of the first moiety and/or second moiety. In some embodiments, the pseudo-analyte is present in the complexing solution at a concentration of, of about, or of at least 2, 3, 4, 5, 6, 7, 8, 9, 10, 20, 30, 40, 50, 60, 70, 80, 90, 100, 1000, 5000, 10,000 fold or more higher than the concentration of the first moiety and/or second moiety, or optionally is present in the complexing solution at a concentration that is higher than the concentration of the first moiety and/or second moiety by a fold amount in a range defined by any two of the preceding values (e.g., 2-10 fold, 10-50 fold, 50-100 fold, 100-1000 fold, 1000-10,000 fold, etc.). In some embodiments, the pseudo-analyte is present in the complexing solution at a concentration of, of about, or of at least 2, 3, 4, 5, 6, 7, 8, 9, 10, 20, 30, 40, 50, 60, 70, 80, 90, 100, 1000, 5000, 10,000 fold or more lower than the concentration of the first moiety and/or second moiety, or optionally is present in the complexing solution at a concentration that is lower than the concentration of the first moiety and/or second moiety by a fold amount in a range defined by any two of the preceding values (e.g., 2-10 fold, 10-50 fold, 50-100 fold, 100-1000 fold, 1000-10,000 fold, etc.).

In some embodiments, reducing or interfering with a binding interaction between the analyte and the first moiety or second moiety comprises: preparing the complexing solution by: i) combining in the solution the first construct and the second construct with the sample in the presence of a pseudo-analyte; ii) contacting the first construct with a sample in the presence of a pseudo-analyte; and combining in a solution the sample-contacted construct and the second construct; or iii) contacting the second construct with a sample in the presence of a pseudo-analyte; and combining in a solution the sample-contacted second construct with the first construct.

In some embodiments, reducing or interfering with a binding interaction between the analyte and the first moiety or second moiety comprises: the first moiety (e.g., a capture moiety attached to a solid support) is a lower affinity binding moiety (e.g., has weaker binding) for the analyte than a higher affinity binding moiety for the analyte, wherein a detected signal for the amount of the analyte assayed is lower when the first moiety is the lower affinity binding moiety for the analyte (for example, when used under otherwise comparable assay conditions, e.g., concentrations of analyte, first moiety, second moiety, splint oligonucleotides, etc.). In some embodiments, the lower affinity binding moiety has a binding affinity for the analyte of, of at most, or of about, 1, 2, 5, 10, 100, 200, 500, 1,000, 2,000, 5,000, 10,000, 50,000, 100,000 nM or higher, or optionally the lower affinity binding moiety has a binding affinity for the analyte in a range defined by any two of the preceding values (e.g., 1-100,000 nM, 1-10 nM, 10⁻¹⁰⁰ nM, 100-500 nM, 500-1,000 nM, 1,000-5,000 nM, 5,000-10,000 nM, 10,000-100,000 nM, etc.). In some embodiments, the lower affinity binding moiety has a binding affinity that is lower than the binding affinity of the higher affinity binding moiety by, by at least, or by about, 10, 20, 30, 40, 50, 60, 70, 80, 90, 95, 98, 99, 99.9, 99.99, 99.999%, or higher, and optionally the lower affinity binding moiety has a binding affinity for the analyte that is lower than the binding affinity of the higher affinity binding moiety by a percentage in a range defined by any two of the preceding values (e.g., 10-99.999%, 10-50%, 50-80%, 80-90%, 90-95%, 95-99%, 99-99.9%, 99-99.99%, 99-99.999%, etc.). In some embodiments, the detected signal is reduced by, by about, or by at least, 10, 15, 20, 25, 30, 35, 40, 45, 50, 55, 60, 65, 70, 75, 80, 85, 90, 95%, or about 100% when the first moiety is the lower affinity binding moiety for the analyte than when it is the higher binding moiety for the analyte, and optionally the detected signal is reduced by a percentage in a range defined by any two of the preceding values (e.g., 10⁻⁹⁵%, 15-90%, 40-90%, 50-95%, 10-30%, etc.). In some embodiments, the dynamic range for detecting the analyte does not change, or does not change substantively (e.g., by no more than 10% or 15%), when using the lower affinity binding moiety compared to the higher affinity binding moiety. In some embodiments, the detected signal for the amount of the analyte assayed is saturated when the first moiety is the higher affinity binding moiety for the analyte, and is not saturated when the first moiety is the lower affinity binding moiety for the analyte. In some embodiments, the detected signal for the amount of the analyte assayed is saturated when the analyte is present in the sample at high abundance (e.g., in the order of 10⁻⁸, 10⁻⁷, or 10⁻⁶ g/mL, or higher) and when the first moiety is the higher affinity binding moiety for the analyte, and is not saturated when the first moiety is the lower affinity binding moiety for the analyte. In some embodiments, the method includes sequencing one or more extension products generated. In some embodiments, the sequencing includes high-throughput sequencing.

In some embodiments, suppressing on-target interactions between the second splint oligonucleotide and the first splint oligonucleotide comprises providing a 3′ hybridizing region having a first length that is shorter than a second length, wherein a detected signal for the amount of the analyte assayed is lower when the analyte is assayed with splint oligonucleotides having a 3′ hybridization region having the first length than having the second length. In some embodiments, the detected signal is reduced by, by about, or by at least, 10, 15, 20, 25, 30, 35, 40, 45, 50, 55, 60, 65, 70, 75, 80, 85, 90, 95%, or about 100% when the analyte is assayed with splint oligonucleotides having a 3′ hybridization region having the first length than when they have the second length, and optionally the detected signal is reduced by a percentage in a range defined by any two of the preceding values (e.g., 10⁻⁹⁵%, 15-90%, 40-90%, 50-95%, 10-30%, etc.). In some embodiments, the dynamic range for detecting the analyte does not change, or does not change substantively (e.g., by no more than 10% or 15%), when using splint oligonucleotides having a 3′ hybridization region having the first length than when they have the second length. In some embodiments, the detected signal for the amount of the analyte assayed is saturated when the 3′ hybridizing region has the second length, and is not saturated when the 3′ hybridizing region has the first length. In some embodiments, the detected signal for the amount of the analyte assayed is saturated when the analyte is present in the sample at high abundance (e.g., in the order of 10⁻⁸, 10⁻⁷, or 10⁻⁶ g/mL, or higher) and when the 3 hybridization region has the second length, and is not saturated when the 3′ hybridizing region has the first length. In some embodiments, suppressing on-target interactions between the second splint oligonucleotide and the first splint oligonucleotide comprises providing a 3′ hybridizing region having a first hybridization energy (e.g., in kcal/mol) that is greater (e.g., less negative) than a second hybridization energy, wherein a detected signal for the amount of the analyte assayed is lower when the analyte is assayed with splint oligonucleotides having a 3′ hybridization region having the first hybridization energy than having the second hybridization energy. In some embodiments, the hybridization energy is increased by, by about, or by at least, 0.1, 0.2, 0.3, 0.4, 0.5, 0.6, 0.7, 0.8, 0.9, 1, 1.2, 1.4, 1.6, 1.8, 2, 2.5, 3, 3.5, 4 kcal/mol or greater, when the analyte is assayed with splint oligonucleotides having a 3′ hybridization region having the first hybridization energy than having the second hybridization energy, and optionally the detected signal is reduced by a percentage in a range defined by any two of the preceding values (e.g., 10⁻⁹⁵%, 15-90%, 40-90%, 50-95%, 10-30%, etc.). In some embodiments, the dynamic range for detecting the analyte does not change, or does not change substantively (e.g., by no more than 10% or 15%), when using splint oligonucleotides having a 3′ hybridization region having the first hybridization energy than having the second hybridization energy. In some embodiments, the detected signal for the amount of the analyte assayed is saturated when the 3′ hybridizing region has the second hybridization energy, and is not saturated when the 3′ hybridizing region has the first hybridization energy. In some embodiments, the detected signal for the amount of the analyte assayed is saturated when the analyte is present in the sample at high abundance (e.g., in the order of 10⁻⁸, 10⁻⁷, or 10⁻⁶ g/mL, or higher) and when the 3 hybridization region has the second hybridization energy, and is not saturated when the 3′ hybridizing region has the first hybridization energy. In some embodiments, the method includes sequencing one or more extension products generated. In some embodiments, the sequencing includes high-throughput sequencing. The first length can include any suitable number of nucleotides. In some embodiments, the first length is 4, 5, 6, 7, 8, or 9 nucleotides long, or longer. In some embodiments, the first length is 6 nucleotides long. In some embodiments, the first length is 7 nucleotides long. In some embodiments, the first length is 5 nucleotides long. In some embodiments, the first length is shorter than the second length by 1, 2, 3 or more nucleotides. In some embodiments, the first length is shorter than the second length by 1 nucleotide.

In some embodiments, suppressing on-target interactions between the second splint oligonucleotide and the first splint oligonucleotide comprises: the first construct comprises a solid support (e.g., a bead) comprising: the first moiety attached to the solid support; and the first splint oligonucleotide attached to the solid support, wherein the first splint oligonucleotide is attached to the first moiety via the solid support, wherein the first splint oligonucleotide is attached to the solid support at a first ratio of the first splint oligonucleotide to the first moiety (or solid support having a standard amount of the first moiety attached thereto) that is lower than a second ratio of the first splint oligonucleotide to the first moiety (or solid support having a standard amount of the first moiety attached thereto), wherein a detected signal for the amount of the analyte assayed is lower when the first splint oligonucleotide and the first moiety is present on the solid support at the first ratio than at the second ratio (for example, when used under otherwise comparable assay conditions, e.g., concentrations of analyte, first moiety, second moiety, splint oligonucleotides, etc.). In some embodiments, the detected signal for the amount of the analyte assayed is saturated when the first splint oligonucleotide and the first moiety is present on the solid support at the second ratio, and is not saturated when the first splint oligonucleotide and the first moiety is present on the solid support at the first ratio. In some embodiments, the detected signal is reduced by, by about, or by at least, 10, 15, 20, 25, 30, 35, 40, 45, 50, 55, 60, 65, 70, 75, 80, 85, 90, 95%, or about 100% when the first splint oligonucleotide and the first moiety is present on the solid support at the first ratio than at the second ratio, and optionally the detected signal is reduced by a percentage in a range defined by any two of the preceding values (e.g., 10-95%, 15-90%, 40-90%, 50-95%, 10-30%, etc.). In some embodiments, the dynamic range for detecting the analyte does not change, or does not change substantively (e.g., by no more than 10% or 15%), when using the first splint oligonucleotide and the first moiety present on the solid support at the first ratio than at the second ratio. In some embodiments, the detected signal for the amount of the analyte assayed is saturated when the analyte is present in the sample at high abundance (e.g., in the order of 10⁻⁸, 10⁻⁷, or 10⁻⁶ g/mL, or higher) and the first moiety is present on the solid support at the second ratio, and is not saturated when the first splint oligonucleotide and the first moiety is present on the solid support at the first ratio. In some embodiments, the method includes sequencing one or more extension products generated. In some embodiments, the sequencing includes high-throughput sequencing.

The first ratio can be lower than the second ratio by any suitable degree. In some embodiments, the first ratio is lower than the second ratio by, by about, or by at least 2, 3, 4, 5, 10, 20, 30, 50, 100, 200, 500, 1,000, 2,000, 5,000, 10,000 fold or more, or optionally by a fold amount in a range defined by any two of the preceding values (e.g., 2-10,000 fold, 2-5,000 fold, 3-3,000 fold, 10-2,000 fold, etc.). In some embodiments, the first ratio is lower than the second ratio by about 3 to about 3000 fold. In some embodiments, the first ratio is lower than the second ratio by about 10, 100 or 1,000 fold. In some embodiments, the first ratio is lower than the second ratio by about 30 fold.

### III. DETERMINING THE PRESENCE AND/OR AMOUNT, OR THE ABSENCE OF, THE RELEASED ON-TARGET EXTENSION PRODUCT

In some embodiments, determining the presence and/or amount, or the absence of, the released on-target extension product comprises performing qPCR on one or more extension products (e.g., generated at block 1050 and released from the solid support at block 1060, with reference to FIG. 1, or generated at block 28050 and released at block 28060, with reference to FIG. 28, or generated at block 30050 and optionally released at block 30060, with reference to FIG. 30). In some embodiments, determining the presence and/or amount, or the absence of the released on-target extension product comprises obtaining sequencing data of one or more extension products (e.g., generated at block 1050 and released from the solid support at block 1060, with reference to FIG. 1, or generated at block 28050 and released at block 28060, with reference to FIG. 28, or generated at block 30050 and optionally released at block 30060, with reference to FIG. 30). In some embodiments, sequencing data is obtained by sequencing the one or more released extension products. Any suitable option can be used to sequence the released extension products. In some embodiments, sequencing includes, without limitation, high-throughput sequencing, Sanger sequencing, nanopore sequencing, capillary sequencing, sequencing by synthesis, sequencing by ligation, or a combination thereof.

In some embodiments, determining the presence and/or amount, or the absence of, the released on-target extension product comprises amplifying the one or more extension products (e.g., generated at block 1050 and released from the solid support at block 1060, with reference to FIG. 1, or generated at block 28050 and released at block 28060, with reference to FIG. 28, or generated at block 30050 and optionally released at block 30060, with reference to FIG. 30). Any suitable option can be used to amplify the extension products. In some embodiments, amplifying includes using PCR, multiple displacement amplification (MDA), transcription-mediated amplification (TMA), nucleic acid sequence-based amplification (NASBA), strand displacement amplification (SDA), real-time SDA, rolling circle amplification, circle-to-circle amplification, or a combination thereof. In some embodiments, the sequencing data includes sequences of the amplicons of the extension products. In some embodiments, sequencing includes sequencing the amplicons of the extension products.

In some embodiments, determining the presence and/or amount, or the absence of, the released on-target extension product comprises: combining the one or more extension products with a control oligonucleotide in an amplification mixture; amplifying the one or more extension products and the control oligonucleotide in the amplification mixture, to generate extension product amplicons and control oligonucleotide amplicons, detecting a level of the extension product amplicons and the control oligonucleotide amplicons; and normalizing the detected level of the extension product amplicons based on the detected level of the control oligonucleotide amplicons. In some embodiments, a control oligonucleotide (or “spike-in oligo”) is spiked in with the extension products. In some embodiments, the control oligonucleotide allows normalization of the detection of amplicons across different partitions (e.g., different wells) in which the assay is run. In some embodiments, the control oligonucleotide is a synthesized analog of the extension product.

In some embodiments, the extension product amplicons comprise adapter sequences that hybridize to one or more sequencing primers (e.g., sequencing primers). In some embodiments, the extension product amplicons comprise one or more indexing sequences. In some embodiments, the indexing sequence identifies the sample from which the extension product amplicons were derived. In some embodiments, the method includes analyzing samples from more than one source (e.g., clinical samples from more than one patient), pooling the extension product amplicons that include the indexing sequence before determining the presence or absence of the analyte, and using the indexing sequence to identify the source of the on-target extension product sequence in the sequencing data.

In some embodiments, the control oligonucleotide includes one or more unique molecular identifiers (UMI). In some embodiments, the control oligonucleotide includes a UMI, and detecting the level of the control oligonucleotide comprises obtaining sequence information of the UMI in the control oligonucleotide amplicons, and the detected level of the control oligonucleotide is based on the number of the UMI with distinct sequences associated with the sequence of the control oligonucleotide in the sequencing data. Any suitable UMI sequence can be used in the control oligonucleotide. In some embodiments, the UMI is at least 5, at least 6, at least 7, at least 8, at least 9, at least 10, at least 11, at least 12, at least 13, at least 14, at least 15, at least 16, at least 17, or at least 18 nucleotides long. In some embodiments, the UMI is about 8-16 nucleotides long. In some embodiments, a collection of control oligonucleotides having the UMI includes a diverse collection of UMI nucleotide sequences such that no two control oligonucleotide molecules in the collection of control oligonucleotides have the same UMI sequence (or such that the likelihood that any two control oligonucleotide molecules in the collection of control oligonucleotides have the same sequence is low enough to uniquely label all control oligonucleotides that are sequenced). In some embodiments, a collection of control oligonucleotides having the UMI includes a random sequence of nucleotides in each UMI.

In some embodiments, the sequencing analysis of the extension products involves determining the sequencing depth (or average sequencing depth). In some embodiments, where the control oligonucleotide includes the UMI, sequencing depth is determined based on an analysis of the UMI. In some embodiments, the UMI is provided in the extension product (e.g., the capture oligonucleotide and/or detection oligonucleotide includes one or more UMI, as provided herein).

In some embodiments, determining the presence and/or amount, or the absence of, the released on-target extension product involves determining that a sequence in the sequencing data corresponds to the on-target extension product. In some embodiments, the method includes identifying a sequence of an extension product in the sequencing data as being associated with the on-target extension product based on a pairing of: (A) one or more subsequences in the sequence associated with one splint oligonucleotide of a paired combination (e.g., the detection oligonucleotide or the second splint oligonucleotide), wherein the one or more subsequences identify the binding target of the analyte-binding moiety (e.g., the detection moiety or the second moiety) attached to the splint oligonucleotide, with (B) one or more subsequences in the sequence associated with the other splint oligonucleotide of the paired combination (e.g., the capture oligonucleotide or the first splint oligonucleotide), wherein the one or more subsequences identify the same binding target of the analyte-binding moiety (e.g., capture moiety or first moiety) associated with the other splint oligonucleotide. In some embodiments, the method includes identifying a sequence of an extension product in the sequencing data as being associated with the on-target extension product based on a pairing of: (A) one or more subsequences in the sequence associated with the detection oligonucleotide, wherein the one or more subsequences identify the binding target of the detection moiety attached to the detection oligonucleotide, with (B) one or more subsequences in the sequence associated with the capture oligonucleotide, wherein the one or more subsequences identify the same binding target of the capture moiety that is attached to the solid support to which the capture oligonucleotide is attached. By way of example and without limitation, a sequence of an extension product in the sequencing data may include a subsequence in the portion of the sequence attributed to the detection oligonucleotide that indicates that the detection moiety attached to the detection oligonucleotide binds to a given binding target, and may include a subsequence in the portion of the sequence attributed to the capture oligonucleotide that indicates that the capture moiety that is attached to the solid support to which the capture oligonucleotide is attached binds to the same binding target (e.g., the extension product was generated due to an on-target arrangement of the detection oligonucleotide and the capture oligonucleotide). In some embodiments, a sequence in the sequencing data is identified as an on-target extension product based on the pairing of the detection oligonucleotide sequence and the capture oligonucleotide sequence in the sequence of the extension product that indicates that the extension product was generated by an on-target arrangement of the detection oligonucleotide and the detection oligonucleotide. In some embodiments, the method includes identifying a sequence of an extension product in the sequencing data as being associated with the on-target extension product based on a pairing of: (A) one or more subsequences in the sequence associated with the second splint oligonucleotide, wherein the one or more subsequences identify the binding target of the second moiety attached to the second splint oligonucleotide, with (B) one or more subsequences in the sequence associated with the first splint oligonucleotide, wherein the one or more subsequences identify a binding target of the first moiety attached to the first splint oligonucleotide that is the same as the binding target of the second moiety.

In some embodiments, the subsequence is a barcode sequence, as provided herein. In some embodiments, the one or more subsequences in the sequence associated with the detection oligonucleotide comprises a first barcode sequence, and the one or more subsequences in the sequence associated with the capture oligonucleotide comprises a second barcode sequence. In some embodiments, the one or more subsequences in the sequence associated with the first splint oligonucleotide comprises a first barcode sequence, and the one or more subsequences in the sequence associated with the second splint oligonucleotide comprises a second barcode sequence.

In some embodiments, the barcode sequence is used to identify the binding target of the analyte-binding moiety associated with the splint oligonucleotide (e.g., capture oligonucleotides and/or detection oligonucleotides, or the first splint oligonucleotides and/or second splint oligonucleotides) that contains the barcode sequence. In some embodiments, the barcode sequence is used to identify the binding target of the detection or capture moiety associated with the detection oligonucleotide or the detection oligonucleotide, respectively. In some embodiments, the 3′ hybridizing region is used to identify the binding target of the detection or capture moiety associated with the detection oligonucleotide or the capture oligonucleotide, respectively. In some embodiments, the 3′ hybridizing region identifies the binding target of the detection oligonucleotide or capture oligonucleotide. In some embodiments, the 3′ hybridizing region identifies the binding target of the first splint oligonucleotide or second splint oligonucleotide.

In some embodiments, identifying a sequence of an extension product in the sequencing data as being associated with the on-target extension product is based on a length of the sequenced extension product, wherein an on-target extension product is associated with a sequenced extension product having a length consistent with the length expected for the on-target extension product. For example, an extension product may be generated through mis-priming of the 3′ hybridizing region of the splint oligonucleotides (e.g., the detection oligonucleotide and/or the capture oligonucleotide, or the first splint oligonucleotides and/or second splint oligonucleotides), and such an extension product will likely have a different length than expected from the intended hybridization interaction of the 3′ hybridizing regions. In some embodiments, an off-target extension product is associated with a sequenced extension product having a length that is different from the length expected for the on-target extension product. In some embodiments, the off-target extension product has a sequence that is shorter than the length expected for the on-target extension product. In some embodiments, the off-target extension product has a sequence that is at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 15 nucleotides, at least 20 nucleotides, at least 25 nucleotides, at least 30 nucleotides, or a length in a range defined by any two preceding values (e.g., 1-30 nucleotides, 2-25 nucleotides, 3 20 nucleotides, 5-30 nucleotides, 10⁻³⁰ nucleotides, etc.) shorter than the length expected for the on-target extension product.

In some embodiments, the expected length of the on-target extension product is the same length for on-target extension products for different analytes. In some embodiments, the expected length of the on-target extension product is the same length for on-target extension products for all analytes being assayed.

In some embodiments, identifying the sequence in the sequencing data as being associated with the on-target extension product comprises analyzing a length distribution of the one or more extension products. In some embodiments, the sequencing data is first analyzed for the length of the sequenced extension products (e.g., where the expected length of the on-target extension product is the same length for on-target extension products for different (or for all) analytes, and then analyzing the sequence of the extension products for pairing of the splint oligonucleotides (e.g., pairing of the detection oligonucleotide and the capture oligonucleotide, or pairing of the first splint oligonucleotide and second splint oligonucleotide).

In some embodiments, determining the presence and/or amount, or the absence of, the released on-target extension product involves determining that a sequence in the sequencing data corresponds to an off-target extension product. In some embodiments, a sequence in the sequencing data is identified as an off-target extension product based on a mispairing between: (A) the one or more subsequences in the sequence associated with the detection oligonucleotide, wherein the one or more subsequences identify the binding target of the detection moiety attached to the detection oligonucleotide, and (B) the one or more subsequences in the sequence associated with the capture oligonucleotide, wherein the one or more subsequences identify a different binding target of a different capture moiety that is attached to a different solid support to which the capture oligonucleotide is attached. In some embodiments, the method includes removing from the sequencing data sequences associated with off-target extension products (e.g., identified based on length and/or mis-pairing of the splint oligonucleotides (e.g., the detection oligonucleotide and the capture oligonucleotide, or the first splint oligonucleotide and second splint oligonucleotide). In some embodiments, a sequence in the sequencing data is identified as an off-target extension product based on a mispairing between: (A) the one or more subsequences in the sequence associated with the second splint oligonucleotide, wherein the one or more subsequences identify the binding target of the second moiety attached to the second splint oligonucleotide, and (B) the one or more subsequences in the sequence associated with the first splint oligonucleotide, wherein the one or more subsequences identify a different binding target of a different first moiety that is attached to a different solid support to which the first splint oligonucleotide is attached. In some embodiments, the subsequence is a barcode sequence, as provided herein.

In some embodiments, the on-target extension product and the spike-in oligo are amplified linearly during all subsequent amplification steps, e.g., adapter PCR and index PCR steps, thereby preserving the linear relationship between analyte concentration and the detected level of the extension product amplicons. However, there are a finite number of reads available for all the amplicons in a sample set being analyzed on most next generation sequencing platforms. Multiple samples (e.g., patients, wells, etc.) may be merged into a single NGS run to increase throughput. During library preparation, these samples are indexed with a sequencing barcode unique to that sample (e.g., patient, well, etc.) prior to pooling and sequencing. The reads associated with each sample (e.g., patient, well, etc.) can later be determined by de-multiplexing the indices. Due to the finite number of reads available for all amplicons in the pooled sample, the indices (i.e., well/patient/sample) that are more dilute will be under-represented during sequencing. Conversely, the indices (i.e., well/patient/sample) that are more concentrated will be over-represented and sequester a greater fraction of the reads. This can create a number of informatic problems, including but not limited to: lack of read depth for dilute samples, over-quantitation of amplicons in more concentrated samples, etc. Provided herein are various methods of normalizing the number of sequencing reads associated with each sample (e.g., patient, well, etc.).

In some embodiments, partitions that produce, or are expected to produce, a large number of extension product amplicons can be diluted prior to one or more subsequent amplification step, thereby producing a more uniform distribution of amplicon concentrations across different partitions (e.g., different wells) in which the assay is run and preserve the sensitivity and dynamic range of the assay. In some embodiments, qPCR using PCR primers common to all amplicon molecules in a partition can be performed to measure the total on-target extension product concentration in each partition (also known as empirical normalization); all partitions can then be diluted by various amounts before amplification such that each partition produces a more uniform distribution of extension product amplicons, thereby normalizing the number of sequencing reads attributed to each partition and increasing the sensitivity and dynamic range. In some embodiments, a number of sequencing reads attributed to a first diluted partition is, or is expected to be, within, within about, or within at most 100, 90, 80, 60, 50, 40, 30, 20, 10, 5, 2-fold the concentration of a number of sequencing reads attributed to a second diluted partition, or optionally the number of sequencing reads attributed to a first diluted partition is, or is expected to be, a fold amount of the concentration of a number of sequencing reads attributed to a second diluted partition in a range defined by any two of the preceding values (e.g., about 2-100 fold, about 10⁻⁹⁰ fold, 20-50 fold, etc.).

In some embodiments, when the number of extension product amplicons in a partition can be predicted (e.g., in the case of the calibration curve wells), the samples can be normalized without measuring the total amplicon produced in each partition (also known as blind normalization). For example, for a 4-fold dilution series, Standard 1 can be diluted 4 steps (256-fold, to yield approximately the same amplicon concentration as standard 5), Standard 2 can be diluted 64-fold, Standard 3 can be diluted 16-fold, and Standard 4 can be diluted 4-fold. Assuming the on-target extension product concentration in each partition is linearly proportional to the concentration of analyte/calibrator, then this would reduce the total amount of extension produce amplicons generated from the calibration curve partitions by ˜260-fold in the overall sequencing library, while preserving the same dynamic range of the calibration curve. The balanced utilization of sequencing reads across all standards would improve sensitivity as it would preserve considerably more reads for samples other than the highest standards.

In some embodiments, the adapter PCR amplification may be performed such that the adapter primers are present as a limiting reagent in the reaction. As such, PCR will terminate once the primers are depleted in samples that contain a high on-target extension product concentration, but the PCR process will continue linearly for additional cycles in samples where the on-target extension product concentration was lower and thus has not yet depleted the primers from the PCR reaction. This will result in well-to-well normalization equal to the limiting concentration of primers in the PCR reaction. At a minimum, the adapter PCR reaction should be run for a minimum of 2 cycles before primer depletion is reached to ensure that a sufficient number of amplicons have been synthesized to include the adapter tail for sequencing. In some embodiments, the concentration of the adapter primers used in the adapter PCR amplification is sufficient to allow the samples that contain the highest on-target extension product concentration to proceed through at least 2, 3, 4, 5, 6, 7, 8, 9, or 10 cycles of amplification, and at most 15, 14, 13, 12, 11, 10, 9, 8, 7, 6, or 5 cycles of amplification before primer depletion. Since the spike-in oligo is included at a fixed level for all samples/partitions, the ratio of spike-in to analyte amplicon in each well can be used to back calculate the actual number of reads and analyte molecules in a sample. In some embodiments, multiple spike-in oligos are used to provide a more rigorous correction system.

In some embodiments, the index PCR amplification may be performed such that the index primers are present as a limiting reagent in the reaction. As such, PCR will terminate once the primers are depleted in samples that contained a high on-target extension product concentration and thus a high adapter PCR extension product concentration, but the PCR process will continue linearly for additional cycles in samples where the on-target extension product concentration was lower and thus has not yet depleted the primers from the PCR reaction. This will result in well-to-well normalization equal to the limiting concentration of primers in the PCR reaction. At a minimum, the index PCR reaction should be run for a minimum of 2 cycles before primer depletion is reached to ensure that a sufficient number of amplicons have been synthesized to include the index tail for sequencing. In some embodiments, the concentration of the index primers used in the index PCR amplification is sufficient to allow the samples that contain the highest adapter PCR extension product concentration to proceed through at least 2, 3, 4, 5, 6, 7, 8, 9, or 10 cycles of amplification, and at most 15, 14, 13, 12, 11, 10, 9, 8, 7, 6, or 5 cycles of amplification before primer depletion. Since the spike-in oligo is included at a fixed level for all samples/partitions, the ratio of spike-in to analyte amplicon in each well can be used to back calculate the actual number of reads and analyte molecules in a sample. In some embodiments, multiple spike-in oligos are used to provide a more rigorous correction system.

In some embodiments, virtual calibration points generated from historical data can be combined with empirical calibration points from measured standards to generate a full calibration curve while reducing the proportion of sequencing reads used by standards and reducing number of partitions (e.g., wells) required for the standard curves. In some embodiments, virtual calibration points are determined by (a) preparing standard curves (e.g., 8-point standard curves) for all assays in singleplex; (b) running PESD assays on the standards; (c) quantifying the amplicons by qPCR; and (d) fitting the standard curves (e.g., curve fitting with 4-parameter logistic curve (4PL)); thereby the parameters are extracted and saved for future use. In some embodiments, virtual calibration points are determined by (a) preparing standard curves (e.g., 8-point standard curves) for all assays in multiplex; (b) running PESD assays on the standards; (c) quantifying the amplicons using an NGS-based readout; and (d) fitting the standard curves (e.g., with 4PL); thereby the parameters are extracted and saved for future use. The slope and midpoint parameters, which are intrinsic properties of the assay, should be identical regardless of which method is used for readout. The top and bottom parameters for the 4PL fit depend on the scale of the readout. In NGS these will vary from run to run depending on the number of total reads produced in the NGS run, the number of assays, samples, and amount of amplicon per sample. As such, the bottom and top parameters need to be scaled using the empirical data from a truncated standard curve, which can be prepared, for example, using the lowest 4 points (e.g., blank and standards 7, 6, and 5) of a typical 8-point curve. The complete calibration curve may be constructed by scaling and offsetting the historical calibration curve to minimize error between the 4 empirical points and the 4 lowest points of the previously measured/virtual curve. For example, the bottom parameter is the signal in the empirical blank; the slope and midpoint parameters are taken directly from the historical data; top is calculated by scaling the historical top parameter using a scaling factor alpha. The scaling factor alpha may be calculated by taking the ratio of the signal at the highest empirical calibration point, minus the blank, in the empirical data, to the corresponding points in the historical calibration curve.

### IV. METHODS OF SCREENING FOR BINDING MOIETY PAIRS

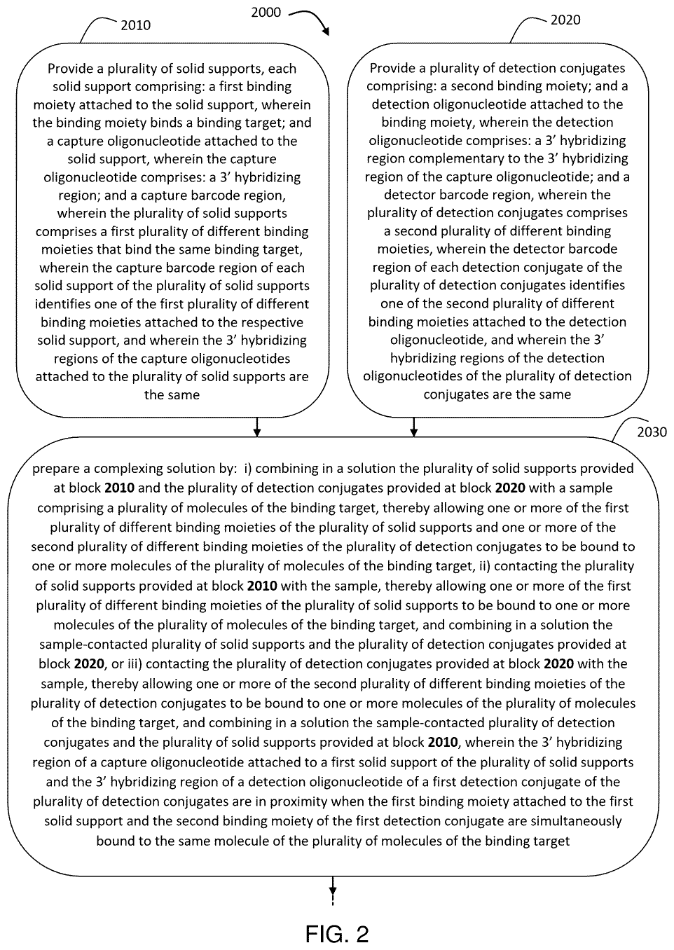

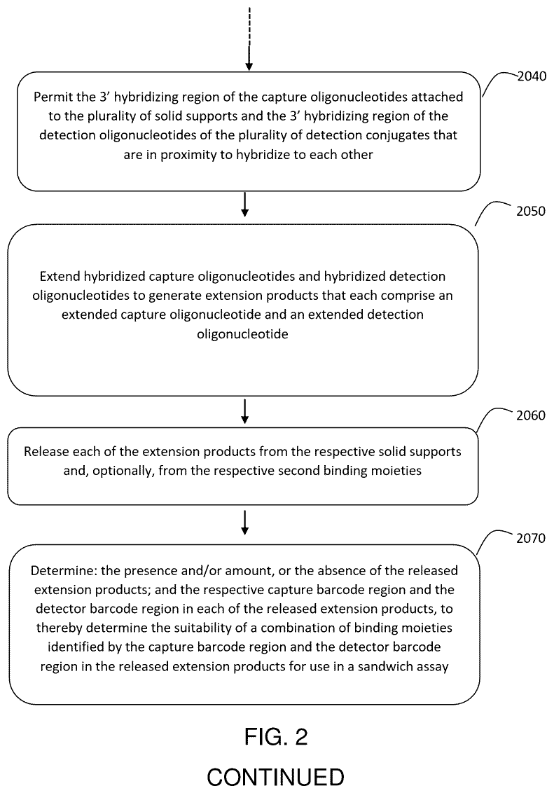

With reference to FIG. 2, a non-limiting example of a method 2000 of identifying a pairwise combination of binding moieties that can both be bound (or can be simultaneously bound) to a binding target is provided (which for convenience may be referred to herein as a “screening method”). The method can include, at block 2010, providing a plurality of solid supports, each solid support comprising: a first binding moiety attached to the solid support, wherein the binding moiety binds a binding target; and a capture oligonucleotide attached to the solid support, wherein the capture oligonucleotide is attached to the solid support independently of the binding moiety, and wherein the capture oligonucleotide comprises: a 3′ hybridizing region; and a capture barcode region, wherein the plurality of solid supports comprises a first plurality of different binding moieties that bind the same binding target, wherein the capture barcode region of each solid support of the plurality of solid supports identifies one of the first plurality of different binding moieties attached to the respective solid support, and wherein the 3′ hybridizing regions of the capture oligonucleotides attached to the plurality of solid supports are the same. The method can also include, at block 2020, providing a plurality of detection conjugates comprising: a second binding moiety; and a detection oligonucleotide attached to the binding moiety, wherein the detection oligonucleotide comprises: a 3′ hybridizing region complementary to the 3′ hybridizing region of the capture oligonucleotide; and a detector barcode region, wherein the plurality of detection conjugates comprises a second plurality of different binding moieties, wherein the detector barcode region of each detection conjugate of the plurality of detection conjugates identifies one of the second plurality of different binding moieties attached to the detection oligonucleotide, and wherein the 3′ hybridizing regions of the detection oligonucleotides of the plurality of detection conjugates are the same. The method can also include, at block 2030, preparing a complexing solution by: i) combining in a solution the plurality of solid supports provided at block 2010 and the plurality of detection conjugates provided at block 2020 with a sample comprising a plurality of molecules of the binding target, thereby allowing one or more of the first plurality of different binding moieties of the plurality of solid supports and one or more of the second plurality of different binding moieties of the plurality of detection conjugates to be bound to one or more molecules of the plurality of molecules of the binding target, ii) contacting the plurality of solid supports provided at block 2010 with the sample, thereby allowing one or more of the first plurality of binding moieties of the plurality of solid supports to be bound to one or more molecules of the plurality of molecules of the binding target, and combining in a solution the sample-contacted plurality of solid supports and the plurality of detection conjugates provided at block 2020, or iii) contacting the plurality of detection conjugates provided at block 2020 with the sample, thereby allowing one or more of the second plurality of different binding moieties of the plurality of detection conjugates to be bound to one or more molecules of the plurality of molecules of the binding target, and combining in a solution the sample-contacted plurality of detection conjugates and the plurality of solid supports provided at block 2010, wherein the 3′ hybridizing region of a capture oligonucleotide attached to a first solid support of the plurality of solid supports and the 3′ hybridizing region of a detection oligonucleotide of a first detection conjugate of the plurality of detection conjugates are in proximity when the first binding moiety attached to the first solid support and the second binding moiety of the first detection conjugate are both bound (or are simultaneously bound) to the same molecule of the plurality of molecules of the binding target. The method can further include, at block 2040, permitting the 3′ hybridizing region of the capture oligonucleotides attached to the plurality of solid supports and the 3′ hybridizing region of the detection oligonucleotides of the plurality of detection conjugates that are in proximity to hybridize to each other. At block 2050, the method can include extending hybridized capture oligonucleotides and/or hybridized detection oligonucleotides to generate extension products that each comprise an extended capture oligonucleotide and an extended detection oligonucleotide. The method can include, at block 2060, releasing each of the extension products from the respective solid supports and, optionally, from the respective second binding moieties. At block 2070, the method can include determining: the presence and/or amount, or the absence of the released extension products; and the respective capture barcode region and the detector barcode region in each of the released extension products, to thereby determine the suitability of a combination of binding moieties identified by the capture barcode region and the detector barcode region in the released extension products for use in a sandwich-type assay. As used herein, “sandwich assay” or “sandwich-type assay” denotes any in-vitro procedure that involves using the ability of two binding moieties to both be bound (or to be simultaneously bound) to a binding target to detect the presence and/or amount of the binding target in a sample.

In some embodiments, the plurality of solid supports comprises approximately equal amounts of each different binding moiety of the plurality of different binding moieties, and the plurality of detection conjugates comprises approximately equal amounts of each different binding moiety of the plurality of different binding moieties.

In some embodiments, the screening method allows screening for pairwise combinations of binding moieties that recognize the binding target from different sources and/or at different concentrations. In some embodiments, binding targets may have structural variations based on the source of the binding target (e.g., post-translation modifications, association with other proteins or factors, variation in protein folding, etc.). In some embodiments, the method includes providing a plurality of variants of the binding target; and for each of the plurality of variants of the binding target, contacting the plurality of solid supports or the plurality of detection conjugates, or both with a sample comprising the variant of the binding target. In some embodiments, the plurality of variants of the binding target comprises binding targets from different species and/or different sources. In some embodiments, the binding target is obtained from one or more of a recombinant source (e.g., recombinant bacteria, insect cell line, mammalian cell line, etc.), or a native sample (e.g., a clinical sample). In some embodiments, the method includes contacting the plurality of solid supports or the plurality of detection conjugates, or both with a plurality of samples, each comprising the plurality of molecules of the binding target at different concentrations. In some embodiments, the first plurality of different binding moieties and the second plurality of different binding moieties are the same set of different binding moieties. In some embodiments, all of the different binding moieties in the first plurality of different binding moieties are in the second plurality of different binding moieties. In some embodiments, the first plurality of different binding moieties and the second plurality of different binding moieties are different sets of different binding moieties (e.g., overlapping sets, or non-overlapping sets).

In some embodiments, the screening method 2000 includes the use of a strand-displacing polymerase to extend the and release the extension product. In some embodiments, in each solid support of the plurality of solid supports, the capture oligonucleotide is attached to the solid support via hybridization to a first tether oligonucleotide attached to the solid support. In some embodiments, in each detection conjugate of the plurality of detection conjugates, the detection oligonucleotide is attached to the detection moiety via hybridization to a second tether oligonucleotide attached to the detection moiety. In some embodiments, the extending at block 2050 comprises treating the plurality of solid support with a strand-displacing polymerase under conditions sufficient to extend hybridized capture oligonucleotides and hybridized detection oligonucleotides, and wherein the releasing at block 2060 comprises allowing the strand-displacing polymerase to displace the first tether oligonucleotide hybridized to the capture oligonucleotide during extension, and optionally to displace the second tether oligonucleotide hybridized to the detection oligonucleotide during extension.

In some embodiments, the capture moiety and/or the detection moiety is or includes an antibody or binding fragment thereof, a lectin, a receptor, a cofactor, a polynucleotide, an aptamer, a single chain protein binder, a peptide, a modified enzyme substrate, or a suicide inhibitor. In some embodiments, the capture moiety and/or the detection moiety is or includes an antibody or binding fragment thereof (e.g., scFv, Fab, F(ab′)2, etc.). In some embodiments, the capture moiety and/or the detection moiety is or includes a full-length antibody.

In some embodiments, the binding target is or includes an antibody, a polypeptide, a small molecule. In some embodiments, the binding target is or includes a cytokine. In some embodiments, the binding target is a pro-inflammatory cytokine. In some embodiments, the binding target is or includes an antibody. In some embodiments, the binding target is or includes an antibody that is an IgA, IgG, IgM.

The capture barcode region and the detector barcode region can be designed using any suitable option. In some embodiments, the capture barcode region and the detector barcode region are designed in silico. In some embodiments, designing the capture barcode region and the detector barcode region takes into account the calculated hybridization energy, e.g., between the capture or detection oligonucleotides. In some embodiments, barcode sequences that do not introduce bias in the present methods (e.g., screening methods) are designed using any suitable option. In some embodiments, designing capture barcode region and the detector barcode region suitable for use in a multiplex assay format of the present methods (e.g., screening methods) includes screening for lack of bias introduced by candidate capture barcode regions and the detector barcode regions. In some embodiments, screening for lack of bias introduced by candidate capture barcode regions and the detector barcode regions includes using a method of designing barcode regions as provided herein.

Also provided is a method of determining hybridization specificity of a hybridization region for a proximity-based extension assay (which for convenience may be referred to herein as a “hybridization specificity method”), e.g., for selecting 3′ hybridizing regions for use in the analyte detection methods of the present disclosure. The method can include providing a plurality of paired oligonucleotides, each paired oligonucleotide comprising: a capture oligonucleotide comprising a 3′ hybridizing region; and a detection oligonucleotide comprising a 3′ hybridizing region complementary to the 3′ hybridizing region of the capture oligonucleotide. The method can include, for at least one pair of the plurality of paired oligonucleotides: providing a first solid support comprising: a first capture moiety attached to the first solid support, wherein the first capture moiety binds an analyte; and a first capture oligonucleotide of the at least one pair of the plurality of paired oligonucleotides, wherein the first capture oligonucleotide is attached to the first solid support via hybridization to a first tether oligonucleotide attached to the first solid support. The method can also include providing a first detection conjugate comprising: a first detection moiety that binds the analyte; and a first detection oligonucleotide of the at least one pair of the plurality of paired oligonucleotides, wherein the first detection oligonucleotide is attached to the detection moiety. The method can also include providing either or both: one or more second solid supports, each comprising: a second capture moiety attached to the second solid support, wherein the second capture moiety binds the analyte, wherein the first capture moiety and second capture moiety have the same binding specificity; and a second capture oligonucleotide of a pair of the plurality of paired oligonucleotides that is different from the at least one pair of the plurality of paired oligonucleotides, wherein the second capture oligonucleotide is attached to the second solid support via hybridization to a first tether oligonucleotide attached to the second solid support; and one or more second detection conjugates, each comprising: a second detection moiety that binds the analyte, wherein the first detection moiety and second detection moiety have the same binding specificity; and a second detection oligonucleotide of a pair of the plurality of paired oligonucleotides that is different from the at least one pair of the plurality of paired oligonucleotides, wherein the second detection oligonucleotide is attached to the second detection moiety via hybridization to a second tether oligonucleotide attached to the second detection moiety. The method can also include preparing a complexing solution comprising: the first solid support and the first detection conjugate, wherein the first capture moiety attached to the first solid support and the first detection moiety of the detection conjugate are both bound (or are simultaneously bound) to the analyte, thereby forming a plurality of solid support-analyte-detection conjugate complexes such that: the 3′ hybridizing regions of the first capture oligonucleotide and the first detection oligonucleotide are in proximity to hybridize to each other; and either or both: the 3′ hybridizing regions of the first capture oligonucleotide and each of the one or more second detection oligonucleotides are in proximity; and the 3′ hybridizing regions of the first detection oligonucleotide and each of the one or more second capture oligonucleotides are in proximity. The method can further include permitting the 3′ hybridizing regions of the first capture and first detection oligonucleotides that are in proximity to hybridize to each other. The method can also include extending the hybridized first capture oligonucleotide and/or the hybridized first detection oligonucleotide in the presence of the plurality of solid support-analyte-detection conjugate complexes to generate: at least an on-target extension product comprising the extended first capture oligonucleotide and the extended first detection oligonucleotide; and any off-target extension product comprising either or both: the extended first capture oligonucleotide and an extended second detection oligonucleotide, and an extended second capture oligonucleotide and the extended first detection oligonucleotide. The method can further include determining the presence and/or amount of the on-target extension product, and the presence and/or amount or the absence of any off-target extension product, thereby determining hybridizing specificity of the 3′ hybridizing regions of the capture and detection oligonucleotides of the at least one pair of the plurality of paired oligonucleotides.

Also provided is a method of designing barcode regions (which for convenience may be referred to herein as a “barcode design method”), e.g., for use in the analyte detection method or screening method of the present disclosure. A method of identifying a capture barcode and/or a detector barcode suitable for use in a proximity-based extension assay can include providing a plurality of solid supports, each solid support comprising: a capture moiety attached to the solid support, wherein the capture moiety binds a binding target; and a capture oligonucleotide attached to the solid support, wherein the capture oligonucleotide comprises: a 3′ hybridizing region; and a capture barcode region, wherein the plurality of solid supports comprises a plurality of different capture barcode regions in the respective capture oligonucleotides, the plurality of solid supports comprises the same capture moiety, and wherein the 3′ hybridizing regions of the capture oligonucleotides attached to the plurality of solid supports are the same. The method can also include providing a plurality of detection conjugates comprising: a detection moiety that binds the binding target, wherein either the capture moiety or the detection moiety, or both, specifically binds to the binding target; and a detection oligonucleotide attached to the binding moiety, wherein the detection oligonucleotide comprises: a 3′ hybridizing region complementary to the 3′ hybridizing region of the capture oligonucleotide; and a detector barcode region, wherein the plurality of detection conjugates comprises a plurality of different detector barcode regions in the respective detection oligonucleotides, the plurality of detection conjugates comprises the same detection moiety, and wherein the 3′ hybridizing regions of the detection oligonucleotides of the plurality of detection conjugates are the same. The method can further include preparing a complexing solution comprising: the plurality of solid supports; the plurality of detection conjugates; and a population of the binding targets, wherein different combinations of a capture moiety of the plurality of solid supports and a detection moiety of the plurality of detection conjugates are bound (or simultaneously bound) to a binding target of the population of the binding targets such that the combination of capture oligonucleotide and detection oligonucleotide of the respective different combinations of solid support and detection conjugate are in proximity. The method can also include permitting the 3′ hybridizing region of the capture oligonucleotide and the 3′ hybridizing region of the detection oligonucleotide of the respective different combinations of solid support and detection conjugate that are in proximity to hybridize to each other. The method can include extending the hybridized capture oligonucleotides and/or hybridized detection oligonucleotides to generate extension products that each comprise an extended capture oligonucleotide and/or an extended detection oligonucleotide. The method can also include releasing one or both strands of each of the extension products from the respective solid supports and, optionally, from the respective detection moieties. The method can further include determining: the presence and/or amount, or the absence of the released extension products; and the respective capture barcode region and the detector barcode region in each of the released extension products, to thereby determine the suitability for use in a proximity-based extension assay of either or both a capture barcode region and detector barcode region in the released extension products.

In some embodiments, a capture barcode region or a detector barcode region that underperforms in generating extension products when paired with a plurality of detector barcode regions or a plurality of capture barcode regions, respectively, is identified as not being suitable for use in a proximity-based extension assay. In some embodiments, either a capture barcode region or a detector barcode region in a combination of capture barcode region and detector barcode region that overperforms in generating extension products is identified as not being suitable for use in a proximity-based extension assay.

### V. COMPOSITIONS

Also provided are compositions that include pairs of oligonucleotides that can be suitable for use in the methods (e.g., analyte detection methods) of the present disclosure. Provided herein is a composition that includes a plurality of (e.g., at least 30, or 30-200, or 30-500, or 30-1600, or 90-400, or 300-2000, or 200-5000, 1000-10000, etc.) pairs of oligonucleotides, each pair comprising: a capture oligonucleotide comprising: a 3′ hybridizing region and a 5′ tethering region; and a detection oligonucleotide comprising: a 3′ hybridizing region complementary to the 3′ hybridizing region of the capture oligonucleotide, and a 5′ tethering region, wherein the 3′ hybridizing region of the capture oligonucleotide and detection oligonucleotide of each pair of oligonucleotides is not complementary to the 3′ hybridizing region of the detection oligonucleotide and the capture oligonucleotide, respectively, of any other pair of the plurality of (e.g., at least 30, or 30-200, or 100-500, or 30-1600, or 90-400, or 300-2000, or 200-5000, 1000-10000, etc.) pairs of oligonucleotides, wherein each of the plurality of (e.g., at least 30, or 30-200, etc.) pairs of oligonucleotides has a mis-pairing rate of at most about 1% in the presence of the other plurality of (e.g., at least 30, or 30-200, or 100-500, or 30-1600, or 90-400, or 300-2000, or 200-5000, 1000-10000, etc.) pairs of oligonucleotides in a proximity-based extension assay. In certain embodiments, the capture oligonucleotide is at least 25 nucleotides long and its 3′ hybridizing region is at most 10 nucleotides long, and its 5′ tethering region is at least 15 nucleotides long; and the detection oligonucleotide is at least 25 nucleotides long and its 3′ hybridizing region is at most 10 nucleotides long and complementary to the 3′ hybridizing region of the capture oligonucleotide, and its 5′ tethering region is at least 15 nucleotides long, wherein the 3′ hybridizing region of the capture oligonucleotide and detection oligonucleotide of each pair of oligonucleotides is not complementary to the 3′ hybridizing region of the detection oligonucleotide and the capture oligonucleotide, respectively, of any other pair of the plurality of (e.g., at least 30, or 30-200, or 100-500, or 30-1600, or 90-400, or 300-2000, or 200-5000, or 1000-10000, etc.) pairs of oligonucleotides, wherein each of the plurality of (e.g., at least 30, or 30-200, or 100-500, or 30-1600, or 90-400, or 300-2000, or 200-5000, or 1000-10000, etc.) pairs of oligonucleotides has a mis-pairing rate of at most about 1% in the presence of the other plurality of (e.g., at least 30, or 30-200, or 100-500, or 30-1600, or 90-400, or 300-2000, or 200-5000, or 1000-10000, etc.) pairs of oligonucleotides in a proximity-based extension assay. As used herein, “mis-pairing” denotes hybridization of the 3′ hybridizing region of the capture oligonucleotide or detection oligonucleotide of one pair of the oligonucleotides with the 3′ hybridizing region of a different pair (e.g., where the 3′ hybridizing regions of the mis-paired oligonucleotides do not have complementary sequences). In some embodiments, the mis-pairing rate is, is about, or is at most 1%, 0.8%, 0.5%, 0.2%, 0.1%, 0.05%. 0.01%, 0.005%, 0.001% or less in the presence of the other at least 30 pairs of oligonucleotides in a proximity-based extension assay, or optionally, the mis-pairing rate is in a range defined by any two of the preceding values (e.g., 0.001-1%, 0.005-1%, 0.01-0.1%, etc.), in the presence of the other plurality of (e.g., at least 30, or 30-200, or 100-500, or 30-1600, or 90-400, or 300-2000, or 200-5000, or 1000-10000, etc.) pairs of oligonucleotides in a proximity-based extension assay. In some embodiments, oligonucleotides of the composition include one or more stabilization regions, e.g., a stabilization region between the 3′ hybridizing region and a barcode region as described herein, and/or a stabilization region between the barcode region and the 5′ tethering region.

Proximity-based extension assay can be any suitable assay that uses a binding moiety pair that are both bound (or are simultaneously bound) to an analyte to bring two oligonucleotides in proximity such that hybridization between the oligonucleotides generates a template for an extension reaction. In some embodiments, the proximity-based extension assay is a proximity extension strand displacement (PESD) assay (e.g., as provided herein). In some embodiments, the 5′ tethering region of the capture oligonucleotide is the same for all of the plurality of (e.g., at least 30, or 30-200, or 30-500, or 90-400, or 30-1600, or 300-2000, or 200-5000, or 1000-10000, etc.) pairs of oligonucleotides. In some embodiments, the 5′ tethering region of the detection oligonucleotide is the same for all of the plurality of (e.g., at least 30, or 30-200, or 30-500, 90-400, or 30-1600, or 300-2000, or 200-5000, or 1000-10000, etc.) pairs of oligonucleotides. In some embodiments, the 5′ tethering region of the capture oligonucleotides is different from the 5′ tethering region of the detection oligonucleotides.

The composition can include any suitable number of pairs of the oligonucleotides. In some embodiments, the composition includes, or includes at least, 5, 10, 15, 20, 25, 30, 35, 40, 45, 50, 55, 60, 65, 70, 75, 80, 85, 90, 95, 100, 120, 140, 160, 180, 190, 200, 250, 300, 400, 500, 1000, 1500, 2000, 3000, 5000, 10000 or a number in a range defined by any two of the preceding values (e.g., 5-500, 10-400, 15-300, 30-200, 40-190, 50-160, 30-100, 30-500, 90-400, 300-1500, 1000-5000, 1000-10000, etc.) pairs of the oligonucleotides. In some embodiments, the composition includes at least 30, at least 35, at least 40, at least 45, at least 50, at least 55, at least 60, at least 65, at least 70, at least 75, at least 80, at least 85, at least 90, at least 95, at least 100, at least 120, at least 140, at least 160, at least 180, at least 190, at least 200 or more, or a number in a range defined by any two of the preceding values (e.g., 30-200, 40-190, 50-160, 30-100, 30-500, 90-400, etc.) pairs of the oligonucleotides. In some embodiments, the composition includes about 30-50 pairs of the oligonucleotides. In some embodiments, the composition includes about 100-200 pairs of the oligonucleotides. In some embodiments, the composition includes about 300-500 pairs of the oligonucleotides.

In some embodiments, the capture oligonucleotide and detection oligonucleotide is each independently 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 nucleotides long, or longer, or a length in a range defined by any two of the preceding values (e.g., 15-100 nucleotides, 20-90 nucleotides, 30-50 nucleotides, 40-80 nucleotides, 50-100 nucleotides, etc.). In some embodiments, the capture oligonucleotide and detection oligonucleotide are each independently about 30-50 nucleotides long.

In some embodiments, the tethering regions of the capture oligonucleotide and detection oligonucleotide are each independently 15, 16, 17, 18, 19, 20, 21, 22, 23, 24, 25, 26, 27, 28, 29, 30, 35, 40, 45, 50, 55, 60 nucleotides long, or longer, or a length in a range defined by any two of the preceding values (e.g., 15-60, 20-55, 15-50, 30-40 nucleotides long, etc.).

In some embodiments, the 3′ hybridizing region of the capture oligonucleotide and/or the detection oligonucleotide is at most 10, 9, 8, 7, 6, 5, or 4 nucleotides long, or has a length in a range defined by any two of the preceding values (e.g., 4-10 nucleotides long, 5-7 nucleotides long, 6-9 nucleotides long, etc.). In some embodiments, the 3′ hybridizing region of the capture oligonucleotide and/or the detection oligonucleotide is 5-7 nucleotides long. In some embodiments, the 3′ hybridizing region of the capture oligonucleotide and/or the detection oligonucleotide is 6 or 7 nucleotides long.

In some embodiments, the capture oligonucleotide comprises a barcode sequence at least 6 nucleotides long between the 5′ tethering region and the 3′ hybridizing region, and/or the detection oligonucleotide comprises a barcode sequence at least 6 nucleotides long between the 5′ tethering region and the 3′ hybridizing region. In some embodiments, the 5′ tethering region and barcode sequence are adjacent to each other in the capture oligonucleotide, and the barcode sequence and the 3′ hybridizing region are adjacent to each other in the capture oligonucleotide. In some embodiments, the 5′ tethering region and barcode sequence are adjacent to each other in the detection oligonucleotide, and the barcode sequence and the 3′ hybridizing region are adjacent to each other in the detection oligonucleotide. In some embodiments, the capture oligonucleotide comprises a unique molecular identifier (UMI) at least 4 nucleotides long between the 5′ tethering region and the 3′ hybridizing region, and the detection oligonucleotide comprises a UMI at least 4 nucleotides long between the 5′ tethering region and the 3′ hybridizing region. In some embodiments, the 3′ hybridizing region of the capture oligonucleotide and the detection oligonucleotide in each of the plurality of (e.g., at least 30, or 30-200, 30-500, 90-400, etc.) pairs of oligonucleotides is 6 or 7 nucleotides long. In some embodiments, oligonucleotides of the composition include a subpool barcode, as described herein.

In some embodiments, the 3′ hybridizing region of the capture oligonucleotide and/or detection oligonucleotide in each of the plurality of (e.g., at least 30, or 30-200, 30-500, 90-400, etc.) pairs of oligonucleotides has a Hamming distance of at least 2, at least 3, at least 4, at least 5, or at least 6 relative to the 3′ hybridizing region of the capture oligonucleotide and/or the detection oligonucleotide, respectively, of any other pair of the plurality of (e.g., at least 30, or 30-200, 30-500, 90-400, etc.) pairs of oligonucleotides.

In some embodiments, a first calculated ΔG of hybridization between the 3′ hybridizing region of the capture oligonucleotide and detection oligonucleotide in each of the plurality of (e.g., at least 30, or 30-200, 30-500, 90-400, etc.) pairs of oligonucleotides is, or is about −4 kcal/mol or more negative, e.g., about −5 kcal/mol, about −6 kcal/mol, or more negative than a second calculated ΔG of hybridization between: (1) the 3′ hybridizing region of the capture oligonucleotide of the corresponding pair of oligonucleotides and the 3′ hybridizing region of the detection oligonucleotide of any other pair (or each of the other pairs) of the plurality of (e.g., at least 30, or 30-200, 30-500, 90-400, etc.) pairs of oligonucleotides; and/or (2) the 3′ hybridizing region of the detection oligonucleotide of the corresponding pair of oligonucleotides and the 3′ hybridizing region of the capture oligonucleotide of any other pair (or each of the other pairs) of the plurality of (e.g., at least 30, or 30-200, 30-500, 90-400, etc.) pairs of oligonucleotides. In some embodiments, a first calculated ΔG of hybridization between the 3′ hybridizing region of the capture oligonucleotide and detection oligonucleotide in each of the plurality of pairs of oligonucleotides is less than a second calculated ΔG of hybridization between each of: (1) the 3′ hybridizing region of the capture oligonucleotide of the corresponding pair of oligonucleotides and the 3′ hybridizing region of the detection oligonucleotide of each of the other pairs of the plurality of pairs of oligonucleotides; and (2) the 3′ hybridizing region of the detection oligonucleotide of the corresponding pair of oligonucleotides and the 3′ hybridizing region of the capture oligonucleotide of each of the other pairs of the plurality of pairs of oligonucleotides, by at least 4 kcal/mol. In some embodiments, a first calculated ΔG of hybridization between the 3′ hybridizing region of the capture oligonucleotide and detection oligonucleotide in each of the plurality of (e.g., at least 30, or 30-200, 30-500, 90-400, etc.) pairs of oligonucleotides is, or is about −4 kcal/mol or more negative, e.g., about −5 kcal/mol, about −6 kcal/mol, or more negative than a second calculated ΔG of hybridization between: (1) the 3′ hybridizing region of the capture oligonucleotide of the corresponding pair of oligonucleotides and the 3′ hybridizing region of the detection oligonucleotide of at least one, e.g., at least 1, 2, 3, 4, 5, 6, 7, 8, 9, 10, 15, 20, 25, 30, 40, 50, 60, 70, 80, 90, 100, 200, 300, 400, 500, 1000, 1500, 2000, 5000, or 10000 other pairs, or a number in a range defined by any two of the preceding values (e.g., 1-200, 10-100, 5-50, 5-40, 30-500, 90-400, 300-1500, 1000-5000, etc.), or of each of the other pairs, of the plurality of (e.g., at least 30, or 30-200, 30-500, 90-400, 300-1500, 1000-5000, etc.) pairs of oligonucleotides; and/or (2) the 3′ hybridizing region of the detection oligonucleotide of the corresponding pair of oligonucleotides and the 3′ hybridizing region of the capture oligonucleotide of at least one, e.g., at least 1, 2, 3, 4, 5, 6, 7, 8, 9, 10, 15, 20, 25, 30, 40, 50, 60, 70, 80, 90, 100, 200, 300, 400, 500, 1000, 1500, 2000, 5000, or 10000 other pairs, or a number in a range defined by any two of the preceding values (e.g., 1-200, 10-100, 5-50, 5-40, 30-500, 90-400, 300-1500, 1000-5000, etc.), or of each of the other pairs, of the plurality of (e.g., at least 30, or 30-200, 30-500, 90-400, 300-1500, 1000-5000, etc.) pairs of oligonucleotides. In some embodiments, a first calculated ΔG of hybridization between the 3′ hybridizing region of the capture oligonucleotide and detection oligonucleotide in each of the plurality of (e.g., at least 30, or 30-200, 30-500, 90-400, etc.) pairs of oligonucleotides is, or is about −4 kcal/mol or more negative, e.g., about −5 kcal/mol, about −6 kcal/mol, or more negative than a second calculated ΔG of hybridization between each of: (1) the 3′ hybridizing region of the capture oligonucleotide of the corresponding pair of oligonucleotides and the 3′ hybridizing region of the detection oligonucleotide of at least one, e.g., at least 1, 2, 3, 4, 5, 6, 7, 8, 9, 10, 15, 20, 25, 30, 40, 50, 60, 70, 80, 90, 100, 200, 300, 400, 500, 1000, 1500, 2000, 5000, or 10000 other pairs, or a number in a range defined by any two of the preceding values (e.g., 1-200, 10-100, 5-50, 5-40, 30-500, 90-400, 300-1500, 1000-5000, etc.), or of each of the other pairs, of the plurality of (e.g., at least 30, or 30-200, 30-500, 90-400, 300-1500, 1000-5000, etc.) pairs of oligonucleotides; and (2) the 3′ hybridizing region of the detection oligonucleotide of the corresponding pair of oligonucleotides and the 3′ hybridizing region of the capture oligonucleotide of at least one, e.g., at least 1, 2, 3, 4, 5, 6, 7, 8, 9, 10, 15, 20, 25, 30, 40, 50, 60, 70, 80, 90, 100, 200, 300, 400, 500, 1000, 1500, 2000, 5000, or 10000 other pairs, or a number in a range defined by any two of the preceding values (e.g., 1-200, 10-100, 5-50, 5-40, 30-500, 90-400, 300-1500, 1000-5000, etc.), or of each of the other pairs, of the plurality of (e.g., at least 30, or 30-200, 30-500, 90-400, 300-1500, 1000-5000, etc.) pairs of oligonucleotides.

Also provided is a composition comprising: a first pool of a plurality of (e.g., at least 30, or 30-200, 30-500, 90-400, 300-1500, 1000-5000, etc.) solid supports, each solid support comprising: a capture moiety attached to the solid support, wherein the capture moiety binds an analyte; and a capture oligonucleotide attached to the solid support, wherein the capture oligonucleotide comprises a 3′ hybridizing region; and a second pool of a plurality of (e.g., at least 30, or 30-200, 30-500, 90-400, 300-1500, 1000-5000, etc.) detection conjugates, each detection conjugate comprising: a detection moiety that binds the analyte; and a detection oligonucleotide attached to the detection moiety, wherein the detection oligonucleotide comprises a 3′ hybridizing region complementary to the 3′ hybridizing region of the capture oligonucleotide, wherein each of the plurality of (e.g., at least 30, or 30-200, 30-500, 90-400, 300-1500, 1000-5000, etc.) solid supports forms a paired combination with a corresponding detection conjugate of the plurality of (e.g., at least 30, or 30-200, 30-500, 90-400, 300-1500, 1000-5000, etc.) detection conjugates, wherein a binding target of the capture moiety and detection moiety of each paired combination is the same, and wherein different paired combinations have different binding targets, wherein the 3′ hybridizing region of the capture oligonucleotide and detection oligonucleotide of each paired combination is not complementary to the 3′ hybridizing region of the detection oligonucleotide and the capture oligonucleotide, respectively, of any other paired combination. In some embodiments, each of the paired combinations has a mis-pairing rate of at most about 1% in the presence of the other paired combinations in a proximity-based extension assay (e.g., PESD as described herein). In some embodiments, the mis-pairing rate is, is about, or is at most 1%, 0.8%, 0.5%, 0.2%, 0.1%, 0.05%. 0.01%, 0.005%, 0.001% or less, or a rate in a range defined by any two of the preceding values (e.g., 0.001-1%, 0.005-1%, 0.01-0.1%, etc.), in the presence of the other paired combinations in a proximity-based extension assay (e.g., PESD as described herein).

In some embodiments, the number of solid supports and detection conjugates is, or is at least, 5, 10, 15, 20, 25, 30, 35, 40, 45, 50, 55, 60, 65, 70, 75, 80, 85, 90, 95, 100, 120, 140, 160, 180, 190, 200, 250, 300, 400, 500, 1000, 1500, 2000, 5000, or 10000 or a number in a range defined by any two of the preceding values (e.g., 5-500, 10-400, 10-200, 30-200, 40-190, 50-160, 30-100, 30-500, 90-400, 300-1500, 1000-5000, etc.), where each of the solid supports pair with a corresponding detection conjugate to provide a paired combination. In some embodiments, the number of solid supports and detection conjugates is each at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 120, at least 140, at least 160, at least 180, at least 190, at least 200, at least 300, at least 400, at least 500, at least 1500 or more, or a number in a range defined by any two of the preceding values (e.g., 10-200, 30-200, 40-190, 50-160, 30-100, 30-500, 90-400, 300-1500, 1000-5000, etc.), where each of the solid supports pairs with a corresponding detection conjugate to provide a paired combination. In some embodiments, the composition includes about 30-50 of each of the solid supports and detection conjugates. In some embodiments, the composition includes about 100-200 of each of the solid supports and detection conjugates. In any composition, in some embodiments, each of the solid supports provide for a paired combination with a corresponding detection conjugate.

In some embodiments, the first pool and the second pool are each in partitions that are spatially distinct from each other (e.g., in different wells, microtubes, slides, etc.). In some embodiments, the composition includes a plurality of analytes comprising binding targets of the capture moieties and detection moieties. In some embodiments, the first pool includes the plurality of analytes. In some embodiments, the second pool includes the plurality of analytes. In some embodiments, the first pool and the second pool are in the same partition (e.g., same well, microtube, slide, etc.).

Also provided is a composition comprising: a solid support comprising: a capture moiety attached to the solid support, wherein the capture moiety binds an analyte; and a capture oligonucleotide attached to the solid support, wherein the capture oligonucleotide comprises a 3′ hybridizing region; a detection conjugate comprising: a detection moiety that binds the analyte; and a detection oligonucleotide attached to the detection moiety, wherein the detection oligonucleotide comprises a 3′ hybridizing region complementary to the 3′ hybridizing region of the capture oligonucleotide, wherein the capture moiety and the detection moiety are both bound (or are simultaneously bound) to the analyte such that the 3′ hybridizing region of the capture oligonucleotide and the 3′ hybridizing region of the detection oligonucleotide are in proximity to allow hybridization to each other. In some embodiments, placing the 3′ hybridizing regions of the capture oligonucleotide and detection oligonucleotide in proximity provides hybridization between the 3′ hybridizing regions sufficient to allow extension (e.g., allows a polymerase to use the hybridized region as substrate).

In some embodiments, the capture oligonucleotide is attached to the solid support via hybridization to a first tether oligonucleotide attached to the solid support, and/or the detection oligonucleotide is attached to the detection moiety via hybridization to a second tether oligonucleotide attached to the detection moiety. In some embodiments, the capture oligonucleotide comprises a 5′ tethering region that hybridizes to the first tether oligonucleotide. In some embodiments, the detection oligonucleotide comprises a 5′ tethering region that hybridizes to the second tether oligonucleotide.

Also provided is a composition comprising a plurality of partially double-stranded nucleic acids, each partially double-stranded nucleic acid comprising: a capture oligonucleotide hybridized at the 5′ end with a first tether oligonucleotide and the capture oligonucleotide comprising a 3′ hybridizing region of at most 10 nucleotides; and a detection oligonucleotide hybridized at the 5′ end with a second tether oligonucleotide and the detection oligonucleotide comprising a 3′ hybridizing region of at most 10 nucleotides, wherein the 3′ hybridizing region of the capture oligonucleotide is hybridized to the 3′ hybridizing region of the detection oligonucleotide. In certain embodiments, each partially double-stranded nucleic acid comprises: a capture oligonucleotide hybridized at the 5′ end with a first tether oligonucleotide of 15-25 or 15-30 nucleotides in length and comprising a 3′ hybridizing region of at most 10 nucleotides; a detection oligonucleotide hybridized at the 5′ end with a second tether oligonucleotide of 15-25 or 15-30 nucleotides in length and comprising a 3′ hybridizing region of at most 10 nucleotides, wherein the 3′ hybridizing region of the capture oligonucleotide is hybridized to the 3′ hybridizing region of the detection oligonucleotide. In some embodiments, the composition comprises a solid support attached to the first tether oligonucleotide.

Also provided herein is a composition comprising: a first pool of a plurality of first conjugates, each first conjugate comprising: a first moiety that binds an analyte; and a first splint oligonucleotide attached to the first moiety, wherein the first splint oligonucleotide comprises a 3′ hybridizing region; and a second pool of a plurality of second conjugates, each second conjugate comprising: a second moiety that binds the analyte; and a second splint oligonucleotide attached to the second moiety, wherein the second splint oligonucleotide comprises a 3′ hybridizing region complementary to the 3′ hybridizing region of the capture oligonucleotide, wherein each of the plurality of first conjugates forms a paired combination with a corresponding second conjugate of the plurality of second conjugates, wherein a binding target of the first moiety and second moiety of each paired combination is the same, and wherein different paired combinations have different binding targets, wherein for each paired combination, the first splint oligonucleotide is attached to the first moiety via hybridization to a first tether oligonucleotide attached to the first moiety, and/or the second splint oligonucleotide is attached to the second moiety via hybridization to a second tether oligonucleotide attached to the second moiety, wherein for each paired combination, the first and/or the second splint oligonucleotide comprises a barcode sequence that identifies the moiety to which the splint oligonucleotide is attached and/or a binding target thereof, wherein the 3′ hybridizing region of the first splint oligonucleotide and second splint oligonucleotide of each paired combination is not complementary to the 3′ hybridizing region of the second splint oligonucleotide and the first splint oligonucleotide, respectively, of any other paired combination.

In some embodiments, the plurality of first conjugates and the plurality of second conjugates provides, or provides at least, 10, 15, 20, 25, 30, 35, 40, 45, 50, 55, 60, 65, 70, 75, 80, 85, 90, 95, 100, 120, 140, 160, 180, 190, 200, 300, 500, 1500, 2000, 5000, 10000 or more, or a number in a range defined by any two of the preceding values (e.g., 10-200, 30-200, 40-190, 50-160, 30-100, 300-500, 1500-2000, etc.) paired combinations. In some embodiments, the plurality of first conjugates and the plurality of second conjugates provide at least 30 paired combinations. In some embodiments, each of the paired combinations has a mis-hybridization rate of at most about 1% in the presence of the other paired combinations in a proximity-based extension assay (e.g., PESD as described herein). In some embodiments, the mis-pairing rate is, is about, or is at most 1%, 0.8%, 0.5%, 0.2%, 0.1%, 0.05%. 0.01%, 0.005%, 0.001% or less, or a rate in a range defined by any two of the preceding values (e.g., 0.001-1%, 0.005-1%, 0.01-0.1%, etc.), in the presence of the other paired combinations in a proximity-based extension assay (e.g., PESD as described herein).

In some embodiments, the first pool and the second pool are each in partitions that are spatially distinct from each other (e.g., in different wells, microtubes, slides, etc.). In some embodiments, the composition includes a plurality of analytes comprising binding targets of the first moieties and second moieties. In some embodiments, the first pool includes the plurality of analytes. In some embodiments, the second pool includes the plurality of analytes. In some embodiments, the first pool and the second pool are in the same partition (e.g., same well, microtube, slide, etc.).

### VI. ADDITIONAL EMBODIMENTS

Additional non-limiting embodiments of the present disclosure are provided.

Provided herein are proximity-based assays that, in some non-limiting embodiments, employ capture surfaces and detection antibodies that engage in Proximity Extension Strand Displacement (PESD) to produce unique amplicons in the presence of a specific analyte (e.g., protein). Using these methods, the presence and/or identification of a protein in a sample can be detected and concentration quantified by sequencing the resulting amplicons or performing PCR.

In some embodiments, a PESD assay of the present disclosure involves:
hybridization of 2 overlapping oligonucleotides in close proximity to each other when a target analyte is present; one oligo is immobilized to a surface (e.g., a bead or culture plate well) and the other is attached to an antibody that specifically binds to the target analyte;
extension of the overlapping, hybridized oligonucleotides to produce a double-stranded (ds) nucleic acid;
release and amplification of the ds nucleic acid;
analysis of the resulting amplicons by PCR and/or NGS.

In some embodiments, a method of the present disclosure provides one or more of the following advantages:
Extension and amplicon release can occur in the same step, which can reduce assay time as compared to methods that employ a separate step to release amplicons after their formation;
Antibody conjugates can share a common “tether,” which can reduce oligonucleotide cost and simplify conjugations;
In some embodiments, unextended oligoucleotides (e.g., on bead or on detection conjugate) are not released into the sample/extension supernatant, which can reduce or effectively eliminate downstream PCR interference.

In some embodiments, during a PESD assay of the present disclosure, the extension and release steps are coupled because a single enzyme (e.g., a polymerase with strong strand displacement activity) performs both actions. This can improve the simplicity of the workflow and/or the efficiency of converting immunosandwiches (capture antibody—analyte—detection antibody) into amplifiable product.

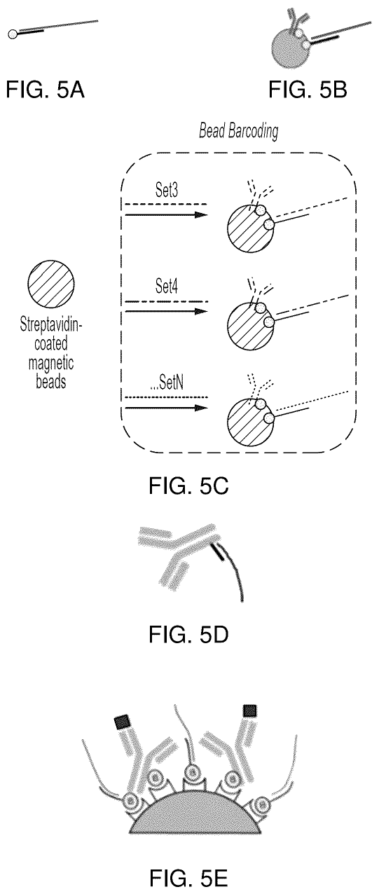

In some embodiments, a PESD method of the present disclosure employs the following components:
1) a Detection Complex (FIG. 3A) comprising (i) a detection antibody, and (ii) a common detection tether (oligonucleotide)
   a. the detection antibody is specific for an analyte of interest
   b. the common detection tether is conjugated to the detection antibody
2) a Detection oligonucleotide (also referred to as a detection oligo) that comprises a unique detector barcode sequence, a hybridization (or hybridizing) region sequence, and a sequence that is complementary to the detection tether sequence (e.g., a tethering region).
   a. the detection tether complement sequence region (e.g., tethering region) of the detection oligo is complementary to, and hybridizes to, the common detection tether that is conjugated to the detection antibody, thereby attaching the detection oligo to the detection antibody to form a detection conjugate (FIG. 5D);
   b. the complementary region of the detection tether and the detection oligo preferably comprise a PCR primer sequence that can be used to amplify the double stranded product after extension;
   c. the hybridization (or hybridizing) region sequence of the detection oligo can be unique (e.g., different for each analyte) or common (e.g., same sequence used for multiple different analytes). In some embodiments, using unique hybridization (or hybridizing) region sequences of the detection oligo can result in fewer non-productive amplicons formed. When unique hybridization (or hybridizing) regions are employed, the hybridization (or hybridizing) region on the detection oligo can also be used as the unique barcode for that oligo, allowing a shorter construct which may be favorable for sequencing.
3) A Bead (FIG. 3B) comprising (i) a capture antibody (cAby), and (ii) a bead tether (oligonucleotide), and
4) a Capture oligo that comprises a unique bead barcode sequence, a hybridization (or hybridizing) region that is the complement of the hybridization (or hybridizing) region sequence in the detection oligo, and a region that is complementary to the bead tether sequence (e.g., a tethering region).
   a. the bead tether complement region (e.g., tethering region) of the capture oligo is complementary to, and hybridizes to, the bead tether that is immobilized on the bead surface, thereby attaching the capture oligo to the bead surface;
   b. the complementary region of the bead tether and the capture oligo preferably comprise a PCR primer;
   c. the sequence of the capture oligo hybridization (or hybridizing) region can be unique (e.g., different for each analyte) or common (e.g., the same sequence used for multiple different analytes). In some embodiments, using unique sequences of the capture oligo hybridization (or hybridizing) region can result in better assay performance since there are less non-productive amplicons formed;
   d. the bead tether may be directly attached to the bead surface or it may be conjugated to a member of a binding pair (e.g., biotin) which binds to the other member of the binding pair (e.g., streptavidin) that is coated on the bead surface.
   e. the capture antibody may be conjugated to a member of a binding pair (e.g., biotin), which binds to the other member of the binding pair (e.g., streptavidin) that is coated on the bead, thus attaching the cAby to the bead surface.

In some embodiments, the detection tether (e.g., a second tether oligonucleotide) and/or the bead tether (e.g., a first tether oligonucleotide) is 15-50 nucleotides (nt). In some embodiments, the complementary region of the detection tether and the detection oligo include a PCR primer, as does the complementary region of the bead tether and the capture oligo, which can be at least ˜20 nt. In some embodiments, the unique detector barcode sequence and/or the unique bead barcode sequence is 5-15 nt. The length of the barcode sequence can depend on the number of unique barcodes needed and the error tolerance. In some embodiments, longer barcodes allow greater Hamming distance between adjacent barcodes, reducing the likelihood of misidentifying a barcode due to substitution errors during oligo synthesis or sequencing. In some embodiments, the common or unique hybridization region may be 4-10 nt. The inventors have found ideal lengths to be 5-7 bp. Hybridization (or hybridizing) regions that are too long may lead to increased background due to hybridization in the absence of analyte. Hybridization (or hybridizing) regions that are too short lead to low extension efficiency and too few unique sequences.

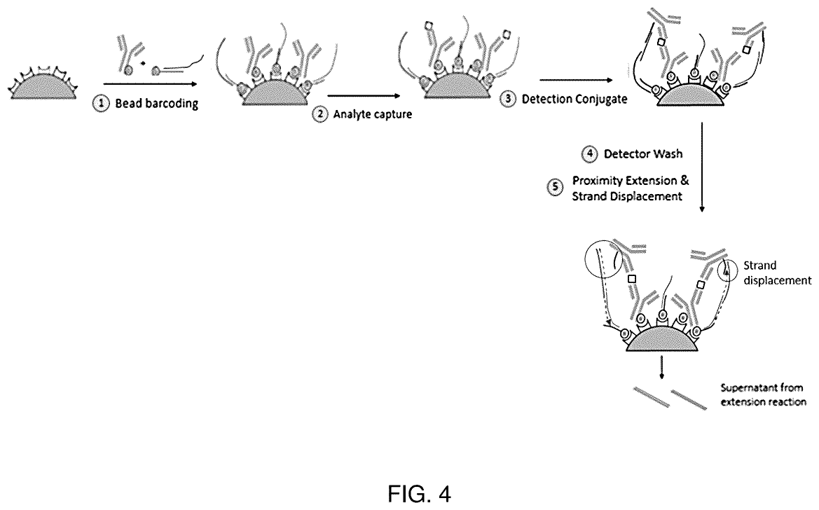

In some embodiments, an overview of a PESD method protocol of the present disclosure includes the following steps:
1. coat beads with cAby and also with bead tether hybridized to capture oligo; wash beads (FIG. 4, step 1);
2. add sample to coated beads (analyte capture) (FIG. 4, step 2); wash beads;
3. add detection conjugate with hybridized detection oligo to beads (FIG. 4, step 3; FIG. 5C); wash beads (FIG. 4, step 4);
4. add Klenow Fragment (3→5′ exo-) polymerase with strand displacement activity to beads (PESD) (FIG. 4, step 5);
5. Supernatant from extension reaction can be used for:
   qPCR total amplicon quantitation; and/or
   NGS Library preparation and sequencing.

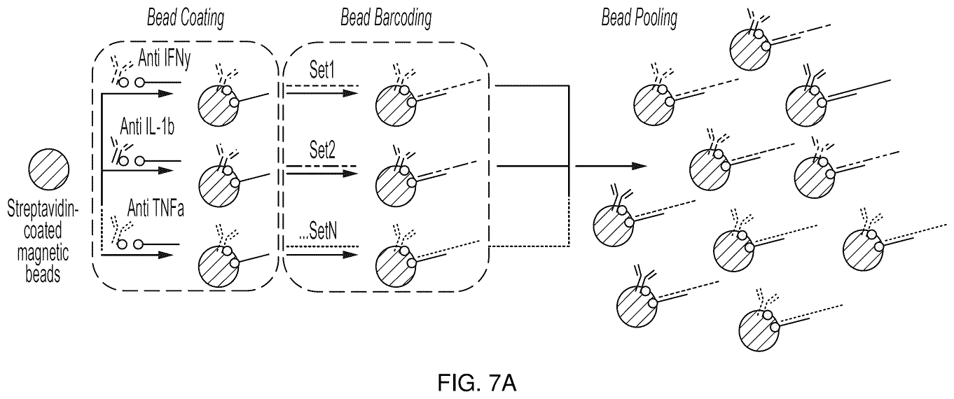

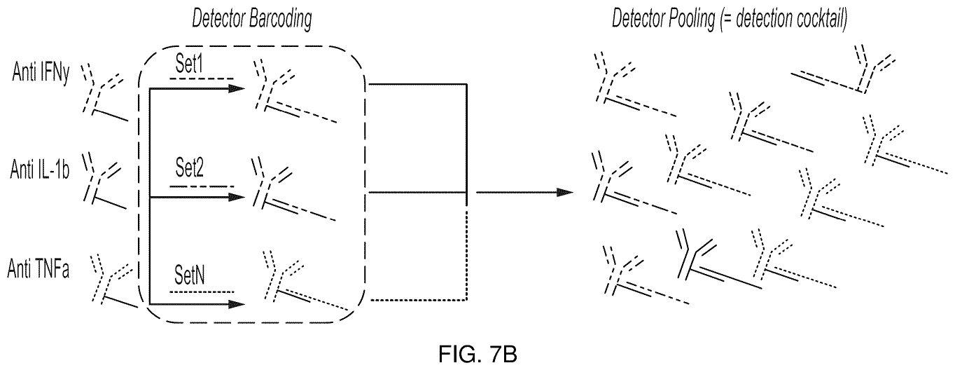

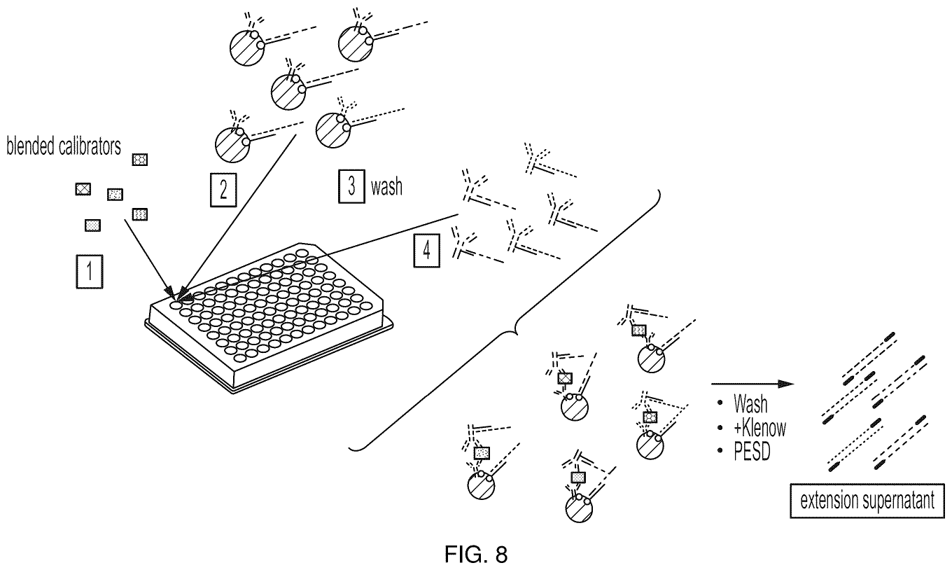

In some embodiments, an overview of a multiplex PESD assay of the present disclosure is provided in FIGS. 7A, 7B, and 8.

Using a polymerase with strong strand displacement activity (e.g., Klenow Fragment (3′→5′ exo-) to extend the oligos during primer extension (PE)s can allow the strand displacement (SD) activity to selectively release the full-length amplicons of interest. Using the Klenow/SD PESD method can release more amplicon into the supernatant than other proximity-based extension assays (e.g., PEA) that release amplicon using enzymatic treatment (e.g., Proteinase K (ProK)). About 10-fold more amplicon copies on average can be released in PESD vs. PEA with ProK.

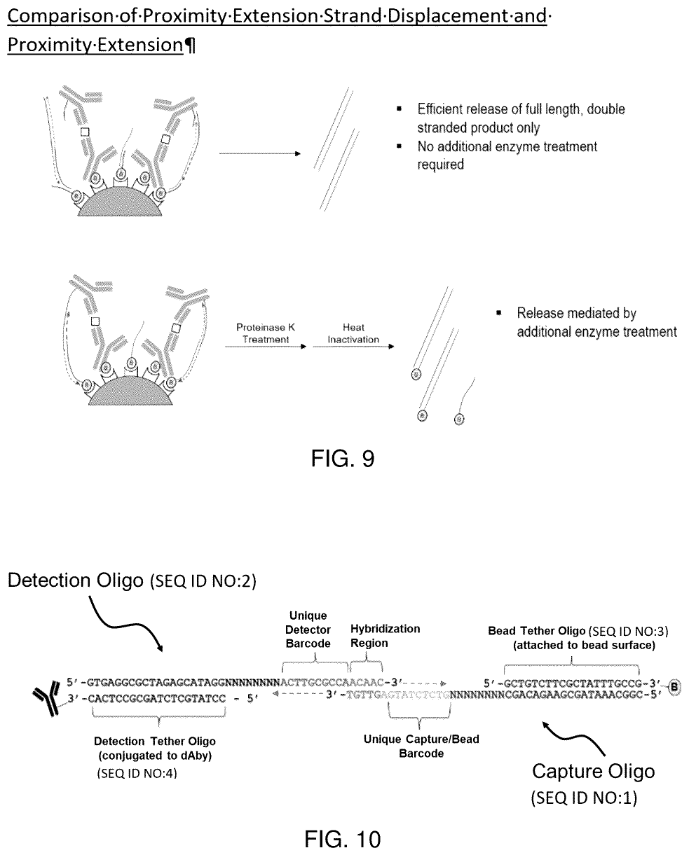

With PESD, extension and amplicon release can occur in the same step; this can result in reduced assay time relative to other proximity-based extension assays that release amplicon using enzymatic treatment, e.g., a ProK method. In addition, the PESD method can be simpler than PEA with ProK (which can include an additional ProK reagent and incubation and inactivation steps) (FIG. 9).

In some embodiments, a PEA method protocol of the present disclosure in singleplex format, includes using proteinase K (ProK) to digest streptavidin (SA) and releasing the bound, extended oligos from bead surfaces after extension (for subsequent sequencing or PCR). In some embodiments, the ProK may release all oligos that were on the beads including non-extended oligos or other components of the system. This can affect multiplexed assays as the non-amplicon oligos that are released by the ProK may interfere with downstream PCR by acting as a primer or by sequestering primer.

### VII. SPECIFICITY OF PESD ASSAY

In some embodiments, after the PESD reaction there can be at least one and possibly several mixtures of reaction products that were generated from each sample. The number of mixtures per sample can depend on whether or not the sample was split into multiple dilutions and run on assay subpools, and whether these subpools were recombined before the extension reaction.

In some embodiments, each reaction product mixture contains n distinct species of double stranded DNA, having the proper length and having a pair of correctly matched barcodes, resulting from a correctly formed molecular sandwich, where n is the number of analytes to be measured by the multiplex assay. In some embodiments, each of these species has a PCR primer at each end and thus is PCR amplifiable. In some embodiments, the PESD process can minimize truncated products because it relies on the strand displacement activity of the polymerase linked to its polymerization activity to read through the tethering oligo, which is also the PCR primer sequence. In some embodiments, DNA released from the solid phase has primers on both ends. In some embodiments, the mixture contains many contaminant species that are either not the proper length, or contain mismatched barcodes.

For example, mismatched barcodes may occur when an incorrect detector antibody is held in proximity to a capture antibody though means other than a correctly formed molecular sandwich, e.g. through non-specific binding of a detection antibody for analyte X to a bead with capture antibodies for analyte Y, or due to biological cross reactivity of the detector for X with analyte Y, or vice versa. In some embodiments, hybridization regions at the 3′ ends of the forward and reverse oligos are complementary only to their intended paired oligo, and have distinct non-pairing sequences to all other oligos in the set, thus destabilizing the hybridization between these off-target pairings and limiting the ability of the polymerase to extend oligos of mismatched pairs. In some embodiments, this represents a first level of added specificity, relative to a standard multiplex immunoassay. In some embodiments, the detectors do not have the same label and can prevent non-specifically bound detectors, e.g., those bound to the wrong spot, from creating signal just as efficiently as specifically bound ones. In some embodiments, in PESD assays, non-specifically bound detectors create products inefficiently compared to specifically bound ones, such as >100-fold less efficiently.

In some embodiments, low level of extension even between oligos with mispaired hybridization regions can be identified. In some embodiments, where these represent a small fraction of the total reaction product, their effect on the assay performance is minimized by a second mechanism of specificity, which results from the digital nature of the assay readout. In some embodiments, once the sequences of each reaction product are determined, those products with non-matched barcodes are eliminated from the analysis, eliminating any assay background due to mispaired antibodies and ensuring that the background for each assay doesn't depend on the number of analytes in the multiplex. In some embodiments, this added specificity is provided in high multiplex assays where the total amount of non-specifically bound detector antibody can scale with the number of analytes. In some embodiments, some biological cross reactivity between analytes or non-specificity of antibody binding can be tolerated. In some embodiments, the dual mechanism of specificity in the PESD assay due to both the orthogonal hybridization regions and the digital filtering of mismatched barcodes enables much higher assay specificity than standard multiplexing approaches, even if there may be some off-target binding of antibodies.

In some embodiments, improper length amplicons generated either by sequencing errors, usually one or two base deletions or insertions, or by mispriming of the hybridization regions by partial hybridization to an incorrect sequence either at the 3′ ends or elsewhere in the construct are identified. In some embodiments, oligo design and stringent reaction conditions for the extension minimize mispriming products. In some embodiments, once the sequence of each reaction product has been determined, the misprimed products that are formed are eliminated digitally by eliminating all products of the incorrect length. In some embodiments, this will also eliminate any products with sequencing indels, even if they resulted from a correctly formed sandwich. In some embodiments, these are recaptured by the use of error tolerant barcode sets. In some embodiments, a Hamming distance of 4 is used to allow barcodes to be identified even with one or two indels.

### VIII. USE OF BLOCKER NUCLEOTIDES IN PESD ASSAYS

Also provided herein is use of blockers to improve immunosequencing assays (e.g., PESD). In some embodiments, the method includes options to minimize off-target interactions in highly multiplexed immunoassays. In some embodiments, oligonucleotide blockers are included to minimize off-target interactions in highly multiplexed immunoassays, such as PESD assays. In some embodiments, the use of two different oligonucleotide blockers reduces 1) the pulldown of detector through the complementary hybridization regions in the absence of analyte, and 2) mispriming of extension through hybridization of the 3′ terminus of an oligonucleotide hybridization region with the barcode of another oligonucleotide. In some embodiments, the “hybridization blocker” is salt-labile and is removed via stringent, low-salt wash prior to extension. In some embodiments, the “barcode blocker” is non-labile, and is removed via polymerase strand displacement during extension. In some embodiments, additional “stabilization regions” are added to the forward and reverse PESD oligonucleotide sequences to better stabilize the hybridization blocker (e.g., under moderate salt concentrations, for example, 137 mM Na⁺), and the barcode blocker (under suitable salt concentrations).

In some embodiments, blocking oligonucleotides may be used to hybridize to the single-stranded regions of the forward and reverse oligonucleotides (e.g., subpool barcode and forward barcode). Thus, in some embodiments, the need for barcoded tether oligonucleotides is eliminated. This also eliminates, in some embodiments, the need for asymmetrically dividing the subpool barcode between detection and capture sides. In the case of blocking oligonucleotides, in some embodiments, 5′→3′ extension of the blocker into the tether sequence is eliminated with 3′ oligonucleotide modifications (e.g., 3′ phosphate, 3′ inverted dT), or by any other suitable option (e.g., destabilization of polymerization with multiple 3′ base pair mismatches).

In some embodiments, “blocker stabilization regions” are added to the forward and reverse oligonucleotides, that allow for additional base pairing between blocker and barcode, or blocker and hybridization region. In some embodiments, the hybridization region is 5-7 nt long, which would not create enough hybridization energy to stably duplex with a blocker under most conditions. Thus, additional neighboring bases are added to the sequence that “stabilize” the blocker:hyb duplex. In some embodiments, the hybridization blocker stabilization region is 4-6 additional bases. Similarly, in some embodiments, the barcode regions are 10 nt, and although 10 nt may form a stable duplex under some conditions, the blocker:barcode duplex is stable under all salt conditions, thus making it non-salt labile (unlike the blocker:hybridization region duplex). In some embodiments, the barcode blocker stabilization region is 10-12 additional nucleotides.

In some embodiments, barcode stabilization regions also encodes additional assay information; for example subpool barcodes, as described herein.

In some embodiments, modified bases may be used in the blocker sequences to provide additional stability to the duplexes, including, but not limited to, Locked Nucleic Acids (LNAs).

In some embodiments, blockers are added to their respective forward and reverse oligonucleotides prior to use, and excess (unbound) blocker would be removed via bead washing. In some embodiments, blockers on the capture side are added to the reverse oligonucleotide during the thermal annealing step containing: 1) biotin-tether and 2) reverse oligonucleotide. In some embodiments, performing the hybridization as a thermal anneal (as opposed to a room temperature process) improves hybridization efficiency and reduces the need for large stoichiometric excesses of blockers. In some embodiments, unbound blocker is removed in the first wash after clonal bead preparation, prior to bead pooling. In some embodiments, blockers on the detector side are added during the detector barcode step, prior to detector cocktail pooling, and detector-sample incubation.

In some embodiments, unbound blocker is removed in the first wash after detector incubation. In some embodiments, bound hybridization blocker is removed by stringent, low salt wash after the detection step. In some embodiments, bound barcode blocker is removed by strand displacement that occurs in the extension step.

In some embodiments, if displacement of barcode blocker into the reaction supernatant is detrimental to downstream processes, a polymerase with 5→3 exonuclease activity may be used in place of an enzyme with strand displacement activity. In some embodiments, 3′ termini of the blockers are modified/capped, or otherwise inaccessible to polymerases, such that the polymerase cannot extend in the 5′→3′ direction to displace the forward or reverse oligonucleotide from the tether/anchor on the bead or the detection antibody. In some embodiments, modified bases including, but not limited to: 3′ phosphorylation, 3′ inverted dT, 3′ dideoxyC (ddC). In some embodiments, a tailed blocker that has intentionally mismatched bases at its 3′ terminus to inhibit 5′→3′ extension from the blocker “tail”. In some embodiments, a polymerase without strong 3′→5′ exonuclease “proofreading” activity is used for extension. In some embodiments, a polymerase without 3′→5′ exonuclease “proofreading” activity is used for extension.

In some embodiments, blockers may be used asymmetrically only on one side of the assay sandwich. For example, only the capture side, or only on the detector side.

In some embodiments, the use of both of the blocker types described herein reduces the formation of DNA hairpins and self-dimers in the forward and reverse oligonucleotides, which will improve the performance of some oligonucleotide sets in PESD.

In some embodiments, the hybridization (“Hyb”) blocker is approximately 7-13 nucleotides long. In some embodiments, the hybridization regions is less than 7 nucleotides and an optional stabilization region on the 5′ side of the hyb region is added to allow better stability for blocker binding to the hyb region. In some embodiments, the stabilization region may be common or unique to each assay/oligo.

The hybridization blocker can be labile (e.g., removable before the extension step of PESD). The hybridization blocker can be removed by a variety of chemical conditions that promote the denaturation of weakly bound DNA; the simplest of which is low salt “stringent” wash buffers. The hybridization blocker can be thermally stable or transient, e.g., forms a stable duplex at room temperature, or does not.

In some embodiments, a hybridization blocker may be used at stoichiometric equivalence to the oligo that it is blocking, or at super stoichiometric excess to the oligo that it is designed to block, depending on whether the blocker is thermally stable or transiently stable. In some embodiments, mix of different blocker lengths (and stabilities) are used in a multiplexed reaction. In some embodiments, a reaction mixture includes one or more stable blockers and one or more transient blockers.

In some embodiments, the more stable the blocker:hyb duplex is, the more effective the blocker. However, it is harder to remove and hybridization blocker that remains behind during extension will inhibit product formation and thus needs to be removed for maximum signal generation. Blockers that remain may be extended by polymerase, forming off-target extension products that may interfere with later steps of the assay or library preparation. In some embodiments, this can be mitigated by using various blocking groups to inhibit extension, e.g. 3′ phosphate, inverted dT, or other chemical modification.

In some embodiments, the barcode blocker is approximately 14 nucleotides long or longer. The barcode blocker may not be labile under the same conditions as the hybridization blocker, e.g., cannot be removed via stringent wash. In some embodiments, the barcode regions is less than 12 nucleotides (e.g., 10 nt) and an optional stabilization region is added on the 5′ side of the barcode sequence to allow better stability for blocker binding to the barcode. The stabilization region may be common or unique to each assay/oligo.

In some embodiments, barcode blockers are removed from the system during the extension step incubation. In some embodiments, blockers are present during extension and are configured to inhibit (5→3) extension and falsely extending/displacing the oligonucleotide being blocked. Any suitable option can be used to inhibit extension, including without limitation, 3′ terminal modification: e.g., phosphate, ddC, or invdT group.

In some embodiments, the barcode blocker includes an overhang tail that includes an extra non-base paired stretch of DNA at the 3′ end of the blocker that lack base pairing required for extension by a polymerase.

### IX. SUBPOOLING AND USE OF SUBPOOL BARCODES

Provided herein are methods for adding subpool barcodes in NGS-based highly multiplexed immunoassays.

The subpooling scheme provided herein can in some embodiments be dictated by the expected endogenous levels of analyte present in a normal patient sample. High concentrations of a small number of analytes in a patient sample can create a disproportionate number of amplicons in the sequencing library and can account for a disproportionate number of reads in any NGS run with a finite number of reads. Therefore, analytes can in some embodiments be grouped by their anticipated concentrations in panels and patient samples diluted in those panels an appropriate amount (e.g., 1:1, 1:20, 1:400, 1:2000) such that the resulting amplicon concentrations (on a per analyte basis) are approximately equal.

In some embodiments, the placement of analytes into subpools depends not just on the expected concentration of the analytes in subpools but the amount of analyte required to saturate the capture surface (which may depend on capture antibody affinity) and the amplicon expected to be generated (which may depend on capture and detector antibodies). In some embodiments, paired combinations of capture and detector antibodies with lower sensitivity may be used in a subpool with less dilution even though the analyte is expected to be higher than other analytes for which there are paired combinations of capture and detector antibodies with higher sensitivity.

In some embodiments, two subpools are run at the same dilution, but using different diluent compositions. In some embodiments, a low-abundance analyte is assayed in an undiluted (neat) subpool, and serum containing diluents or high salt are used. In some embodiments, another analyte is assayed in another undiluted (neat) subpool without serum containing diluents due to cross reactivity.

In some embodiments, subpools are started in parallel with their sample capture step (e.g., with different dilution levels of patient sample, with different diluents), and they are merged back together prior to library prep and sequencing. In some embodiments, there are several steps at which the subpools can be merged back together: Post capture, Post detect, or Post extension. In some embodiments, use of a solid support provides for combining the subpools at different stages of the assay.

In some embodiments, in highly multiplexed NGS-based immunoassays, such as Proximity Extension Strand Displacement (PESD), the orthogonality of oligonucleotide sequences (e.g., hybridization regions and barcodes) is important to create high quality assays. Each assay, comprising of a capture antibody and detection antibody specific for different epitopes on the same analyte, in a highly multiplexed panel can have oligonucleotides that contain a pair of complementary hybridization region sequences (e.g., 5-7 nt long) that are different than (and non-interacting with) all other hybridization region sequences for the other assays in the panel. In some embodiments, where the length of the hybridization region is restricted (e.g., 5-7 nt), there is a limited number of sequence pairs that are orthogonal from all others. For example, there is a theoretical maximum of 8192 complementary pairs of sequences that are 7 base pairs in length. For highly multiplexed NGS-based immunoassays, in silico screening may be used to evaluate the complementary pairs of sequences for among other aspects: Hamming distance, self-complementarity, 3-base repeats. Additionally, empirical testing reveals that orthogonal hybridization region sequence pairs, which passed in silico screening, can produce off-target interactions with other hybridization regions. The availability of unique hybridization regions can create a practical limit on the number of assays that can be multiplexed concurrently. In some embodiments, the number of orthogonal complementary pairs is from 100 to 200 pairs, and thus a single sample well includes 100-200 different immunoassays. In some embodiments, an additional barcode sequence, herein referred to as a “subpool barcode,” is added to allow for the repeated use of the 100-200 most orthogonal hybridization pairs across multiple (sub)pools or panels. In some embodiments, subpools are designed, and analytes grouped, based on predicted dilution levels for each group of analytes in a particular biofluid. In some embodiments, 4 different subpools are used for 384-plex testing in plasma.

Provided herein is an adaptable subpool design in a PESD assay. In some embodiments, a PESD assay involves flexibility in organizing the panels, as the relative quantities of analytes may vary in different biofluids/biosamples (e.g., plasma, saliva, cell culture supernatant). Analytes may be moved from one subpool to another, based on a number of experimental factors, including but not limited to the biofluid being tested. Individual assays/antibodies are not conjugated with a predetermined subpool barcode.

In some embodiments, a barcode is used to differentiate between a small number of subpools, and thus minimal code-space is required and short sequences (e.g., 4-8 nt) can be used. In some embodiments, 4 subpools are used, based on the anticipated dilution levels to measure 384+ analytes to 1) preserve the dynamic range, and 2) to balance signal proportionally across all analytes for analytes in biofluids; for example neat, 1:20, 1:200, 1:1000.

In some embodiments, ‘top performing’ hybridization+barcode combinations are recycled: oligos that have been exhaustively screened and optimized for PESD can be “recycled” across multiple subpools. In some embodiments, a “top performing” pair may include 1) minimization of off target interactions (e.g. between hybridization regions and barcodes), 2) orthogonality of hybridization pairs, and/or 3) maximization of extension efficiency.

In some embodiments, the additional code introduced by the subpool barcode allows for the same 2 barcodes (e.g., forward and reverse barcode) to be identified as different assays during sequencing and demultiplexing. Additionally, where applicable, detection antibodies that mispair with the wrong capture antibodies during extension, but share the same hybridization region across subpools, can be distinguished as a mispaired amplicon and the associated NGS reads discarded.

In some embodiments, a subpool barcode is a split subpool barcode. In some embodiments, the barcode is asymmetrically split across the two sides of the immunoassay. In some embodiments, the combination of the two correctly matched subpool barcode halves form a complete barcode corresponding to a particular subpool. In some embodiments, regions of the barcode are buried in the capture-associated oligo and in the detector-associated oligo. In some embodiments, all or most of the subpool barcode is present in double-stranded form, which will minimize off-target interactions with other sequences. In some embodiments, the subpool barcode is added to the forward and reverse oligonucleotides as the next bases (in the 5′→3′ direction) after the tether/anchor sequence. In some embodiments, the tether/anchor is common across all assays in PESD.

In some embodiments, a barcode region is present on both the capture and detector side to ensure that cross reactivity between captures or detectors from different subpools that share the same primary barcodes can be disambiguated. These can be, for example, 1-8 nt long on each side. 1 nt on each side may be sufficient for encoding 4 subpools, however there would be no error tolerance. A substitution error in sequencing either of the two barcodes may lead to an unrecognizable barcode combination (small penalty) or when a substitution error occurs on a mismatched detector (e.g., in the case of non-specific binding or cross reactivity), it could lead to incorrect identification of a barcode combination (severe penalty). In some embodiments, using at least 2 nt on each side can avoid or reduce the incorrect identification as it requires two or more simultaneous substitution errors (which may be very low probability). In some embodiments, a tether oligo includes a sequence complementary to the subpool barcode on capture oligo (bury subpool barcode in duplex DNA on the capture side, decreases risk of mispriming).

In some embodiments, a new barcode is created for 4 subpools, and up to 4 new non-universal anchors are provided. In some embodiments, on the capture-associated oligo, the anchor is bound by biotin:streptavidin interaction and not covalently conjugated to the capture antibody. In some embodiments, 4 subpool-barcoded tethers are synthesized as biotinylated oligonucleotides and integrated into existing PESD assays. In some embodiments, the capture-associated subpool barcode is double-stranded, and the detection-associated subpool barcode remains single-stranded. In some embodiments, disproportionally dividing the subpool barcode bases to favor the capture side is preferred. In some embodiments, a split of 2 bases on the detection side and 4 bases on the capture side is provided.

In some embodiments, the number of assays in the multiplex is scaled by dividing assays into ‘subpools’ or ‘panels’ and amplicon in that subpool/panel is given a unique code for later NGS identification and demultiplexing. In some embodiments, a plurality of suitable barcode and hybridization sequences are provided, where the hybrid length is restricted to ˜7 nt and barcode length is restricted to ˜10 nt.

In some embodiments, approximately 4 additional bases are added to the ‘capture’ side of the amplicon. These 4 new nucleotides can duplex with a new biotin-anchor oligo that contains the complement to that 4 nt subpool barcode. In some embodiments, approximately 2 additional bases are added to the ‘detector side’ of the amplicon. These may not be duplexes and can remain as singled-stranded portion of the oligonucleotide. In some embodiments, the 6 nt subpool ‘barcode’ is split across the amplicon such that any mis-matches in the subpool barcode are digitally filtered during analysis as illegitimate reads. In some embodiments, the subpools are merged post-capture (e.g., prior to the detection step).

In some embodiments, the addition of 4 bp (double stranded) on the capture side and 2 nt (single stranded) on the detection side does not give rise to off-target oligo:oligo interactions. In some embodiments, blockers are designed and implemented to mitigate new off-target effects due to addition of subpool barcodes. For example, adding a subpool barcode into the anchor region/5′ tethering region has the benefit of ensuring the new barcode is double stranded and will not present additional opportunity for mispriming of hyb regions. In some embodiments, subpool barcodes are adjacent either 1) the barcode or 2) the hybridization region. In some embodiments, these could be used as a part of the blocker stabilization regions. In some embodiments, blockers are used to cover the newly added subpool barcodes, and they may be lengthened to create additional code space, thus accommodating a larger number of possible subpools. In some embodiments, dividing the multiplex assay into several subpools allows use of the same barcode+hyb sequences (and combinations) for more analytes than possible without subpooling. For example, 500 unique barcode combinations×4 subpools=2000 analytes.

In some embodiments, use of subpool barcodes (whether blocked or unblocked) includes demultiplexing the amplicons to determine 1) which subpool that amplicon was derived from and 2) which analyte within that subpool it was derived from.

### X. OLIGONUCLEOTIDE TRIMMING

In some embodiments, a Proximity Extension Strand Displacement (PESD) assay includes two oligonucleotides that are held in close proximity through the formation of an immunocomplex (e.g., an antibody:analyte:antibody ‘sandwich’). In some embodiments, the two, paired oligonucleotides have a short stretch of complementarity in their 3′ ends (e.g., 5-7 nt). In some embodiments, relatively weak base-pairing between these two oligonucleotides (<10 bp) can lead to transient interactions between the two oligonucleotides. When the oligos are proximity confined, and weakly interacting, the presence of an appropriate polymerase will extend both oligos from their 3′ ends to form a double stranded product. This weak hybridization energy (from a short hybridization overlap) contributes to generating the extension product. However, there can be trade-offs between hybridization lengths in proximity-based assays. Shorter hybridization lengths are generally weaker than longer hybridization lengths. Shorter hybridization lengths may suffer from poorer extension efficiencies than longer hybridization lengths. However, a hybridization length that is too long, and therefore has too strong of hybridization energy, may form extension products independent of proximity confinement. Therefore, in some embodiments, 1) the proper length of hybridization region, and 2) the specific sequences to be used in a proximity extension-based assay are balanced.

In some embodiments, a highly multiplexed proximity extension based assay with NGS readout includes having unique hybridization overlaps for each of the oligonucleotides pairs that comprise each side of each assay. The availability of unique, informatic code space for shorter hybridization lengths is fundamentally smaller than longer for hybridization lengths. In some embodiments, the use of longer hybridization lengths allows for more stringent selection of oligonucleotide pairs when searching for maximum of orthogonality, and minimum off-target oligo:oligo interactions. In some embodiments, longer hybridization lengths are used. However, the longer hybridization lengths may create more proximity-independent pairs.

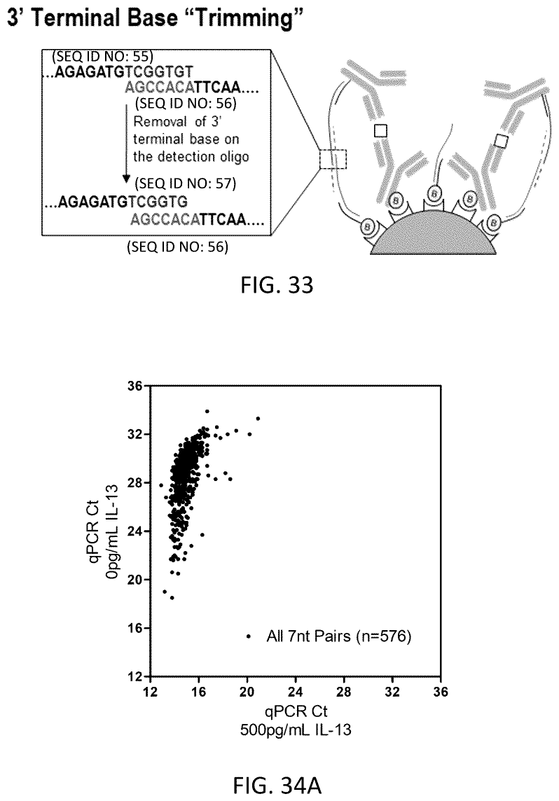

In some embodiments, selectively ‘trimming’ one base from only one side of a two-oligonucleotide proximity extension-based assay preserves the code space and sequence orthogonality that is generated from the longer ‘untrimmed’ sequence. Trimming one base from the 3′ end of one of the oligonucleotides results in a hybridization length that is now N−1, where N is the original length for that construct. For example, an oligonucleotide design that features 7 nt hybridization lengths, but has been trimmed, will now have 6 bp when hybridized to its untrimmed counterpart (FIG. 33). In some embodiments, trimming reduces the hybridization energy of strong 7 nt pairs by knocking down to 6 bp.

In some embodiments, one or more oligonucleotide sets in a pool of oligos and assays is trimmed. In some embodiments, sequence pairs exist in the same multiplex pool that are untrimmed, with the original hybridization length of 7 nt.

With this approach, in some embodiments, only one of the two sequences in the pair is modified, which results in the full-length product amplicon being formed following enzymatic extension. In some embodiments, trimming can be performed on either side of the immunoassay sandwich. In some embodiments, trimming takes into account the G-C content differences in the trimmed versus untrimmed sequences. In some embodiments, trimming from the ‘capture side’ or ‘detector side’ has the same effect on performance. That is, for example, if a pair of untrimmed sequences are both terminated at their 3′ ends by guanosine, then the effect of trimming the capture side versus the reverse side should essentially be identical.

In some embodiments, trimming allows fine-tuning the energetics of a pool of oligonucleotide pairs in a multiplexed assay. In some embodiments, a pool of unique oligonucleotides with shorter hybridization overlaps (e.g., 5 bp) can vary greatly in their transient energies and may suffer from a broad variability of product extension efficiencies. In some embodiments, this variance is greatly suppressed when slightly longer hybridization lengths are used (e.g., 6 or 7 bp), due to the marginally increased hybridization energy. In some embodiments, there is variance among 7 bp hybridization regions because of the inherently transient nature of those individual proximity interactions. In some embodiments, trimming the stronger 7 bp interactions on either oligo provides greater control and selectivity in matching extension efficiencies by tapping into a much larger pool of available hybridization energies: 7 nt, 6 nt (‘detector’ oligo trimmed), or 6 nt (‘capture’ oligo trimmed).

In some embodiments, for any number of oligo sets in a large multiplex, a select number of oligos receives ‘trimming’. ‘Trimming’ can denote the oligos are intentionally designed and synthesized with one less oligo on the 3′ end. In some embodiments, one nucleotide is ‘trimmed’ off the 3′ end of either capture-side or detect-side oligonucleotide (but not both), to yield a hybridization overlap that is N−1, where N is the starting hyb length for the untrimmed oligo sets.

In some embodiments, oligos can be trimmed selectively on some pairs (e.g., 7 nt pairs) with the highest hybridization energy. In some embodiments, oligo sets that have too strong of base pairing, and show high non-specific detector pulldown, are selected for trimming. In some embodiments, trimming reduces the energy of only that one oligo set (not the entire pool of oligos), and decreases the non-specific detector pulldown at the minimal expense of proximity extension efficiency. In some embodiments, the product amplicon is still the same length as the other oligonucleotide sets in the multiplex. In some embodiments, trimming reduces secondary structure involving the 3′ terminal base. In some embodiments, trimming is useful to balance the hybridization/extension energetics of a large pool of oligonucleotides with N and N−1 hybridization lengths. In some embodiments, signal-to-background improves by minimizing oligo:oligo pulldown with minimal effect on specific signal generation.

### XI. ATTENUATION

Non-limiting examples of options to accommodate higher analyte concentrations without increasing the amount of amplicon generated and still preserve the linear behavior of the assay are provided. In some embodiments, the number of amplicon molecules across each analyte (regardless of the endogenous analyte concentration) is balanced by attenuating the signal from high abundance assays.

1. Increasing solid support surface area (number of capture beads) and associated amount of capture antibody to accommodate higher analyte levels while attenuating the amplicon produced by a given level of analyte. In some embodiments, attenuating amplicon includes: Selection of very low efficiency oligo pairs—e.g., use those with lower hybridization energy to attenuate the PESD extension reaction. In some embodiments, 5 nt overlap oligos have much lower efficiency than 6 nt overlap, and most 6 nt overlaps have lower efficiency than 7 nt (see Example 15). Secondary structures formed by self-complementarity, e.g. hairpins, can also be used to reduce the availability of the hybridization region, which may attenuate the efficiency of the PESD reaction. In some embodiments, attenuating amplicon includes: Reducing the concentration of reverse oligonucleotide on the beads. The amount of reverse oligonucleotide per bead is titrated such that the average spacing between oligonucleotides is greater than the spacing between capture antibodies. In some embodiments, some molecules of analyte may be captured by antibodies that are not close enough to a reverse oligonucleotide to allow the PESD proximity reaction to occur. Once the average spacing of oligos is below that of the antibodies, the amplicon/[analyte] ratio decreases linearly with the surface density of oligos. This relationship was observed over a 243-fold range, suggesting that the signal can be attenuated to accommodate a wide range of analyte concentrations.

In some embodiments, clonal beads are prepared with a small amount of capture-side oligonucleotide on the bead surface. In some embodiments, the smaller amount of oligonucleotide on the beads decreases the reaction efficiency of the extension reaction and a proportionally reduced amount of amplicon is generated at the end of the assay.

2. Add a second set of beads for each analyte with no reverse oligonucleotide. In some embodiments, the method includes preparing ‘cold’ beads with the same capture antibody as the clonal beads, but without any capture-side oligo, and blending with the clonal beads with capture oligo. These “cold” beads can capture a fraction of the analyte but may be unable to generate any PESD products due to the lack of reverse oligo. The ratio of “cold” beats to normal or “hot” beads for each analyte may set the attenuation ratio for that assay (e.g., 99% cold beads and 1% hot beads may reduce the amplicon produced at a given analyte concentration by 100-fold). In some embodiments, the ratio of cold beads to traditional beads can be custom blended based on the desired level of attenuation. During the capture step, the highly abundant analyte will distribute among the cold beads and traditionally prepared beads. In some embodiments, during the extension step, only the analytes bound to the traditionally prepared beads will generate amplicon. In some embodiments, the total bead mass (traditional beads+cold beads) is scaled up appropriately for high abundance analytes, thereby preventing a truncated dynamic range.

In some embodiments, the method includes increasing the amount of capture antibody (and capture surface area) to preserve dynamic range. In some embodiments, the method includes increasing the amount of beads to capture analyte (e.g., at the high end of the concentration range), in order to avoid saturation and preserve the dynamic range of the assay. In some embodiments, increasing the amount of beads includes adding more (clonal) capture beads for those particular assays to the multiplex pool.

In some embodiments, the method includes using an un-labeled soluble version of the capture antibody (“cold capture”) as a competitor to prevent binding of a fraction of the analyte to the beads. In some embodiments, “cold capture” antibody (or “cold” antibody) is an identical clone to the capture antibody immobilized on the beads. In some embodiments, the “cold” antibody is added to the diluent used in the analyte capture step. In some embodiments, the amount of cold antibody added can be varied based on the desired degree of attenuation. In some embodiments, the unlabeled antibody competes with the capture antibody immobilized on beads for the binding epitopes of the analyte in solution. In some embodiments, a reduced quantity of analyte is captured on the beads. In some embodiments, unbound analyte and cold antibody are washed away during the post-capture wash step. The assay then proceeds as normal, but with a reduced amount of the initial analyte captured on bead resulting in a reduced amount of amplicon at the assay end point.

In some embodiments, adding “cold capture” antibody (or “cold” antibody) to attenuate very abundant analytes may be more practical than increasing the bead concentration. In some embodiments, the relationship between capture antibody on the beads and soluble competitor “hot/cold” ratio and the attenuation factor may not be linear, as the binding kinetics may be different for the soluble and surface bound capture antibodies. In some embodiments, the attenuation factor of a “cold capture” antibody is empirically determined. In some embodiments, the oligo amount on the beads is the same with or without the soluble “cold capture” antibody. In some embodiments, the number of capture analytes bound is attenuated through competition when using the soluble “cold capture” antibody.

In some embodiments, any two or more of the options above are combined in any combination to produce even larger attenuation ratios or to fine-tune the ratio.

In some embodiments, attenuating the amount of analyte captured includes using lower affinity capture antibodies. In some embodiments, the lower affinity antibodies have lower on-rate and lower off-rate.

### XII. EXEMPLARY, NUMBERED EMBODIMENTS

Additional non-limiting embodiments of the present disclosure are provided in the following numbered embodiments.

1. A method of analyzing a sample for an analyte, comprising:
a) providing a solid support comprising:
   a capture moiety attached to the solid support, wherein the capture moiety binds an analyte; and
   a capture oligonucleotide attached to the solid support, wherein the capture oligonucleotide comprises a 3′ hybridizing region;
b) providing a detection conjugate comprising:
   a detection moiety that binds the analyte; and
   a detection oligonucleotide attached to the detection moiety, wherein the detection oligonucleotide comprises a 3′ hybridizing region complementary to the 3′ hybridizing region of the capture oligonucleotide;
c) preparing a complexing solution by:
   i) combining in a solution the solid support provided in a) and the detection conjugate provided in b) with a sample, thereby allowing the capture moiety of the solid support and the detection moiety of the detection conjugate to be bound to the analyte if present in the sample;
   ii) contacting the solid support provided in a) with a sample, thereby allowing the capture moiety of the solid support to be bound to the analyte if present in the sample, and combining in a solution the sample-contacted solid support and the detection conjugate provided in b); or
   iii) contacting the detection conjugate provided in b) with a sample, thereby allowing the detection moiety to be bound to the analyte if present in the sample, and combining in a solution the sample-contacted detection conjugate with the solid support provided in a),
thereby allowing the capture moiety and the detection moiety in the complexing solution to both be bound (or to be simultaneously bound) to the analyte if present such that the capture oligonucleotide and detection oligonucleotide are in proximity if the analyte is present in the sample;
d) permitting the 3′ hybridizing region of the capture oligonucleotide and the 3′ hybridizing region of the detection oligonucleotide that are in proximity to hybridize to each other;
e) extending the hybridized capture oligonucleotide and/or the hybridized detection oligonucleotide to generate an on-target extension product that comprises the extended capture oligonucleotide and/or the extended detection oligonucleotide;
f) releasing the on-target extension product from the solid support and, optionally, from the detection moiety; and
g) determining the presence and/or amount, or the absence of the released on-target extension product to thereby determine the presence and/or amount, or the absence, of the analyte in the sample.

2. The method of embodiment 1, wherein preparing the complexing solution in c) comprises i) combining in a solution the solid support provided in a) and the detection conjugate provided in b) with a sample, thereby allowing the capture moiety of the solid support and the detection moiety of the detection conjugate to be bound to the analyte if present in the sample.

3. The method of embodiment 1, wherein preparing the complexing solution in c) comprises ii) contacting the solid support provided in a) with a sample, thereby allowing the capture moiety of the solid support to be bound to the analyte if present in the sample, and combining in a solution the sample-contacted solid support and the detection conjugate provided in b).

4. The method of embodiment 3, comprising removing the sample before combining in the solution the sample-contacted solid support and the detection conjugate provided in b), thereby removing analyte if present that is not bound to the capture moiety.

5. The method of embodiment 4, wherein removing the sample comprises washing the solid support.

6. The method of embodiment 1, wherein preparing a complexing solution in c) comprises iii) contacting the detection conjugate provided in b) with a sample, thereby allowing the detection moiety to be bound to the analyte if present in the sample, and combining in a solution the sample-contacted detection conjugate with the solid support provided in a).

7. The method of any one of embodiments 1-6, comprising releasing the on-target extension product from the detection moiety in f).

8. The method of any one of the preceding embodiments, comprising:
after preparing the complexing solution in c), removing the solution from the solid support, thereby removing a detection conjugate whose detection moiety is not bound (or simultaneously bound) with the capture moiety of the solid support to the analyte if present; and in e), extending at least one of the hybridized capture oligonucleotide and the hybridized detection oligonucleotide on the solid support removed from the solution.

9. The method of embodiment 8, wherein removing the sample or the solution from the solid support comprises washing the solid support.

10. The method of any one of the preceding embodiments, wherein releasing the on-target extension product comprises treating the solid support with a strand-displacing polymerase, a restriction enzyme, a protease, and/or a high-stringency wash.

11. The method of any one of the preceding embodiments, wherein the capture oligonucleotide is attached to the solid support independently of the capture moiety.

12. The method of any one of the preceding embodiments, comprising in e), extending the hybridized capture oligonucleotide and the hybridized detection oligonucleotide.

13. The method of any one of the preceding embodiments, wherein extending and releasing are performed by a single enzyme.

14. The method of embodiment 13, wherein the single enzyme comprises a strand-displacing polymerase.

15. The method of any one of the preceding embodiments, wherein the capture oligonucleotide is attached to the solid support via hybridization to a first tether oligonucleotide attached to the solid support.

16. The method of any one of the preceding embodiments, wherein the detection oligonucleotide is attached to the detection moiety via hybridization to a second tether oligonucleotide attached to the detection moiety.

17. The method of embodiment 15 or 16, wherein the extending step e) comprises treating the solid support with a strand-displacing polymerase under conditions sufficient to extend the hybridized capture oligonucleotide and/or the hybridized detection oligonucleotide, and
wherein the releasing in f) comprises allowing the strand-displacing polymerase to displace the first tether oligonucleotide hybridized to the capture oligonucleotide during extension, and optionally to displace the second tether oligonucleotide hybridized to the detection oligonucleotide during extension.

18. The method of embodiment 17, wherein the strand-displacing polymerase is allowed to displace the second tether oligonucleotide hybridized to the detection oligonucleotide during extension.

19. The method of embodiment any one of embodiments 14-18, wherein the strand-displacing polymerase comprises an exo-Klenow fragment.

20. The method of any one of the preceding embodiments, wherein the releasing in f) is performed at a temperature between, or in a range of, 10⁻³⁷° C.

21. The method of any one of embodiments 13-20, wherein the capture oligonucleotide comprises a 5′ tethering region that hybridizes to the first tether oligonucleotide.

22. The method of embodiment 21, wherein the 5′ tethering region of the capture oligonucleotide comprises a nucleotide sequence 15-30 nucleotides long that is complementary to at least a portion of the first tether oligonucleotide.

23. The method of any one of embodiments 13-22, wherein the detection oligonucleotide comprises a 5′ tethering region that hybridizes to the second tether oligonucleotide.

24. The method of embodiment 23, wherein the 5′ tethering region of the detection oligonucleotide comprises a nucleotide sequence 15-30 nucleotides long that is complementary to at least a portion of the second tether oligonucleotide.

25. The method of any one of embodiments 13-24, wherein the first tether oligonucleotide is covalently attached to the solid support, or is adsorbed onto the solid support.

26. The method of any one of embodiments 13-25, wherein the first tether oligonucleotide is covalently attached to:
(1) a first member of a binding pair that binds to a second member of the binding pair, wherein the second member is attached to the solid support, or
(2) the solid support.

27. The method of any one of embodiments 13-26, wherein providing the solid support comprises hybridizing the capture oligonucleotide to the first tether oligonucleotide.

28. The method of any one of embodiments 13-27, wherein providing the solid support comprises attaching the first tether oligonucleotide to the solid support.

29. The method of embodiment 28, wherein the first tether oligonucleotide is attached to the solid support before hybridizing the capture oligonucleotide to the first tether oligonucleotide.

30. The method of embodiment 28, wherein the first tether oligonucleotide is attached to the solid support after hybridizing the capture oligonucleotide to the first tether oligonucleotide.

31. The method of any one of embodiments 13-30, wherein providing the detection conjugate comprises hybridizing the detection oligonucleotide to the second tether oligonucleotide.

32. The method of embodiment 31, wherein hybridizing the detection oligonucleotide to the second tether oligonucleotide comprises combining in a solution the detection oligonucleotide with the second tether oligonucleotide at a molar ratio of at least about 1:1.

33. The method of any one of embodiments 13-32, wherein the second tether oligonucleotide is covalently attached to:
(1) a first member of a binding pair that binds to a second member of the binding pair, wherein the second member is attached to the detection moiety, or
(2) the detection moiety.

34. The method of any one of embodiments 13-33, wherein providing the detection conjugate comprises attaching the second tether oligonucleotide to the detection moiety.

35. The method of embodiment 34, wherein the second tether oligonucleotide is attached to the detection moiety before hybridizing the detection oligonucleotide to the second tether oligonucleotide.

36. The method of embodiment 35, wherein the hybridizing the detection oligonucleotide to the second tether oligonucleotide comprises combining in a solution the detection moiety comprising the second tether oligonucleotide with the detection oligonucleotide, wherein the detection moiety is at a concentration in a range of about 5 nM to about 10 M.

37. The method of embodiment 34, wherein the second tether oligonucleotide is attached to the detection moiety after hybridizing the detection oligonucleotide to the second tether oligonucleotide.

38. The method of any one of embodiments 1-12, wherein
the capture oligonucleotide is attached to the solid support via a bonding interaction that is independent of the nucleotide sequence comprised in the capture oligonucleotide, and/or
the detection oligonucleotide is attached to the detection moiety via a bonding interaction that is independent of the nucleotide sequence comprised in the detection oligonucleotide.

39. The method of embodiment 38, wherein the detection oligonucleotide is covalently attached to the detection moiety.

40. The method of embodiment 38 or 39, wherein the releasing in f) comprises cleaving the covalent attachment of the on-target extension product from the detection moiety.

41. The method of any one of embodiments 38-40, wherein the capture oligonucleotide is covalently attached to a first member of a binding pair bound to a second member of the binding pair, wherein the second member is attached to the solid support.

42. The method of embodiment 41, wherein the releasing in f) comprises cleaving the first or second member of the binding pair.

43. The method of any one of embodiments 38-42, wherein the releasing in f) comprises treating the solid support with a protease.

44. The method of any one of the preceding embodiments, wherein the solid support comprises a plurality of the capture moieties and a plurality of the capture oligonucleotides attached to the solid support,
wherein providing the detection conjugate comprises providing a plurality of the detection conjugates, and
wherein the method further comprises following preparing the complexing solution in c) and prior to the extending in e), removing any detection conjugate that is not bound to an analyte that is bound (or simultaneously bound) to a capture moiety.

45. The method of embodiment 44, wherein said removing comprises washing the solid support under high stringency conditions.

46. The method of any one of the preceding embodiments, wherein the solid support comprises a plurality of capture moieties and a plurality of capture oligonucleotides.

47. The method of any one of the preceding embodiments, wherein the number and/or density of capture moieties on the solid support is greater than the number and/or density of capture oligonucleotides on the solid support.

48. The method of embodiment 47, wherein the solid support comprises a ratio of the number and/or density of the capture moiety to the capture oligonucleotide of, of about, or of at least, 2:1, 2.5:1, 3:1, 3.5:1, 4:1, 4.5:1, 5:1, 6:1, 7:1, 8:1, 9:1, 10:1, 15:1, 20:1, 25:1, 30:1, 40:1, 45:1, 50:1, 55:1, 60:1, 70:1, 80:1, 90:1, 100:1, 150:1, 200:1, 250:1, 300:1, 400:1, 500:1, 1,000:1, 2,000:1, 5,000:1, 10,000:1, 50,000:1, 100,000:1, or optionally wherein the solid support comprises a ratio of the number and/or density of the capture moiety to the capture oligonucleotide in a range defined by any two of the preceding values (e.g., 2:1-100,000:1, 5:1-5,000:1, 10:1-1,000:1, 5:1-500:1, etc.), optionally about 2:1 to about 50:1, about 2:1 to about 10:1, about 3:1 to about 7:1, or about 5:1, optionally about 50:1.

49. The method of embodiment 47, wherein the solid support comprises a ratio of the capture moiety to the capture oligonucleotide of about 50:1, or about 5:1.

50. The method of any one of the preceding embodiments, wherein the capture moiety is covalently attached to a first member of a binding pair that binds to a second member of the binding pair, wherein the second member is attached to the solid support.

51. The method of any one of embodiments 26-50, wherein the first member of the binding pair comprises biotin, and the second member of the binding pair comprises streptavidin.

52. The method of any one of embodiments 1-49, wherein the capture moiety is covalently attached to the solid support, or is adsorbed onto the solid support.

53. The method of any one of the preceding embodiments, comprising attaching the capture moiety and/or the capture oligonucleotide to the solid support.

54. The method of any one of the preceding embodiments, comprising concurrently attaching the capture moiety and the capture oligonucleotide to the solid support.

55. The method of any one of embodiments 1-53, comprising attaching the capture moiety to the solid support, then attaching the capture oligonucleotide to the solid support, or attaching the capture oligonucleotide to the solid support, then attaching the capture moiety to the solid support.

56. The method of any one of the preceding embodiments, wherein both the capture moiety and the detection moiety specifically bind to the analyte.

57. The method of any one of the preceding embodiments, wherein the 3′ hybridizing region of the capture oligonucleotide and/or the detection oligonucleotide is 10 nucleotides long or shorter.

58. The method of embodiment 57, wherein the 3′ hybridizing region of the capture oligonucleotide and/or the detection oligonucleotide is 6 or 7 nucleotides long.

59. The method of any one of the preceding embodiments, wherein the capture oligonucleotide comprises a first barcode sequence that identifies a binding target of the capture moiety, and wherein the detection oligonucleotide comprises a second barcode sequence that identifies a binding target of the detection moiety.

60. The method of any one of the preceding embodiments, wherein the capture oligonucleotide comprises a first barcode sequence that identifies the capture moiety attached to the solid support to which the capture oligonucleotide is attached.

61. The method of any one of the preceding embodiments, wherein the detection oligonucleotide comprises a second barcode sequence that identifies the detection moiety to which the detection oligonucleotide is attached.

62. The method of any one of the preceding embodiments, wherein the capture oligonucleotide and/or detection oligonucleotide comprises, from 5′ to 3′: a tethering region, a barcode sequence, and the 3′ hybridizing region.

63. The method of any one of the preceding embodiments, wherein the capture oligonucleotide and the detection oligonucleotide each comprises a primer binding region configured to bind a primer pair for amplifying the released on-target extension product.

64. The method of embodiment 63, wherein the capture oligonucleotide and the detection oligonucleotide each comprises a 5′ tethering region, wherein the 5′ tethering region comprises the primer binding region or a portion thereof.

65. The method of any one of the preceding embodiments, comprising generating a calibration curve of the analyte by:
generating a plurality of serially diluted calibrator samples, each calibrator sample comprising a known amount of the analyte in a dilution series; and
determining the presence and/or amount, or the absence of the released on-target extension product in each of the plurality of serially diluted calibrator samples.

66. The method of any one of the preceding embodiments, comprising:
extending the hybridized capture oligonucleotide and/or the hybridized detection oligonucleotide via a primer extension reaction; and
isolating a supernatant fraction of the primer extension reaction, the supernatant fraction comprising the one or both strands of the on-target extension product released from at least the solid support; and
determining the presence and/or amount, or the absence of the released on-target extension product in the supernatant fraction.

67. The method of any one of the preceding embodiments, comprising providing:
a plurality of paired combinations of the solid supports and the detection conjugates, wherein a binding target of the capture moiety and detection moiety of each paired combination is the same, and wherein at least some, and optionally all, of the different paired combinations of the plurality of paired combinations have different binding targets.

68. The method of embodiment 67, wherein the 3′ hybridizing region of the capture oligonucleotide and detection oligonucleotide of each of the plurality of paired combinations of the solid supports and detection conjugates identifies the binding target of the corresponding paired combination.

69. The method of embodiment 67 or 68, wherein the 3′ hybridizing region of the capture oligonucleotide and detection oligonucleotide of a paired combination of the plurality of paired combinations of the solid supports and detection conjugates identifies a binding target that is different from a binding target identified by the 3′ hybridizing region of the capture oligonucleotide and detection oligonucleotide of at least one other paired combination of the plurality of paired combinations.

70. The method of any one of embodiments 67-69, wherein the 3′ hybridizing region of the capture oligonucleotide and detection oligonucleotide of a first paired combination of the plurality of paired combinations is not complementary to the 3′ hybridizing region of the detection oligonucleotide and capture oligonucleotide, respectively, of at least one other paired combination of the plurality of paired combinations.

71. The method of embodiment 70, wherein the 3′ hybridizing region of the capture oligonucleotide and/or detection oligonucleotide of the first paired combination has a Hamming distance of at least 2 relative to the 3′ hybridizing region of the capture oligonucleotide and/or detection oligonucleotide, respectively, of at least one other paired combination of the plurality of paired combinations.

72. The method of embodiment 70 or 71, wherein a first calculated ΔG of hybridization between the 3′ hybridizing regions of the capture oligonucleotide and detection oligonucleotide of the first paired combination is about −4 kcal/mol or more negative than a second calculated ΔG of hybridization between:
(1) the 3′ hybridizing region of the capture oligonucleotide of the first paired combination and the 3′ hybridizing region of the detection oligonucleotide of any (or each) of the at least one other paired combination of the plurality of paired combinations; and/or
(2) the 3′ hybridizing region of the detection oligonucleotide of the first paired combination and the 3′ hybridizing region of the capture oligonucleotide of any (or each) of the at least one other paired combination of the plurality of paired combinations.

73. The method of any one of embodiments 67-72, wherein preparing the complexing solution in c) comprises:
(i) combining in a solution the solid supports and the detection conjugates of the plurality of paired combinations with the sample; or
(ii) contacting the solid supports of the plurality of paired combinations with the sample, and then combining in a solution the sample-contacted solid supports and the detection conjugates of the plurality of paired combinations; or
(iii) contacting the detection conjugates of the plurality of paired combinations with the sample, and then combining in a solution the sample-contacted detection conjugates and the solid supports of the plurality of paired combinations.

74. The method of any one of embodiments 67-73, comprising providing a plurality of spatially distinct partitions, each comprising at least one detection conjugate of the plurality of paired combinations of the solid supports and the detection conjugates, wherein the detection moiety of the at least one detection conjugate in a partition of the plurality of spatially distinct partitions has a different binding target from the detection moiety of the at least one detection conjugate in a different partition of the plurality of spatially distinct partitions.

75. The method of embodiment 74, comprising pooling the detection conjugates in the plurality of spatially distinct partitions before preparing the complexing solution in c).

76. The method of any one of embodiments 67-75, comprising diluting an original sample comprising one or more analytes (e.g., one or more analytes that can be bound by the plurality of paired combinations of the solid supports and the detection conjugates), thereby generating the sample comprising the one or more analytes at a lower concentration than the original sample.

77. The method of any one of embodiments 67-76, comprising providing a plurality of spatially distinct partitions, each comprising at least one solid support of the plurality of paired combinations of the solid supports and the detection conjugates, wherein the capture moiety attached to a solid support in a partition of the plurality of spatially distinct partitions has a different binding target from the capture moiety attached to a solid support in a different partition of the plurality of spatially distinct partitions.

78. The method of embodiment 77, comprising pooling the solid supports in the plurality of spatially distinct partitions before preparing the complexing solution in c).

79. The method of embodiment 77 or 78, wherein preparing the complexing solution in c) comprises:
contacting portions of the sample with each of the solid supports in the plurality of spatially distinct partitions, then pooling the solid supports in the plurality of spatially distinct partitions, and then contacting the pooled solid supports with the detection conjugates of the plurality of paired combinations.

80. The method of any one of embodiments 74-79, wherein the plurality of spatially distinct partitions comprises a plurality of microtubes, microwells, and/or microfluidic chambers.

81. The method of any one of embodiments 67-80, wherein each capture oligonucleotide attached to a solid support of the plurality of the solid supports comprises a barcode sequence that identifies a binding target of the capture moiety attached to the respective solid support, optionally, wherein barcode sequences of capture oligonucleotides that are attached to solid supports attached to capture moieties having the same binding target have the same barcode sequence, and/or
wherein each detection oligonucleotide attached to a detection moiety of the plurality of the detection conjugates comprises a barcode sequence that identifies a binding target of the respective detection moiety.

82. The method of any one of embodiments 67-81, wherein barcode sequences of detection oligonucleotides attached to detection moieties that have the same binding target are identifiable based on the barcode sequence, or
a barcode sequence of a detection oligonucleotide attached to a detection moiety that has the same binding target as a capture moiety is identifiable based on the barcode sequence of a capture oligonucleotide attached to a solid support to which the capture moiety is attached, and/or a barcode sequence of a capture oligonucleotide attached to a solid support to which a capture moiety having the same binding target as a detection moiety is identifiable based on the barcode sequence of a detection oligonucleotide attached to the detection moiety.

83. The method of any one of embodiments 67-82, wherein two different barcode sequences of capture oligonucleotides (e.g., barcode sequences of two different capture oligonucleotides) that are attached to solid supports that are attached to capture moieties having different binding targets have a Hamming distance of at least 3, and/or
wherein two different barcode sequences of detection oligonucleotides (e.g., barcode sequences of two different detection oligonucleotides) attached to detection moieties having different binding targets have a Hamming distance of at least 3.

84. The method of any one of embodiments 67-83, wherein the plurality of paired combinations of the solid supports and the detection conjugates comprises at least 5 paired combinations.

85. The method of any one of embodiments 67-84, wherein the on-target extension product from each paired combination of the plurality of paired combinations is at least about 10 times more abundant than off-target extension products.

86. The method of any one of the preceding embodiments, wherein the presence of the analyte in the sample is determined with a specificity of about 99 parts in 100 or higher.

87. The method of any one of the preceding embodiments, wherein determining the presence and/or amount, or the absence of the released on-target extension product comprises performing qPCR on one or more extension products generated at e) and released from the solid support.

88. The method of any one of the preceding embodiments, wherein determining the presence and/or amount, or the absence of the released on-target extension product comprises obtaining sequencing data of one or more extension products generated at e) and released from the solid support.

89. The method of embodiment 88, comprising sequencing the one or more released extension products to obtain the sequencing data of the one or more released extension products.

90. The method of embodiment 89, wherein the sequencing comprises high-throughput sequencing.

91. The method of any one of embodiments 87-90, wherein determining the presence and/or amount, or the absence of the released on-target extension product comprises amplifying the one or more extension products generated at e) and released from the solid support.

92. The method of embodiment 91, wherein determining the presence and/or amount, or the absence of the released on-target extension product comprises:
combining the one or more extension products with a control oligonucleotide in an amplification mixture;
amplifying the one or more extension products and the control oligonucleotide in the amplification mixture, to generate extension product amplicons and control oligonucleotide amplicons,
detecting a level of the extension product amplicons and the control oligonucleotide amplicons; and
normalizing the detected level of the extension product amplicons based on the detected level of the control oligonucleotide amplicons.

93. The method of embodiment 92, wherein the control oligonucleotide comprises a unique molecular identifier (UMI), wherein detecting the level of the control oligonucleotide comprises obtaining sequence information of the UMI in the control oligonucleotide amplicons, and wherein the detected level of the control oligonucleotide is based on the number of the UMI with distinct sequences associated with a sequence of the control oligonucleotide in the sequencing data.

94. The method of embodiment 92 or 93, wherein the extension product amplicons comprise adapter sequences that hybridize to one or more sequencing primers.

95. The method of any one of embodiments 92-94, wherein the extension product amplicons comprise one or more indexing sequences.

96. The method of any one of embodiments 88-95, wherein the capture oligonucleotide and/or detection oligonucleotide comprises a unique molecular identifier (UMI).

97. The method of embodiment 96, wherein determining the presence and/or amount, or the absence of the released on-target extension product comprises determining the number of UMI with distinct sequences associated with the capture oligonucleotide and/or detection oligonucleotide.

98. The method of any one of embodiments 88-97, comprising identifying a sequence of an extension product in the sequencing data as being associated with the on-target extension product based on a pairing of: (A) one or more subsequences in the sequence associated with the detection oligonucleotide, wherein the one or more subsequences identify the binding target of the detection moiety attached to the detection oligonucleotide, with (B) one or more subsequences in the sequence associated with the capture oligonucleotide, wherein the one or more subsequences identify the same binding target of the capture moiety that is attached to the solid support to which the capture oligonucleotide is attached.

99. The method of embodiment 98, wherein at least one of the one or more subsequences in the sequence associated with the detection oligonucleotide and the one or more subsequences in the sequence associated with the capture oligonucleotide is a barcode sequence.

100. The method of embodiment 99, wherein the one or more subsequences in the sequence associated with the detection oligonucleotide comprises a first barcode sequence, and the one or more subsequences in the sequence associated with the capture oligonucleotide comprises a second barcode sequence.

101. The method of embodiment 99 or 100, wherein the 3′ hybridizing region identifies the binding target of the detection oligonucleotide or capture oligonucleotide.

102. The method of any one of embodiments 98-101, wherein a sequence in the sequencing data is identified as an off-target extension product based on a mispairing between: (A) the one or more subsequences in the sequence associated with the detection oligonucleotide, wherein the one or more subsequences identify the binding target of the detection moiety attached to the detection oligonucleotide, and (B) the one or more subsequences in the sequence associated with the capture oligonucleotide, wherein the one or more subsequences identify a different binding target of a different capture moiety that is attached to a different solid support to which the capture oligonucleotide is attached.

103. The method of any one of embodiments 98-102, wherein identifying the sequence in the sequencing data as being associated with the on-target extension product is further based on a length of the sequenced extension product, wherein an on-target extension product is associated with a sequenced extension product having a length consistent with the length expected for the on-target extension product.

104. The method of embodiment 103, wherein identifying the sequence in the sequencing data as being associated with the on-target extension product comprises analyzing a length distribution of the one or more extension products.

105. The method of any one of embodiments 102-104, comprising removing from the sequencing data sequences associated with off-target extension products.

106. The method of any one of the preceding embodiments, wherein the capture moiety and/or the detection moiety comprises or is an antibody or binding fragment thereof, a lectin, a receptor, a cofactor, a polynucleotide, an aptamer, a single chain protein binder, a peptide, a modified enzyme substrate, and/or a suicide inhibitor.

107. The method of any one of the preceding embodiments, wherein the capture moiety and/or the detection moiety comprises or is an antibody or binding fragment thereof.

108. The method of any one of the preceding embodiments, wherein the solid support comprises a bead, a slide, or a microwell.

109. The method of any one of the preceding embodiments, wherein the solid support comprises a magnetic, paramagnetic or superparamagnetic bead.

110. The method of any one of the preceding embodiments, wherein the solid support comprises a polymer selected from: polystyrene, polypropylene, polyethylene, polydimethylsiloxane (PDMS), silicone, agarose, gelatin.

111. The method of any one of the preceding embodiments, wherein the sample comprises plasma, serum, blood, stool, urine, cerebral spinal fluid, and/or saliva.

112. The method of any one of the preceding embodiments, wherein the analyte comprises an antibody, a polypeptide, or a small molecule.

113. The method of any one of the preceding embodiments, wherein the oligonucleotides comprises DNA, RNA, or analogues and derivatives thereof.

114. The method of any one of the preceding embodiments, wherein
the sample comprises the analyte,
the detection moiety and capture moiety are both bound (or are simultaneously bound) to the analyte such that the capture oligonucleotide and detection oligonucleotide are in proximity after c),
the 3′ hybridizing region of the capture oligonucleotide and the 3′ hybridizing region of the detection oligonucleotide are hybridized to each other in d);
the hybridized capture oligonucleotide and the hybridized detection oligonucleotide are extended to generate on-target extension product that comprises the extended capture oligonucleotide and the extended detection oligonucleotide in e), and
one or both strands of the on-target extension product is released from the solid support and/or the detection moiety in f), and
wherein the method comprises in g), determining the presence and/or amount of the released extension product to determine the presence and/or amount of the analyte in the sample.

114a. The method of any one of embodiments 1 to 114, wherein the detection oligonucleotide is attached to the detection moiety in a deterministic manner.

114b. The method of embodiment 114a, wherein only one detection oligonucleotide is attached to each detection moiety.

114c. The method of embodiment 114a, wherein only two detection oligonucleotides are attached to each detection moiety.

115. A composition comprising a plurality of (e.g., at least 30, or 30-200, etc.) pairs of oligonucleotides, each pair comprising:
a capture oligonucleotide at least 25 nucleotides long, comprising:
   a 3′ hybridizing region at most 10 nucleotides long; and
   a 5′ tethering region at least 15 nucleotides long; and
a detection oligonucleotide at least 25 nucleotides long, comprising:
   a 3′ hybridizing region at most 10 nucleotides long and complementary to
the 3′ hybridizing region of the capture oligonucleotide; and
   a 5′ tethering region at least 15 nucleotides long,
wherein the 3′ hybridizing region of the capture oligonucleotide and detection oligonucleotide of each pair of oligonucleotides is not complementary to the 3′ hybridizing region of the detection oligonucleotide and the capture oligonucleotide, respectively, of any other pair of the plurality of (e.g., at least 30, or 30-200, etc.) pairs of oligonucleotides,
wherein each of the plurality of (e.g., at least 30, or 30-200, etc.) pairs of oligonucleotides has a mis-pairing rate of at most about 1% in the presence of the other plurality of (e.g., at least 30, or 30-200, etc.) pairs of oligonucleotides in a proximity-based extension assay.

116. The composition of embodiment 115, wherein the proximity-based extension assay is a proximity extension strand displacement (PESD) assay.

117. The composition of embodiment 115 or 116, wherein the 5′ tethering region of the capture oligonucleotide is the same for all of the plurality of (e.g., at least 30, or 30-200, etc.) pairs of oligonucleotides.

118. The composition of any one of embodiments 115-117, wherein the 5′ tethering region of the detection oligonucleotide is the same for all of the plurality of (e.g., at least 30, or 30-200, etc.) pairs of oligonucleotides.

119. The composition of any one of embodiments 115-118, wherein the capture oligonucleotide comprises a barcode sequence at least 6 nucleotides long between the 5′ tethering region and the 3′ hybridizing region, and/or the detection oligonucleotide comprises a barcode sequence at least 6 nucleotides long between the 5′ tethering region and the 3′ hybridizing region.

120. The composition of any one of embodiments 115-119, wherein the 5′ tethering region and barcode sequence are adjacent each other in the capture oligonucleotide, and the barcode sequence and the 3′ hybridizing region are adjacent each other in the capture oligonucleotide.

121. The composition of any one of embodiments 115-120, wherein the 5′ tethering region and barcode sequence are adjacent each other in the detection oligonucleotide, and the barcode sequence and the 3′ hybridizing region are adjacent each other in the detection oligonucleotide.

122. The composition of any one of embodiments 115-119, wherein the capture oligonucleotide comprises a unique molecular identifier (UMI) at least 4 nucleotides long between the 5′ tethering region and the 3′ hybridizing region, and the detection oligonucleotide comprises a UMI at least 4 nucleotides long between the 5′ tethering region and the 3′ hybridizing region.

123. The composition of any one of embodiments 115-122, wherein the 3′ hybridizing region of the capture oligonucleotide and the detection oligonucleotide in each of the plurality of (e.g., at least 30, or 30-200, etc.) pairs of oligonucleotides is 6 or 7 nucleotides long.

124. The composition of any one of embodiments 115-123, wherein the 3′ hybridizing region of the capture oligonucleotide and/or detection oligonucleotide in each of the plurality of (e.g., at least) 30 pairs of oligonucleotides has a Hamming distance of at least 2 relative to the 3′ hybridizing region of the capture oligonucleotide and/or the detection oligonucleotide, respectively, of any other pair of the plurality of (e.g., at least 30, or 30-200, etc.) pairs of oligonucleotides.

125. The composition of any one of embodiments 115-124, wherein a first calculated ΔG of hybridization between the 3′ hybridizing region of the capture oligonucleotide and detection oligonucleotide in each of the plurality of (e.g., at least 30, or 30-200, etc.) pairs of oligonucleotides is, or is at least, about −4 kcal/mol or more negative than a second calculated ΔG of hybridization between:
(1) the 3′ hybridizing region of the capture oligonucleotide of the corresponding pair of oligonucleotides and the 3′ hybridizing region of the detection oligonucleotide of any other pair (or each of the other pairs) of the plurality of (e.g., at least 30, or 30-200, etc.) pairs of oligonucleotides; and/or
(2) the 3′ hybridizing region of the detection oligonucleotide of the corresponding pair of oligonucleotides and the 3′ hybridizing region of the capture oligonucleotide of any other pair (or each of the other pairs) of the plurality of (e.g., at least 30) pairs of oligonucleotides.

126. A composition comprising:
a first pool of a plurality of (e.g., at least 30, or 30-200, etc.) solid supports, each solid support comprising:
   a capture moiety attached to the solid support, wherein the capture moiety binds an analyte; and
   a capture oligonucleotide attached to the solid support, wherein the capture oligonucleotide comprises a 3′ hybridizing region; and
a second pool of a plurality of (e.g., at least 30, or 30-200, etc.) detection conjugates, each detection conjugate comprising:
   a detection moiety that binds the analyte; and
   a detection oligonucleotide attached to the detection moiety, wherein the detection oligonucleotide comprises a 3′ hybridizing region complementary to the
3′ hybridizing region of the capture oligonucleotide,
wherein each of the plurality of (e.g., at least 30, or 30-200, etc.) solid supports forms a paired combination with a corresponding detection conjugate of the plurality of (e.g., at least 30, or 30-200, etc.) detection conjugates, wherein a binding target of the capture moiety and detection moiety of each paired combination is the same, and wherein different paired combinations have different binding targets,
wherein the 3′ hybridizing region of the capture oligonucleotide and detection oligonucleotide of each paired combination is not complementary to the 3′ hybridizing region of the detection oligonucleotide and the capture oligonucleotide, respectively, of any other paired combination,
optionally wherein each of the paired combinations has a mis-hybridization rate of at most about 1% in the presence of the other paired combinations in a proximity-based extension assay.

126a. The composition of embodiment 126, wherein the plurality of detection conjugates comprises a plurality of (e.g., at least 30, or 30-200, etc.) detection conjugates, and the plurality of solid supports comprises a plurality of (e.g., at least 30, or 30-200, etc.) solid supports, and wherein each of the plurality of (e.g., at least 30, or 30-200, etc.) solid supports forms the paired combination with the corresponding detection conjugate of the plurality of (e.g., at least 30, or 30-200, etc.) detection conjugates.

126b. The composition of embodiment 126 or 126a, wherein each of the paired combinations has a mis-hybridization rate of at most about 1% in the presence of the other paired combinations in a proximity-based extension assay.

126c. The composition of embodiment 126, 126a, or 126b, further comprising a plurality of analytes comprising binding targets of the capture moieties and detection moieties 127. A composition comprising:
a solid support comprising:
   a capture moiety attached to the solid support, wherein the capture moiety binds an analyte; and
   a capture oligonucleotide attached to the solid support, wherein the capture oligonucleotide comprises a 3′ hybridizing region;
a detection conjugate comprising:
   a detection moiety that binds the analyte; and
   a detection oligonucleotide attached to the detection moiety, wherein the detection oligonucleotide comprises a 3′ hybridizing region complementary to the 3′ hybridizing region of the capture oligonucleotide,
wherein the capture moiety and the detection moiety are both bound (or are simultaneously bound) to the analyte such that the 3′ hybridizing region of the capture oligonucleotide and the 3′ hybridizing region of the detection oligonucleotide are in proximity to allow hybridization to each other.

128. The composition of embodiment 127, wherein the capture oligonucleotide is attached to the solid support via hybridization to a first tether oligonucleotide attached to the solid support, and/or the detection oligonucleotide is attached to the detection moiety via hybridization to a second tether oligonucleotide attached to the detection moiety.

129. The composition of embodiment 128, wherein the capture oligonucleotide comprises a 5′ tethering region that hybridizes to the first tether oligonucleotide.

130. The composition of embodiment 128 or 129, wherein the detection oligonucleotide comprises a 5′ tethering region that hybridizes to the second tether oligonucleotide.

130a. The composition of any one of embodiments 126 to 130, wherein the detection oligonucleotide is attached to the detection moiety in a deterministic manner.

130b. The composition of embodiment 130a, wherein only one detection oligonucleotide is attached to each detection moiety.

130c. The composition of embodiment 130a, wherein only two detection oligonucleotides are attached to each detection moiety.

131. A composition comprising a plurality of partially double-stranded nucleic acids, each partially double-stranded nucleic acid comprising: a capture oligonucleotide hybridized at the 5′ end with a first tether oligonucleotide of 15-25 nucleotides in length and comprising a 3′ hybridizing region of at most 10 nucleotides; a detection oligonucleotide hybridized at the 5′ end with a second tether oligonucleotide of 15-25 nucleotides in length and comprising a 3′ hybridizing region of at most 10 nucleotides, wherein the 3′ hybridizing region of the capture oligonucleotide is hybridized to the 3′ hybridizing region of the detection oligonucleotide.

132. The composition of embodiment 131, wherein the composition comprises a solid support attached to the first tether oligonucleotide.

133. A method of identifying a pairwise combination of binding moieties that can both be bound (or can be simultaneously bound) to a binding target, comprising:
a) providing a plurality of solid supports, each solid support comprising:
   a first binding moiety attached to the solid support, wherein the binding moiety binds a binding target; and
   a capture oligonucleotide attached to the solid support, wherein the capture oligonucleotide comprises:
      a 3′ hybridizing region; and
      a capture barcode region,
wherein the plurality of solid supports comprises a first plurality of different binding moieties that bind the same binding target, wherein the capture barcode region of each solid support of the plurality of solid supports identifies one of the first plurality of different binding moieties attached to the respective solid support, and wherein the 3′ hybridizing regions of the capture oligonucleotides attached to the plurality of solid supports are the same;
b) providing a plurality of detection conjugates comprising:
   a second binding moiety; and
   a detection oligonucleotide attached to the binding moiety, wherein the detection oligonucleotide comprises:
      a 3′ hybridizing region complementary to the 3′ hybridizing region of the capture oligonucleotide; and
      a detector barcode region,
wherein the plurality of detection conjugates comprises a second plurality of different binding moieties, wherein the detector barcode region of each detection conjugate of the plurality of detection conjugates identifies one of the second plurality of different binding moieties attached to the detection oligonucleotide, and wherein the 3′ hybridizing regions of the detection oligonucleotides of the plurality of detection conjugates are the same;
c) preparing a complexing solution by:
   i) combining in a solution the plurality of solid supports provided in a) and the plurality of detection conjugates provided in b) with a sample comprising a plurality of molecules of the binding target, thereby allowing one or more of the first plurality of different binding moieties of the plurality of solid supports and one or more of the second plurality of different binding moieties of the plurality of detection conjugates to be bound to one or more molecules of the plurality of molecules of the binding target,
   ii) contacting the plurality of solid supports provided in a) with the sample, thereby allowing one or more of the first plurality of different binding moieties of the plurality of solid supports to be bound to one or more molecules of the plurality of molecules of the binding target, and combining in a solution the sample-contacted plurality of solid supports and the plurality of detection conjugates provided in b), or
   iii) contacting the plurality of detection conjugates provided in b) with the sample, thereby allowing one or more of the second plurality of different binding moieties of the plurality of detection conjugates to be bound to one or more molecules of the plurality of molecules of the binding target, and combining in a solution the sample-contacted plurality of detection conjugates and the plurality of solid supports provided in a),
wherein the 3′ hybridizing region of a capture oligonucleotide attached to a first solid support of the plurality of solid supports and the 3′ hybridizing region of a detection oligonucleotide of a first detection conjugate of the plurality of detection conjugates are in proximity when the first binding moiety attached to the first solid support and the second binding moiety of the first detection conjugate are both bound (or are simultaneously bound) to the same molecule of the plurality of molecules of the binding target;
d) permitting the 3′ hybridizing region of the capture oligonucleotides attached to the plurality of solid supports and the 3′ hybridizing region of the detection oligonucleotides of the plurality of detection conjugates that are in proximity to hybridize to each other;
e) extending hybridized capture oligonucleotides and/or hybridized detection oligonucleotides to generate extension products that each comprise an extended capture oligonucleotide and/or an extended detection oligonucleotide;
f) releasing each of the extension products from the respective solid supports and, optionally, from the respective second binding moieties; and
g) determining:
   the presence and/or amount, or the absence of the released extension products; and
   the respective capture barcode region and the detector barcode region in each of the released extension products,
   to thereby determine the suitability of a combination of binding moieties identified by the capture barcode region and the detector barcode region in the released extension products for use in a sandwich-type assay.

134. The method of embodiment 133, wherein the plurality of solid supports comprises approximately equal amounts of each different binding moiety of the plurality of different binding moieties, and the plurality of detection conjugates comprises approximately equal amounts of each different binding moiety of the plurality of different binding moieties.

135. The method of embodiment 133 or 134, comprising:
providing a plurality of variants of the binding target; and
for each of the plurality of variants of the binding target, contacting the plurality of solid supports or the plurality of detection conjugates, or both with a sample comprising the variant of the binding target.

136. The method of embodiment 135, wherein the plurality of variants of the binding target comprises binding targets from different species and/or different sources.

137. The method of embodiment 135 or 136, comprising contacting the plurality of solid supports or the plurality of detection conjugates, or both with a plurality of samples, each comprising the plurality of molecules of the binding target at different concentrations.

138. The method of any one of embodiments 133-137, wherein in each solid support of the plurality of solid supports, the capture oligonucleotide is attached to the solid support via hybridization to a first tether oligonucleotide attached to the solid support.

139. The method of any one of embodiments 133-138, wherein in each detection conjugate of the plurality of detection conjugates, the detection oligonucleotide is attached to the detection moiety via hybridization to a second tether oligonucleotide attached to the detection moiety.

140. The method of embodiment 138 or 139, wherein the extending at e) comprises treating the plurality of solid support with a strand-displacing polymerase under conditions sufficient to extend hybridized capture oligonucleotides and hybridized detection oligonucleotides, and
wherein the releasing at f) comprises allowing the strand-displacing polymerase to displace the first tether oligonucleotide hybridized to the capture oligonucleotide during extension, and optionally to displace the second tether oligonucleotide hybridized to the detection oligonucleotide during extension.

141. The method of any one of embodiments 133-140, wherein the capture moiety and/or the detection moiety comprises or is an antibody or binding fragment thereof, a lectin, a receptor, a cofactor, a polynucleotide, an aptamer, a single chain protein binder, a peptide, a modified enzyme substrate, and/or a suicide inhibitor.

142. The method of any one of embodiments 133-141, wherein the binding target comprises or is an antibody, a polypeptide, and/or a small molecule.

142a. The method of any one of embodiments 133 to 140, wherein the detection oligonucleotide is attached to the second binding moiety in a deterministic manner.

142b. The method of embodiment 142a, wherein only one detection oligonucleotide is attached to each second binding moiety.

142c. The method of embodiment 142a, wherein only two detection oligonucleotides are attached to each second binding moiety.

143. A method of analyzing a sample for an analyte, comprising:
a) providing a first conjugate comprising:
   a first moiety that binds an analyte; and
   a first splint oligonucleotide attached to the first moiety, wherein the first splint oligonucleotide comprises a 3′ hybridizing region;
b) providing a second conjugate comprising:
   a second moiety that binds the analyte; and
   a second splint oligonucleotide attached to the second moiety, wherein the second splint oligonucleotide comprises a 3′ hybridizing region complementary to the 3′ hybridizing region of the first splint oligonucleotide;
   wherein the first splint oligonucleotide is attached to the first moiety via hybridization to a first tether oligonucleotide attached to the first moiety, and/or the second splint oligonucleotide is attached to the second moiety via hybridization to a second tether oligonucleotide attached to the second moiety;
   wherein the first and/or the second splint oligonucleotide comprises a barcode sequence that identifies the moiety to which the splint oligonucleotide is attached and/or a binding target thereof;
c) preparing a complexing solution by:
   i) combining in a solution the first conjugate provided in a) and the second conjugate provided in b) with a sample, thereby allowing the first conjugate and the second conjugate to be bound to the analyte if present in the sample; or
   ii) contacting the first conjugate provided in a) with a sample, thereby allowing the first moiety of the first conjugate to be bound to the analyte if present in the sample, and combining in a solution the sample-contacted first conjugate and the second conjugate provided in b);
thereby allowing the first moiety and the second moiety in the complexing solution to both be bound to the analyte if present such that the first splint oligonucleotide and second splint oligonucleotide are in proximity if the analyte is present in the sample;
d) permitting the 3′ hybridizing region of the first splint oligonucleotide and the 3′ hybridizing region of the second splint oligonucleotide that are in proximity to hybridize to each other;
e) extending the hybridized first splint oligonucleotide and/or the hybridized second splint oligonucleotide to generate an on-target extension product that comprises the extended first splint oligonucleotide and/or the extended second splint oligonucleotide;
f) releasing the on-target extension product from the first moiety and/or the second moiety; and
g) determining the presence and/or amount, or the absence of the on-target extension product to thereby determine the presence and/or amount, or the absence, of the analyte in the sample.

144. The method of embodiment 143, wherein the extending in e) is performed by a strand-displacing polymerase.

145. The method of embodiment 143 or 144, wherein releasing the on-target extension product comprises treating the on-target extension product with a restriction enzyme, a protease, and/or a high-stringency wash.

146. The method of any one of embodiments 143-145, wherein the first splint oligonucleotide is attached to the first moiety via hybridization to a first tether oligonucleotide attached to the first moiety, and the second splint oligonucleotide is attached to the second moiety via hybridization to a second tether oligonucleotide attached to the second moiety.

147. The method of embodiment 146, wherein the releasing in f) is performed at a temperature in a range of 10⁻³⁷° C.

148. The method of any one of embodiments 143-147, wherein extending and releasing are performed by a single enzyme.

149. The method of embodiment 148, wherein the single enzyme comprises a strand-displacing polymerase.

150. The method of any one of embodiments 143-149, wherein the extending in e) comprises contacting the hybridized first splint oligonucleotide and/or the hybridized second splint oligonucleotide with a strand-displacing polymerase under conditions sufficient to extend the hybridized first splint oligonucleotide and/or the hybridized second splint oligonucleotide, and
wherein the releasing in f) comprises allowing the strand-displacing polymerase to displace the first tether oligonucleotide hybridized to the first splint oligonucleotide during extension, and/or to displace the second tether oligonucleotide hybridized to the second splint oligonucleotide during extension.

151. The method of embodiment 150, wherein the strand-displacing polymerase is allowed to displace the first tether oligonucleotide hybridized to the first splint oligonucleotide during extension, and to displace the second tether oligonucleotide hybridized to the second splint oligonucleotide during extension.

152. The method of any one of embodiments 143-145, wherein
the first splint oligonucleotide is attached to the first moiety via a bonding interaction that is independent of the nucleotide sequence comprised in the first splint oligonucleotide, and/or
the second splint oligonucleotide is attached to the second moiety via a bonding interaction that is independent of the nucleotide sequence comprised in the second splint oligonucleotide.

153. The method of embodiment 152, wherein either the first splint oligonucleotide is covalently attached to the first moiety, or the second splint oligonucleotide is covalently attached to the second moiety.

154. The method of embodiment 152 or 153, wherein the releasing in f) comprises cleaving a covalent attachment of the on-target extension product to the first or second moiety.

155. The method of any one of embodiments 152-154, wherein the releasing in f) comprises contacting the on-target extension product with a protease.

156. The method of any one of embodiments 144-155, wherein the strand-displacing polymerase comprises an exo-Klenow fragment.

157. The method of any one of embodiments 143-156, comprising providing a plurality of the first and second conjugates, wherein the method further comprises following releasing the on-target extension product from the first moiety and/or the second moiety in f) and before determining the presence and/or amount, or the absence of the on-target extension product in g), separating the released on-target extension product from first conjugates of the plurality of first conjugates comprising the first splint oligonucleotide and/or from second conjugates of the plurality of second conjugates comprising the second splint oligonucleotide.

158. The method of embodiment 157, comprising separating the released on-target extension product from first conjugates of the plurality of first conjugates comprising an unextended first splint oligonucleotide and/or from second conjugates of the plurality of second conjugates comprising an unextended second splint oligonucleotide.

159. The method of embodiment 157 or 158, wherein the separating comprises size-exclusion chromatography, affinity chromatography, ion-exchange chromatography, and/or solid-phase reversible immobilization (SPRI).

160. The method of any one of embodiments 143-159, wherein the first tether oligonucleotide is covalently attached to:
(1) a first member of a binding pair that binds to a second member of the binding pair, wherein the second member is attached to the first moiety, or
(2) the first moiety.

161. The method of any one of embodiments 143-160, wherein the second tether oligonucleotide is covalently attached to:
(1) a first member of a binding pair that binds to a second member of the binding pair, wherein the second member is attached to the second moiety, or
(2) the second moiety.

162. The method of any one of embodiments 143-161, wherein either the first conjugate is attached to a solid support, or the second conjugate is attached to a solid support.

163. The method of embodiment 162, wherein the first conjugate is attached to a first member of a binding pair that binds to a second member of the binding pair, wherein the second member is attached to the solid support, or wherein the second conjugate is attached to a first member of a binding pair that binds to a second member of the binding pair, wherein the second member is attached to the solid support.

164. The method of any one of embodiments 160-163, wherein the first member of the binding pair comprises biotin, and the second member of the binding pair comprises streptavidin.

165. The method of embodiment 162, wherein the first conjugate is covalently attached to or is adsorbed onto the solid support, or wherein the second conjugate is covalently attached to or is adsorbed onto the solid support.

166. The method of any one of embodiments 162-165, comprising providing a first plurality of the first conjugates, and providing a second plurality of the second conjugates, wherein the solid support comprises the first plurality of the first conjugates or the second plurality of the second conjugates,
wherein the method further comprises following preparing the complexing solution in c) and prior to the extending in e), removing any first conjugate that is not bound to an analyte bound to a second conjugate attached to the solid support, or removing any second conjugate that is not bound to an analyte bound to a first conjugate attached to the solid support.

167. The method of embodiment 166, wherein the removing comprises washing the solid support under high stringency conditions.

168. The method of any one of embodiments 162-167, wherein the solid support comprises a bead, a slide, or a microwell.

169. The method of any one of embodiments 162-168, wherein the solid support comprises a magnetic, paramagnetic or superparamagnetic bead.

170. The method of any one of embodiments 162-169, wherein the solid support comprises a polymer selected from: polystyrene, polypropylene, polyethylene, polydimethylsiloxane (PDMS), silicone, agarose, gelatin.

171. The method of any one of embodiments 143-170, wherein preparing the complexing solution in c) comprises i) combining in a solution the first conjugate provided in a) and the second conjugate provided in b) with a sample, thereby allowing the first conjugate and the second conjugate to be bound to the analyte if present in the sample.

172. The method of any one of embodiments 143-170, wherein preparing the complexing solution in c) comprises ii) contacting the first conjugate provided in a) with a sample, thereby allowing the first moiety of the first conjugate to be bound to the analyte if present in the sample, and combining in a solution the sample-contacted first conjugate and the second conjugate provided in b).

173. The method of embodiment 172, comprising removing the sample before combining in the solution the sample-contacted first conjugate and the second conjugate provided in b), thereby removing analyte if present that is not bound to the first moiety.

174. The method of any one of embodiments 143-173, wherein the first and the second splint oligonucleotides each comprises a barcode sequence that identifies the moiety to which the splint oligonucleotide is attached and/or a binding target thereof.

175. The method of any one of embodiments 143-174, wherein the first splint oligonucleotide and/or second splint oligonucleotide comprises, from 5′ to 3′: a tethering region, the barcode sequence, and the 3′ hybridizing region.

176. The method of any one of embodiments 143-175, wherein the first splint oligonucleotide and the second splint oligonucleotide each comprises a primer binding region configured to bind a primer pair for amplifying the released on-target extension product.

177. The method of embodiment 176, wherein the first splint oligonucleotide and the second splint oligonucleotide each comprises a 5′ tethering region, wherein the 5′ tethering region comprises the primer binding region or a portion thereof.

178. The method of any one of embodiments 143-177, comprising providing:
a plurality of paired combinations of the first conjugates and the second conjugates, wherein a binding target of the first moiety and second moiety of each paired combination is the same, and wherein different paired combinations of the plurality of paired combinations have different binding targets.

179. The method of embodiment 178, wherein the 3′ hybridizing region of the first splint oligonucleotide and second splint oligonucleotide of each of the plurality of paired combinations of the first conjugates and the second conjugates identifies the binding target of the corresponding paired combination.

180. The method of embodiment 178 or 179, wherein the 3′ hybridizing region of the first splint oligonucleotide and second splint oligonucleotide of a paired combination of the plurality of paired combinations of first conjugates and the second conjugates identifies a binding target that is different from a binding target identified by the 3′ hybridizing region of the first splint oligonucleotide and second splint oligonucleotide of at least one other paired combinations of the plurality of paired combinations.

181. The method of any one of embodiments 178-180, wherein the 3′ hybridizing region of the first splint oligonucleotide and second splint oligonucleotide of a first paired combination of the plurality of paired combinations is not complementary to the 3′ hybridizing region of the second splint oligonucleotide and first splint oligonucleotide, respectively, of at least one other paired combination of the plurality of paired combinations.

182. The method of embodiment 181, wherein the 3′ hybridizing region of the first splint oligonucleotide and/or second splint oligonucleotide of the first paired combination has a Hamming distance of at least 2 relative to the 3′ hybridizing region of the first splint oligonucleotide and/or second splint oligonucleotide, respectively, of at least one other paired combination of the plurality of paired combinations.

183. The method of embodiment 181 or 182, wherein a first calculated ΔG of hybridization between the 3′ hybridizing regions of the first splint oligonucleotide and second splint oligonucleotide of the first paired combination is about −4 kcal/mol or more negative than a second calculated ΔG of hybridization between:
(1) the 3′ hybridizing region of the first splint oligonucleotide of the first paired combination and the 3′ hybridizing region of the second splint oligonucleotide of each of the at least one other paired combination of the plurality of paired combinations; and
(2) the 3′ hybridizing region of the second splint oligonucleotide of the first paired combination and the 3′ hybridizing region of the first splint oligonucleotide of each of the at least one other paired combination of the plurality of paired combinations.

184. The method of any one of embodiments 178-183, comprising providing a plurality of spatially distinct partitions, each comprising at least one first or second conjugate of the plurality of paired combinations of the first conjugates and the second conjugates, wherein the first or second moiety of the at least one first or second conjugate in a partition of the plurality of spatially distinct partitions has a different binding target from the first or second moiety of the at least one first or second conjugate in a different partition of the plurality of spatially distinct partitions.

185. The method of embodiment 184, comprising pooling the first or second conjugates in the plurality of spatially distinct partitions before preparing the complexing solution in c).

186. The method of embodiment 184 or 185, wherein preparing the complexing solution in c) comprises:
contacting portions of the sample with each of the first or second conjugates in the plurality of spatially distinct partitions, then pooling the first or second conjugates in the plurality of spatially distinct partitions, and then contacting the pooled first conjugates with the second conjugates of the plurality of paired combinations or contacting the pooled second conjugates with the first conjugates of the plurality of paired combinations.

187. The method of any one of embodiments 178-186, comprising diluting an original sample comprising one or more analytes bound by the plurality of paired combinations of the first conjugates and the second conjugates, thereby generating the sample comprising the one or more analytes at a lower concentration than the original sample.

188. The method of any one of embodiments 184-187, wherein the plurality of spatially distinct partitions comprises a plurality of microtubes, microwells, and/or microfluidic chambers.

189. The method of any one of embodiments 178-188, wherein each first splint oligonucleotide attached to a first moiety of the plurality of the first conjugates comprises a barcode sequence that identifies a binding target of the first moiety, and/or wherein each second splint oligonucleotide attached to a second moiety of the plurality of the second conjugates comprises a barcode sequence that identifies a binding target of the second moiety.

190. The method of any one of embodiments 178-189, wherein a barcode sequence of a second splint oligonucleotide attached to a second moiety that has the same binding target as a first moiety is identifiable based on the barcode sequence of a first splint oligonucleotide attached the first moiety, and/or
a barcode sequence of a first splint oligonucleotide attached to a first moiety that has the same binding target as a second moiety is identifiable based on the barcode sequence of a second splint oligonucleotide attached the second moiety.

191. The method of any one of embodiments 178-190, wherein barcode sequences of two different first splint oligonucleotides that are attached to first moieties having different binding targets have a Hamming distance of at least 3, and/or
wherein barcode sequences of two different second splint oligonucleotides that are attached to second moieties having different binding targets have a Hamming distance of at least 3.

192. The method of any one of embodiments 178-191, wherein the plurality of paired combinations of the first conjugates and the second conjugates comprises at least 5 paired combinations.

193. The method of any one of embodiments 143-192, wherein the first moiety and/or the second moiety is an antibody or binding fragment thereof, a lectin, a receptor, a cofactor, a polynucleotide, an aptamer, a single chain protein binder, a peptide, a modified enzyme substrate, or a suicide inhibitor.

194. The method of any one of embodiments 143-193, wherein the first moiety and/or the second moiety is an antibody or binding fragment thereof.

195. The method of any one of embodiments 143-194, wherein the sample comprises plasma, serum, blood, stool, urine, saliva.

196. The method of any one of embodiments 143-195, wherein the analyte comprises an antibody, a polypeptide, or a small molecule.

197. The method of any one of embodiments 143-196, wherein the oligonucleotides comprises DNA, RNA, or analogues and derivatives thereof.

198. The method of any one embodiments 143-197, wherein determining the presence and/or amount, or the absence of the on-target extension product comprises performing qPCR on one or more extension products generated at e) and released in f).

199. The method of any one of embodiments 143-198, wherein determining the presence and/or amount, or the absence of the on-target extension product comprises obtaining sequencing data of one or more extension products generated at e) and released in f).

200. The method of embodiment 199, comprising sequencing the one or more released extension products to obtain the sequencing data of the one or more released extension products.

201. The method of embodiment 200, wherein the sequencing comprises high-throughput sequencing.

202. The method of any one of embodiments 199-201, wherein determining the presence and/or amount, or the absence of the released on-target extension product comprises amplifying the one or more extension products generated at e) and released in f).

203. The method of embodiment 202, wherein determining the presence and/or amount, or the absence of the released on-target extension product comprises:
combining the one or more extension products with a control oligonucleotide in an amplification mixture;
amplifying the one or more extension products and the control oligonucleotide in the amplification mixture, to generate extension product amplicons and control oligonucleotide amplicons,
detecting a level of the extension product amplicons and the control oligonucleotide amplicons; and
normalizing the detected level of the extension product amplicons based on the detected level of the control oligonucleotide amplicons.

203a. The method of embodiment 203, wherein the released on-target extension products are distributed among multiple partitions, wherein determining the presence and/or amount, or the absence, of the released on-target extension products in the multiple partition comprises:
determining the concentration of extension products in each partition;
diluting the extension products in each partition to normalize the concentration of extension products in each partition;
combining the diluted extension products in each partition with a control oligonucleotide in an amplification mixture;
amplifying the diluted extension products and the control oligonucleotide in the amplification mixture, to generate extension product amplicons and control oligonucleotide amplicons;
detecting a level of the extension product amplicons and the control oligonucleotide amplicons in each partition by performing high throughput sequencing; and
normalizing the detected level of the extension product amplicons based on the detected level of the control oligonucleotide amplicons.

203b. The method of embodiment 203a, wherein determining the concentration of extension products in each partition comprises performing qPCR using PCR primers common to all amplicon molecules in each partition.

203c. The method of embodiment 203a, wherein the released on-target extension products are distributed among multiple partitions, wherein determining the presence and/or amount, or the absence, of the released on-target extension products in the multiple partition comprises:
combining the diluted extension products in each partition with a control oligonucleotide in an amplification mixture;
amplifying the diluted extension products and the control oligonucleotide in the amplification mixture, to generate extension product amplicons and control oligonucleotide amplicons, wherein one of the PCR primers in the amplification mixture is present at a concentration such that it will be depleted in at least one partition prior to the completion of the amplification reaction;
detecting a level of the extension product amplicons and the control oligonucleotide amplicons in each partition by performing high throughput sequencing; and
normalizing the detected level of the extension product amplicons based on the detected level of the control oligonucleotide amplicons.

204. The method of embodiment 203, 203a, 203b, or 203c, wherein the control oligonucleotide comprises a unique molecular identifier (UMI), wherein detecting the level of the control oligonucleotide comprises obtaining sequence information of the UMI in the control oligonucleotide amplicons, and wherein the detected level of the control oligonucleotide is based on the number of the UMI with distinct sequences associated with a sequence of the control oligonucleotide in the sequencing data.

205. The method of embodiment 203, 203a, 203b, 203c or 204, wherein the extension product amplicons comprise adapter sequences that hybridize to one or more sequencing primers.

206. The method of any one of embodiments 203-205, wherein the extension product amplicons comprise one or more indexing sequences.

207. The method of any one of embodiments 199-206, wherein the capture oligonucleotide and/or detection oligonucleotide comprises a unique molecular identifier (UMI), or wherein the first splint oligonucleotide and/or second splint oligonucleotide comprises a unique molecular identifier (UMI).

208. The method of embodiment 207, wherein determining the presence and/or amount, or the absence of the released on-target extension product comprises determining the number of UMI with distinct sequences associated with the capture oligonucleotide and/or detection oligonucleotide, or determining the number of UMI with distinct sequences associated with the first splint oligonucleotide and/or second splint oligonucleotide.

209. The method of any one of embodiments 199-208, comprising identifying a sequence of an extension product in the sequencing data as being associated with the on-target extension product based on a pairing of: (A) one or more subsequences in the sequence associated with the detection oligonucleotide, wherein the one or more subsequences identify the binding target of the detection moiety attached to the detection oligonucleotide, with (B) one or more subsequences in the sequence associated with the capture oligonucleotide, wherein the one or more subsequences identify a binding target of the capture moiety attached to the solid support to which the capture oligonucleotide is attached that is the same as the binding target of the detection moiety, or
identifying a sequence of an extension product in the sequencing data as being associated with the on-target extension product based on a pairing of: (A) one or more subsequences in the sequence associated with the second splint oligonucleotide, wherein the one or more subsequences identify the binding target of the second moiety attached to the second splint oligonucleotide, with (B) one or more subsequences in the sequence associated with the first splint oligonucleotide, wherein the one or more subsequences identify a binding target of the first moiety attached to the first splint oligonucleotide that is the same as the binding target of the second moiety.

210. The method of embodiment 209, wherein at least one of the one or more subsequences in the sequence associated with the detection oligonucleotide and the one or more subsequences in the sequence associated with the capture oligonucleotide is a barcode sequence, or wherein at least one of the one or more subsequences in the sequence associated with the first splint oligonucleotide and the one or more subsequences in the sequence associated with the second splint oligonucleotide is a barcode sequence.

211. The method of embodiment 210, wherein the one or more subsequences in the sequence associated with the detection oligonucleotide comprises a first barcode sequence, and the one or more subsequences in the sequence associated with the capture oligonucleotide comprises a second barcode sequence, or wherein the one or more subsequences in the sequence associated with the first splint oligonucleotide comprises a first barcode sequence, and the one or more subsequences in the sequence associated with the second splint oligonucleotide comprises a second barcode sequence.

212. The method of embodiment 210 or 211, wherein the 3′ hybridizing region identifies the binding target of the detection oligonucleotide or capture oligonucleotide, or wherein the 3′ hybridizing region identifies the binding target of the first splint oligonucleotide or second splint oligonucleotide.

213. The method of any one of embodiments 209-212, wherein identifying the sequence in the sequencing data as being associated with the on-target extension product is further based on a length of the sequenced extension product, wherein an on-target extension product is associated with a sequenced extension product having a length consistent with the length expected for the on-target extension product.

214. The method of any one of embodiments 209-213, wherein a sequence in the sequencing data is identified as an off-target extension product based on a mispairing between: (A) the one or more subsequences in the sequence associated with the detection oligonucleotide, wherein the one or more subsequences identify the binding target of the detection moiety attached to the detection oligonucleotide, and (B) the one or more subsequences in the sequence associated with the capture oligonucleotide, wherein the one or more subsequences identify a different binding target of a different capture moiety that is attached to a different solid support to which the capture oligonucleotide is attached, or wherein a sequence in the sequencing data is identified as an off-target extension product based on a mispairing between: (A) the one or more subsequences in the sequence associated with the second splint oligonucleotide, wherein the one or more subsequences identify the binding target of the second moiety attached to the second splint oligonucleotide, and (B) the one or more subsequences in the sequence associated with the first splint oligonucleotide, wherein the one or more subsequences identify a different binding target of a different first moiety that is attached to a different solid support to which the first splint oligonucleotide is attached.

215. The method of embodiment 214, wherein identifying the sequence in the sequencing data as being associated with the on-target extension product comprises analyzing a length distribution of the one or more extension products.

216. The method of embodiment 214 or 215, comprising removing from the sequencing data sequences associated with off-target extension products.

217. The method of any one of the preceding embodiments, wherein the strand-displacing polymerase is a 3′→5′ exo-polymerase.

217a. The method of any one of embodiments 143 to 270, wherein i) the first splint oligonucleotide is attached to the first moiety in a deterministic manner, and/or ii) the second splint oligonucleotide is attached to the second moiety in a deterministic manner.

217b. The method of embodiment 217a, wherein only one first splint oligonucleotide is attached to each first moiety and/or wherein only one second splint oligonucleotide is attached to each second moiety.

217c. The method of embodiment 217a, wherein only two first splint oligonucleotides are attached to each first moiety and/or only two second splint oligonucleotides are attached to each second moiety.

218. A composition comprising:
a first pool of a plurality of first conjugates, each first conjugate comprising:
   a first moiety that binds an analyte; and
   a first splint oligonucleotide attached to the first moiety, wherein the first splint oligonucleotide comprises a 3′ hybridizing region; and
a second pool of a plurality of second conjugates, each second conjugate comprising:
   a second moiety that binds the analyte; and
   a second splint oligonucleotide attached to the second moiety, wherein the second splint oligonucleotide comprises a 3′ hybridizing region complementary to the 3′ hybridizing region of the capture oligonucleotide,
wherein each of the plurality of first conjugates forms a paired combination with a corresponding second conjugate of the plurality of second conjugates, wherein a binding target of the first moiety and second moiety of each paired combination is the same, and
wherein different paired combinations have different binding targets, wherein for each paired combination, the first splint oligonucleotide is attached to the first moiety via hybridization to a first tether oligonucleotide attached to the first moiety, and/or the second splint oligonucleotide is attached to the second moiety via hybridization to a second tether oligonucleotide attached to the second moiety,
wherein for each paired combination, the first and/or the second splint oligonucleotide comprises a barcode sequence that identifies the moiety to which the splint oligonucleotide is attached and/or a binding target thereof,
wherein the 3′ hybridizing region of the first splint oligonucleotide and second splint oligonucleotide of each paired combination is not complementary to the 3′ hybridizing region of the second splint oligonucleotide and the first splint oligonucleotide, respectively, of any other paired combination.

219. The composition of embodiment 218, wherein the plurality of first conjugates and the plurality of second conjugates provide a plurality of (e.g., at least 30, or 30-200, etc.) paired combinations.

220. The composition of embodiment 218 or 219, wherein each of the paired combinations has a mis-hybridization rate of at most about 1% in the presence of the other paired combinations in a proximity-based extension assay.

221. The composition of any one of embodiments 218-220, wherein the first pool and the second pool are each in partitions that are spatially distinct from each other.

222. The composition of any one of embodiments 218-221, further comprising a plurality of analytes comprising binding targets of the first moieties and second moieties.

222a. The composition of any one of embodiments 218 to 222, wherein i) the first splint oligonucleotide is attached to the first moiety in a deterministic manner, and/or ii) the second splint oligonucleotide is attached to the second moiety in a deterministic manner.

222b. The composition of embodiment 222a, wherein only one first splint oligonucleotide is attached to each first moiety and/or wherein only one second splint oligonucleotide is attached to each second moiety.

222c. The composition of embodiment 222a, wherein only two first splint oligonucleotides are attached to each first moiety and/or only two second splint oligonucleotides are attached to each second moiety.

223. A method of analyzing a sample for an analyte, comprising:
a) providing a first construct comprising:
   a first moiety that binds an analyte; and
   a first splint oligonucleotide attached to the first moiety, wherein the first splint oligonucleotide comprises a 3′ hybridizing region;
b) providing a second construct comprising:
   a second moiety that binds the analyte; and
   a second splint oligonucleotide attached to the second moiety, wherein the second splint oligonucleotide comprises a 3′ hybridizing region complementary to the 3′ hybridizing region of the first splint oligonucleotide;
c) preparing a complexing solution by:
   i) combining in a solution the first construct provided in a) and the second construct provided in b) with a sample, thereby allowing the first moiety and the second moiety to be bound to the analyte if present in the sample;
   ii) contacting the first construct provided in a) with a sample, thereby allowing the first moiety to be bound to the analyte if present in the sample, and combining in a solution the sample-contacted construct and the second construct provided in b); or
   iii) contacting the second construct provided in b) with a sample, thereby allowing the second moiety to be bound to the analyte if present in the sample, and combining in a solution the sample-contacted construct with the first construct provided in a),
thereby allowing the first moiety and the second moiety in the complexing solution to both be bound to the analyte if present such that the first splint oligonucleotide and second splint oligonucleotide are in proximity if the analyte is present in the sample;
d) permitting the 3′ hybridizing region of the first splint oligonucleotide and the 3′ hybridizing region of the second splint oligonucleotide that are in proximity to hybridize to each other;
e) extending the hybridized first splint oligonucleotide and/or the hybridized second splint oligonucleotide to generate an on-target extension product that comprises the extended first splint oligonucleotide and/or the extended second splint oligonucleotide;
f) optionally, releasing the on-target extension product from the first construct and/or the second construct; and
g) determining the presence and/or amount, or the absence of the on-target extension product to thereby determine the presence and/or amount, or the absence, of the analyte in the sample,
wherein the method comprises one or more of the following:
   (I) the complexing solution comprises one or more blocker oligonucleotides, wherein each blocker oligonucleotide hybridizes to a subpart of the first splint oligonucleotide and/or of the second splint oligonucleotide;
   (II) the method comprises analyzing the sample for a first analyte and a second analyte, wherein preparing the complexing solution in c) comprises:
      preparing the complexing solution in a plurality of subpools comprising a first subpool and a second subpool, wherein the first moiety and second moiety in the prepared complexing solution of the first subpool bind the first analyte, and the first moiety and second moiety in the prepared complexing solution of the second subpool bind the second analyte; and
      combining the plurality of subpools before determining the presence and/or amount, or the absence of the on-target extension product in g);
   (III) providing a plurality of paired combinations of the first construct and second construct, wherein the plurality of paired combinations comprises one or more trimmed paired combinations comprising splint oligonucleotides having a 3′ hybridizing region that is 1, 2, 3 or more nucleotides shorter than the 3′ hybridizing region of the splint oligonucleotides of at least one other paired combination of the plurality of paired combinations, wherein the 3′ hybridizing regions of the splint oligonucleotides of the at least one other paired combination of the plurality of paired combinations is different from and is not complementary to any contiguous stretch of the 3′ hybridizing region of the splint oligonucleotides of the one or more trimmed paired combinations, optionally wherein the on-target extension products generated from substantially all (e.g., at least 95%) of the plurality of paired combinations of the first construct and second construct have the same length; or
   (IV) attenuating an amount of amplification products by reducing or interfering with a binding interaction between the analyte and the first moiety or the second moiety, and/or suppressing on-target interactions between the conjugate splint oligonucleotide and the first splint oligonucleotide when the first moiety and the second moiety are both bound to the analyte.

224. The method of embodiment 223, wherein the method comprises (I).

225. The method of embodiment 224, wherein the one or more blocker oligonucleotides reduce an analyte-independent interaction between the first splint oligonucleotide and the second splint oligonucleotide, and/or reduce an off-target interaction between the first splint oligonucleotide and the second splint oligonucleotide.

226. The method of embodiment 224 or 225, wherein the first splint oligonucleotide and/or second splint oligonucleotide in the complexing solution is, is about, or is at most 40, 35, 30, 25, 20, 15, 10, 5, 1% or 0% single-stranded, or optionally wherein it is a percentage in a range defined by any two of the preceding values (e.g., 1-40%, 5-35%, 10-25%, 1-10%, etc.) single-stranded, or optionally wherein it is about 0% single-stranded, along its length upon hybridization of the one or more blocker oligonucleotides to the first splint oligonucleotide and/or second splint oligonucleotide.

227. The method of any one of embodiments 224-226, comprising:
providing a plurality of the first constructs comprising a plurality of first splint oligonucleotides in a); and
providing a plurality of the second constructs comprising a plurality of second splint oligonucleotides in b),
wherein at least 80%, optionally at least 95%, of the plurality of first splint oligonucleotide and/or second splint oligonucleotides are each, are each about, or are each at most 40, 35, 30, 25, 20, 15, 10, 5, 1%, or 0% single-stranded, or optionally wherein it is a percentage in a range defined by any two of the preceding values (e.g., 1-40%, 5-35%, 10-25%, 1-10%, etc.) single-stranded, or optionally it is about 0% single-stranded along its length when the one or more blocker oligonucleotides are hybridized to one or more subparts of the at least 80%, optionally at least 95%, of the plurality of first splint oligonucleotides and/or second splint oligonucleotides.

228. The method of any one of embodiments 224-227, wherein the subpart to which a first blocker oligonucleotide of the one or more blocker oligonucleotides hybridizes comprises the 3′ hybridizing region or a portion thereof of the first splint oligonucleotide or second splint oligonucleotide, wherein the first blocker oligonucleotide competes with:
the 3′ hybridizing region of the first splint oligonucleotide for binding to the 3′ hybridizing region of the second splint oligonucleotide; or
the 3′ hybridizing region of the second splint oligonucleotide for binding to the 3′ hybridizing region of the first splint oligonucleotide.

229. The method of embodiment 228, comprising removing the first blocker oligonucleotide bound to the 3′ hybridizing region of the first splint oligonucleotide or second splint oligonucleotide after preparing the complexing solution in c) and before the extending in e).

230. The method of embodiment 229, wherein removing the first blocker oligonucleotide comprises:
washing the first construct comprising the first moiety bound to the analyte and/or the second construct comprising the second moiety bound to the analyte; and/or
contacting the first construct comprising the first moiety bound to the analyte and/or the second construct comprising the second moiety bound to the analyte with a nuclease specific to the first blocker oligonucleotide bound to the 3′ hybridizing region of the first splint oligonucleotide and/or second splint oligonucleotide.

231. The method of any one of embodiments 228-230, wherein an at least 5 nucleotide sequence of the first blocker oligonucleotide hybridizes to the subpart of the first splint oligonucleotide and/or of the second splint oligonucleotide.

232. The method of any one of embodiments 228-231, wherein the first blocker oligonucleotide is about 5 to about 13 nucleotides long, optionally about 7 to about 13 nucleotides long.

233. The method of any one of embodiments 228-232, wherein the subpart of the first splint oligonucleotide or second splint oligonucleotide to which the first blocker oligonucleotide of the one or more blocker oligonucleotides hybridizes comprises one or more 5′ residues adjacent the 3′ hybridizing region of the first splint oligonucleotide or second splint oligonucleotide, optionally wherein the one or more 5′ residues adjacent the 3′ hybridizing region does not comprise a barcode region of the first splint oligonucleotide or second splint oligonucleotide or a part thereof.

234. The method of any one of embodiments 228-233, wherein the first blocker oligonucleotide is present in the complexing solution at a concentration in a range of about 10 nM to about 10 μM, and/or at a concentration that is at about the same or greater than the concentration of the first splint oligonucleotide and/or second splint oligonucleotide to which the first blocker oligonucleotide hybridizes.

235. The method of any one of embodiments 228-234, comprising:
providing a plurality of the first constructs comprising a plurality of first splint oligonucleotides in a); and
providing a plurality of the second constructs comprising a plurality of second splint oligonucleotides in b),
wherein the plurality of first splint oligonucleotides comprises two or more different first splint oligonucleotides and/or the plurality of second splint oligonucleotides comprises two or more different second splint oligonucleotides, wherein the one or more blocker oligonucleotides comprises at least one first blocker oligonucleotide that hybridizes to the 3′ hybridizing region of at least one of the two or more different first splint oligonucleotides or of at least one of the two or more different second splint oligonucleotides.

236. The method of any one of embodiments 228-235, comprising providing a plurality of paired combinations of the first construct and second construct, wherein a binding target of the first moiety and second moiety of each paired combination is the same, and wherein different paired combinations of the plurality of paired combinations have different binding targets,
wherein the 3′ hybridizing region of the first splint oligonucleotide and second splint oligonucleotide of a paired combination of the plurality of paired combinations is different from and is not complementary to at least one other paired combination of the plurality of paired combinations having a different binding target, or
wherein all paired combinations of the plurality of paired combinations have a common 3′ hybridizing region.

237. The method of any one of embodiments 224-236, wherein the first splint oligonucleotide and/or second splint oligonucleotide comprises a barcode sequence, and wherein the subpart to which a second blocker oligonucleotide of the one or more blocker oligonucleotides hybridizes comprises the barcode sequence.

238. The method of embodiment 237, wherein the second blocker oligonucleotide comprises a sequence of at least 12 nucleotides that hybridizes to the subpart of the first splint oligonucleotide and/or of the second splint oligonucleotide.

239. The method of embodiment 237 or 238, wherein the second blocker oligonucleotide is about 12 to about 30 nucleotides long, optionally wherein the second blocker oligonucleotide is about 12 to about 25 nucleotides long.

240. The method of any one of embodiments 237-239, wherein the subpart to which the second blocker oligonucleotide of the one or more blocker oligonucleotides hybridizes comprises one or more 5′ and/or 3′ residues adjacent the barcode region, optionally wherein the one or more 5′ residues adjacent the barcode region does not comprise a tethering region of the first splint oligonucleotide or second splint oligonucleotide or a part thereof.

241. The method of any one of embodiments 237-240, wherein the second blocker oligonucleotide comprises one or more 3′ overhang nucleotides, optionally wherein the one or more 3′ overhang nucleotides comprises about 1-10 nucleotides.

242. The method of any one of embodiments 237-241, wherein hybridizing of the second blocker oligonucleotide to the first splint oligonucleotide and/or second splint oligonucleotide renders the first splint oligonucleotide and/or second splint oligonucleotide double-stranded along at least 80%, optionally at least 95%, of its length other than the 3′ hybridization region, optionally wherein at most 1 or 2 nucleotides of the first splint oligonucleotide and/or second splint oligonucleotide is single-stranded 5′ of the 3′ hybridization region upon hybridizing of the second blocker oligonucleotide to the first splint oligonucleotide and/or second splint oligonucleotide.

243. The method of any one of embodiments 237-242, comprising removing the second blocker oligonucleotide from the first splint oligonucleotide and/or second splint oligonucleotide by contacting the first splint oligonucleotide and/or second splint oligonucleotide with a 5′→3′ exonuclease.

244. The method of any one of embodiments 237-243, wherein extending the hybridized first splint oligonucleotide and/or the hybridized second splint oligonucleotide at e) comprises contacting the hybridized first splint oligonucleotide and/or the hybridized second splint oligonucleotide with a strand-displacing polymerase under conditions sufficient to extend the hybridized first splint oligonucleotide and/or the hybridized second splint oligonucleotide,
whereby the strand-displacing polymerase displaces the second blocker oligonucleotide from the first splint oligonucleotide and/or second splint oligonucleotide.

245. The method of any one of embodiments 224-244, wherein the one or more blocker oligonucleotides comprise one or more chemically modified nucleotides, optionally wherein the one or more chemically modified nucleotides comprise a 3′ phosphate or inverted dT, and/or a backbone modification, optionally wherein the backbone modification comprises a locked nucleic acid (LNA).

246. The method of any one of embodiments 224-245, wherein providing the first construct comprises annealing the one or more blocker oligonucleotides to the first splint oligonucleotide, whereby the first construct comprises the one or more blocker oligonucleotides, and/or wherein providing the second construct comprises annealing the one or more blocker oligonucleotides to the second splint oligonucleotide, whereby the second construct comprises the one or more blocker oligonucleotides.

247. The method of any one of embodiments 223-246, wherein the method comprises (II).

248. The method of embodiment 247, wherein the first subpool and the second subpool comprise different amounts of the sample, optionally wherein the first subpool and the second subpool comprise different dilutions of the sample.

249. The method of embodiment 248, comprising:
providing a plurality of paired combinations of the first construct and second construct, wherein a first paired combination of the plurality of paired combinations comprises the first moiety and second moiety that bind to the first analyte, and a second paired combination of the plurality of paired combinations comprises the first moiety and second moiety that bind to the second analyte; and
preparing the complexing solution in the plurality of subpools in c), wherein the amount of sample in the first subpool is higher than the amount of sample in the second subpool,
   wherein the complexing solution in the first subpool comprises the second paired combination of the plurality of paired combinations, and
   wherein the complexing solution in the second subpool comprises the first paired combination of the plurality of paired combinations, optionally wherein the amount of sample in the first subpool is, is about, or is at least 2, 3, 4, 5, 6, 7, 8, 9, 10, 15, 20, 30, 40, 50, 75, 100, 150, 200, 300, 400, 500, 750, 1,000, 2,000, 4,000, 8,000, 10,000, 20,000, 50,000, 100,000, 200,000, 500,000, 10⁶ fold or more higher, or optionally wherein the amount of sample in the first subpool is higher by a fold amount in a range defined by any two of the preceding values (e.g., about 2-10⁶ fold, about 10-100,000 fold, about 20-8,000 fold, about 4-400 fold, etc.), or optionally wherein the amount of sample in the first subpool is than the amount of sample in the second subpool by a fold amount in the range of about 5 to about 100,000.

250. The method of embodiment 248 or 249,
wherein the concentration of the first analyte in the sample is expected to be higher than the concentration of the second analyte in the sample, or
wherein the concentration of the first analyte in the sample is expected to be higher than a threshold concentration and the concentration of the second analyte in the sample is expected to be lower than the threshold concentration, or
wherein the first construct and second construct of the first subpool have a first sensitivity for generating on-target extension product from the first analyte, and the first construct and second construct of the second subpool have a second sensitivity for generating on-target extension product from the second analyte, wherein the first sensitivity is higher than the second sensitivity, or the first sensitivity is higher than a threshold sensitivity and the second sensitivity is lower than the threshold sensitivity.

251. The method of any one of embodiments 247-250, comprising generating a dilution series of the sample before preparing the complexing solution in a plurality of subpools in c), wherein the complexing solution in the first subpool comprises a different dilution of the sample than the second subpool, optionally wherein the dilution series of the sample comprises a, an about, or an at least 10, 20, 50, 100, 200, 400, 1,000, 2,000, 4,000, 8,000, 20,000, 50,000, or 100,000 fold dilution, or optionally wherein the dilution series of the sample comprises a dilution by a fold amount in a range defined by any two of the preceding values (e.g., about 10-100,000 fold, about 20-8,000 fold, about 40-400 fold, etc.), of the sample between the first subpool and second subpool.

252. The method of any one of embodiments 247-251, wherein a concentration of the first analyte in the complexing solution of the second subpool is within, within about, or within at most 100, 90, 80, 60, 50, 40, 30, 20, 10, 5, 2-fold the concentration of the second analyte, or optionally wherein the concentration of the first analyte is a fold amount of the concentration of the second analyte in a range defined by any two of the preceding values (e.g., about 10⁻¹⁰⁰ fold, about 20-90 fold, 20-50 fold, etc.), in the sample or in the first subpool.

253. The method of any one of embodiments 247-252, wherein the detected amount of extension product for the first analyte is within, within about, or within at most 100, 90, 80, 60, 50, 40, 30, 20, 10, 5, 2-fold the concentration of the second analyte, or optionally wherein the concentration of the first analyte is a fold amount of the concentration of the second analyte in a range defined by any two of the preceding values (e.g., about 10⁻¹⁰⁰ fold, about 20-90 fold, 20-50 fold, etc.), optionally wherein the detected amount of extension product for the first analyte is, or is expected to be, within or within about 20 fold the detected amount of extension product for the second analyte.

254. The method of any one of embodiments 247-253, wherein the complexing solution in the first subpool comprises a first diluent and wherein the complexing solution in the second subpool comprises a second diluent that is different from the first diluent.

255. The method of embodiment 254, wherein the first diluent comprises one or more components to which the first moiety and/or second moiety in the prepared complexing solution of the second subpool bind.

256. The method of any one of embodiments 247-255, wherein the plurality of subpools comprises 2, 3, 4, 5, 6, 7, 8, 9, 10, 15, 20, 50, 100 or more subpools, or optionally wherein the plurality of subpools comprises a number of subpools in a range defined by any two of the preceding values (e.g., 2-100 subpools, 4-20 subpools, 5-10 subpools, etc.).

257. The method of any one of embodiments 247-256, wherein one or more of the splint oligonucleotides of the plurality of paired combinations of the first construct and second construct each comprises a subpool barcode, or a part thereof, that identifies a subpool of the plurality of subpools.

258. The method of embodiment 257, wherein the first splint oligonucleotide of a paired combination of the plurality of paired combinations comprises a first subpool barcode, or a part thereof, that identifies the subpool of the plurality of subpools, and the second splint oligonucleotide of the paired combination of the plurality of paired combinations comprises a second subpool barcode, or a part thereof, that identifies the subpool of the plurality of subpools.

259. The method of embodiment 258, wherein the first splint oligonucleotide of a first subpool of the plurality of subpools is the same as the first splint oligonucleotide of a second subpool of the plurality of subpools, except for the subpool barcode.

260. The method of embodiment 258 or 259, wherein the first splint oligonucleotide is attached to the first construct via hybridization to a first tether oligonucleotide attached to the first construct, and/or the second splint oligonucleotide is attached to the second moiety via hybridization to a second tether oligonucleotide attached to the second moiety, wherein the first splint oligonucleotide and/or second splint oligonucleotide comprises a 5′ tethering region that hybridizes to the first tether oligonucleotide and/or second tether oligonucleotide, wherein the 5′ tethering region comprises the subpool barcode.

261. The method of any one of embodiments 257-260, wherein the first splint oligonucleotide comprises a first subpool barcode that is 1-8 nucleotides long, optionally wherein the first subpool barcode is 2-4 nucleotides long, and/or
wherein the second splint oligonucleotide comprises a second subpool barcode that is 1-8 nucleotides long, optionally wherein the second subpool barcode is 2-4 nucleotides long.

262. The method of any one of embodiments 257-261, wherein combining the plurality of subpools is performed:
(1) in c-ii), after contacting the first construct provided in a) with the sample, and before combining in the solution the sample-contacted construct and the second construct provided in b);
(2) in c-iii), after contacting the second construct provided in a) with the sample, and before combining in the solution the sample-contacted construct and the first construct provided in a);
(3) after preparing the complexing solution in c) and before the extending in e); or
(4) after the extending in e) and before determining the presence and/or amount, or the absence of the on-target extension product in g).

263. The method of any once of embodiments 223-262, wherein the method comprises (III).

264. The method of embodiment 263, wherein the one or more trimmed paired combinations comprises, comprises about, or comprises at most 10, 20, 30, 40, 50, 60, 70, 80, 90%, of the plurality of paired combinations, or optionally wherein the one or more trimmed paired combinations comprises a percentage in a range defined by any two of the preceding values (e.g., about 10⁻⁹⁰%, about 20-80%, about 40-60%, about 20-40%, etc.) of the plurality of paired combinations, or optionally wherein the one or more trimmed paired combinations comprises a third of the plurality of paired combinations.

265. The method of embodiment 263 or 264, wherein:
(A) a frequency of analyte-independent on-target interactions between the splint oligonucleotides of the plurality of paired combinations is reduced, and/or
(B) a total variation in a calculated ΔG of hybridization between the 3′ hybridizing regions among the plurality of paired combinations is reduced,
compared to when the 3′ hybridizing region of the splint oligonucleotides of the one or more trimmed paired combinations is the same length as the 3′ hybridizing region of the splint oligonucleotides of the at least one other paired combination of the plurality of paired combinations.

266. The method of any one of embodiments 263-265, wherein a calculated ΔG of hybridization between the 3′ hybridizing region of each of the plurality of paired combinations of the first splint oligonucleotide and second splint oligonucleotide is in a range of about −3 to about −4 kcal/mol.

267. The method of any one of embodiments 263-266, wherein a difference in the length of the 3′ hybridization regions among all of the splint oligonucleotides of the plurality of paired combinations is no more than 1, 2, or 3 nucleotides.

268. The method of any one of embodiments 263-267, wherein the length of the 3′ hybridization regions of the splint oligonucleotides of the plurality of paired combinations is in a range of 4-9 nt.

269. The method of any one of embodiments 223-268, wherein the method comprises (IV).

270. The method of embodiment 269, wherein reducing or interfering with a binding interaction between the analyte and the first moiety or second moiety comprises:
preparing the complexing solution at c) in the presence of:
   an attenuating moiety that competes for binding to the analyte with the first moiety and/or second moiety, wherein the attenuating moiety is not attached to a splint oligonucleotide comprising a 3′ hybridization region complementary to the 3′ hybridizing region of the first splint oligonucleotide or second splint oligonucleotide; and/or
   a pseudo-analyte that can be bound by either one of the first moiety or second moiety but not by the other.

271. The method of embodiment 269 or 270, wherein reducing or interfering with a binding interaction between the analyte and the first moiety or second moiety comprises, at c):
i) combining in the solution the first construct provided in a) and the second construct provided in b) with the sample in the presence of: an attenuating moiety that binds the analyte and is not attached to a splint oligonucleotide comprising a 3′ hybridization region complementary to the 3′ hybridizing region of the first splint oligonucleotide or second splint oligonucleotide, wherein the attenuating moiety competes for binding to the analyte with the first moiety and/or second moiety; and/or a pseudo-analyte;
ii) contacting the first construct provided in a) with a sample in the presence of: an attenuating moiety that binds the analyte and is not attached to a splint oligonucleotide comprising a 3′ hybridization region complementary to the 3′ hybridizing region of the second splint oligonucleotide, wherein the attenuating moiety competes for binding to the analyte with the first moiety; and/or a pseudo-analyte; and combining in a solution the sample-contacted construct and the second construct provided in b); or
iii) contacting the second construct provided in b) with a sample in the presence of: a attenuating moiety that binds the analyte and is not attached to a splint oligonucleotide comprising a 3′ hybridization region complementary to the 3′ hybridizing region of the second splint oligonucleotide, wherein the attenuating moiety competes for binding to the analyte with the first moiety; and/or a pseudo-analyte; and combining in a solution the sample-contacted second construct with the first construct provided in a).

272. The method of embodiment 270 or 271, wherein the attenuating moiety is attached to a solid support, wherein the solid support is not attached to the splint oligonucleotide comprising the 3′ hybridization region complementary to the 3′ hybridizing region of the first splint oligonucleotide or second splint oligonucleotide.

273. The method of any one of embodiments 269-272, wherein suppressing on-target interactions between the second splint oligonucleotide and the first splint oligonucleotide comprises providing a 3′ hybridizing region having a first length that is shorter than a second length, wherein a detected signal for the amount of the analyte determined at g) is lower when the analyte is assayed with first and second splint oligonucleotides comprising a 3′ hybridization region of the first length than when the 3′ hybridization region has the second length, optionally wherein determining at g) comprises sequencing one or more extension products generated at e), optionally wherein the sequencing comprises high-throughput sequencing.

274. The method of embodiment 273, wherein the detected signal for the amount of the analyte determined at g) is saturated when the 3′ hybridizing region has the second length, and is not saturated when the 3′ hybridizing region has the first length.

275. The method of any one of embodiments 269-274, wherein the first moiety is a lower affinity binding moiety for the analyte than a higher affinity binding moiety for the analyte, wherein a detected signal for the amount of the analyte determined at g) is lower when the first moiety is the lower affinity binding moiety for the analyte, optionally wherein determining at g) comprises sequencing one or more extension products generated at e), optionally wherein the sequencing comprises high-throughput sequencing.

276. The method of embodiment 275, wherein the lower affinity binding moiety has a binding affinity for the analyte of, of at most, or of about, 1, 2, 5, 10, 100, 200, 500, 1,000, 2,000, 5,000, 10,000, 50,000, 100,000 nM or higher, or optionally, the er affinity binding moiety has a binding affinity for the analyte in a range defined by any two of the preceding values (e.g., 1-100,000 nM, 1-10 nM, 10⁻¹⁰⁰ nM, 100-500 nM, 500-1,000 nM, 1,000-5,000 nM, 5,000-10,000 nM, 10,000-100,000 nM, etc.).

277. The method of embodiment 275 or 276, wherein the detected signal for the amount of the analyte determined at g) is saturated when the first moiety is the higher affinity binding moiety for the analyte, and is not saturated when the first moiety is the lower affinity binding moiety for the analyte.

278. The method of any one of embodiments 223-277, wherein suppressing on-target interactions between the second splint oligonucleotide and the first splint oligonucleotide comprises providing a 3′ hybridizing region having a first hybridization energy that is greater than a second hybridization energy, wherein a detected signal for the amount of the analyte assayed is lower when the analyte is assayed with splint oligonucleotides having a 3′ hybridization region having the first hybridization energy than having the second hybridization energy, optionally wherein determining at g) comprises sequencing one or more extension products generated at e), optionally wherein the sequencing comprises high-throughput sequencing.

279. The method of any one of embodiments 223-278, wherein the first construct comprises a solid support comprising:
the first moiety attached to the solid support; and
the first splint oligonucleotide attached to the solid support, wherein the first splint oligonucleotide is attached to the first moiety via the solid support,
wherein
(A) the method comprises (I), wherein the subpart to which a first blocker oligonucleotide of the one or more blocker oligonucleotides hybridizes comprises the 3′ hybridizing region or a portion thereof of the first splint oligonucleotide, wherein the blocker oligonucleotide competes with the 3′ hybridizing region of the second splint oligonucleotide for binding to the 3′ hybridizing region of the first splint oligonucleotide, and/or
(B) the method comprises (IV), wherein the first splint oligonucleotide is attached to the solid support at a first ratio of the first splint oligonucleotide to the first moiety that is lower than a second ratio of the first splint oligonucleotide to the first moiety, wherein a detected signal for the amount of the analyte determined at g) has a smaller detected signal when the first splint oligonucleotide and the first moiety is present on the solid support at the second ratio than at the first ratio, optionally wherein determining at g) comprises sequencing one or more extension products generated at e), optionally wherein the sequencing comprises high-throughput sequencing.

280. The method of embodiment 279, wherein the detected signal for the amount of the analyte determined at g) is saturated when the first splint oligonucleotide and the first moiety is present on the solid support at the second ratio.

281. The method of any one of embodiments 223-280, wherein the second splint oligonucleotide is directly attached to the second moiety.

282. The method of any one of embodiments 223-281, wherein the second splint oligonucleotide is attached to a first member of a binding pair that binds to a second member of the binding pair, wherein the second member is attached to a first moiety.

283. The method of any one of embodiments 223-282, wherein the first construct comprises a solid support comprising:
the first construct attached to the solid support; and
the first splint oligonucleotide attached to the solid support,
wherein the method comprises releasing the on-target extension product from the first construct and/or the second construct at (f).

284. The method of any one of embodiments 223-283, wherein extending and releasing are performed by a single enzyme, optionally wherein the single enzyme comprises a strand-displacing polymerase.

285. The method of embodiment 284, wherein the first splint oligonucleotide is attached to the solid support via hybridization to a first tether oligonucleotide attached to the solid support, and wherein the first splint oligonucleotide is attached to the solid support independently of the first moiety.

286. The method of embodiment 284 or 285, wherein the second splint oligonucleotide is attached to the second moiety via hybridization to a second tether oligonucleotide attached to the second moiety.

287. The method of any one of embodiments 284-286, wherein the extending in e) comprises treating the solid support with a strand-displacing polymerase under conditions sufficient to extend the hybridized first splint oligonucleotide and/or the hybridized second splint oligonucleotide, and
wherein the releasing in f) comprises allowing the strand-displacing polymerase to displace the first tether oligonucleotide hybridized to the first splint oligonucleotide during extension, and optionally to displace the second tether oligonucleotide hybridized to the second splint oligonucleotide during extension.

288. The method of any one of embodiments 223-283, wherein the releasing in f) comprises cleaving a covalent attachment of the first and/or splint oligonucleotide with the first and/or second moiety, respectively.

289. The method of any one of embodiments 223-288, wherein determining the presence and/or amount, or the absence of the on-target extension product comprises obtaining sequencing data of one or more extension products generated at e) and released in f).

290. The method of embodiment 289, comprising sequencing the one or more released extension products to obtain the sequencing data of the one or more released extension products.

291. The method of embodiment 290, wherein the sequencing comprises high-throughput sequencing.

292. The method of any one of embodiments 289-291, wherein determining the presence and/or amount, or the absence of the released on-target extension product comprises amplifying the one or more extension products generated at e) and released in f).

293. The method of embodiment 292, wherein determining the presence and/or amount, or the absence of the released on-target extension product comprises:
combining the one or more extension products with a control oligonucleotide in an amplification mixture;
amplifying the one or more extension products and the control oligonucleotide in the amplification mixture, to generate extension product amplicons and control oligonucleotide amplicons,
detecting a level of the extension product amplicons and the control oligonucleotide amplicons; and
normalizing the detected level of the extension product amplicons based on the detected level of the control oligonucleotide amplicons.

294. The method of embodiment 293, wherein the released on-target extension products are distributed among multiple partitions, wherein determining the presence and/or amount, or the absence, of the released on-target extension products in the multiple partition comprises:
diluting the extension products in at least one partition to normalize the concentration of extension products in each partition;
combining the diluted extension products in each partition with a control oligonucleotide in an amplification mixture;
amplifying the diluted extension products and the control oligonucleotide in the amplification mixture, to generate extension product amplicons and control oligonucleotide amplicons;
detecting a level of the extension product amplicons and the control oligonucleotide amplicons in each partition by performing high throughput sequencing; and
normalizing the detected level of the extension product amplicons based on the detected level of the control oligonucleotide amplicons.

294a. The method of embodiment 294, further comprising adjusting the detected level of the extension product amplicons within each partition to account for the dilution of the extension products in the partition.

294b. The method of embodiment 293, wherein the released on-target extension products are distributed among multiple partitions, wherein determining the presence and/or amount, or the absence, of the released on-target extension products in the multiple partition comprises:
adding a control oligonucleotide to each partition;
diluting the extension products and the control oligonucleotide in at least one partition to normalize the concentration of extension products in each partition;
amplifying the diluted extension products and the control oligonucleotide to generate extension product amplicons and control oligonucleotide amplicons;
detecting a level of the extension product amplicons and the control oligonucleotide amplicons in each partition by performing high throughput sequencing; and
normalizing the detected level of the extension product amplicons based on the detected level of the control oligonucleotide amplicons.

294c. The method of embodiment 293 or 294b, wherein the diluting of the extension products is performed without determining the concentrations of the extension products prior to the diluting.

294d. The method of embodiment 293 or 294b, further comprising determining the concentration of the extension products prior to the diluting.

294e. The method of embodiment 294d, wherein determining the concentration of extension products in each partition comprises performing qPCR using PCR primers common to all amplicon molecules in each partition.

295. The method of embodiment 294, wherein determining the concentration of extension products in each partition comprises performing qPCR using PCR primers common to all amplicon molecules in each partition.

296. The method of embodiment 292, wherein the released on-target extension products are distributed among multiple partitions, wherein determining the presence and/or amount, or the absence, of the released on-target extension products in the multiple partition comprises:
combining the diluted extension products in each partition with a control oligonucleotide in an amplification mixture;
amplifying the diluted extension products and the control oligonucleotide in the amplification mixture, to generate extension product amplicons and control oligonucleotide amplicons, wherein one of the PCR primers in the amplification mixture is present at a concentration such that it will be depleted in at least one partition prior to the completion of the amplification reaction;
detecting a level of the extension product amplicons and the control oligonucleotide amplicons in each partition by performing high throughput sequencing; and
normalizing the detected level of the extension product amplicons based on the detected level of the control oligonucleotide amplicons.

297. The method of any one of embodiments 293-296, wherein the control oligonucleotide comprises a unique molecular identifier (UMI), wherein detecting the level of the control oligonucleotide comprises obtaining sequence information of the UMI in the control oligonucleotide amplicons, and wherein the detected level of the control oligonucleotide is based on the number of the UMI with distinct sequences associated with a sequence of the control oligonucleotide in the sequencing data.

298. The method of any one of embodiments 293-297, wherein the extension product amplicons comprise adapter sequences that hybridize to one or more sequencing primers.

299. The method of any one of embodiments 293-298, wherein the extension product amplicons comprise one or more indexing sequences.

300. The method of any one of embodiments 289-299, wherein the capture oligonucleotide and/or detection oligonucleotide comprises a unique molecular identifier (UMI), or wherein the first splint oligonucleotide and/or second splint oligonucleotide comprises a unique molecular identifier (UMI).

301. The method of embodiment 300, wherein determining the presence and/or amount, or the absence of the released on-target extension product comprises determining the number of UMI with distinct sequences associated with the capture oligonucleotide and/or detection oligonucleotide, or determining the number of UMI with distinct sequences associated with the first splint oligonucleotide and/or second splint oligonucleotide.

302. The method of any one of embodiments 289-301, comprising identifying a sequence of an extension product in the sequencing data as being associated with the on-target extension product based on a pairing of: (A) one or more subsequences in the sequence associated with the detection oligonucleotide, wherein the one or more subsequences identify the binding target of the detection moiety attached to the detection oligonucleotide, with (B) one or more subsequences in the sequence associated with the capture oligonucleotide, wherein the one or more subsequences identify a binding target of the capture moiety attached to the solid support to which the capture oligonucleotide is attached that is the same as the binding target of the detection moiety, or
identifying a sequence of an extension product in the sequencing data as being associated with the on-target extension product based on a pairing of: (A) one or more subsequences in the sequence associated with the second splint oligonucleotide, wherein the one or more subsequences identify the binding target of the second moiety attached to the second splint oligonucleotide, with (B) one or more subsequences in the sequence associated with the first splint oligonucleotide, wherein the one or more subsequences identify a binding target of the first moiety attached to the first splint oligonucleotide that is the same as the binding target of the second moiety.

303. The method of embodiment 302, wherein at least one of the one or more subsequences in the sequence associated with the detection oligonucleotide and the one or more subsequences in the sequence associated with the capture oligonucleotide is a barcode sequence, or wherein at least one of the one or more subsequences in the sequence associated with the first splint oligonucleotide and the one or more subsequences in the sequence associated with the second splint oligonucleotide is a barcode sequence.

304. The method of embodiment 303, wherein the one or more subsequences in the sequence associated with the detection oligonucleotide comprises a first barcode sequence, and the one or more subsequences in the sequence associated with the capture oligonucleotide comprises a second barcode sequence, or wherein the one or more subsequences in the sequence associated with the first splint oligonucleotide comprises a first barcode sequence, and the one or more subsequences in the sequence associated with the second splint oligonucleotide comprises a second barcode sequence.

305. The method of embodiment 303 or 304, wherein the 3′ hybridizing region identifies the binding target of the detection oligonucleotide or capture oligonucleotide, or wherein the 3′ hybridizing region identifies the binding target of the first splint oligonucleotide or second splint oligonucleotide.

306. The method of any one of embodiments 302-305, wherein identifying the sequence in the sequencing data as being associated with the on-target extension product is further based on a length of the sequenced extension product, wherein an on-target extension product is associated with a sequenced extension product having a length consistent with the length expected for the on-target extension product.

307. The method of any one of embodiments 302-306, wherein a sequence in the sequencing data is identified as an off-target extension product based on a mispairing between: (A) the one or more subsequences in the sequence associated with the detection oligonucleotide, wherein the one or more subsequences identify the binding target of the detection moiety attached to the detection oligonucleotide, and (B) the one or more subsequences in the sequence associated with the capture oligonucleotide, wherein the one or more subsequences identify a different binding target of a different capture moiety that is attached to a different solid support to which the capture oligonucleotide is attached, or wherein a sequence in the sequencing data is identified as an off-target extension product based on a mispairing between: (A) the one or more subsequences in the sequence associated with the second splint oligonucleotide, wherein the one or more subsequences identify the binding target of the second moiety attached to the second splint oligonucleotide, and (B) the one or more subsequences in the sequence associated with the first splint oligonucleotide, wherein the one or more subsequences identify a different binding target of a different first moiety that is attached to a different solid support to which the first splint oligonucleotide is attached.

308. The method of embodiment 307, wherein identifying the sequence in the sequencing data as being associated with the on-target extension product comprises analyzing a length distribution of the one or more extension products.

309. The method of embodiment 307 or 308, comprising removing from the sequencing data sequences associated with off-target extension products.

309a. The method of any one of embodiments 223 to 309, wherein i) the first splint oligonucleotide is attached to the first moiety in a deterministic manner, and/or ii) the second splint oligonucleotide is attached to the second moiety in a deterministic manner.

309b. The method of embodiment 309a, wherein only one first splint oligonucleotide is attached to each first moiety and/or wherein only one second splint oligonucleotide is attached to each second moiety.

309c. The method of embodiment 309a, wherein only two first splint oligonucleotides are attached to each first moiety and/or only two second splint oligonucleotides are attached to each second moiety.

310. A method of analyzing a sample for an analyte, comprising:
a) combining:
   i) a solid support comprising:
      a capture moiety attached to the solid support, wherein the capture moiety specifically binds an analyte; and
      a capture oligonucleotide attached to the solid support independently of the capture moiety, wherein the capture oligonucleotide comprises a 3′ hybridizing region;
   ii) a detection conjugate comprising:
      a detection moiety that specifically binds the analyte; and a detection oligonucleotide attached to the detection moiety, wherein the detection oligonucleotide comprises a 3′ hybridizing region complementary to the 3′ hybridizing region of the capture oligonucleotide; and
   iii) a sample;
   wherein, if the analyte is present in the sample, it is bound to the solid support by the capture moiety and to the detection conjugate by the detection moiety to form a capture complex in which the capture oligonucleotide and the detection oligonucleotide are in proximity;
b) permitting the 3′ hybridizing region of the capture oligonucleotide and the 3′ hybridizing region of the detection oligonucleotide that are in proximity to hybridize to each other;
c) extending the hybridized capture oligonucleotide and/or the hybridized detection oligonucleotide to generate an on-target extension product;
d) releasing the on-target extension product from the solid support; and
e) determining the presence or the absence of the released on-target extension product to determine the presence or the absence of the analyte in the sample.

311. The method of embodiment 310, wherein step a) comprises first combining the solid support and the sample such that the capture moiety of the solid support is bound to the analyte, if present in the sample, and then combining the detection conjugate.

312. The method of embodiment 310, wherein step a) comprises first combining the detection conjugate and the sample such that the detection moiety is bound to the analyte, if present in the sample, and then combining the solid support.

313. The method of any one of embodiments 310 to 311, further comprising removing unbound components of the sample prior to combining the detection conjugate.

314. The method of any one of embodiments 310 to 313, further comprising washing the capture complex to remove components that are not part of the capture complex.

315. The method of any one of embodiments 310 to 314, wherein the capture oligonucleotide comprises a first 5′ tethering region and is attached to the solid support via hybridization to a first tether oligonucleotide attached to the solid support, and the detection oligonucleotide comprises a second 5′ tethering region and is attached to the detection moiety via hybridization to a second tether oligonucleotide attached to the detection moiety.

316. The method of embodiment 315, wherein the hybridized capture oligonucleotide and the hybridized detection oligonucleotide are extended by a strand-displacing DNA polymerase, and the on-target extension product is released from the solid support by the strand-displacing DNA polymerase.

317. The method of embodiment 316, wherein the strand-displacing DNA polymerase comprises a Klenow fragment.

318. The method of any one of embodiment 310 to 317, wherein the capture moiety is covalently attached to a first member of a binding pair that binds to a second member of the binding pair, wherein the second member is attached to the solid support.

319. The method of embodiment 318, wherein the first member of the binding pair comprises biotin, and the second member of the binding pair comprises streptavidin.

320. The method of any one of embodiments 315 to 317, wherein the first tether oligonucleotide is covalently attached to a first member of a binding pair that binds to a second member of the binding pair, wherein the second member is attached to the solid support.

321. The method of embodiment 320, wherein the first member of the binding pair comprises biotin, and the second member of the binding pair comprises streptavidin.

322. The method of embodiment 310, wherein the capture oligonucleotide comprises a first barcode sequence that identifies a binding target of the capture moiety, and wherein the detection oligonucleotide comprises a second barcode sequence that identifies a binding target of the detection moiety.

323. The method of any one of embodiments 310 to 322, wherein the capture oligonucleotide and detection oligonucleotide each comprise, from 5′ to 3′: a tethering region, a primer binding region configured to bind a primer for amplifying the released on-target extension product, a barcode sequence, and the 3′ hybridizing region.

324. The method of any one of embodiment 310 to 323, wherein the solid support is a magnetically responsive bead.

325. The method of any one of embodiments 310 to 324, wherein the capture moiety and the detection moiety are independently an antibody or an antibody fragment.

326. The method of any one of embodiment 310 to 325, wherein the combining in step a) further comprises combining one or more blocker oligonucleotides, wherein each blocker oligonucleotide specifically hybridizes to a subpart of one or both of the capture oligonucleotide and/or the detection oligonucleotide.

327. The method of embodiment 326, wherein the blocker oligonucleotide is from 5 to 13 nucleotides long.

328. The method of embodiments 326 or 327, wherein the subpart to which a first blocker oligonucleotide of the one or more blocker oligonucleotides hybridizes to the 3′ hybridizing region or a portion thereof of the capture oligonucleotide or the detection oligonucleotide, and wherein the first blocker oligonucleotide competes with: the 3′ hybridizing region of the detection oligonucleotide for binding to the 3′ hybridizing region of the capture oligonucleotide; or the 3′ hybridizing region of the capture oligonucleotide for binding to the 3′ hybridizing region of the detection oligonucleotide.

329. The method of any one of embodiments 326 to 328, comprising removing the first blocker oligonucleotide after the combining in step a) and before the extending in step c).

330. The method of embodiments 328 or 329, wherein the capture oligonucleotide comprises a first barcode sequence that identifies a binding target of the capture moiety, wherein the detection oligonucleotide comprises a second barcode sequence that identifies a binding target of the detection moiety, and wherein the subpart to which a second blocker oligonucleotide of the one or more blocker oligonucleotides hybridizes comprises the first barcode sequence and the subpart to which a third blocker oligonucleotide of the one or more blocker oligonucleotides hybridizes comprises the second barcode sequence.

331. The method of any one of embodiments 310 to 330, wherein the capture oligonucleotide comprises a unique molecular identifier (UMI) at least 4 nucleotides long between the 5′ tethering region and the 3′ hybridizing region.

332. The method of any one of embodiments 310 to 331, wherein the detection oligonucleotide comprises a unique molecular identifier (UMI) at least 4 nucleotides long between the 5′ tethering region and the 3′ hybridizing region.

333. The method of any one of embodiments 310 to 332, wherein the 3′ hybridizing region of the detection oligonucleotide and the 3′ hybridizing region of the capture oligonucleotide are each 6 to 8 nucleotides in length.

334. The method of any one of embodiments 310 to 333, wherein determining the presence or the absence of the released on-target extension product comprises performing qPCR.

335. The method of any one of embodiment 310 to 334, wherein determining the presence or the absence of the released on-target extension product comprises sequencing the released on-target extension product.

336. The method of any one of embodiments 310 to 336, comprising providing a plurality of paired combinations of the solid support and the detection conjugate, wherein binding targets of the capture moiety and the detection moiety of each paired combination are the same, and wherein different paired combinations of the plurality of paired combinations have different binding targets.

337. The method of embodiment 336, wherein the different binding targets are different analytes.

338. The method of embodiment 336, wherein the different binding targets are different epitopes on the same analyte.

339. The method of any one of embodiments 336 to 338, wherein the 3′ hybridizing region of the capture oligonucleotide and detection oligonucleotide of a first paired combination of the plurality of paired combinations is not complementary to the 3′ hybridizing region of the detection oligonucleotide and capture oligonucleotide, respectively, of at least one other paired combination of the plurality of paired combinations.

340. The method of any one of embodiments 336 to 339, wherein each capture oligonucleotide attached to a solid support of the plurality of the solid supports comprises a barcode sequence that identifies a binding target of the capture moiety attached to the respective solid support.

341. The method of any one of embodiments 336 to 340, wherein each detection oligonucleotide attached to a detection moiety of the plurality of the detection conjugates comprises a barcode sequence that identifies a binding target of the respective detection moiety.

341a. The method of any one of embodiments 310 to 341, wherein the sample is divided into a plurality of subpools comprising at least a first subpool and a second subpool, and, optionally, wherein (i) the sample concentration in different subpools of the plurality of subpools differ, (ii) the buffers in different subpools of the plurality of subpools differ, (iii) different subpools of the plurality of subpools are combined with the solid support and/or the detection conjugate for different amounts of time, (iv) the first subpool is combined with the solid support and/or the detection conjugate for from one minute to thirty minutes longer than the amount of time the second subpool is combined with the solid support and/or the detection conjugate, and/or (v) the different subpools of the plurality of subpools are combined prior to determining the presence or the absence of the released on-target extension product.

341b. The method of any one of embodiments 310 to 341a, wherein the capture oligonucleotide is attached to the detection moiety in a deterministic manner.

341c. The method of embodiment 341b, wherein only one capture oligonucleotide is attached to each detection moiety.

341d. The method of embodiment 341b, wherein only two capture oligonucleotides are attached to each capture moiety.

342. A composition comprising:
a) a first pool comprising at least 30 different populations of solid supports, each solid support comprising:
   i) a capture moiety attached to the solid support, wherein the capture moiety specifically binds an analyte;
   ii) a tether oligonucleotide attached to the solid support independent of the capture moiety; and
   iii) a capture oligonucleotide comprising from 5′ to 3′: a tethering region, a capture barcode region, and a 3′ hybridizing region, wherein the capture oligonucleotide is attached to the solid support via hybridization of the tethering region to the tether oligonucleotide attached to the solid support; and
b) a second pool comprising at least 30 different populations of detection conjugates, each detection conjugate comprising:
   i) a detection moiety that specifically binds the analyte;
   ii) a tether oligonucleotide attached to the detection moiety; and
   ii) a detection oligonucleotide comprising from 5′ to 3′: a tethering region, a detection barcode region, and a 3′ hybridizing region, wherein the detection oligonucleotide is attached to the detection moiety via hybridization of the tethering region to the tether oligonucleotide attached to the detection moiety,
wherein each of the at least 30 different populations of solid supports forms a paired combination with a corresponding detection conjugate of the at least 30 different populations of detection conjugates, wherein the analyte bound by the capture moiety and detection moiety of each paired combination is the same, wherein the analytes bound by different paired combinations are different, wherein the 3′ hybridizing region of the capture oligonucleotide and detection oligonucleotide of each paired combination is not complementary to the 3′ hybridizing region of the detection oligonucleotide and the capture oligonucleotide, respectively, of any other paired combination, and wherein each paired combination can be distinguished from any other paired combination by one or both of the sequence of the capture barcode region and/or the sequence of the detection barcode region.

343. The composition of embodiment 342, further comprising at least 30 different populations of blocker oligonucleotides, wherein each of the different populations of blocker oligonucleotides specifically hybridizes to the 3′ hybridizing region or a portion thereof of the capture oligonucleotide or the detection oligonucleotide of each paired combination.

344. The composition of embodiment 343, wherein the blocker oligonucleotide is from 5 to 13 nucleotides long.

345. The composition of any one of embodiments 342 to 344, wherein the first pool comprises no more than 500 different populations of solid supports, and no more than 500 different populations of detection conjugates.

346. The composition of any one of embodiments 342 to 345, wherein the solid support is a magnetically responsive bead.

347. The composition of any one of embodiments 342 to 345, wherein the capture moiety and the detection moiety are independently an antibody or an antibody fragment.

348. The composition of any one of embodiments 342 to 347, further comprising a strand-displacing DNA polymerase.

348a. The composition of any one of embodiments 342 to 348, wherein the capture oligonucleotide is attached to the detection moiety in a deterministic manner.

348b. The composition of embodiment 348a, wherein only one capture oligonucleotide is attached to each detection moiety.

34c. The composition of embodiment 348a, wherein only two capture oligonucleotides are attached to each capture moiety.

349. A method for multiplexed analysis of analytes in a sample, the method comprising:
a) combining the composition of any one of embodiments 342 to 348 and a sample;
b) permitting the 3′ hybridizing region of the capture oligonucleotide and the 3′ hybridizing region of the detection oligonucleotide of each paired combination if the analyte of the paired combination is present in the sample;
c) extending the hybridized capture oligonucleotide and the hybridized detection oligonucleotide to generate an on-target extension product for each paired combination for which the analyte of the paired combination is present in the sample;
d) releasing the on-target extension product for each paired combination for which the analyte of the paired combination is present in the sample from the solid support; and
e) determining the presence or the absence of the released on-target extension product to determine the presence or the absence of the analyte of each paired combination in the sample.

350. The method of embodiment 349, wherein the combining in step a) comprises combining the sample and the first pool comprising at least 30 different populations of solid supports, and then combining the second pool comprising at least 30 different populations of detection conjugates with the sample and the first pool.

351. The method of embodiment 349, wherein the combining in step a) comprises combining the first pool comprising at least 30 different populations of solid supports and the second pool comprising at least 30 different populations of detection conjugates, and then combining the sample with the first pool and the second pool.

352. The method of any one of embodiments 349 to 351, wherein the hybridized capture oligonucleotide and the hybridized detection oligonucleotide are extended by a strand-displacing polymerase, and the on-target extension product is released from the solid support by the strand-displacing DNA polymerase.

353. The method of any one of embodiments 349 to 352, wherein determining the presence or the absence of the released on-target extension product comprises sequencing at least a portion of the released on-target extension product.

354. The method of any one of embodiments 349 to 353, wherein the sample is divided into a plurality of subpools comprising at least a first subpool and a second subpool.

355. The method of embodiment 354, wherein the sample concentration in the second subpool is diluted from 20-fold to 8,000-fold as compared to the sample concentration in the first subpool and/or wherein the first subpool comprises a different buffer than the second subpool.

356. The method of embodiment 354 or 355, wherein the combining in step a) comprises combining the composition of any one of embodiments 342 to 353 and the sample such that a first paired combination of the at least 30 different populations of solid supports and the corresponding detection conjugate of the at least 30 different populations of detection conjugates is combined with the sample of the first subpool and not with the sample of the second subpool, and at least a second paired combination of the at least 30 different populations of solid supports and the corresponding detection conjugate of the at least 30 different populations of detection conjugates is combined with the sample of the second subpool and not with the sample of the first subpool.

357. The method of claim 354, wherein the combining in step a) comprises:
i) combining a first population of solid supports of the at least 30 different populations of solid supports with the first subpool and not with the second subpool;
ii) combining a second population of solid supports of the at least 30 different populations of solid supports with the second subpool and not with the first subpool;
iii) combining the first subpool and the second subpool to form a pooled solution; and
iv) combining the detection conjugates of the at least 30 different populations of detection conjugates corresponding to the first population of solid supports and the second population of solid supports with the pooled solution.

357a. The method of any one of embodiments 349 to 357, wherein the capture oligonucleotide is attached to the detection moiety in a deterministic manner.

357b. The method of embodiment 357a, wherein only one capture oligonucleotide is attached to each detection moiety.

357c. The method of embodiment 357a, wherein only two capture oligonucleotides are attached to each capture moiety.

358. A method of analyzing a sample for an analyte, comprising:
a) combining:
   i) a first conjugate comprising:
      a first moiety that specifically binds an analyte;
      a first tether region attached to the first moiety; and
      a first splint oligonucleotide comprising a 5′ tethering region, a first moiety barcode, and a 3′ hybridizing region, wherein the 5′ tethering region of the first splint oligonucleotide is hybridized to the first tether region attached to the first moiety;
   ii) a second conjugate comprising:
      a second moiety that specifically binds the analyte;
      a second tether region attached to the second moiety; and
      a second splint oligonucleotide comprising a 5′ tethering region, a second moiety barcode, and a 3′ hybridizing region, wherein the 5′ tethering region of the second splint oligonucleotide is hybridized to the second tether region attached to the second moiety, and wherein the 3′ hybridizing region of the second splint oligonucleotide complementary to the 3′ hybridizing region of the first splint oligonucleotide; and
   iii) a sample;
d) permitting the 3′ hybridizing region of the first splint oligonucleotide and the 3′ hybridizing region of the second splint oligonucleotide that are in proximity to hybridize to each other if the analyte is present in the sample;
e) extending the hybridized first splint oligonucleotide and the hybridized second splint oligonucleotide using a strand-displacing DNA polymerase to generate an on-target extension product that comprises the extended first splint oligonucleotide and/or the extended second splint oligonucleotide and release by strand displacement the on-target extension product from the first moiety and the second moiety; and
f) determining the presence or the absence of the on-target extension product to thereby determine the presence or the absence of the analyte in the sample.

359. The method of embodiment 358, wherein the first moiety and the second moiety specifically bind different epitopes on the same analyte.

360. The method of embodiment 358, wherein the first moiety specifically binds with a first analyte and the second moiety specifically binds with a second analyte, wherein the first analyte and the second analyte interact.

361. The method of embodiment 360, wherein the first analyte is an enzyme and the second analyte is a substrate for the enzyme.

362. The method of any one of embodiments 358 to 361, wherein the combining in a) comprises combining the first conjugate and the sample, thereby allowing the first moiety of the first conjugate to be bound to the analyte if present in the sample, and then combining the second conjugate with the first conjugate and the sample.

363. The method of any one of embodiments 358 to 362, comprising providing: a plurality of paired combinations of the first conjugates and the second conjugates, wherein the analyte bound by the first moiety and second moiety of each paired combination is the same, and wherein different paired combinations of the plurality of paired combinations bind different analytes.

363a. The method of any one of embodiments 358 to 363, wherein i) the first splint oligonucleotide is attached to the first moiety in a deterministic manner, and/or ii) the second splint oligonucleotide is attached to the second moiety in a deterministic manner.

363b. The method of embodiment 363a, wherein only one first splint oligonucleotide is attached to each first moiety and/or wherein only one second splint oligonucleotide is attached to each second moiety.

363c. The method of embodiment 363a, wherein only two first splint oligonucleotides are attached to each first moiety and/or only two second splint oligonucleotides are attached to each second moiety.

364. A kit comprising:
a) at least 30 different populations of solid supports, each solid support comprising:
   i) a capture moiety attached to the solid support, wherein the capture moiety specifically binds an analyte; and
   ii) a tether oligonucleotide attached to the solid support independent of the capture moiety;
b) at least 30 different populations of capture oligonucleotides comprising from 5′ to 3′: a tethering region, a capture barcode region, and a 3′ hybridizing region, wherein the tethering region of each population of capture oligonucleotides is complementary to the tether oligonucleotide attached to a corresponding solid support of the at least 30 different populations of solid supports;
c) at least 30 different populations of detection moieties that specifically binds the analyte, wherein each detection moiety comprises a tether oligonucleotide attached to the detection moiety, wherein the tether oligonucleotides within a population have the same nucleotide sequence, and the tether oligonucleotides in different populations have different nucleotide sequences;
d) at least 30 different populations of detection oligonucleotide comprising from 5′ to 3′: a tethering region, a detection barcode region, and a 3′ hybridizing region, wherein the tethering region of each population of detection oligonucleotides is complementary to the tether oligonucleotide attached to a corresponding detection moiety of the at least 30 different populations of detection moieties; and
f) a strand-displacing DNA polymerase.

365. A method of determining a pairwise combination of binding moieties that can simultaneously bind to a binding target, the method comprising:
a) combining
   i) a plurality of different populations of solid supports, each solid support comprising:
      a first binding moiety attached to the solid support; and a capture oligonucleotide independently attached to the solid support, wherein the capture oligonucleotide comprises a 3′ hybridizing region and a capture barcode region, wherein the 3′ hybridizing regions of the capture oligonucleotides are the same in the plurality of different populations of solid supports, and wherein the capture barcode regions of the capture oligonucleotides are different for each different population of solid supports and the capture barcode region identifies the binding moiety attached to the same solid support, and;
   ii) a plurality of different populations of detection conjugates comprising:
      a second binding moiety; and
      a detection oligonucleotide attached to the second binding moiety, wherein the detection oligonucleotide comprises a 3′ hybridizing region complementary to the 3′ hybridizing region of the capture oligonucleotide and a detector barcode region, wherein the detector barcode regions of the detection oligonucleotide are different for each different population of detection conjugates and identifies the second binding moiety to which it is attached; and
   iii) one or more binding targets;
wherein, if a binding target is present in the sample, and the binding target is specifically bound by the first binding moiety and the second binding moiety, a capture complex is formed in which the capture oligonucleotide and the detection oligonucleotide are in proximity;
b) permitting the 3′ hybridizing region of the capture oligonucleotide and the 3′ hybridizing region of the detection oligonucleotide to hybridize to each other;
e) extending at least one of the hybridized capture oligonucleotide and the hybridized detection oligonucleotide to generate an extension product that comprises i) an extended capture oligonucleotide and a complement of at least a part of the detection oligonucleotide, and/or ii) an extended detection oligonucleotide and a complement of at least a part of the capture oligonucleotide;
f) releasing the extension product from the solid support; and
g) determining the capture barcode region and the detector barcode region of the released extension product to thereby determine a combination of binding moieties that can simultaneously bind to the binding target.

366. A method of determining interaction between two moieties, the method comprising:
a) combining a plurality of different populations of moieties, each moiety comprising a splint oligonucleotide attached to the moiety, wherein the splint oligonucleotide comprises a 3′ hybridizing region and a barcode region, wherein the 3′ hybridizing regions of the splint oligonucleotides are the same in the plurality of different populations of moieties, and wherein the barcode regions of the splint oligonucleotides are different for each different population of moieties and the barcode region identifies the moiety, and wherein, if a first moiety of the plurality of different populations of moieties interacts with a second moiety of the plurality of different populations of moieties, a capture complex is formed in which the splint oligonucleotide of the first moiety and the splint oligonucleotide of the second moiety are in proximity;
b) permitting the 3′ hybridizing region of the splint oligonucleotide of the first moiety and the 3′ hybridizing region of the splint oligonucleotide of the second moiety to hybridize to each other;
e) extending at least one of the splint oligonucleotide of the first moiety and the splint oligonucleotide of the second moiety to generate an extension product that comprises i) an extended splint oligonucleotide of the first moiety and a complement of at least a part of the splint oligonucleotide of the second moiety, and/or ii) an extended splint oligonucleotide of the second moiety and a complement of at least a part of the splint oligonucleotide of the first moiety;
f) releasing the extension product from the solid support; and
g) determining the barcode region of the splint oligonucleotide of the first moiety and the barcode region of the splint oligonucleotide of the second moiety of the released extension product to thereby determine an interaction between two moieties.

367. The method of embodiment 366, wherein the first moiety and the second moiety are proteins.

368. The method of embodiment 366, wherein the first moiety is an enzyme and the second moiety is a substrate.

369. The method of embodiment 366, wherein the first moiety is a protein and the second moiety is an aptamer.

369a. The method of any of embodiments 366 to 369, wherein the combining is performed in a plurality of subpools and the ratio of the concentration of the first moiety to the second moiety differs in different subpools of the plurality of subpools.

369b. The method of embodiment 369a, wherein the different subpools of the plurality of subpools are combined prior to determining the barcode region of the splint oligonucleotide of the first moiety and the barcode region of the splint oligonucleotide of the second moiety of the released extension product to thereby determine an interaction between two moieties.

369c. The method of any one of embodiments 366 to 369b, wherein the splint oligonucleotide of the first moiety is attached to the first moiety in a deterministic manner and/or the splint oligonucleotide of the second moiety is attached to the second moiety in a deterministic manner.

369d. The method of embodiment 369c, wherein only one splint oligonucleotide is attached to each first moiety and/or only one splint oligonucleotide is attached to each second moiety.

369e. The method of embodiment 369c, wherein only two splint oligonucleotides are attached to each second moiety.

370. A composition comprising at least 30 different populations of solid supports, each solid support comprising:
i) a capture moiety attached to the solid support, wherein the capture moiety specifically binds an analyte;
ii) a tether oligonucleotide attached to the solid support independent of the capture moiety; and
iii) a capture oligonucleotide comprising from 5′ to 3′: a tethering region, a capture barcode region, and a 3′ hybridizing region, wherein the capture oligonucleotide is attached to the solid support via hybridization of the tethering region to the tether oligonucleotide attached to the solid support.

371. The composition of embodiment 370, further comprising at least 30 different populations of detection conjugates, each detection conjugate comprising:
i) a detection moiety that specifically binds an analyte;
ii) a tether oligonucleotide attached to the detection moiety; and
ii) a detection oligonucleotide comprising from 5′ to 3′: a tethering region, a detection barcode region, and a 3′ hybridizing region, wherein the detection oligonucleotide is attached to the detection moiety via hybridization of the tethering region to the tether oligonucleotide attached to the detection moiety,
wherein each of the at least 30 different populations of solid supports forms a paired combination with a corresponding detection conjugate of the at least 30 different populations of detection conjugates, wherein the analyte bound by the capture moiety and detection moiety of each paired combination is the same, wherein the analytes bound by different paired combinations are different, wherein the 3′ hybridizing region of the capture oligonucleotide and detection oligonucleotide of each paired combination is not complementary to the 3′ hybridizing region of the detection oligonucleotide and the capture oligonucleotide, respectively, of any other paired combination, and wherein each paired combination can be distinguished from any other paired combination by one or both of the sequence of the capture barcode region and/or the sequence of the detection barcode region.

372. The composition of embodiment 370 or 371, further comprising at least 30 different populations of blocker oligonucleotides, wherein each of the different populations of blocker oligonucleotides specifically hybridizes to the 3′ hybridizing region or a portion thereof of the capture oligonucleotide.

373. The composition of any one of embodiments 370 to 372, further comprising at least 30 different populations of blocker oligonucleotides, wherein each of the different populations of blocker oligonucleotides specifically hybridizes to the 3′ hybridizing region or a portion thereof of the capture oligonucleotide or the detection oligonucleotide of each paired combination.

374. The composition of embodiment 372 or 373, wherein the blocker oligonucleotide is from 5 to 13 nucleotides long.

375. The composition of any one of embodiments 370 to 374, comprising from 30 to 1600 different paired combinations.

376. The composition of any one of embodiments 370 to 375, wherein the solid support is a magnetically responsive bead.

377. The composition of any one of embodiments 370 to 376, wherein the capture moiety and the detection moiety are independently an antibody or an antibody fragment.

378. The composition of any one of embodiments 370 to 377, further comprising a strand-displacing DNA polymerase.

379. The composition of any one of embodiments 370 to 378, further comprising a sample, the sample comprising one or more analytes.

379a. The composition of any one of embodiments 371 to 379, wherein the capture oligonucleotide is attached to the detection moiety in a deterministic manner.

379b. The composition of embodiment 379a, wherein only one capture oligonucleotide is attached to each detection moiety.

379c. The composition of embodiment 379a, wherein only two capture oligonucleotides are attached to each capture moiety.

380. A method of analyzing a sample for an analyte, comprising:
a) combining:
   i) a solid support comprising:
      a capture moiety attached to the solid support, wherein the capture moiety specifically binds an analyte; and
      a capture oligonucleotide attached to the solid support independently of the capture moiety;
   ii) a detection conjugate comprising:
      a detection moiety that specifically binds the analyte; and
      a detection oligonucleotide attached to the detection moiety;
   iii) a first splint oligonucleotide comprising:
      a first portion complementary to A) a 3′ hybridization region of the capture oligonucleotide, or B) a 5′ hybridization region of the capture oligonucleotide; and
      a second portion complementary to A) a 5′ hybridization region of the detection oligonucleotide if the first portion is complementary to the 3′ hybridization region of the capture oligonucleotide, or B) a 3′ hybridization region of the detection oligonucleotide if the first portion is complementary to the 5′ hybridization region of the capture oligonucleotide; and
   iv) a sample;
   wherein, if the analyte is present in the sample, it is bound to the solid support by the capture moiety and to the detection conjugate by the detection moiety to form a capture complex in which the capture oligonucleotide and the detection oligonucleotide are in proximity;
b) permitting i) the 3′ hybridizing region of the capture oligonucleotide and the 5′ hybridizing region of the detection oligonucleotide that are in proximity to hybridize to the first splint oligonucleotide, or ii) the 5′ hybridizing region of the capture oligonucleotide and the 3′ hybridizing region of the detection oligonucleotide that are in proximity to hybridize to the first splint oligonucleotide;
c) ligating the hybridized capture oligonucleotide and the hybridized detection oligonucleotide to generate an on-target ligation product that comprises the capture oligonucleotide and the detection oligonucleotide;
d) releasing the on-target ligation product from the solid support; and
e) determining the presence or the absence of the released on-target ligation product to determine the presence or the absence of the analyte in the sample.

381. The method of embodiment 380, wherein the combining in step a) further comprises combining a second splint oligonucleotide having a sequence complementary to a third portion of the first splint oligonucleotide.

382. The method of embodiment 381, further comprising permitting the second splint oligonucleotide to hybridize to the first splint oligonucleotide.

383. The method of embodiment 382, wherein the ligating in step c) comprises ligating the hybridized capture oligonucleotide to a first end of the second splint oligonucleotide and ligating the hybridized detection oligonucleotide to a second end of the second splint oligonucleotide to generate an on-target ligation product that comprises the capture oligonucleotide, the second splint oligonucleotide, and the detection oligonucleotide.

384. The method of any one of embodiments 380 to 383, wherein step a) comprises first combining the solid support and the sample such that the capture moiety of the solid support is bound to the analyte, if present in the sample, and then combining the detection conjugate and the first splint oligonucleotide, and wherein the method optionally comprises washing the analyte-bound solid support to remove unbound components of the sample prior to combining the detection conjugate and the first splint oligonucleotide.

385. The method of any one of embodiments 380 to 383, wherein step a) comprises first combining the detection conjugate and the sample such that the detection moiety is bound to the analyte, if present in the sample, and then combining the solid support and the first splint oligonucleotide.

386. The method of any one of embodiments 380 to 384, further comprising removing unbound components of the sample prior to combining the detection conjugate.

387. The method of any one of embodiments 380 to 386, further comprising washing the capture complex to remove components that are not part of the capture complex.

388. The method of any one of embodiments 380 to 387, wherein
i) the capture oligonucleotide comprises the 3′ hybridization region and a first 5′ tethering region and is attached to the solid support via hybridization to a first tether oligonucleotide attached to the solid support of the capture oligonucleotide, and the detection oligonucleotide comprises the 5′ hybridization region and a second 3′ tethering region and is attached to the detection moiety via hybridization to a second tether oligonucleotide attached to the detection moiety; or
ii) the capture oligonucleotide comprises the 5′ hybridization region and a first 3′ tethering region and is attached to the solid support via hybridization to a first tether oligonucleotide attached to the solid support of the capture oligonucleotide, and the detection oligonucleotide comprises the 3′ hybridization region and a second 5′ tethering region and is attached to the detection moiety via hybridization to a second tether oligonucleotide attached to the detection moiety.

389. The method of any one of embodiments 380 to 388, wherein the capture oligonucleotide and the detection oligonucleotide are ligated by a ligase.

390. The method of embodiment 389, wherein the ligase comprises a T4 DNA ligase, T7 DNA ligase, T3 DNA ligase, and/or PBCV-1 DNA ligase.

391. The method of any one of embodiments 380 to 390, wherein the capture oligonucleotide comprises a first barcode sequence that identifies a binding target of the capture moiety, and/or wherein the detection oligonucleotide comprises a second barcode sequence that identifies a binding target of the detection moiety.

392. The method of any one of embodiments 380 to 391, wherein
i) the capture oligonucleotide comprises from 5′ to 3′: a tethering region, a primer binding region configured to bind a primer for amplifying the released on-target ligation product, a barcode sequence, and the 3′ hybridizing region, and wherein the detection oligonucleotide comprises, from 3′ to 5′: a tethering region, a primer binding region configured to bind a primer for amplifying the released on-target ligation product, a barcode sequence, and the 5′ hybridizing region; or
ii) the capture oligonucleotide comprises from 3′ to 5′: a tethering region, a primer binding region configured to bind a primer for amplifying the released on-target ligation product, a barcode sequence, and the 5′ hybridizing region, and wherein the detection oligonucleotide comprises, from 5′ to 3′: a tethering region, a primer binding region configured to bind a primer for amplifying the released on-target ligation product, a barcode sequence, and the 3′ hybridizing region.

393. The method of any one of embodiments 380 to 392, wherein the solid support is a magnetically responsive bead.

394. The method of any one of embodiments 380 to 393, wherein the capture moiety and the detection moiety are independently an antibody or an antibody fragment.

395. The method of any one of embodiment 380 to 394, wherein the combining in step a) further comprises combining one or more blocker oligonucleotides, wherein each blocker oligonucleotide specifically hybridizes to a subpart of one or both of the capture oligonucleotide and/or the detection oligonucleotide.

396. The method of embodiment 395, wherein the blocker oligonucleotide is from 5 to 13 nucleotides long.

397. The method of embodiment 395 or 396, wherein the blocker oligonucleotide competes with the splint oligonucleotide for binding the detection oligonucleotide or the capture oligonucleotide.

398. The method of any one of embodiments 395 to 397, comprising removing the first blocker oligonucleotide after the combining in step a) and before the ligating in step c).

399. The method of any one of embodiments 380 to 398, wherein the capture oligonucleotide comprises a unique molecular identifier (UMI) at least 4 nucleotides long between the 5′ tethering region and the 3′ hybridizing region.

400. The method of any one of embodiments 380 to 399, wherein the detection oligonucleotide comprises a unique molecular identifier (UMI) at least 4 nucleotides long, and wherein the UMI is between the 5′ tethering region and the 3′ hybridizing region if the capture oligonucleotide comprises the 3′ hybridization region and the 5′ tethering region, or the UMI is between the 3′ tethering region and the 5′ hybridizing region if the capture oligonucleotide comprises the 5′ hybridization region and the 3′ tethering region.

401. The method of embodiment 392, wherein i) if the capture oligonucleotide comprises the 3′ hybridizing region and the detection oligonucleotide comprises the 5′ hybridizing region, the 3′ hybridizing region of the capture oligonucleotide and the 5′ hybridizing region of the detection oligonucleotide are asymmetric in length, or ii) if the capture oligonucleotide comprises the 5′ hybridizing region and the detection oligonucleotide comprises the 3′ hybridizing region, the 5′ hybridizing region of the capture oligonucleotide and the 3′ hybridizing region of the detection oligonucleotide are asymmetric in length.

402. The method of embodiment 401, wherein the 3′ hybridizing region of the capture oligonucleotide is from 3 to 10 nucleotides in length and the 5′ hybridizing region of the detection oligonucleotide is from 16 to 24 nucleotides in length.

403. The method of embodiment 402, wherein the 3′ hybridizing region of the capture oligonucleotide is from 3 to 6 nucleotides in length and the 3′ hybridizing region of the detection oligonucleotide is from 18 to 22 nucleotides in length.

404. The method of any one of embodiments 380 to 403, wherein determining the presence or the absence of the released on-target ligation product comprises performing qPCR.

405. The method of any one of embodiments 380 to 404, wherein determining the presence or the absence of the released on-target ligation product comprises sequencing the released on-target ligation product.

406. The method of any one of embodiments 380 to 405, comprising providing a plurality of paired combinations of the solid support and the detection conjugate, wherein binding targets of the capture moiety and the detection moiety of each paired combination are the same, and wherein different paired combinations of the plurality of paired combinations have different binding targets.

407. The method of embodiment 406, wherein the different binding targets are different analytes.

408. The method of embodiment 406, wherein the different binding targets are different epitopes on the same analyte.

409. The method of any one of embodiments 406 to 408, wherein the first splint oligonucleotide of a first paired combination to the detection oligonucleotide and the capture oligonucleotide of at least one other paired combination of the plurality of paired combinations.

410. The method of any one of embodiments 406 to 409, wherein each capture oligonucleotide attached to a solid support of the plurality of the solid supports comprises a barcode sequence that identifies a binding target of the capture moiety attached to the respective solid support; and/or wherein each detection oligonucleotide attached to a detection moiety of the plurality of the detection conjugates comprises a barcode sequence that identifies a binding target of the respective detection moiety.

411. The method of any one of embodiments 380 to 410, wherein the sample is divided into a plurality of subpools comprising at least a first subpool and a second subpool, and, optionally, wherein (i) the sample concentration in different subpools of the plurality of subpools differ, (ii) the detection conjugate concentration in different subpools of the plurality of subpools differ, (iii) the buffers in different subpools of the plurality of subpools differ, (iv) different subpools of the plurality of subpools are combined with the solid support and/or the detection conjugate for different amounts of time, (v) the first subpool is combined with the solid support and/or the detection conjugate for from one minute to thirty minutes longer than the amount of time the second subpool is combined with the solid support and/or the detection conjugate, and/or (vi) the different subpools of the plurality of subpools are combined prior to determining the presence or the absence of the released on-target ligation product.

412. The method of any of embodiments 380 to 411, wherein the first splint oligonucleotide is from 23 to 42 nucleotides in length.

413. The method of any of embodiments 380 to 412, further comprising combining a second splint oligonucleotide comprising a nucleotide sequence complementary to a third portion of the first splint oligonucleotide, wherein the third portion is between the first portion and the second portion of the first splint oligonucleotide.

414. The method of embodiment 413, wherein the permitting in step b) further comprising permitting the second splint oligonucleotide to hybridize to the first splint oligonucleotide; and the ligating in step c) comprising ligating the second splint oligonucleotide to the hybridized capture oligonucleotide and the hybridized detection oligonucleotide to generate an on-target ligation product that comprises the capture oligonucleotide, the second splint oligonucleotide, and the detection oligonucleotide.

415. The method of embodiment 413 or 414, wherein the second splint oligonucleotide comprises a barcode sequence.

416. The method of any one of embodiments 413 to 415, wherein the second splint oligonucleotide is from 4 to 20 nucleotides in length, from 8 to 12 nucleotides in length, or 10 nucleotides in length.

417. The method of any one of embodiments 380 to 416, wherein the releasing in step d) comprising treating the solid support with an enzyme.

418. The method of embodiment 417, wherein the enzyme is a protease.

419. The method of embodiment 417, wherein the enzyme is a restriction endonuclease.

420. The method of any one of embodiments 417 to 419, wherein the method comprises treating the solid support with two enzymes.

421. The method of embodiment 420, wherein the first portion of the splint oligonucleotide is complementary to the 3′ hybridizing region of the capture oligonucleotide and the second portion of the splint oligonucleotide is complementary to the 5′ region of the detection oligonucleotide, and wherein the two enzymes comprise a restriction endonuclease and a strand-displacing polymerase.

422. The method of embodiment 421, wherein the restriction endonuclease comprises EcoRI and the strand-displacing polymerase comprises a Klenow fragment.

423. The method of any one of embodiments 420 to 422, wherein the solid support is treated with the two enzymes sequentially.

424. The method of any one of embodiments 420 to 422, wherein the solid support is treated with the two enzymes simultaneously.

424a. The method of any one of embodiments 380 to 424, wherein the capture oligonucleotide is attached to the detection moiety in a deterministic manner.

424b. The method of embodiment 424a, wherein only one capture oligonucleotide is attached to each detection moiety.

424c. The method of embodiment 424a, wherein only two capture oligonucleotides are attached to each capture moiety.

425. A composition comprising:
a) a first pool comprising at least 30 different populations of solid supports, each solid support comprising:
   i) a capture moiety attached to the solid support, wherein the capture moiety specifically binds an analyte;
   ii) a tether oligonucleotide attached to the solid support independent of the capture moiety; and
   iii) a capture oligonucleotide comprising A) from 5′ to 3′: a tethering region, a capture barcode region, and a 3′ hybridizing region, wherein the capture oligonucleotide is attached to the solid support via hybridization of the tethering region to the tether oligonucleotide attached to the solid support, or B) from 3′ to 5′: a tethering region, a capture barcode region, and a 5′ hybridizing region, wherein the capture oligonucleotide is attached to the solid support via hybridization of the tethering region to the tether oligonucleotide attached to the solid support; and
b) a second pool comprising at least 30 different populations of detection conjugates, each detection conjugate comprising:
   i) a detection moiety that specifically binds the analyte;
   ii) a tether oligonucleotide attached to the detection moiety; and
   ii) a detection oligonucleotide comprising A) from 5′ to 3′: a tethering region, a detection barcode region, and a 3′ hybridizing region, or B) A) from 3′ to 5′: a tethering region, a detection barcode region, and a 5′ hybridizing region, wherein the detection oligonucleotide is attached to the detection moiety via hybridization of the tethering region to the tether oligonucleotide attached to the detection moiety,
c) a third pool comprising at least 30 different populations of first splint oligonucleotides, each first splint oligonucleotide comprising:
   i) a first portion complementary to A) a 3′ hybridization region of the capture oligonucleotide, or B) a 5′ hybridization region of the capture oligonucleotide; and
   ii) a second portion complementary to A) a 5′ hybridization region of the detection oligonucleotide if the first portion is complementary to the 3′ hybridization region of the capture oligonucleotide, or B) a 3′ hybridization region of the detection oligonucleotide if the first portion is complementary to the 5′ hybridization region of the capture oligonucleotide; and
wherein each of the at least 30 different populations of first splint oligonucleotides forms a triad combination with a corresponding solid support of the at least 30 different populations of solid supports and a corresponding detection conjugate of the at least 30 different populations of detection conjugates, wherein the analyte bound by the capture moiety and detection moiety of each triad combination is the same, wherein the analytes bound by different triad combinations are different, wherein the capture oligonucleotide and detection oligonucleotide of each paired combination are not complementary to the first splint oligonucleotide of any other triad combination, and wherein each triad combination can be distinguished from any other triad combination by one or both of the sequence of the capture barcode region and/or the sequence of the detection barcode region.

426. The composition of embodiment 425, further comprising at least 30 different populations of a second splint oligonucleotide comprising a nucleotide sequence complementary to a third portion of a corresponding first splint oligonucleotide of the at least 30 different populations of first splint oligonucleotides, wherein the third portion is between the first portion and the second portion of the first splint oligonucleotide.

426a. The composition of any one of embodiments 425 to 426, wherein the capture oligonucleotide is attached to the detection moiety in a deterministic manner.

426b. The composition of embodiment 426a, wherein only one capture oligonucleotide is attached to each detection moiety.

426c. The composition of embodiment 426a, wherein only two capture oligonucleotides are attached to each capture moiety.

427. A method for multiplexed analysis of analytes in a sample, the method comprising:
a) combining the composition of any one of embodiments 425 or 426 and a sample;
b) permitting the first splint oligonucleotide to hybridize to the capture oligonucleotide and the detection oligonucleotide, and optionally to the second splint oligonucleotide if present, of each triad combination if the analyte of the triad combination is present in the sample;
c) ligating the hybridized capture oligonucleotide and the hybridized detection oligonucleotide to generate an on-target ligation product for each triad combination for which the analyte of the paired combination is present in the sample;
d) releasing the on-target ligation product for each triad combination for which the analyte of the triad combination is present in the sample from the solid support; and
e) determining the presence or the absence of the released on-target ligation product to determine the presence or the absence of the analyte of each paired combination in the sample.

428. The method of embodiment 427, wherein the combining in step a) comprises combining the sample and the first pool comprising at least 30 different populations of solid supports, and then combining the second pool comprising at least 30 different populations of detection conjugates with the first pool, and wherein the method optionally comprises washing the analyte-bound first pool to remove unbound components of the sample prior to combining the second pool with the first pool.

429. The method of embodiment 427, wherein the combining in step a) comprises combining the first pool comprising at least 30 different populations of solid supports and the second pool comprising at least 30 different populations of detection conjugates, and then combining the sample with the first pool and the second pool.

429a. The method of any one of embodiments 427 to 429, wherein the capture oligonucleotide is attached to the detection moiety in a deterministic manner.

429b. The method of embodiment 429a, wherein only one capture oligonucleotide is attached to each detection moiety.

429c. The method of embodiment 429a, wherein only two capture oligonucleotides are attached to each capture moiety.

430. A method of analyzing a sample for an analyte, comprising:
a) combining:
   i) a first conjugate comprising:
      a first moiety that specifically binds an analyte;
      a first tether region attached to the first moiety; and a first splint oligonucleotide comprising A) a 5′ tethering region, a first moiety barcode, and a 3′ hybridizing region, wherein the 5′ tethering region of the first splint oligonucleotide is hybridized to the first tether region attached to the first moiety, or B) a 3′ tethering region, a first moiety barcode, and a 5′ hybridizing region, wherein the 3′ tethering region of the first splint oligonucleotide is hybridized to the first tether region attached to the first moiety;
   ii) a second conjugate comprising:
      a second moiety that specifically binds the analyte;
      a second tether region attached to the second moiety; and
      a second splint oligonucleotide comprising A) a 5′ tethering region, a second moiety barcode, and a 3′ hybridizing region, wherein the 5′ tethering region of the second splint oligonucleotide is hybridized to the second tether region attached to the second moiety, or B) a 3′ tethering region, a second moiety barcode, and a 5′ hybridizing region, wherein the 3′ tethering region of the second splint oligonucleotide is hybridized to the second tether region attached to the second moiety;
   iii) a third splint oligonucleotide comprising:
      a first portion complementary to A) a 3′ hybridization region of the capture oligonucleotide, or B) a 5′ hybridization region of the capture oligonucleotide; and
      a second portion complementary to A) a 5′ hybridization region of the detection oligonucleotide if the first portion is complementary to the 3′ hybridization region of the capture oligonucleotide, or B) a 3′ hybridization region of the detection oligonucleotide if the first portion is complementary to the 5′ hybridization region of the capture oligonucleotide; and
   iv) a sample;
b) permitting the first splint oligonucleotide and the second splint oligonucleotide that are in proximity to hybridize to the third splint oligonucleotide if the analyte is present in the sample;
c) ligating the hybridized first splint oligonucleotide and the hybridized second splint oligonucleotide to generate an on-target ligation product that comprises the first splint oligonucleotide and the extended second splint oligonucleotide;
d) releasing the on-target ligation product by treating the on-target ligation product with a restriction endonuclease and a strand-displacing DNA polymerase; and
e) determining the presence or the absence of the on-target ligation product to thereby determine the presence or the absence of the analyte in the sample.

431. The method of embodiment 430, wherein the combining in a) comprises combining a fourth splint oligonucleotide comprising a sequence complementary to a third portion of the third splint oligonucleotide, wherein the third portion is between the first portion and the second portion.

432. The method of embodiment 431, wherein the permitting in step b) further comprising permitting the fourth splint oligonucleotide to hybridize to the third splint oligonucleotide; and the ligating in step c) comprising ligating the fourth splint oligonucleotide to the hybridized capture oligonucleotide and the hybridized detection oligonucleotide to generate an on-target ligation product that comprises the capture oligonucleotide, the second splint oligonucleotide, and the detection oligonucleotide.

433. The method of embodiment 431 or 432, wherein the first moiety and the second moiety specifically bind different epitopes on the same analyte.

434. The method of embodiment 431 or 432, wherein the first moiety specifically binds with a first analyte and the second moiety specifically binds with a second analyte, wherein the first analyte and the second analyte interact.

435. The method of embodiment 434, wherein the first analyte is an enzyme and the second analyte is a substrate for the enzyme.

436. The method of any one of embodiments 431 to 435, wherein the combining in a) comprises combining the first conjugate and the sample, thereby allowing the first moiety of the first conjugate to be bound to the analyte if present in the sample, and then combining the second conjugate with the first conjugate.

437. The method of embodiment 436, further comprising washing the analyte-bound first conjugate to remove unbound components of the sample prior to combining the second conjugate with the first conjugate.

438. The method of any one of embodiments 431 to 437, comprising providing: a plurality of triad combinations of the first conjugates, the second conjugates, and the third splint oligonucleotides, wherein the analyte bound by the first moiety and second moiety of each traid combination is the same, and wherein different triad combinations of the plurality of triad combinations bind different analytes.

438a. The method of any one of embodiments 430 to 438, wherein the first splint oligonucleotide is attached to the first moiety in a deterministic manner and/or the second splint oligonucleotide is attached to the second moiety in a deterministic manner.

438b. The method of embodiment 438a, wherein only one first splint oligonucleotide is attached to each first moiety and/or only one second splint oligonucleotide is attached to each second moiety.

438c. The method of embodiment 438a, wherein only two first splint oligonucleotides are attached to each first moiety and/or only two second splint oligonucleotides are attached to each second moiety.

439. A method of determining a pairwise combination of binding moieties that can simultaneously bind to a binding target, the method comprising:
a) combining
   i) a plurality of different populations of solid supports, each solid support comprising:
      a first binding moiety attached to the solid support; and a capture oligonucleotide independently attached to the solid support, wherein the capture oligonucleotide comprises a splint hybridizing region and a capture barcode region, wherein the splint hybridizing regions of the capture oligonucleotides are the same in the plurality of different populations of solid supports, and wherein the capture barcode regions of the capture oligonucleotides are different for each different population of solid supports and the capture barcode region identifies the binding moiety attached to the same solid support;
   ii) a plurality of different populations of detection conjugates comprising:
      a second binding moiety; and a detection oligonucleotide attached to the second binding moiety, wherein the detection oligonucleotide comprises a splint hybridizing region and a detector barcode region, wherein the detector barcode regions of the detection oligonucleotide are different for each different population of detection conjugates and identifies the second binding moiety to which it is attached;
   iii) a plurality of different populations of a first splint oligonucleotide comprising:
      a first portion complementary the splint hybridizing region of the capture oligonucleotide, and a second portion complementary to the splint hybridizing region of the detection oligonucleotide; and
   iv) one or more binding targets;
wherein, if a binding target is present in the sample, and the binding target is specifically bound by the first binding moiety and the second binding moiety, a capture complex is formed in which the capture oligonucleotide and the detection oligonucleotide are in proximity;
b) permitting the first splint oligonucleotide to hybridize to the splint hybridizing region of the capture oligonucleotide and the splint hybridizing region of the detection oligonucleotide if the capture complex is formed;
e) ligating the hybridized capture oligonucleotide and the hybridized detection oligonucleotide to generate a ligation product that comprises the capture oligonucleotide and the detection oligonucleotide;
f) releasing the ligation product from the solid support; and
g) determining the capture barcode region and the detector barcode region of the released ligation product to thereby determine a combination of binding moieties that can simultaneously bind to the binding target.

440. The method of embodiment 440, wherein the capture oligonucleotide is attached to the first binding moiety in a deterministic manner and/or the detection oligonucleotide is attached to the second binding moiety in a deterministic manner.

441. The method of embodiment 440, wherein only one capture oligonucleotide is attached to each first binding moiety and/or only one detection oligonucleotide is attached to each second binding moiety.

442. The method of embodiment 440, wherein only two capture oligonucleotides are attached to each first binding moiety and/or only two detection oligonucleotides are attached to each second binding moiety.

### XIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

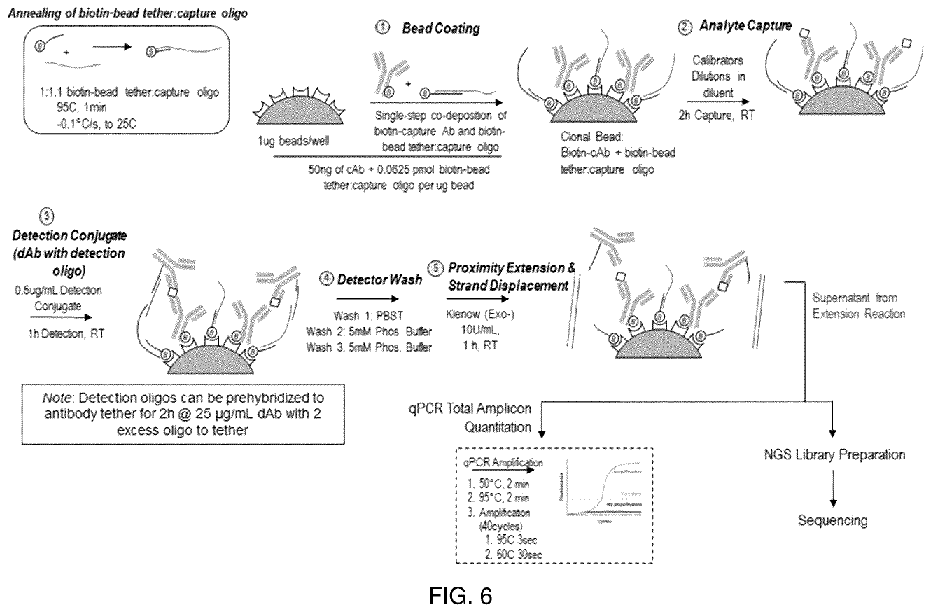

This non-limiting example shows a PESD assay (FIG. 6).
1. Bead coating and barcoding:
   a. (pre-) hybridize capture oligo to bead tether (e.g., the bead tether may be biotinylated) (FIG. 5A);
   b. Attach (i) capture antibody (cAby or cAb) and (ii) bead tether with hybridized capture oligo to bead (e.g., attaching may involve coating the beads with streptavidin (SA) and using biotinylated cAby and biotinylated bead tether) (FIG. 5B, FIG. 6 step 1)).
      i. a minimum amount of oligo and maximum amount of antibody can make the assay more efficient. The amount of oligonucleotide on the bead can be reduced to prevent or reduce pulldown of detector by the oligonucleotide in the absence of analyte, where such pulldown of detector may increase assay background. Various concentrations used for each component (biotin-cAby and biotin-bead tether) and different coating processes (e.g., simultaneous vs sequential coating) can be used. In some embodiments, a single-step co-deposition of biotinylated cAby and biotinylated pre-hybridized bead tether is used.
      ii. For multiplexed assays, this step can be done in clonal fashion, e.g., one antibody species and one unique detection oligo species per set of beads (FIG. 5C).
Hybridization of biotinylated bead tether and capture oligo
   Thermally anneal the biotinylated bead tether oligo and capture oligo at 1.1:1 ratio, at 95° C., 1 min, then −0.1° C./see to 25° C.;
Bead coating, per well conditions:
   1 ng-1 μg of 1 m Pierce Beads per well;
   50 ng biotinylated cAby per g bead;
   0.625 pmol biotinylated bead tether with hybridized capture oligo per ug bead;
   100 μL Diluent A (containing: 2.0% bovine, serum albumin, 2.1% potassium phosphate dibasic, 0.5% potassium phosphate monobasic, 0.04% kathon CG/ICP II, 2.0% sucrose, and 0.022% Triton™ X-100).
2. Wash away unbound oligos and antibodies;
3. Obtain dAby and conjugate it with detection tether (=detection complex) (FIG. 3A);
4. (pre-)hybridize detection oligo to detection tether to make “detector” (also referred to as “detection conjugate” FIG. 5D);
5. add sample to coated, barcoded beads—2 hours at room temperature (FIG. 5E, FIG. 6 step 2);
6. wash away unbound analyte;
7. add 0.01-0.5 μg/mL detector (=detection conjugate including hybridized detection oligo) to beads—incubate 1 hour at room temperature (FIG. 3C, FIG. 6 step 3);
8. wash beads 3× (1^{st}: PBST; 2^{nd}: 5 mM phosphate buffer; 3^{rd}: 5 mM phosphate buffer) (FIG. 6 step 4);
9. add beads to 0.01-1.0 U/mL of 3→5′ exo-Klenow fragment with strand displacement activity (FIG. 6 step 5)—1 hour at room temperature;
   a. enzymes extend detection and capture oligos (bidirectional extension) and then displace them from the tethers, releasing the double stranded product into supernatant; extension and amplicon release occur in the same step;
   b. material (amplicon) can be quantified at this point using qPCR or multiplex qPCR for multiplex assays;
10. perform 2 PCR steps on PESD supernatants for NGS library prep;
   1) Adapter PCR: Preamplification using tailored Adapter primers (P5 and P7 overhang adapter primers)
      Control oligo is spiked into master mix at this point to allow well to well normalization. This controls for variations in recovery during PCR cleanup steps as well as pipetting errors or some PCR bias.
      Cleanup is performed with Solid Phase Reversible Immobilization (SPRI) beads such as AMPure XP beads (Beckman Coulter) to remove excess adapter primers (in post PCR lab).
2) Index PCR (8 cycles) using Nextera unique dual indexing primers (IDT for Illumina Unique Dual Index Kit);
   cleanup with SPRI beads to remove excess index primers (in post PCR lab); SPRI beads quantitatively recover the post-PCR product and preserve the starting amplicon concentration differences (as measured by qPCR).

11. multiple indexed samples are pooled at equal volumes to preserve ratios of amplicon (and therefore the original sample concentration) between wells;
12. complete, pooled library is quantified, loaded on NextSeq instrument (Illumina), and sequenced.

### Example 2

This non-limiting example shows a Singleplex Proximity Extension Strand Displacement (PESD) IL-13 assay.

### Scheme and Sequences Used:

TABLE 2.1

| Name | Sequence (5→3) |
|---|---|
| First Tether Oligo | G CTG TCT TCG CTA TTT GCC G (SEQ ID NO: 3) |
| (“Bead tether”) | |
| Capture Oligo | CGG CAA ATA GCG AAG ACA GCN NNN NNN NGT CTC |
| | TAT GAG TTG T (SEQ ID NO: 1) |
| Second Tether Oligo | CCT ATG CTC TAG CGC CTC AC (SEQ ID NO: 4) |
| (“Detection tether”) | |
| Detection Oligo | GTG AGG CGC TAG AGC ATA GGN NNN NNN NAC TTG |
| | CGC CAA CAA C (SEQ ID NO: 2) |

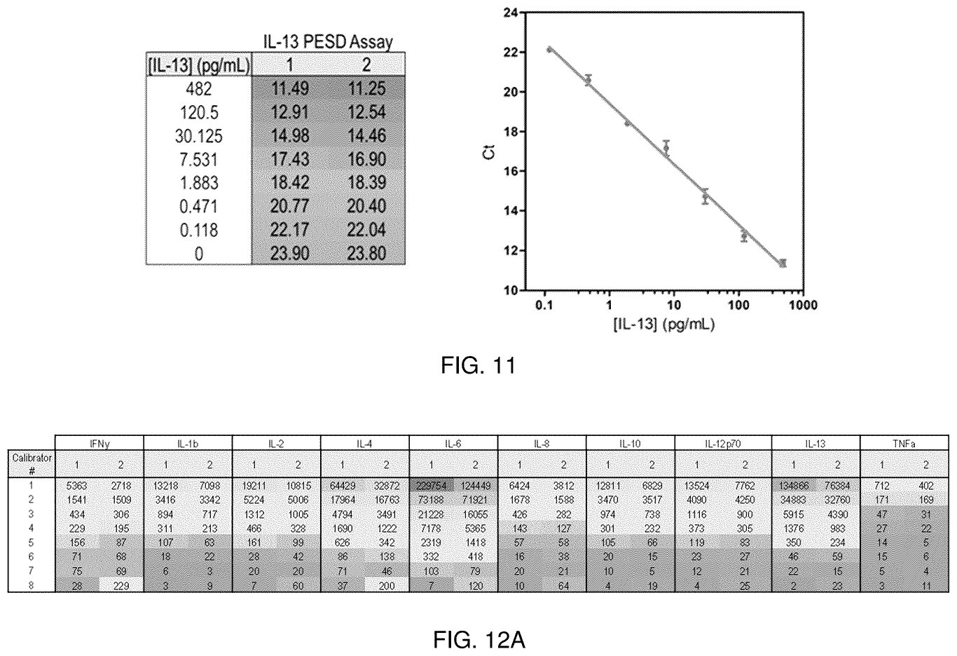

Proximity Extension Strand Displacement (PESD) assays were demonstrated in singleplex form where 2 barcoded oligos on opposite sides of an assay sandwich (one barcoded oligonucleotide linked to a detection antibody (“detection oligo”) and the other barcoded oligonucleotide (“capture oligo”) linked to a bead that is also linked to a capture antibody) extended bidirectionally when held in proximity, and released a double-stranded doubly-barcoded amplicon corresponding to the capture and detector antibodies present in the assay sandwich. The barcoded oligos each contained a unique detector barcode sequence and a 5 nt hybridization region wherein the hybridization region sequence of the capture oligo was complementary to the hybridization region sequence of the detection oligo (FIG. 10, Table 2.1). It was shown that amplicon concentration is directly proportional to analyte concentration, making this a fully quantifiable proximity extension assay system (FIG. 11).

The PESD reactions were performed as shown in FIG. 6. Briefly, beads were washed by adding 76.8 μg of beads to a 1.5 mL microcentrifuge tube and diluting with 1 mL of Diluent A+500 mM NaCl. Beads were pulled down on a magnetic tube rack and the supernatant was removed and discarded.

### Bead Coating

Washed beads were added to a co-solution of 3.85 μg of biotinylated-capture Antibody (biotin-IL-13), 4.8 pmol of biotinylated bead tether pre-hybridized to capture oligonucleotide, and 7,668 μL of Diluent A+500 mM NaCl in a 15 mL centrifuge tube. The solution was mixed on a rotisserie for 1 hour at room temperature, then moved to 4° C. overnight.

### Analyte Capture

A 4-fold serial dilution series was prepared for IL-13 calibrator with a top-of-curve concentration of 482 μg/mL using Diluent A. Calibrator dilutions were transferred to wells of a KingFisher microwell plate (100 μL/well).

The coated beads were washed three times in PBST, and released into wells containing their respective calibrator solutions. Beads were mixed with calibrator for 2 hours to permit analyte capture.

### Addition of Detectors (Immunosandwich Formation)

Detectors were prehybridized by annealing detection oligonucleotides to detection complexes (which consisted of the detection antibody conjugated to a detection tether) at various ratios. Prehybridization was performed at 10 μg/mL of detection antibody in DPBS for 1 hour, before diluting the entire detection solution to a working concentration of 0.5 μg/mL detection antibody in Diluent B (containing: 2.0% sucrose, 2.0% BSA, 2.1% potassium phosphate dibasic, 0.5% potassium phosphate monobasic, 0.04% Kathon CG/ICP II, 0.022% Triton™ X-100, 0.1% mouse IgG, and 0.5% goat IgG).

After 2 h of analyte capture at room temperature, beads were collected, washed, and released into the prehybridized detector solutions. The beads mixed in the detector solution for 1 hour at room temperature.

### Detector Wash

Beads were washed three times to remove unbound detector. The first wash plate contained non-stringent MSD® Wash Buffer (PBS+0.05% polysorbate 20), while the subsequent two washes were with a stringent low-salt buffer (5 mM Phosphate+0.05% polysorbate 20).

### Proximity Extension and Strand Displacement

After the final wash, beads were released into Klenow (exo-) polymerase solution (10 U/mL) and extension and strand displacement occurred at room temperature for 1 hour. Beads were then removed from the extension supernatant containing the released full length amplicons.

qPCR

Working in the PCR prep hood, qPCR Mastermix was prepared with a target “in well” concentration of 0.3 μM forward and reverse primers. 15 μL of the appropriate qPCR master mix was added to the wells of a 0.1 mL MicroAmp optical qPCR plate. For each target analyte, 5 L of extension supernatant (containing amplicons) was transferred a well. The plate was covered with an optical plate seal and centrifuged at 1000 RCF for 1 min to force all the material to the bottom of the well.

qPCR analysis was performed following the parameters below:
1. Uracil deglycosylase (UDG) activation: 50° C. 2 min
2. Dual-Lock DNA Polymerase: 95° C. 2 min
3. Amplification (40 cycles)
   i. Denaturation: 95° C. 3 sec
   ii. Annealing+Extension: 60° C. 30 sec

### Results

This non-limiting example illustrates singleplex PESD. In addition, it shows that bead coating and barcoding can be reduced to a single-step by prehybridizing and thermally annealing the two complementary strands (biotin-bead tether and capture oligo) prior to bead coating. In some embodiments, this single-step consolidation may 1) reduce assay time by eliminating a subsequent hybridization step, 2) minimize waste by lowering stoichiometric excess of capture oligo needed to fully hybridize, and/or 3) improve assay performance by increasing capture barcode surface occupancy on beads. The prehybridzation ratio of detection tether to detection oligo does not adversely affect the assay performance if detection oligo concentration is greater than 1× the detection tether molar concentration and that hybridization efficiency is sufficient when performed at 10 μg/mL dAb in DPBS for 1 h. Hybridization of detectors, with very high occupancy, can be achieved in under 2 h with a slight excess of detection oligo. Excess amounts of unbound detection oligo do not appreciably increase assay background—increasing concentration (e.g., up to 25×[287 nM free oligo]) did not improve reaction efficiency; high occupancy was achieved with 1.Lx excess.

In addition to the IL-13 PESD assay, more than 40 additional antibody pairs were tested and observed to perform successfully in PESD singleplex format (see Table 2.2 below). The robustness of the PESD assay design, with its common capture and detector tethers, allows for many high quality antibody pairs (e.g., a pair containing a capture antibody and detection antibody known to bind simultaneously to the same analyte) to be inserted into PESD for immediate use with little to no optimization required. Below is a list of antibody pairs that have been successfully integrated in the PESD format.

TABLE 2.2
Assays Successfully tested in PESD Format

| | | | |
|---|---|---|---|
| ILN-γ | Eotaxin-3 | IL-15 | IL-2Rα |
| IL-1β | TARC | IL-31 | IL-33 |
| IL-2 | IP-10 | IL-17a | Tie-2 |
| IL-4 | MIP-1α | TNF-β | VEGF-D |
| IL-6 | MCP-1 | VEGF-A | VEGF-C |
| IL-8 | MDC | FLT-1/VEGFB-1 | FGF (basic) |
| IL-10 | MCP-4 | Granzyme A | IL-22 |
| IL-12p70 | GM-CSF | IL-27 | IL-23 |
| IL-13 | IL-1α | IL-18 | |
| MIP-3α | IL-5 | IL-21 | |
| Eotaxin | IL-7 | PIGF | |
| MIP-1β | IL-12/IL-23p40 | IL-29/IFN-λ1 | |

### Example 3

This non-limiting example shows a Multiplexed (10-plex) PESD with Next Generation Sequencing (NGS). In this example, a PESD 10-Plex assay was performed and read-out by NGS. Bead quantity (per assay per well), and detector concentration (per assay) were titrated in this 10-Plex. This demonstrated multiplexed PESD immunoassay followed by NGS read out.

### Bead Coating and Barcoding

Ten unique pairs of barcoded oligos (each pair contained a capture oligo and a detection oligo) were designed and synthesized to go with ten antibody pairs (capture and detect antibodies for ten analytes) from MSD® V-PLEX Proinflammatory Panel 1 Human Kit (Meso Scale Diagnostics, LLC.). Each capture oligo contained a sequence that was complementary to the bead tether oligo, a unique capture barcode sequence specific to a particular analyte, and a 5 nt hybridization sequence (see schematic in FIG. 3D). Each detection oligo contained a sequence that was complementary to the detection tether oligo, a unique detector barcode sequence specific to a particular analyte, and a 5 nt hybridization sequence that is the complement of the capture oligo hybridization sequence.

Intermediate dilutions of biotinylated capture antibodies were prepared to make 20 L of 50 μg/mL each in DPBS for analytes 1-10 (IFNy, IL-1b, IL-2, IL-4, IL-6, IL-8, IL-10, IL-12p70, IL-13, and TNFa). Capture oligo and biotinylated bead tether for each of analytes 1-10 were previously annealed and stored at 10,000 nM. Intermediate dilutions were prepared at 100 nM in PBS.

60 μg (6 μL of 10,000 μg/mL) of 1 um Pierce streptavidin (SA) coated beads was pulled from the vendor tube and washed in 1 mL of Diluent A diluent. The supernatant was discarded and the beads were resuspended in 60 μL of Diluent A to make a washed bead stock of 1,000 μg/mL.

Clonal bead coating: for each of analytes 1-10, 5.3 μL (i.e., 5.3 μg) of washed beads were added to a co-solution of 0.33 pmol biotinylated bead tether annealed to capture oligo, and 0.27 μg of the assigned biotinylated capture antibody in 519 μL of Diluent A in a 1.5 mL centrifuge tube. Each tube was mixed on rotisserie for 1 h at RT, then moved to 4 C overnight.

After coating, clonal beads (533 μL input volume) were washed in 10 different wells using a KingFisher Duo Prime sample purification instrument (Thermo Fisher Scientific, Inc.) via initial collection, and washing through 3 subsequent wells of 1 mL PBST via release and recollection. After the final wash, beads were released into 533 μL of PBST to maintain the same bead concentration of 10 μg/mL. Clonal, barcoded beads were transferred to 1.5 mL tubes and stored at 4° C. overnight.

### Bead Pooling

Washed, clonal, barcoded beads were diluted to working concentrations of 1, 0.1, or 0.01 μg/mL per assay per well (or 0.1, 0.01, or 0.001 μg per well per assay) in a pooled mix of 10 clonal bead types. Pooled beads were distributed to 96 wells of a 200 uL KingFisher Apex plate.

### Detector Barcoding

Individual detection complexes (consisting of detection antibody with attached detection tether) for each assay in the 10-Plex were pre-hybridized to their barcoded detection oligos to produce barcoded detection conjugates. The gross quantity of each detection antibody required for the whole experiment was calculated, then that quantity of detection complex was prehybridized to its detection oligo with an antibody concentration of 25 μg/mL to increase the hybridization rate. A degree of labeling (DOL; the analytical determination of how many tethers are covalently conjugated to each detection antibody) of 3.7 was assumed for each complex, and detection oligos were added at a 2× excess of detection oligo to detection tether, thus each tube contained 161.3 nM of detection antibody, 599 nM detection tether, and 1,193.5 nM detection oligo in DPBS.

### Detector Pooling

After 1 h of detector pre-hybridization, detection cocktails were prepared by pooling the barcoded detector antibody conjugates.

### Calibrator Addition & Capture

MSD® Proinflammatory Panel 1 Human Calibrator Blend (lot AD005280; Meso Scale Diagnostics, LLC.) containing 10 calibrator proteins of analytes 1-10 was reconstituted in 2 mL of Diluent A and vortexed. An eight point serial dilution series was prepared for the blended calibrator using 4-fold steps. Dilutions were prepared with Diluent A. Calibrator 8 (the blank) contained Diluent A only. 100 μL of blended calibrator was added to the appropriate well of a new KingFisher Apex 200 μL plate according to a plate map. Beads were added to wells containing blended calibrator and incubated for 2 hours to permit capture.

### Addition of Detectors

After capture, beads were washed to remove unbound calibrator and released in detector solution containing the pooled detection cocktail (containing each barcoded detector antibody conjugate) in Diluent B. Beads were incubated in detector solution for 1 hour.

Beads were then washed three times to remove unbound barcoded detector antibody conjugates. The first wash was with non-stringent MSD® Wash Buffer (PBS+0.0.5% polysorbate 20), while the subsequent two washes were with a stringent low-salt buffer (5 mM Phosphate+0.05% polysorbate 20) to denature any oligonucleotide hybridization.

### Extension and Strand Displacement

Washed beads were released into Klenow (3->5exo-) polymerase solution.

Proximity extension and strand displacement were performed at RT for 1 hour. During this time, the enzyme extended the detection and capture oligos (bidirectional extension) and then displaced them from the tether oligos, releasing the double stranded product into the supernatant; extension and amplicon release occur in the same step during this process. A KingFisher Apex sample purification instrument was used to remove the beads from the extension supernatant containing the released full length amplicon.

qPCR

qPCR was performed to quantitate the resulting amplicons. Working in the PCR prep hood, qPCR Mastermix was prepared with a target “in well” concentration of 0.3 μM forward and reverse primers (forward and reverse primers were designed to the common primer regions of the bead tether oligo and the detection tether oligo). 15 μL of the appropriate qPCR master mix was added to corresponding well in a 0.1 mL MicroAmp optical qPCR plate. 5 μL of sample was transferred from the Extension Supernatant plate to the corresponding well (e.g., A1 to A1) of the qPCR plate. The plate was covered with an optical plate seal and centrifuged at 1000 RCF for 1 min to force all the material to the bottom of the well.

qPCR analysis was performed following the parameters below:
UDG activation: 50° C. 2 min
Dual-Lock DNA Polymerase: 95° C. 2 min
Amplification (40 cycles)
   Denaturation: 95° C. 3 sec
   Annealing+Extension: 60° C. 30 sec

Based on qPCR data, the amplicons in the wells of columns 1, 2, 4, 7, 8, and 10 of the Supernatant Extension plate were diluted by a unique dilution factor (applied to each unique column) in order to approximately normalize the amplicon concentration for equal representation in the sequencer. The amplicons in the wells of plate columns 3, 5, 6, 9, 11, and 12 were not diluted due to their concentration being equal to or lower than the target total amplicon concentration (the PESD plate was organized with columns on the plate having different amounts of beads and detector; as such they produced different amounts of total amplicon (as analyzed by qPCR)).

### Library Preparation and Sequencing

Two PCR steps were performed on the PESD extension supernatants for NGS library preparation:
1. First, adapter PCR was performed. This preamplification step used tailored adapter primers (P5 and P7 overhang adapter primers) and control oligo was spiked into master mix at this point to allow well to well normalization (controls for variations in recovery during PCR cleanup steps as well as pipetting errors or some PCR bias). Following the PCR, cleanup was performed with SPRI beads to remove excess adapter primers (in post PCR lab).
2. Second, index PCR (8 cycles) was performed using Nextera unique dual indexing primers (Illumina). Cleanup was performed with SPRI beads to remove excess index primers (in post PCR lab). SPRI beads quantitatively recover the post-PCR product and preserve the starting amplicon concentration differences (as measured by qPCR).

All indexed libraries were pooled at equal volumes to preserve ratios of amplicon (and therefore the original sample concentration) between wells, and samples were loaded and sequenced on an Illumina NextSeq sequencing instrument.

### Results & Conclusions

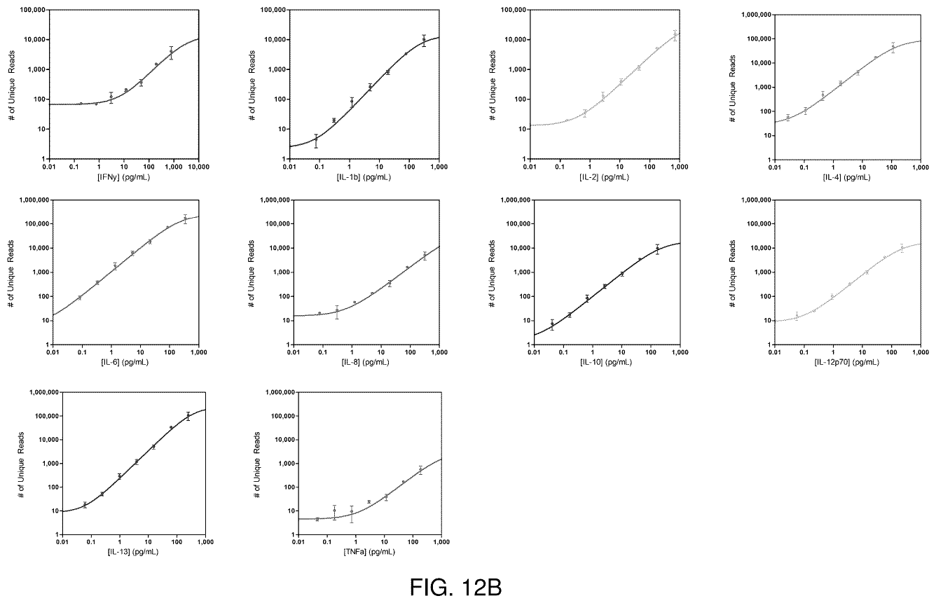

Concentration-dependent signal was collected for each analyte in this multiplex, as the number of unique amplicon reads (collected by NGS) corresponded directly with the input concentration of analyte (FIGS. 12A, 12B). Dynamic ranges for these newly developed PESD assays roughly spanned 3 orders of magnitude. Standard curves were collected for all 10 analytes and performance of the assay with NGS readout typically mirrored what is observed by other analytical methods (e.g., qPCR) (FIGS. 12A, 12B). That is, PESD assay performance was unaffected by readout type and rather was dictated by the antibody set selected for the assay.

### Example 4

This non-limiting example shows a Multiplexed (41-plex) PESD assay.

After selecting 41 analytes to be detected in a multiplex assay and selecting antibody pairs, unique barcode sequences, hybridization region sequences, calibrator controls, and reaction conditions, for each analyte, a 41-Plex PESD assay was performed. An example of two capture/detection oligo pairs is shown in Table 4.1.

TABLE 4.1

| Pair | | SEQ ID |
|---|---|---|
| ID | Sequence | NO: |
| A | CGGCAAATAGCGAAGACAGCNNNNNNNNTCACATACAGCTCGCG | 17 |
| | GTGAGGCGCTAGAGCATAGGNNNNNNNNTGAGTAACGACGCGAG | 18 |
| B | CGGCAAATAGCGAAGACAGCNNNNNNNNTAGACAGAAGCCAATT | 19 |
| | GTGAGGCGCTAGAGCATAGGNNNNNNNNACCATATCGAAATTGG | 20 |

“N” is a degenerate base (A, T, G, or C) randomly added during synthesis, these stretches make up the UMI regions(if used).

### Bead Coating and Barcoding

41 unique pairs of barcoded oligos (each pair contained a capture oligo and a detection oligo similar to those described in Examples 2 and 3) were designed and synthesized to go with 41 antibody pairs (capture and detection antibodies for 41 target analytes). Intermediate dilutions of biotinylated capture antibodies were prepared to make 20 μL of 50 μg/mL each in DPBS for analytes 1-41. Capture oligo and biotinylated bead tether for each of analytes 1-41 were previously annealed and stored at 10,000 nM. Intermediate dilutions were prepared at 100 nM in PBS.

1 m Pierce streptavidin (SA) coated beads were pulled from the vendor tube and washed in 1 mL of Diluent A diluent. The supernatant was discarded, and the beads were resuspended in Diluent A.

Clonal bead coating: for each of analytes 1-41, washed beads were added to a co-solution of biotinylated bead tether (annealed to capture oligo), and the assigned biotinylated capture antibody in Diluent A in a 1.5 mL centrifuge tube. Beads were coated at a ratio of 50 ng of biotin-capture antibody and 0.0625 pmol biotin-bead tether (annealed to capture oligo) per g of bead. Each tube was mixed on rotisserie for 1 hour at room temperature, then moved to 4° C. until used in the assay.

After coating, clonal beads were washed in 41 different wells using a KingFisher Apex sample purification instrument (Thermo Fisher Scientific, Inc.) via initial collection, and washing through 3 subsequent wells of 1 mL PBST via release and recollection. After the final wash, beads were released into PBST.

### Bead Pooling

Washed, clonal, barcoded beads were pooled in equal amounts and then distributed to each well of a 200 uL KingFisher Apex plate such that each well contained 0.1 ug of beads of each clonal bead type. Total bead mass in well was 4.1 ug.

### Detector Barcoding

Individual detection complexes (consisting of detection antibody with attached detection tether) for each assay in the 41-Plex were pre-hybridized to their uniquely barcoded detection oligos to produce barcoded detection antibody conjugates.

### Detector Pooling

After 1 h of detector pre-hybridization, detection cocktails were prepared by pooling the barcoded detector antibody conjugates.

### Calibrator Addition & Capture

A calibrator blend containing 41 calibrator proteins of analytes 1-41 was prepared in Diluent A and vortexed. An eight-point serial dilution series was prepared for the blended calibrator using 4-fold steps. Dilutions were prepared with Diluent A. Calibrator 8 (blank) contained Diluent C (containing: 2.0% bovine serum albumin, 2.1% potassium phosphate dibasic, 0.5% potassium phosphate monobasic, 0.04% kathon CG/ICP II, 2.0% sucrose, 0.022% Triton™ X-100, 0.3% IgG, 500 mM NaCl, and fetal bovine serum) only. Additionally, individual calibrators for each analyte were prepared at concentrations equivalent to Calibrator 2 (0.25× of the highest point in the calibration curve). 50 μL of blended calibrator at the appropriate concentration was added to the appropriate wells of a new KingFisher Apex 200 μL plate, individual calibrators were also added to the appropriate wells on the same KingFisher plate. 50 μL of assay diluent (Diluent C) was added to each well to bring the well volume to 100 μL total. Beads in their plate (prepared during ‘Bead Pooling’ step) were picked up by the magnetic head of the KingFisher and released into the plate containing blended and individual calibrators and incubated for 2 hours to permit analyte capture.

### Addition of Detectors

After capture, beads were washed to remove unbound calibrator and released in detector solution containing the pooled detection cocktail (containing each barcoded detector antibody conjugate) in Diluent B. Beads were incubated in detector solution for 1 hour.

Beads were then washed three times to remove unbound barcoded detector antibody conjugates. The first wash was with non-stringent MSD® Wash Buffer (PBS+0.05% polysorbate 20), while the subsequent two washes were with a stringent low-salt buffer (5 mM Phosphate+0.05% polysorbate 20) to denature any oligonucleotide hybridization.

### Extension and Strand Displacement

Washed beads were released into Klenow (3->5exo-) polymerase solution. Proximity extension and strand displacement were performed at RT for 1 hour. During this time, the enzyme extended the detection and capture oligos (bidirectional extension) and then displaced them from the tether oligos, releasing the double stranded product into the supernatant; extension and amplicon release occur in the same step during this process. A KingFisher Apex sample purification instrument was used to remove the beads from the extension supernatant containing the released full-length amplicon.

### Results

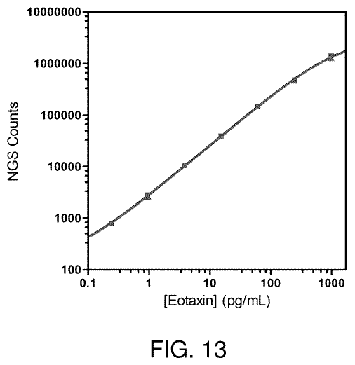

Assays were read-out and demultiplexed by NGS. 37 of 41 assays in this 41-plex performed with excellent concentration dependent signal response. Results for Eotaxin are shown in FIG. 13, and summary performance data from each of the assays is presented in Table 4.2: Average number of NGS reads from the highest calibrator (“TOC”) and blank for 6 replicates, Hill slope, and calculated lower limit of detection (LLOD). Two assays did not return any NGS reads, due to the omission of one of the assay components (e.g., barcoded oligonucleotide on the bead or detector).

FIG. 13: Example representative assay (Eotaxin) from 41-plex. Error bars represent the standard deviation of six replicates.

TABLE 4.2

| | LLOD | | Average | Average |
|---|---|---|---|---|
| Assay | (pg/mL) | Hillslope | TOC (counts) | Blank (counts) |
| Eotaxin | 0.02 | 0.98 | 1335228 | 144 |
| Eotaxin-3 | 0.05 | 1 | 1085112 | 167 |
| FLT-1 | 0.89 | 0.99 | 211194 | 213 |
| GM-CSF | 0.03 | 0.99 | 859697 | 209 |
| Granzyme A | 0.78 | 1.01 | 692962 | 361 |
| IFN-g | 0.52 | 1.02 | 90241 | 94 |
| IL-10 | 0.03 | 1 | 297479 | 219 |
| IL-12p70 | All wells at BG-Likely omission of calibrator | | | |
| IL-13 | Assay failure-Zero Counts-Likely omission of oligo | | | |
| IL-15 | 0.04 | 0.96 | 892206 | 218 |
| IL-17a | 0.23 | 1.01 | 1857758 | 649 |
| IL-18 | 0.17 | 1 | 410102 | 115 |
| IL-1a | 0.02 | 0.9 | 292921 | 98 |
| IL-1b | 0.09 | 0.99 | 678998 | 441 |
| IL-2 | 0.06 | 0.98 | 352072 | 70 |
| IL-21 | 0.14 | 0.98 | 958322 | 160 |
| IL-22 | 0.16 | 0.96 | 1641908 | 850 |
| IL-27 | 24.85 | 1.05 | 506902 | 4079 |
| IL-29 | 0.36 | 1.03 | 608797 | 112 |
| IL-2Ra | 0.07 | 0.99 | 755970 | 94 |
| IL-31 | 0.1 | 0.98 | 1623476 | 1046 |
| IL-33 | 1.55 | 0.99 | 358719 | 720 |
| IL-4 | 0.01 | 0.99 | 914604 | 436 |
| IL-5 | 0.08 | 0.99 | 540225 | 257 |
| IL-6 | 0.01 | 0.98 | 910327 | 100 |
| IL-7 | 0.25 | 1.01 | 525906 | 360 |
| IL-8 | 0.05 | 1.02 | 836163 | 52 |
| IP-10 | 0.01 | 1 | 918322 | 113 |
| MCP-1 | 2.95 | 1.17 | 789781 | 3465 |
| MCP-4 | 0.04 | 1.04 | 537561 | 89 |
| MDC | 2.17 | 0.99 | 107838 | 122 |
| MIP-1a | 1.88 | 1.4 | 211095 | 102 |
| MIP-1b | 0.02 | 1.03 | 1722360 | 140 |
| MIP-3a | 0.07 | 1.03 | 911593 | 333 |
| PIGF | 0.05 | 1.02 | 913308 | 282 |
| TARC | 0.26 | 1.04 | 467788 | 98 |
| Tie-2 | 4.98 | 0.99 | 194381 | 89 |
| TNF-a | 0.87 | 1.13 | 438189 | 199 |
| TNF-b | 0.01 | 1 | 852049 | 66 |
| VEGF-A | 0.11 | 1.01 | 371273 | 152 |
| VEGF-D | 0.35 | 1.11 | 1357297 | 232 |

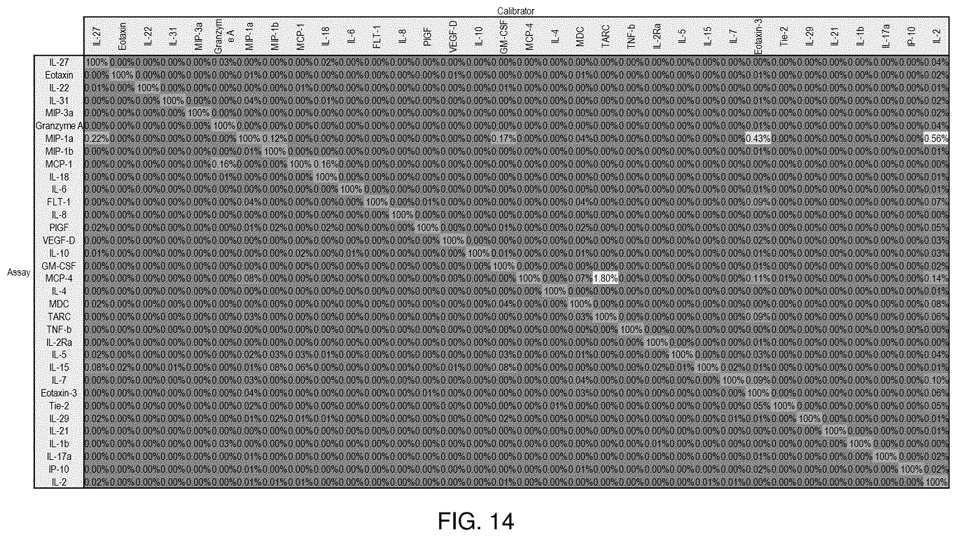

In addition to the 41-plex assay containing mixes of all analytes in the assay wells, the pool of 41 clonal bead types and pool of 41 barcoded detectors were tested against each individual analyte. This allowed measurement of 1) the comparative performance of any assay in single-plex vs multiplex form, and 2) biological cross reactivity for any analyte in an incorrect assay sandwich; for example, IL-4 analyte captured and detected by IFN-γ antibodies. Through this study, it was shown that the singleplex and multiplex quantitation of any individual analyte are very similar. The calculated ratio of individual versus combined/blended analyte signal is presented in Table 4.3 and in FIG. 14, and for most analytes the value was between a tolerable range of 0.8-1.2×. The matrix of off-target interactions and their relative signals is presented. Analytes are ordered against their corresponding antibody sets, creating a diagonal alignment of correct antibody-analyte pairs. Off-target (i.e., off-diagonal) signal generation was calculated relative to the specific signal generate for each assay; by definition the on-target specific interaction is reported as 100%. Nearly all assay combinations produced very low (and nearly undetectable) amounts of cross-reactivity at a threshold of <0.01%. There were, however, a select number of off-target interactions that were uncovered by this method. These off-target interactions included: TARC in the MCP-4 assay, and IL-2 and Eotaxin-3 in the MIP-la assay. This demonstrates the use of PESD for high throughput screening of antibody-antigen cross reactivity.

FIG. 14. Singleplex versus multiplex signal recovery, and cross reactivity matrix for all assays in this 41-plex.

TABLE 4.3

| Assay | Single calibrator/multiplex ratio |
|---|---|
| Eotaxin | 0.92 |
| Eotaxin-3 | 0.71 |
| FLT-1 | 1.12 |
| GM-CSF | 0.84 |
| Granzyme A | 1.05 |
| IFN-g | Prep error |
| IL-10 | 0.97 |
| IL-12p70 | |
| IL-13 | |
| IL-15 | 0.92 |
| IL-17a | 0.93 |
| IL-18 | 1.22 |
| IL-1a | Prep error |
| IL-1b | 0.94 |
| IL-2 | 0.85 |
| IL-21 | 0.81 |
| IL-22 | 0.82 |
| IL-27 | 0.98 |
| IL-29 | 0.94 |
| IL-2Ra | 0.96 |
| IL-31 | 0.83 |
| IL-33 | 1.03 |
| IL-4 | 0.86 |
| IL-5 | 0.99 |
| IL-6 | 1.25 |
| IL-7 | 0.8 |
| IL-8 | 1.08 |
| IP-10 | 0.88 |
| MCP-1 | 0.93 |
| MCP-4 | 0.93 |
| MDC | 0.6 |
| MIP-1a | 0.83 |
| MIP-1b | 0.8 |
| MIP-3a | 1.29 |
| PIGF | 0.83 |
| TARC | 0.89 |
| Tie-2 | 0.86 |
| TNF-a | 1.02 |
| TNF-b | 0.85 |
| VEGF-A | 1.39 |
| VEGF-D | 0.89 |

### Example 5

This non-limiting example shows a design and testing of orthogonal unique hybridization oligonucleotides for multiplex PESD.

Although off-target interactions (e.g., mismatched forward (detection) and reverse (capture) barcodes from different assays) can be filtered and removed during NGS data analysis, there can be a penalty for these mismatches due to the limited number of amplicon reads available during a sequencing run. Thus, to reduce the number of discarded reads and maintain greater depth of specific reads in large scale multiplexing, forward and reverse oligonucleotides corresponding to the same analyte in a multiplex pool can be paired via a complementary hybridization sequence that is unique only to the antibody pair for that analyte. These are designated as “unique hybridization overlaps,” as opposed to “common hybridization overlaps” in which the oligos on the antibody pairs for all analytes share the same hybridization sequence.

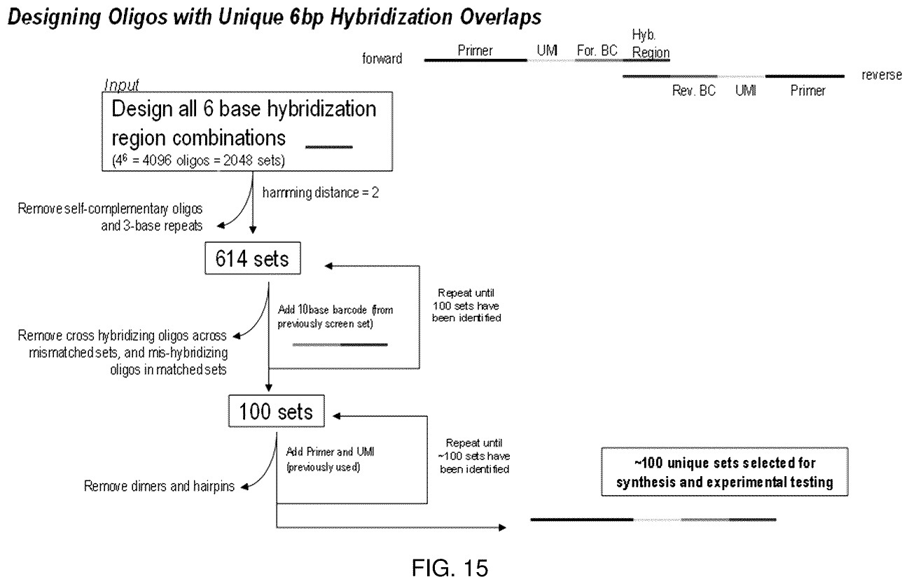

Due to the unique nature of each hybridization sequence, it was theorized that the extension efficiency and amplicon generation might vary based on sequence-specific properties such as GC content, base repeats, terminal base identity, nearest neighbor effects, etc. Further, despite the most stringent, predictive in silico screening of oligonucleotide interactions, there is still possibility for off-target interactions between detection and capture oligonucleotides belonging to different antibody sets. Thus, experimental screening of oligonucleotides with unique hybridization overlaps was performed. First, 96 sets of oligonucleotides for use in a PESD assay (capture and detect oligos) were designed with unique hybridization overlaps that were 6 bp in size. The hybridization region of 6 bp was selected as a compromise between increase in sequence diversity and reduction of non-specific pulldown of antibody through overly strong oligonucleotide-oligonucleotide interactions. For example, a six base hybridization system can have up to 4,096 unique sequences (and 2,048 complementary sets) versus only 1,024 unique sequences (512 complementary sets) if there are only five base pairs in the hybridization region. Additionally, the modest increase in hybridization energy between 5 bp and 6 bp can help to marginally stabilize the Watson-Crick base pairing interactions between forward and reverse strands and diminishes variable effects such as GC content, base repeats, terminal base identity, nearest neighbor effects on the extension efficiency in PESD. A generalized scheme for designing oligonucleotides with 6 bp hybridization overlaps is shown in the FIG. 15. This method was utilized to generate a minimum of 100 sets of paired PESD oligonucleotides, of which 96 sets were ultimately screened in PESD assay format to identify sets that were suitable for use in a PESD multiplexing.

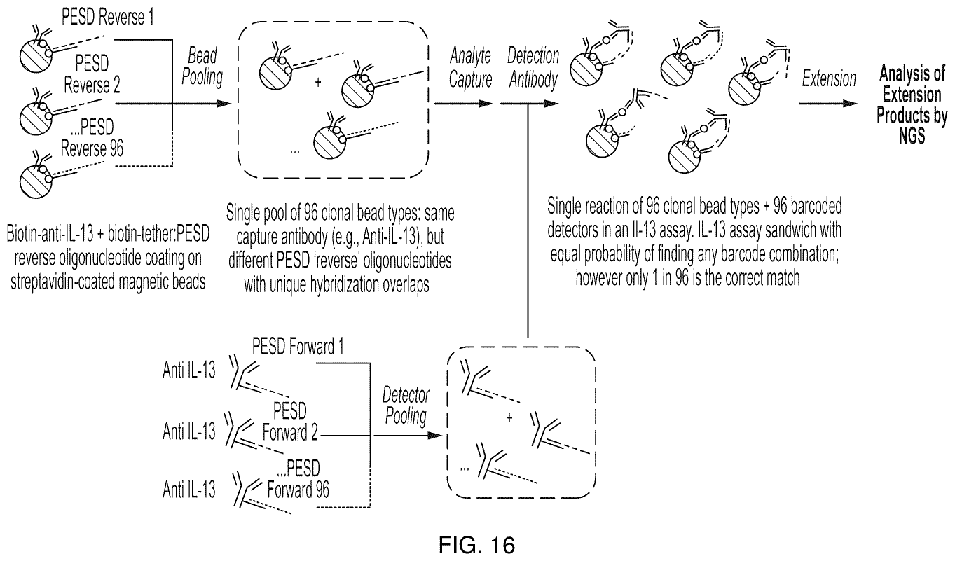

All oligo sets were tested in the presence of all 95 other non-matching sets in a PESD immunoassay format, which was then read out by NGS (FIG. 16). In this way, off-target interactions or poorly performing unique oligonucleotide sets were quickly identified and potentially removed from future consideration in PESD.

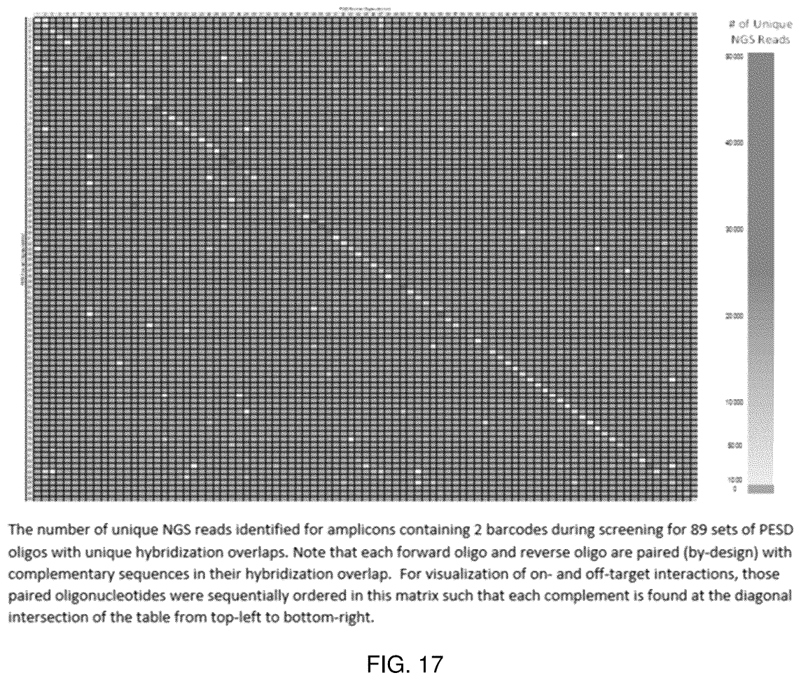

Here, 89 sets of PESD oligonucleotides were screened in the presence of all other oligonucleotide pairs in a single reaction well. The number of unique reads for each set is reported in FIG. 17. In this table, oligonucleotides are sequentially ordered against their designed complement, thus interactions on the diagonal of this table are for matched oligonucleotide interactions and should be completely orthogonal to the presence of other oligonucleotides. A majority of the specific signal in this experiment was found in the properly matched complementary oligonucleotides with unique hybridization overlaps. However, there were a number of off-target interactions identified by this screening method that were not initially predicted. Through this high-throughput screening method, the orthogonality of each unique oligonucleotide set, as well as oligonucleotide sets that display poor extension efficiency, were rapidly identified.

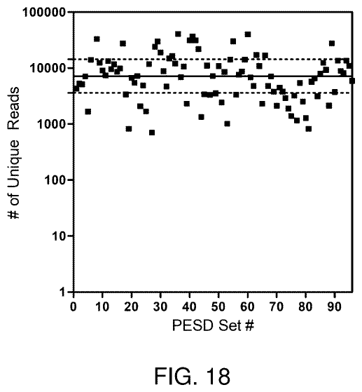

To avoid overrepresentation of amplicons derived from a single assay/analyte in a given sample, it is desirable to create signal balance across all assays in any multiplex of appreciable size. Although these assays can be de-multiplexed and are fully calibrated, it is advantageous to approximately balance the extension efficiency for each unique set of PESD oligonucleotides assigned to any group of assays. Based on the assumption that the number of NGS reads in this system corresponds directly to the extension efficiency of unique oligonucleotide sets, the number of NGS reads can be used to make predictions about the performance of any oligonucleotide set in a PESD assay. Upper and lower bounds of +/−2-fold above the median number of NGS reads was a tolerable range for oligonucleotide sets with unique hybridization overlaps (shown as the two dashed and one solid line in FIG. 18). By this metric, 52 of the 96 exemplar oligo sets fell into this tolerable range and could be used in PESD experiments without imparting significant (e.g., greater than ±2-fold) bias on any extension reaction. Those which fall outside of this range may be re-evaluated for use in PESD studies where the anticipated biological concentration of a given analyte is either disproportionately high or low and respective suppression or enhancement extension efficiency is desired to balance that specific amplicon within a pool of other amplicons in that multiplex.

FIG. 18: All PESD oligo sets with unique hybridization overlaps are plotted along with the # of Unique NGS reads. Using this method, rapid determination of hybridization sequences that over- or under-perform in this group can made and those sets can be omitted, re-purposed, or discarded from future consideration. Dashed lines on the plot indicate +/−2-fold range of the median # of NGS reads across the whole group.

### Example 6

This non-limiting example shows a PESD method for antibody screening to identify capture and detect antibody sandwich pairs, and the design and testing of oligonucleotides with common hybridization overlaps.

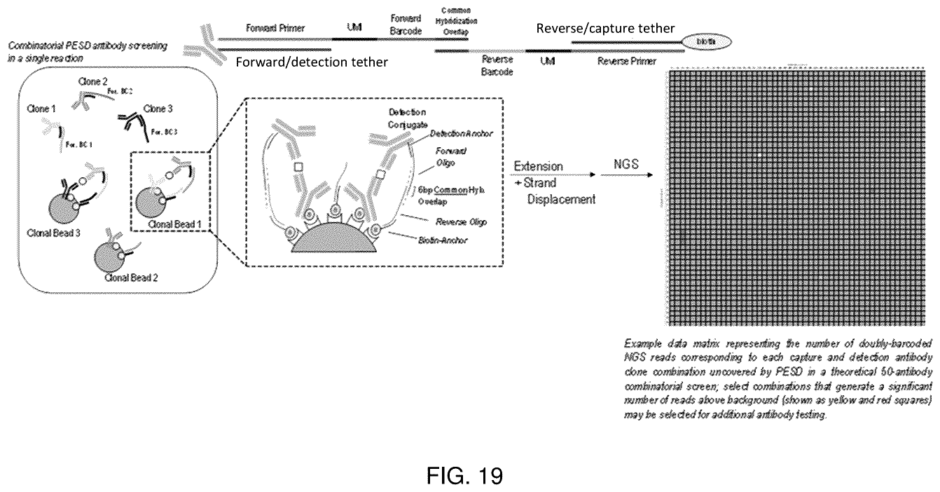

Additional utility of PESD is demonstrated in the rapid, high throughput screening of antibody pairs for use in 2-antibody immunoassays. Similar to the use of PESD for high multiplexing against hundreds or thousands of analytes in a single sample and de-multiplexed through NGS, this technology could be applied to screen hundreds of antibodies in capture and detection positions (e.g., thousands of 2-antibody combinations) against a single analyte in a sample, with the highest affinity pairs being de-coded and quantified by NGS. Conventionally, immunoassay development typically requires dozens to hundreds of antibodies be screened in pairwise fashion in single wells and read out by colorimetric, fluorescent, chemiluminescent, or electrochemiluminescent signal generation. For example, a primary screen of 100 antibodies would thus require an evaluation matrix of 100 antibodies in both capture and detect positions; 100 capture antibodies×100 detection antibodies=10,000 wells per sample. The scheme shown in FIG. 19 displays the PESD antibody screening workflow, along with a theoretical data matrix.

Each antibody in this screen is first prepared in two conjugated forms: 1) biotinylated and 2) forward/detection tether oligo modified. The biotinylated form is coated onto streptavidin-coated beads along with a biotin-reverse tether:reverse/capture barcoded dsDNA duplex. The tether oligo-modified form is hybridized to a forward/detector barcoded oligo. Clonal beads are prepared for all capture antibodies, then pooled together to make a single bead mixture containing all capture antibodies in equal representation. Similarly, after barcoding, the detection conjugates are pooled together to make a single detection cocktail containing all detection antibodies in equal representation. Beads are distributed to wells for analyte capture. For rigorous screening of the antibody pairs, a variety of analytes may be tested across many wells, for example, recombinant protein analytes from many sources (at a variety of concentrations) and/or native samples from a variety of biofluids (at different dilutions). Following analyte capture, the clonal beads are washed to remove unbound protein, then the detection cocktail is added to bind to any accessible protein epitopes in their bead-bound state. Dilutions of the detection cocktail may be performed across wells in order to elucidate information about the relative binding affinity of any particular antibody:antigen interaction. Following detector binding, a final wash step is performed to remove unbound detector, followed immediately by enzyme extension and displacement of oligonucleotides held in proximity through the 2-antibody immunoassay sandwich. Library preparation and NGS of the resulting amplicons may reveal the relative frequency of each 2 barcode (and thus 2 antibody) permutation in this assay, information about limits of detection, affinity, and/or specificity for the analyte.

All barcoded oligonucleotide sets in this embodiment of PESD feature a common, short hybridization region sequence. Due to the combinatorial nature by which this antibody screening is performed, any detection conjugate-associated oligonucleotide is allowed to freely interact with (and extend upon) any bead-associated oligonucleotide, assuming that they are bound in proximity through complexation with the analyte in a 2-antibody “sandwich”. This is a departure from some embodiments of the high multiplexing PESD oligonucleotide design, which features paired forward and reverse oligonucleotides on the detector-side and bead-side of the assay, respectively, which are designed to only interact with each other through a hybridization overlap that is unique to their respective assay.

In order to eliminate bias in the selection of antibody permutations for a given antibody screen, extension efficiency for all oligonucleotide combinations can be made to be comparable to each other and without bias from the specific oligonucleotide sequence. The primer regions and common hybridization sequence between forward and reverse oligos can be identical across all sets. Further there is a random eight nucleotide unique molecular identified (UMI) in each oligonucleotide. However, the forward and reverse barcodes in the forward/detector and reverse/capture oligonucleotides are entirely unique. Thus, variability in extension efficiency between oligonucleotides in an assay derives from the barcodes themselves.

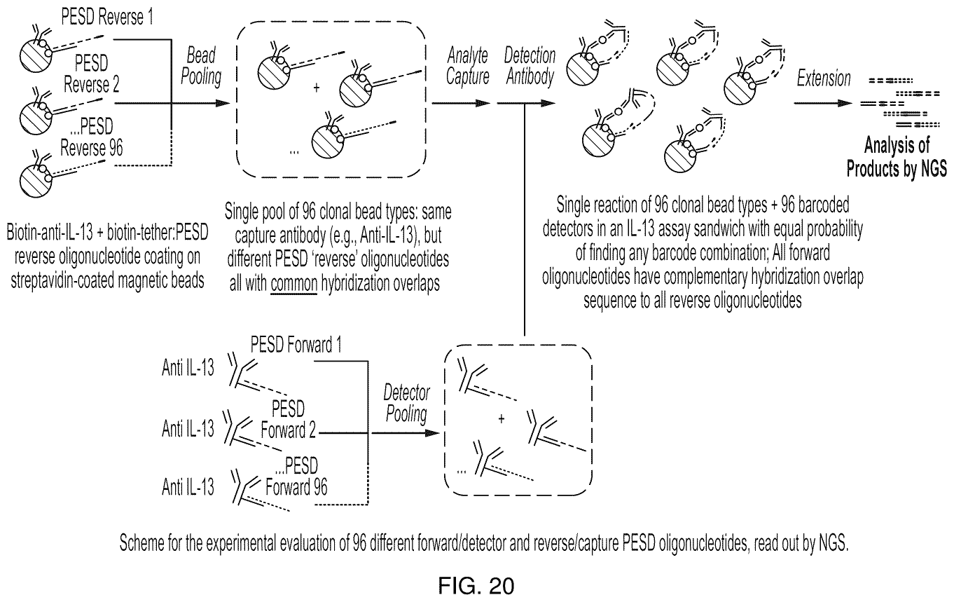

Although the hybridization sequence, binding energy, is the same for all oligonucleotides, the efficiency of adding the next base during extension can be sequence dependent. To address this, 96 “forward” and 96 “reverse” oligonucleotides were designed and then screened against each other in a single immunoassay to identify differences in extension efficiency, as well as rapidly identify off-target binding interactions introduced by the unique barcodes on either strand (FIG. 20).

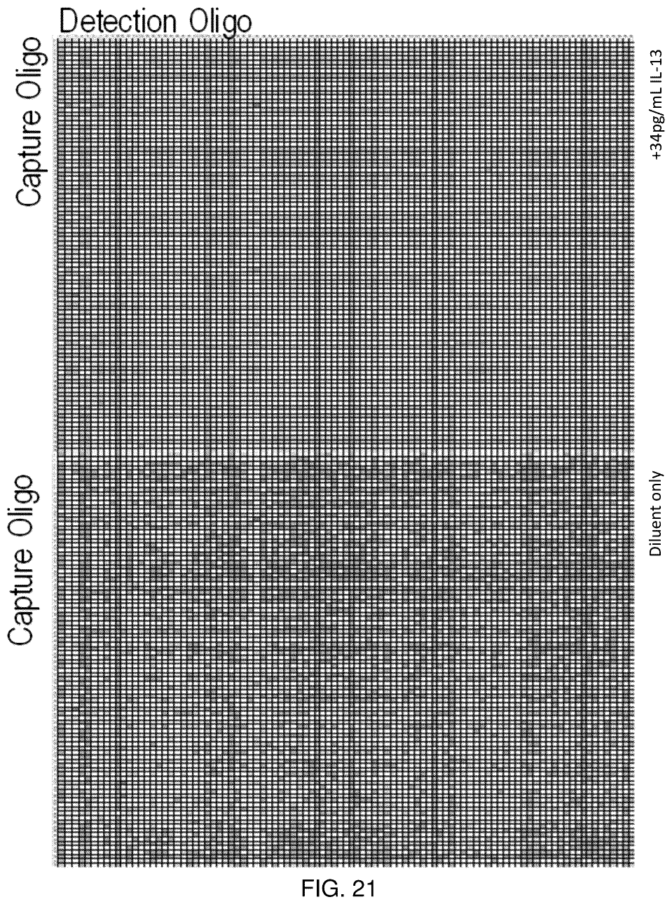

Representative data from an NGS study are shown in FIG. 21. Clear patterns can be observed from this data that indicate 1) oligonucleotide-specific underperformance (top pane, green squares down a column or across a row), which is the result of a hairpin at the 3′ end that was not initially discovered during in silico screening and is adversely affecting extension, or 2) pair-specific overperformance (top and bottom panes, isolated red squares) which are occurring because of off-target oligo-oligo interactions that were not initially discovered during in silico screening and is pulling down detector on beads to generate analyte-independent signal. In scenario 1, that oligonucleotide can be removed from consideration to prevent the undue bias that its inclusion may impart on an antibody screen. In scenario 2, only one of those oligonucleotides (forward or reverse) can be removed to prevent the unintended pairing of those two partners. Removal of one will allow for use of the other.

FIG. 21: Representative data matrix for screening 96 different forward barcoded oligonucleotides against 96 different reverse oligonucleotides in a PESD system. Note all oligonucleotides have a common hybridization sequence such that any forward strand can hybridize and extend along with any reverse strand if held in proximity confinement. Color scheme: Green—low/no NGS reads, Red=high # of NGS reads). Some oligonucleotides are inefficient when paired with any partner (as visible by entire rows or columns that was green), which indicates that there is some sequence dependent effect suppressing the extension efficiency. Post hoc structural analysis of those specific sequences revealed that all of these oligonucleotides have 3′ hairpins that are blocking extension. Indicating that this PESD NGS screening tool can be used to filter out deficient sequences/structures that were not initially filtered out in silico.

From this screening of 96 different forward and reverse barcoded oligonucleotides, oligonucleotides (forward and reverse) that are appropriate for PESD-based antibody screening can be selected.

### Example 7

This non-limiting example shows a singleplex PEA assay with immunoPCR.

These experiments were performed to measure performance of a single PEA assay (e.g., IFNy) in the presence of the other bead types (e.g., a pool of clonal beads) and other detectors (e.g., a pooled detection cocktail).

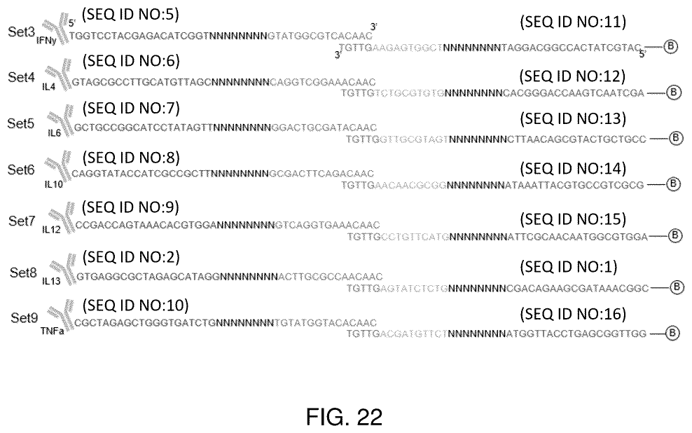

To create the extension sets for each of the seven analytes, antibodies from the MSD® V-PLEX Proinflammatory Panel 1 Human Kit or the S-PLEX Proinflammatory Panel 1 Human Kit (Meso Scale Diagnostics, LLC.) to seven target analytes were conjugated to seven unique oligo sets with unique primer and barcode regions, but a common 5 bp hybridization overlap to produce the sets shown in FIG. 22.

Each of the seven assays was run as a singleplex assay and then as a “multiplex” assay in the presence of pooled clonal beads and a pooled detection cocktail but only one calibrator type (e.g., an IFNγ calibrator was included in the IFNγ “multiplex” assay). All data was read out by qPCR. An exemplary experimental configuration is shown in Table 7.1.

TABLE 7.1

| | IFNγ Assay | |
|---|---|---|
| | Singleplex | Multiplex |
| Beads | IFNγ-Set3 | Pooled Clonal Beads |
| Calibrator | IFNγ | IFNγ |
| Detector | IFNγ | Pooled Detection Conjugates |
| qPCR Primer Set | Set3 | Set3 |

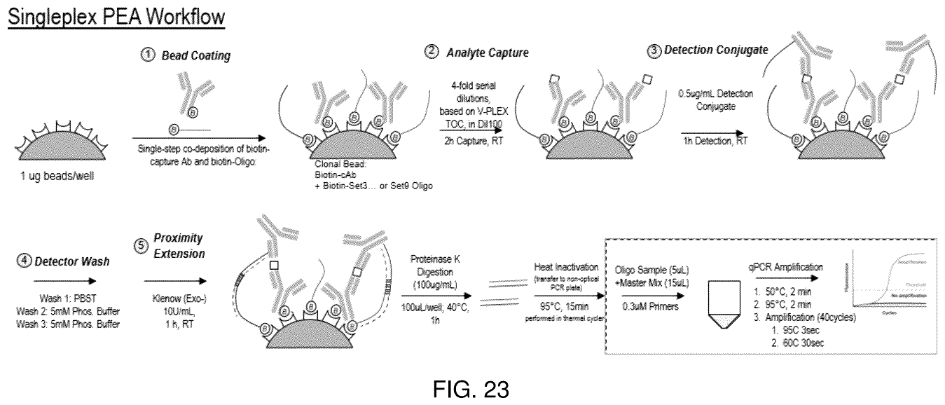

The singleplex PEA workflow was performed as shown in FIG. 23. Briefly, for each analyte, streptavidin coated beads in the wells of a multi-well microplate were coated in a single step co-deposition of biotinylated capture antibody and biotinylated capture oligonucleotide (FIG. 23 step 1). For each analyte, the coated clonal beads were then incubated with their corresponding analyte (4-fold serial dilutions) for 2 hours at room temperature. 0.5 μg/mL detection conjugate consisting of detection antibody plus detection oligonucleotide was then added to the beads and incubated at room temperature for 1 hour (FIG. 23 step 3). Beads were subsequently washed three times to remove unbound detection conjugate (FIG. 23 step 4)—wash 1: PBST/wash 2:5 mM phosphate buffer/wash 3:5 mM phosphate buffer. Beads were released into 10 U/mL of (exo-)Klenow and incubated at room temperature for 1 hour permitting proximity extension to occur (FIG. 23 step 5). During this time, the enzyme extends the detection and capture oligos (bidirectional extension). 100 μL of 100 μg/mL Proteinase K was then added and the solution was incubated for 1 hour at 40° C. to release the double stranded amplicons into solution. After heat inactivation (95° C. for 15 minutes), the oligos were combined with qPCR master mix and 0.3 μM primers and qPCR amplification was performed (2 minutes at 50° C., 2 minutes at 95° C., then 40 cycles of amplification with each cycle comprising 3 seconds at 95° C. followed by 30 seconds at 60° C.).

### Multiplexing

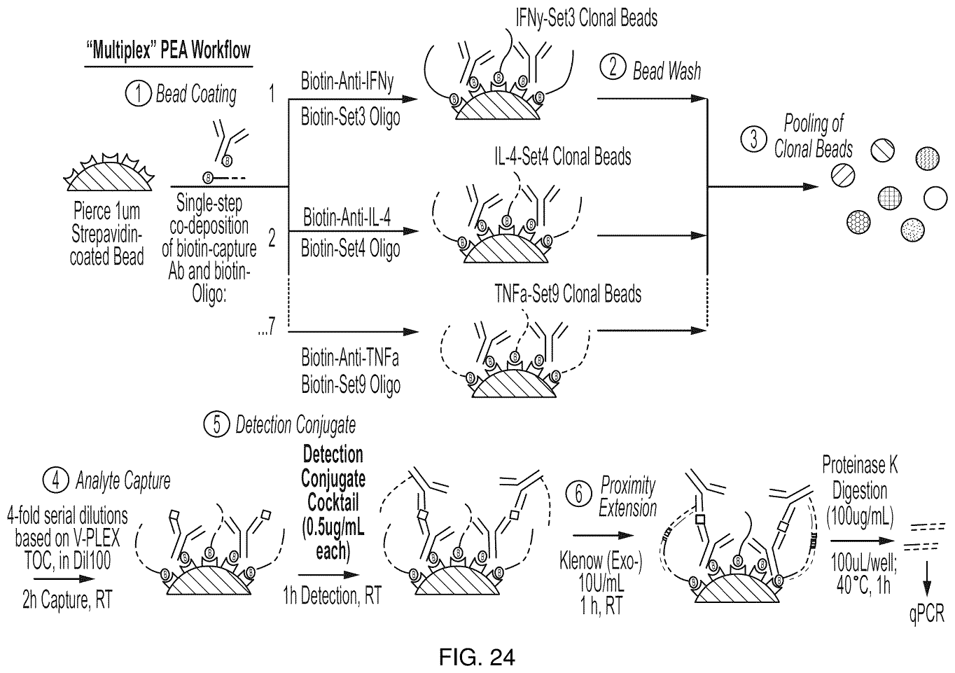

The multiplex workflow was performed as shown in FIG. 24. Briefly, for each of the analytes, Pierce 1 m streptavidin coated beads were coated in a single step co-deposition of biotinylated capture antibody and biotinylated bead oligonucleotide (FIG. 24 step 1). Each of the seven sets of coated clonal beads was washed (FIG. 24 step 2) and all sets were pooled (FIG. 24 step 3). The pooled bead mixture was then incubated with one calibrator analyte (4-fold serial dilutions in Diluent A) for 2 hours at room temperature (FIG. 24 step 4). This was done for each of the seven analytes. A cocktail containing 0.5 μg/mL of each of the seven detection conjugates (each consisting of detection antibody plus detection oligonucleotide) was then added to the beads and incubated at room temperature for 1 hour (FIG. 24 step 5). Beads were washed and combined with 10 U/mL of (exo-)Klenow and incubated at room temperature for 1 hour permitting proximity extension to occur. 100 μL of 100 μg/mL Proteinase K was then added and the solution was incubated for 1 hour at 40° C. to release the double stranded amplicons into solution (FIG. 24 step 6). After heat inactivation (95° C. for 15 minutes), the amplicons were combined with qPCR master mix and 0.3 μM primers and qPCR amplification was performed (2 minutes at 50° C., 2 minutes at 95° C., then 40 cycles of amplification with each cycle comprising 3 seconds at 95° C. followed by 30 seconds at 60° C.).

### Results and Conclusions

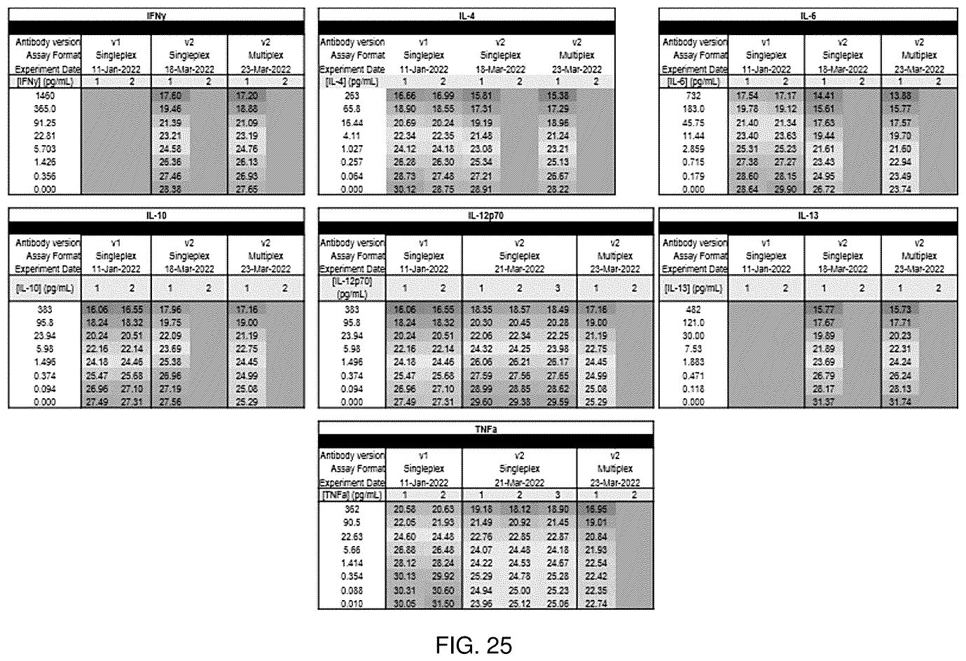

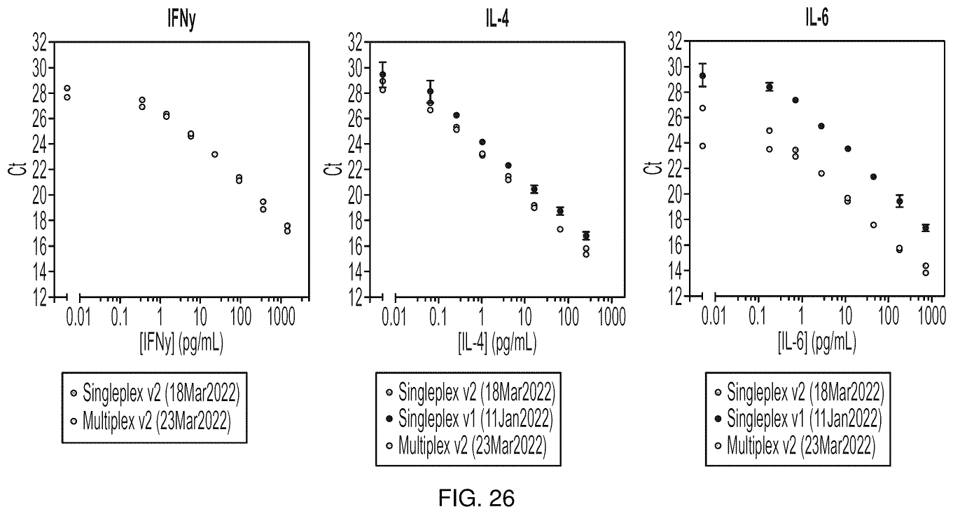

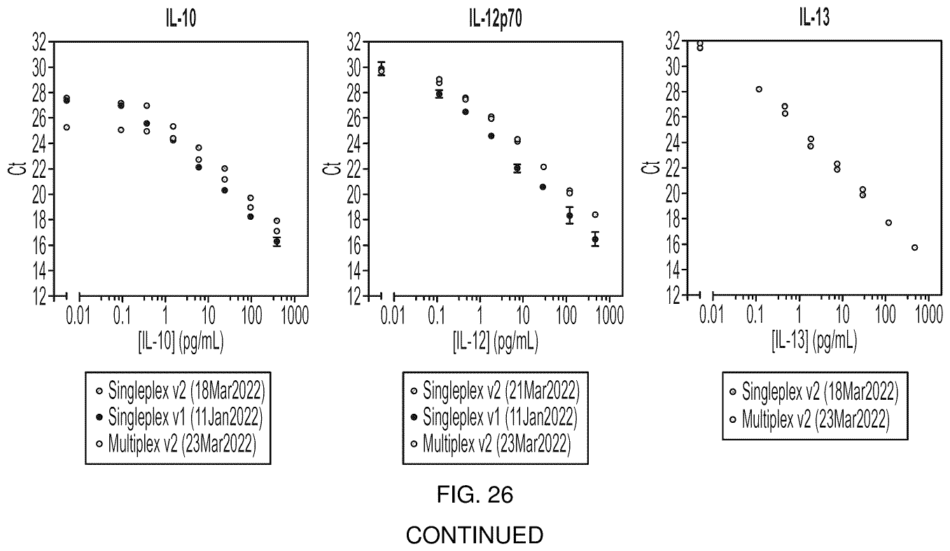

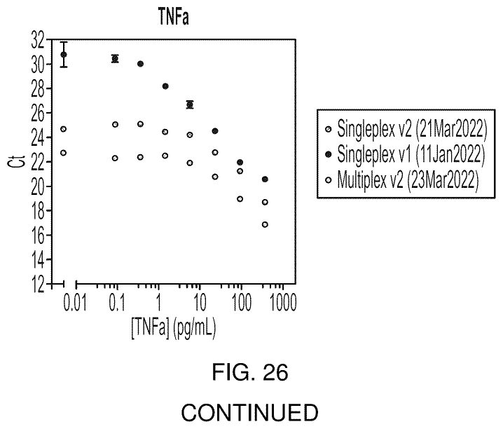

As can be seen from the data in FIGS. 25 and 26, PEA Assays performed similarly, whether in a singleplex or in a mock-multiplex system in the presence of other clonal bead types and detection antibody conjugates. The PEA workflow was robust and performed similarly with multiple pairs of antibodies for a particular target. In addition, each assay typically exhibited good reproducibility between technical replicates. However, the performance of a PEA system, much like any assay, is limited by the quality of antibodies used.

### Example 8

This non-limiting example shows analysis of extension products generated by PESD (or PEA).

### Library Preparation Methods

Reaction product mixtures are prepared for sequencing though a multistep process. First the total amount of amplifiable DNA (amplicon) in each mixture can be measured using a qPCR reaction with primers targeting the primer/tether sites. This gives a first level of quality check to make sure that the PESD reaction occurred as expected. Samples may be normalized at this point based on the amount of total amplicon in each well. A portion of the reaction product is transferred to a PCR reaction mixture (adapter PCR) using tailed primers (adapters) for Illumina sequencing. This reaction mix can contain one or more synthesized analogs of the PESD products (spike-ins). These spike-in oligos can serve two purposes. One is that they can contain one or more UMI sequences—typically 8-16 random bases, which allows determining the average sequencing depth. A depth of ˜10 will result in nearly every molecule in the reaction product mixture being sequenced. For high multiplex assays, the sequencing depth may be less due to the limited number of total reads in each sequencing kit. In this case average sequencing depth below 1 is typically desired, and again the UMIs allow the depth to be ascertained. UMIs could also be incorporated into each of the PESD products by including a string of random bases in either the forward or reverse oligos, or both. This was demonstrated using an 8-base UMI in each of the forward and reverse oligos. Having a UMI in the product allows determination of the number of copies of each starting molecule that were sequenced. This may in turn allow determination of whether PCR-bias is causing over-representation of certain sequences in the library. The UMIs can be used to correct for PCR bias by deep sequencing and eliminating PCR duplicates to only count each molecule once. In some embodiments, no significant PCR-induced bias was observed and UMIs may be unnecessary for correcting for bias. The UMI from each forward and reverse oligo can be removed to reduce mispriming. Products of incorrect length occurred due to mispriming within the UMI region. Eliminating the UMI region reduced the mispriming considerably.

The second purpose of the spike-ins is to allow correction for process variations during the library preparation. If the spike-ins are added to the PCR master mix during the adapter PCR step, an equivalent number of molecules will be added to all samples and thus all samples should produce the same number of sequencing counts for the spike-ins after analysis. Variations in this number, which result from inaccuracy in pipetting during PCR setup or sample transfers, or variation in the yield of PCR cleanup steps, can be corrected in the post sequencing analysis to improve the accuracy of the assays.

After adaptor PCR, the unicorporated primers are preferably removed. This is typically accomplished by using SPRI beads (e.g. AmpureXP or Promega ProNex) and processed using the KingFisher bead washer. A second round of PCR is used to index each sample by adding unique dual index adapters (another set of tailed primers) to each sample. The result is to add specific sequences to each amplicon that can be used to demultiplex the samples after sequencing. Any suitable indexing methods can be used. Once indexed, samples can be recombined before running a second round of primer cleanup, again using SPRI beads. The size and concentration of the resulting product mixture (sequencing library) is measured using gel electrophoresis (e.g. TapeStation) and/or an assay such as the QuBit Picogreen assay. One or more libraries is then sequenced by short read sequencing according to the manufacturer's guidance. For example, Illumina NextSeq2000 or other short read sequencers can be used.

### Data Analysis

The NextSeq2000 performs demultiplexing of samples based on the index adapter sequences. Usually additional adapter trimming is used for sequences that failed the automatic demultiplexing. For each sample a separate FASTQ file is generated and analyzed on its own. The forward and reverse primers are located and trimmed. The length distribution of the remaining sequence is analyzed to determine the amount of mispriming. At very low or zero analyte levels (e.g. blank sample), the number of sequences that are the correct length will be low, ˜20% in the 40-plex. This is because there are no properly formed molecular sandwiches, only non-specific binding of detectors, nonetheless, the correctly paired forward and reverse oligos form product much more efficiently than the mispaired ones. Once an appreciable level of analyte is present, the majority of sequences result from properly formed oligos (90% at top of curve, 80% at midcal for 40-plex with UMIs—this will improve when UMIs are eliminated) because the correct detectors are held in proximity much more often due to antibody specificity, in addition to the increased efficiency of the correct oligo pairing relative to incorrect. Thus in most practical cases, only a small fraction of the total sequences needs to be eliminated due to mispriming (<20%, and having no UMI can reduce this further).

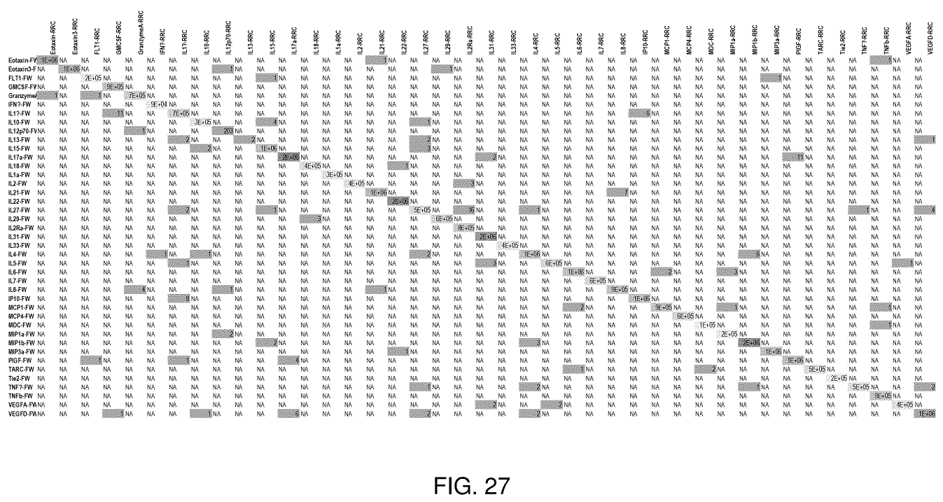

Once the wrong-length sequences are eliminated, the barcodes are located and matched to a lookup table to identify their corresponding antibody specificities. Sequences with correctly paired barcodes are counted while those with mispaired barcodes are eliminated. Again sequences with mispaired barcodes represent a small fraction of the remaining sequences due to the orthogonality of the hybridization regions. The number of counts for a particular mispaired sequence can depend on several factors including the concentration of the target analyte for the capture bead involved, the level of non-specific binding of the off-target antibodies to this bead, relative efficiency of the on- versus off-target extension. At high levels of target analyte, the observed level of mispairing is vanishingly small relative to the properly paired sequences (˜1 ppm). As the target analyte concentration approaches the limit of detection the relative ratio of off-target products is greater but still very low. At the lowest calibrator, only 4 out of 41 assays had any detectable mispaired products and these were ˜1 part in 1000 relative to properly paired sequences. Even in the blank where the number of properly paired sequences was low, representing the true assay background, the mispaired sequences were rare and those that occurred were around 1 part in 200 relative to correctly paired sequences, demonstrating the high degree of orthogonality between oligos (FIG. 27).

The sequences with correctly paired barcodes are treated as the signal and further analyzed. Calibration curves are generated from serially diluted calibrator samples. Typically an 8 point dilution series of calibration samples can be included in an experiment to enable accurate quantitation of all analytes. A 4PL fit is performed on the signal versus concentration for each analyte. This fit is then used to calculate the concentration of that analyte in all other samples in the run.

Sequencing depth can affect the accuracy. The relationship between the concentration of a specific species in the sequencing library and the number of counts produced is expected to approximate a hypergeometric distribution, thus the variance is approximately the mean. At 100 counts, the coefficient of variation due to sequencing noise alone would be 10%. 1% sequencing noise could be achieved at 10,000 counts per assay.

### Example 9

This non-limiting example shows a PESD assay (FIGS. 29A, 29B).
1. A first conjugate is prepared by annealing a first splint oligonucleotide to a first analyte-binding moiety that is attached to a first tether oligonucleotide (FIG. 29A). The first splint oligonucleotide includes a tethering region that hybridizes to the first tether oligonucleotide, a barcode sequence and a 3′ hybridizing region.
2. A second conjugate is prepared by annealing a second splint oligonucleotide to a second analyte-binding moiety that is attached to a second tether oligonucleotide (FIG. 29A). The second splint oligonucleotide includes a tethering region that hybridizes to the second tether oligonucleotide, and a 3′ hybridizing region that is complementary to the 3′ hybridizing region of the first splint oligonucleotide. The second splint oligonucleotide may or may not include a barcode sequence.
3. The first conjugate and the second conjugate are combined with a sample, and incubate at room temperature.
4. Add 3→5′ exo-Klenow fragment with strand displacement activity. The polymerases extends the splint oligos (bidirectional extension) and then displace them from the tethers, releasing the double stranded product; extension and amplicon release occur in the same step (FIG. 29B).
5. Separate released product from conjugates.
6. Analyze released product using qPCR and/or sequencing.

### Example 10

This non-limiting example shows use of blocker oligonucleotides in a PESD assay to reduce off-target extension products and/or analyte-independent on-target interaction between splint oligonucleotides.

A non-limiting example of a paired combination of splint oligonucleotides and associated tether oligonucleotides were designed as shown in FIG. 31A. Each splint oligonucleotide included, from 5′ to 3′, a 5′ tethering region, a blocker stabilization region, a barcode region, a second blocker stabilization region, and a hybridization region. One of the tether oligonucleotides was attached to an antibody (“detection antibody”) that binds to an analyte, and the other tether oligonucleotide was attached to a bead (“B”) that is further coated with another antibody (“capture antibody”) that binds the same analyte as the detection antibody. The splint oligonucleotides can hybridize to each other over their respective hybridization regions, when the splint oligonucleotides of the paired combination are in proximity to each other.

Non-labile and labile blocker oligonucleotides that hybridize to different portions of the splint oligonucleotides were designed as shown in FIG. 31B. The non-labile blocker oligonucleotides (also called barcode blocker oligonucleotides) can hybridize to the barcode region of one of the splint oligonucleotides and optionally to the blocker stabilization region immediately 5′ of the barcode region. When hybridized, the barcode blocker oligonucleotides can prevent or reduce non-specific (or off-target) interaction of another splint oligonucleotide with the barcode region. This can reduce the amount of misprimed or mismatched products upon extension, and reduce uninformative sequencing reads that would need to be discarded.

The barcode blocker oligonucleotide remains hybridized to the splint oligonucleotide during the PESD assay, and is effectively removed only when a strand displacing polymerase successfully extends a paired combination of splint oligonucleotides that are hybridized to each other to make an on-target extension product. If used, the blocker stabilization region adds hybridization energy and stabilizes hybridization of the barcode blocker oligonucleotide to the splint oligonucleotide.

The labile blocker oligonucleotides (also called hyb blocker oligonucleotides) can hybridize to the hybridization region of one of the splint oligonucleotides and optionally to the blocker stabilization region immediately 5′ of the hybridization region. When hybridized, the barcode blocker oligonucleotides can prevent or reduce analyte-independent interaction of a splint oligonucleotide with the hybridization region. This can reduce the background signal from hybridization of the 3′ hybridization region of paired splint oligonucleotides in the absence of analyte.

The hyb blocker oligonucleotide remains hybridized to the splint oligonucleotide during binding of the detection conjugate and the capture antibody (attached to a bead) to the analyte to form a complex that brings the splint oligonucleotides in proximity to each other. The hyb blocker oligonucleotide is effectively removed from the splint oligonucleotides when the beads are washed before the extension step to remove unbound analytes and detection conjugates. If used, the blocker stabilization region adds hybridization energy and stabilizes hybridization of the hyb blocker oligonucleotide to the splint oligonucleotide.

The non-labile (and/or labile) blocker oligonucleotides can be modified at the 3′ end (indicated by “*” for the non-labile in blocker oligonucleotides FIG. 31B) to prevent further extension from the blocker oligonucleotide. For example, the 3′ end of the blocker oligonucleotides can have a 3′ phosphate, a dideoxycytidine (ddC), or an inverted dT. In another embodiment, as shown in FIG. 31C, the non-labile blocker was designed to include a 3′ “tail” that is not complementary to the corresponding region of the splint oligonucleotide 5′ of the stabilization/barcode region.

### Example 11

This non-limiting example shows subpool implementations in a PESD assay.

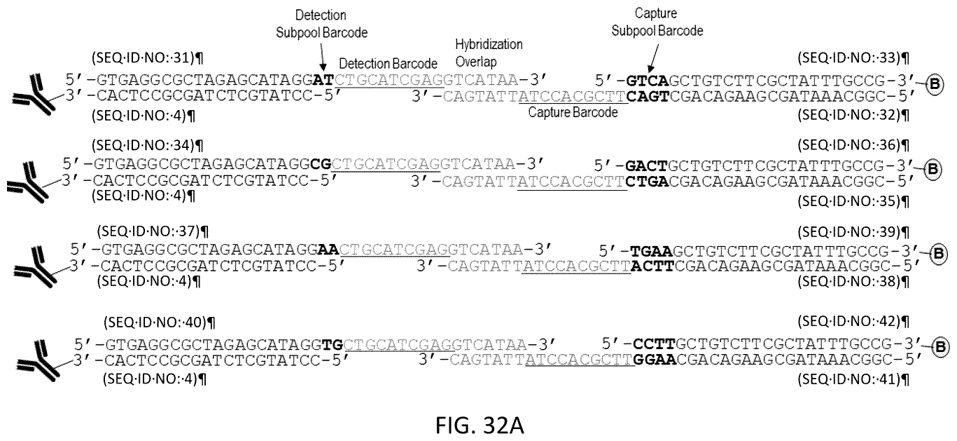

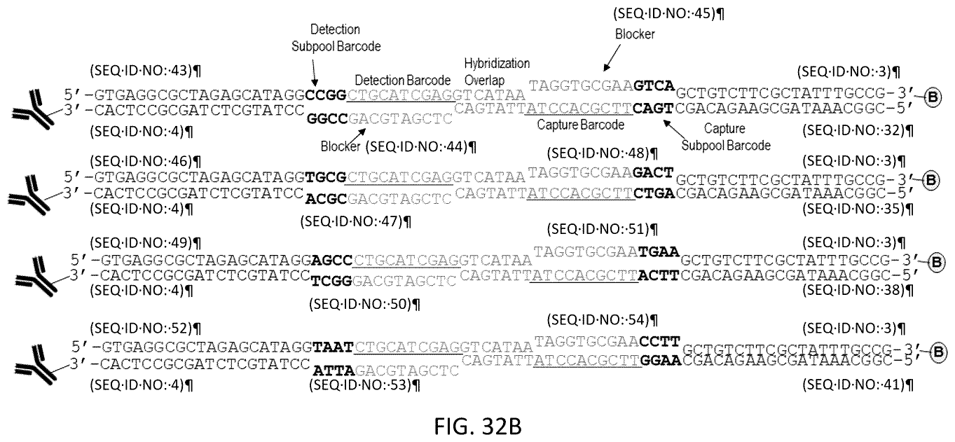

Non-limiting examples of a paired combination of splint oligonucleotides (e.g., capture and detection oligonucleotides) having different subpool barcodes, and associated tether oligonucleotides, were designed as shown in FIG. 32A. Each splint oligonucleotide included, from 5′ to 3′, a 5′ tethering region, a subpool barcode, a barcode region, a second blocker stabilization region, and a hybridization region. On the “detection” side, tether oligonucleotides were attached to an antibody that binds to an analyte, and on the “capture” side, the tether oligonucleotide was attached to a bead (“B”) that is further coated with another antibody that binds the same analyte as the detection antibody for the paired combination in a given subpool as indicated by the subpool barcodes. The subpool barcodes are asymmetrical such that capture subpool barcode is double stranded and 4 bases long, and the detector side is single-stranded but only 2 bases long. The splint oligonucleotides can hybridize to each other over their respective hybridization regions, when the splint oligonucleotides of a paired combination are in proximity to each other.

A PESD assay is set up as described in Example 1, except at step 5, the sample was first split into four subpools, each of which included different dilutions of the sample according to the following dilution scheme: A) 1:1 (undiluted); B) 1:20; C) 1:400; D) 1:2000. Each subpool was combined with beads barcoded with a capture oligonucleotide having a different capture subpool barcode from the other subpools. A paired combination of capture and detection oligonucleotides that are associated with antibodies that bind to an analyte, analyte I, that is expected to be very abundant in the original sample is combined with the most diluted subpool, D. A paired combination of capture and detection oligonucleotides that are associated with antibodies that bind to an analyte, analyte II, that is expected to be abundant in the original sample but less so than analyte I is combined with the next diluted subpool, C. A paired combination of capture and detection oligonucleotides that are associated with antibodies that bind to an analyte, analyte III, that is expected to be less abundant in the original sample than analyte II is combined with the subpool B. A paired combination of capture and detection oligonucleotides that are associated with antibodies that bind to an analyte, analyte IV, that is expected to be at low abundance in the original sample is combined with the undiluted subpool, A.

At step 7, the detection conjugate (detection antibody with tether hybridized to the detection oligonucleotide) with a detection oligonucleotide having the detection subpool barcode that corresponds to the capture subpool barcode used in that subpool is added. The subpools are combined into one pool before sequencing (e.g., before extension (step 9) or before sequencing library prep (step 10).

Upon sequencing the extension products, the combination of the subpool barcodes from the detection side and the capture side in the sequence indicates the subpool from which the paired combination of splint oligonucleotides originated, and in turn identifies the analyte bound by the antibodies associated with the splint oligonucleotides in that subpool.

With reference to FIG. 32B, the subpool barcodes can have the same length between the detection and capture side, and blocker oligonucleotides can be used to keep the subpool barcodes double stranded (see Example 11).

### Example 12

This non-limiting example shows trimming of hybridization region 3′ ends of one of the splint oligonucleotides of a paired combination to reduce background due to excess hybridization energy.

576 unique sets of oligonucleotide pairs with unique 7 nt hybridization regions were designed. These oligonucleotides were then tested independently in identical PESD assays (see Example 1) with a high concentration of a single analyte, or zero analyte. Each oligonucleotide set was used in a singleplex IL-13 assay with two calibrator levels (500 pg/mL of 0 pg/mL).

The signal generated at the high concentration of analyte allowed for ranking based on product conversion efficiency, and the signal generated in the absence of analyte allowed for ranking based on oligo:oligo pulldown (i.e., oligonucleotide-driven assay background).

In an ideal situation, all products would be generated with maximum, uniform efficiency, and the oligo:oligo pulldown would be low. However, there is a tipping point at which efficiency is maximized and any additional hybridization energy between the two oligonucleotides contributes only to the oligo:oligo pulldown and consequent increase in background.

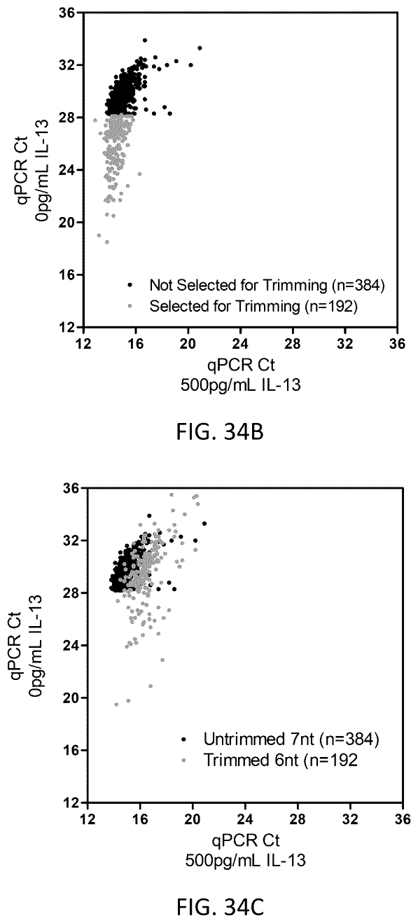

To demonstrate the selectivity of trimming, 192 oligonucleotide sets from the original set of 576 that had the highest background (in this case the lowest qPCR Ct value when 0 pg/mL of IL-13 was used in the assay) were chosen, and the 3′ terminal base from the detector-side oligo was trimmed (FIGS. 34A, 34B; see FIG. 33). All 192 trimmed oligos were then reanalyzed in the same experimental format. The result was a vastly improved pool of oligos that produced much lower background (i.e., higher qPCR Ct values when 0 pg/mL of IL-13 was used in the assay), with very minimal expense to the specific signal generation efficiency in a majority of the pairs (FIG. 34C).

### Example 13

This non-limiting example shows attenuation of PESD assay signal by lowering oligonucleotide density on barcoded beads and reducing the stoichiometry of capture oligonucleotide to detection oligonucleotide.

A PESD singleplex IL-13 assay was set up, varying the amount of tether (anchor) oligonucleotides per bead to which the capture oligonucleotides were hybridized, and the stoichiometry of capture oligonucleotide (presented on the beads) to detection oligonucleotide (associated with the detection conjugate). The performance of the assay was evaluated for specific signal (TOC @ 384 μg/mL IL-13) and non-specific, background signal (control buffer; “NSB”).

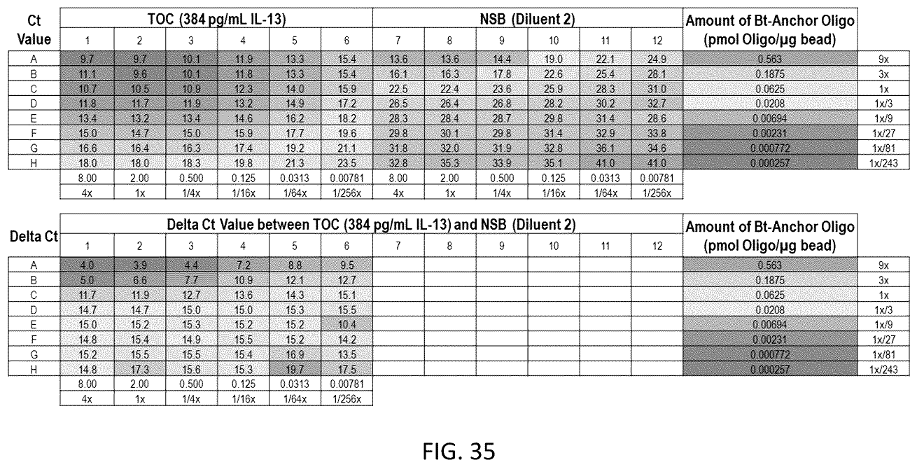

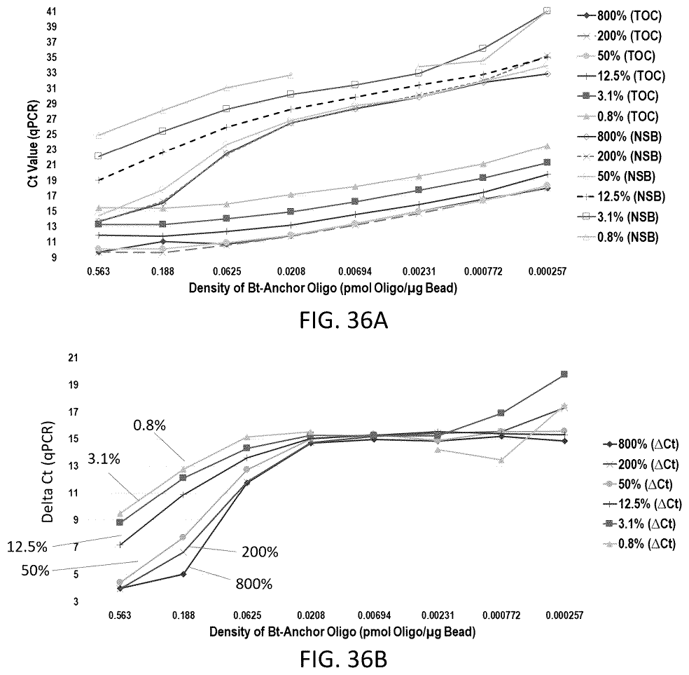

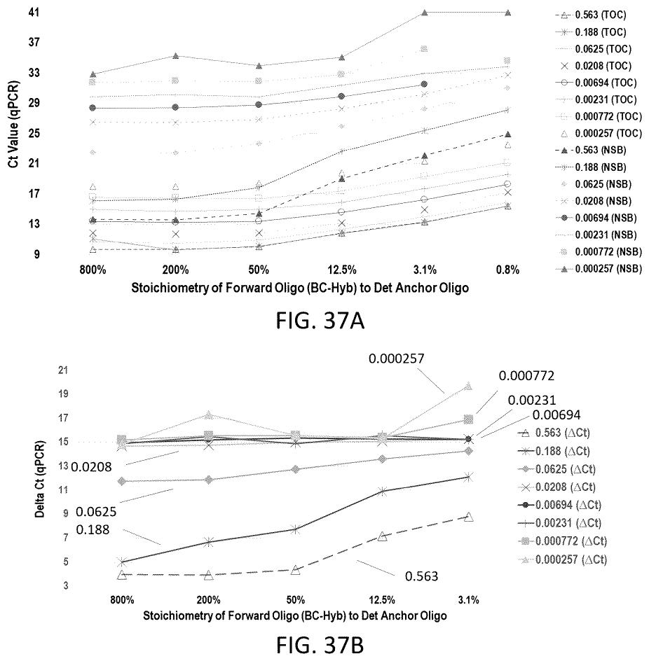

Reducing the density of capture oligonucleotides on beads reduced the background signal to a greater extent than the reduction in specific signal (FIG. 35, top panel; FIGS. 36A-36B). Thus, the difference between the specific signal and the background signal (delta Ct) increased as the density of capture oligonucleotides on beads decreased (FIG. 35, bottom panel). Reducing the stoichiometry of capture oligonucleotide to detection oligonucleotide also generally increased the specific signal over the background (delta Ct) (FIGS. 37A-37B). The data also showed that increasing the amount of capture antibody at any given stoichiometry of capture oligonucleotide to detection oligonucleotide generally increased the specific signal over the background signal (FIGS. 37A-37B).

These results show that attenuating the signal from a high-abundance analyte by reducing the oligonucleotide density on barcoded beads, reducing the stoichiometry of capture oligonucleotide to detection oligonucleotide, and/or increasing the amount of capture antibody (without a corresponding increase in the amount of capture oligonucleotide) can improve the specific signal over background and the dynamic range of the assay.

### Example 14

This non-limiting example shows the effect of oligonucleotide competitors to hybridization regions on efficiency and background signal of a PESD assay.

Short competitor oligonucleotides that target the hybridization regions were designed with 8 nt, 10 nt, or 12 nt lengths. In a multiplex context with NGS readout, 32 pairs of detection/capture oligonucleotides were evaluated. The results showed that the 10 nt competitor oligonucleotides were effective in reducing background signal. These results indicated that hybridization region competitors may have potential application in improving assay performance.

Next, the effect of 9 nt competitor oligonucleotides on efficiency and background signal of a detection/capture oligonucleotide pair having a 7 nt hybridization region in a single-plex format with qPCR readout was evaluated.

Oligo Design: 9 nt competitor oligonucleotides with a chain terminator (3′ InvT) were designed to either the forward (detector) or reverse (capture) oligo, where the competitor sequence was complementary to the hybridization region and a few nucleotides in the adjacent barcode region.

Experiment: Competitor oligonucleotides were added to the assay at different concentrations during incubation of sample-contacted beads with the detection conjugate.

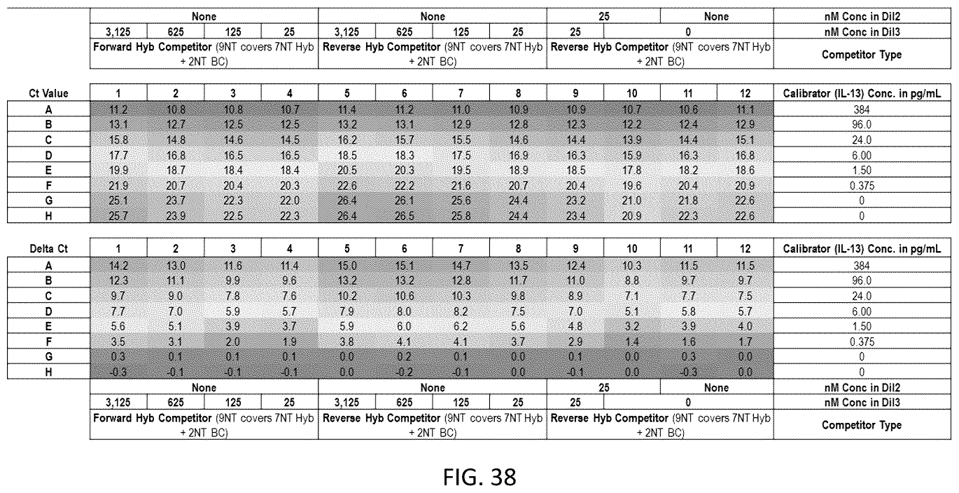

FIG. 38 shows that increasing the competitor concentration reduced the background signal (0 pg/mL IL-13), while the reduction in specific signal was muted. This was reflected in an increase in delta Ct as concentration of competitor oligonucleotide increases.

### Example 15

This non-limiting example shows the effect of the length of the 3′ hybridization sequence on specific signal and non-specific background in a singleplex Proximity Extension Strand Displacement (PESD) IL-13 assay.

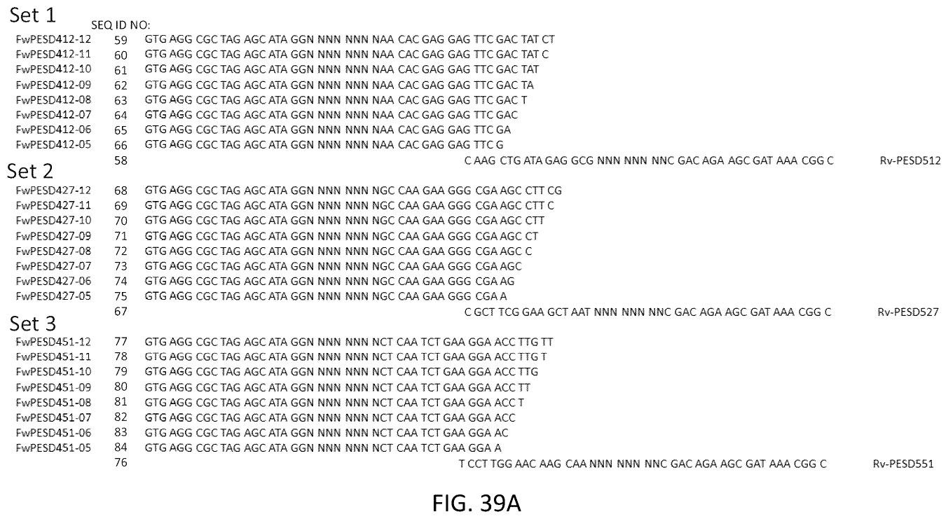

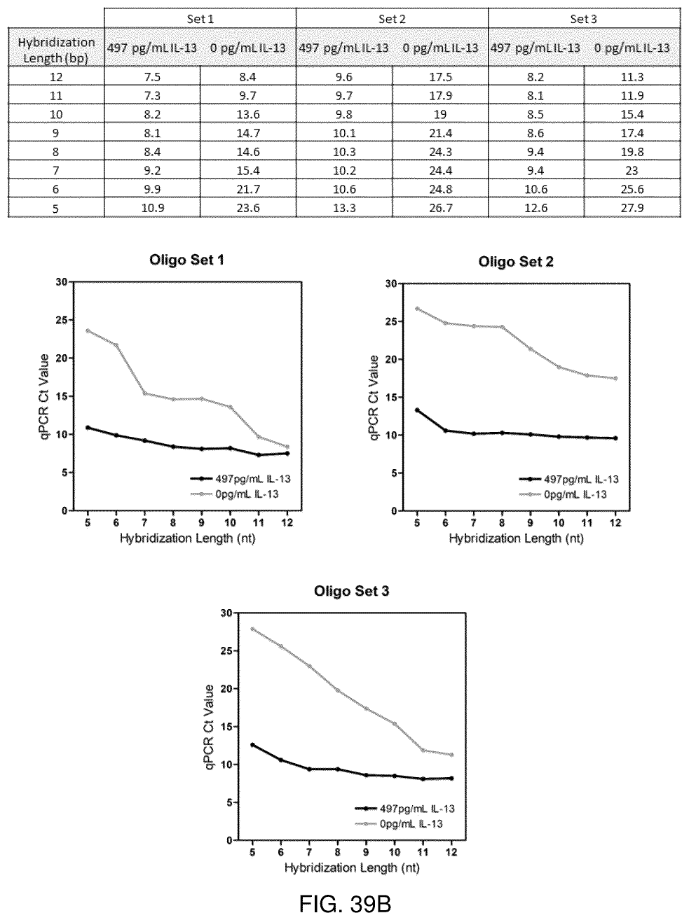

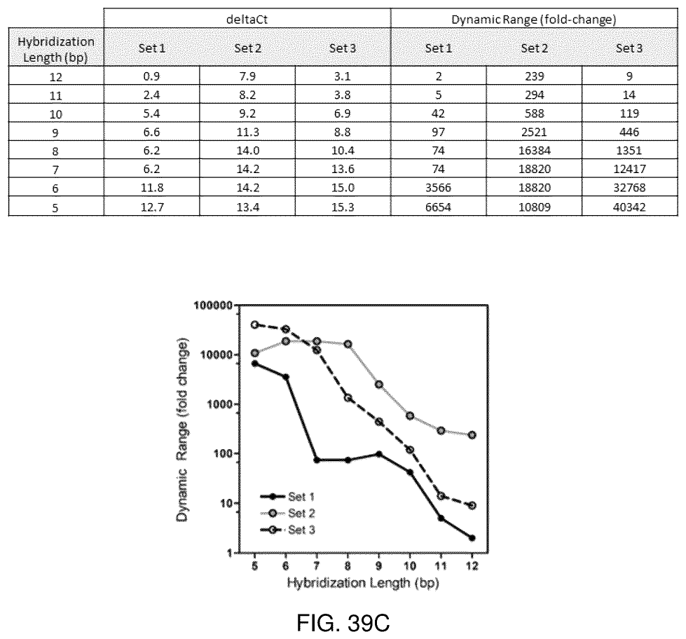

Three sets of capture and detector oligonucleotides were designed with 12 nucleotide hybridization sequences, set 1, 2, and 3 in FIG. 39A. The detector oligonucleotide from each set was sequentially shortened, one base at a time to create a family of forward oligos with various lengths of complementarity to the corresponding capture oligonucleotide. These lengths ranged from 12 nucleotides to 5 nucleotides.

Each detection nucleotide was paired with the full-length capture oligonucleotide in an IL-13 PESD assay and was tested at two concentrations of IL-13 analyte: 497 pg/mL and 0 pg/mL. FIG. 39B shows the specific signal at 497 pg/mL of IL-13 and the background at 0 μg/mL of IL-13. Values are shown as qPCR Ct value for each analyte concentration and hybridization length. These were also shown in graphical form (FIG. 39B). For all three oligo sets, longer hybridization leads to slightly higher specific signal (lower Ct) but much higher background resulting in lower dynamic range. FIG. 39C shows the difference in Ct between the specific signal and background. Each oligo set behaves slightly differently regarding the optimal length to maximize dynamic range and shows different slopes of the signal versus length and background versus length.

This indicates that a range of hybridization lengths (e.g., 6-7), and not just one fixed length for all sequences, may be appropriate for highly multiplexed proximity extension assays. The relationship between specific signal and hybridization length indicates that reducing hybridization length may be used to attenuate the signal of a specific assay. E.g. for set two or three, reducing the hyb length from 6 to 5 reduces the signal by 2 Ct while preserving the same signal to background ratio.

### Example 16

This non-limiting example shows the use of cold-capture antibody to attenuate the signal for a singleplex Proximity Extension Strand Displacement (PESD) IL-13 assay while preserving the assay linearity.

A singleplex IL-13 PESD assay was performed to demonstrate signal attenuation through the addition of unlabeled, unconjugated capture antibody “cold capture” antibody to the sample capture step. Cold antibody was an identical clone to the capture antibody on the bead, and was added to the sample during the analyte capture step. Cold antibody amounts ranged from 0-4,950 ng per well.

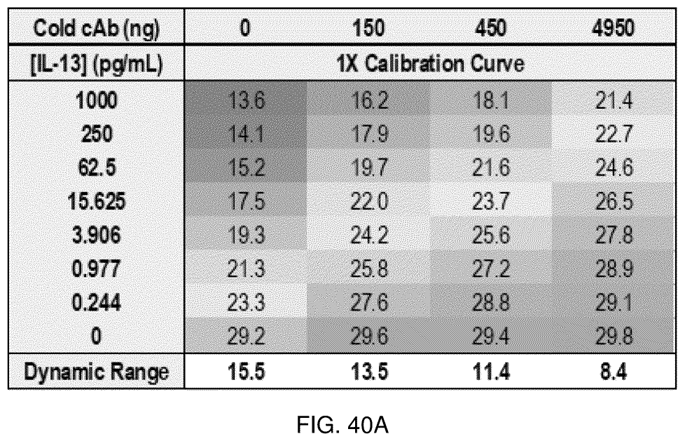

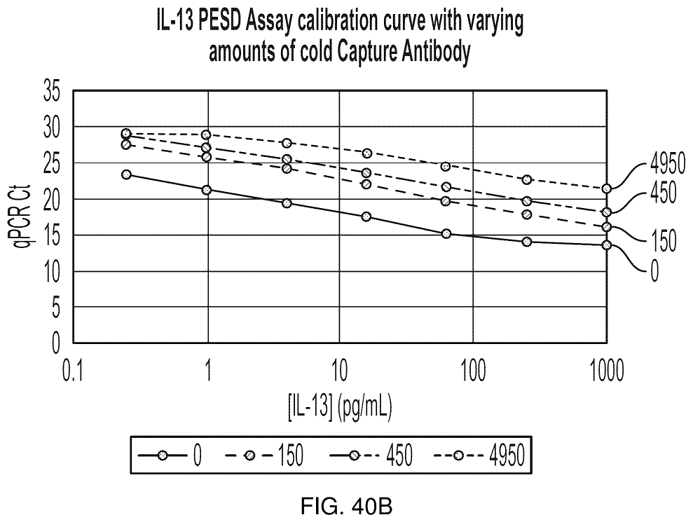

Signals were very efficiently attenuated through the use of cold antibody (FIGS. 40A, 40B). Linearity of the assay was preserved but the specific signal and dynamic range of the assay decreased, in a highly controlled fashion, as a function of cold addition amount (or concentration) (FIG. 40B).

### Example 17

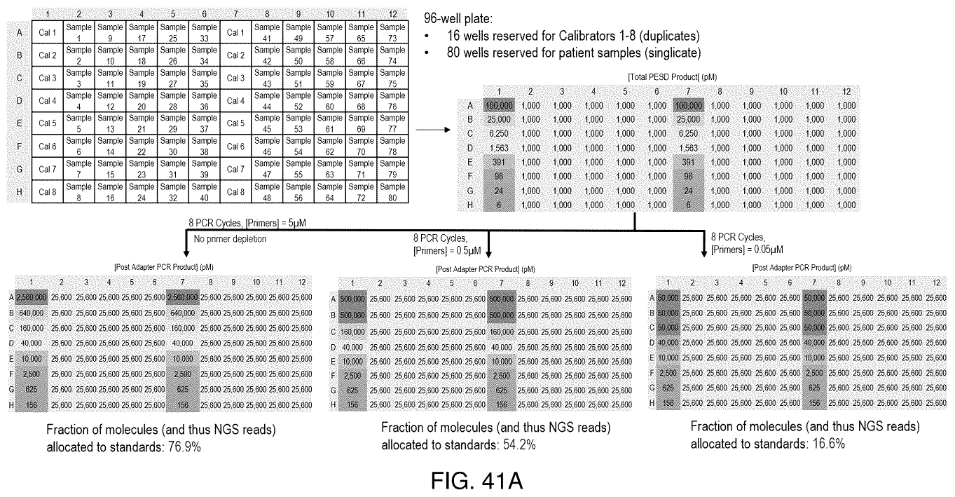

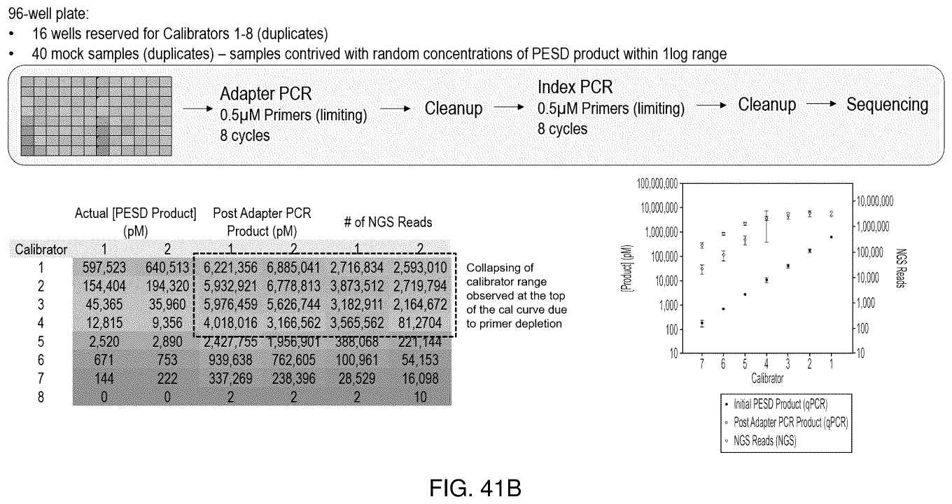

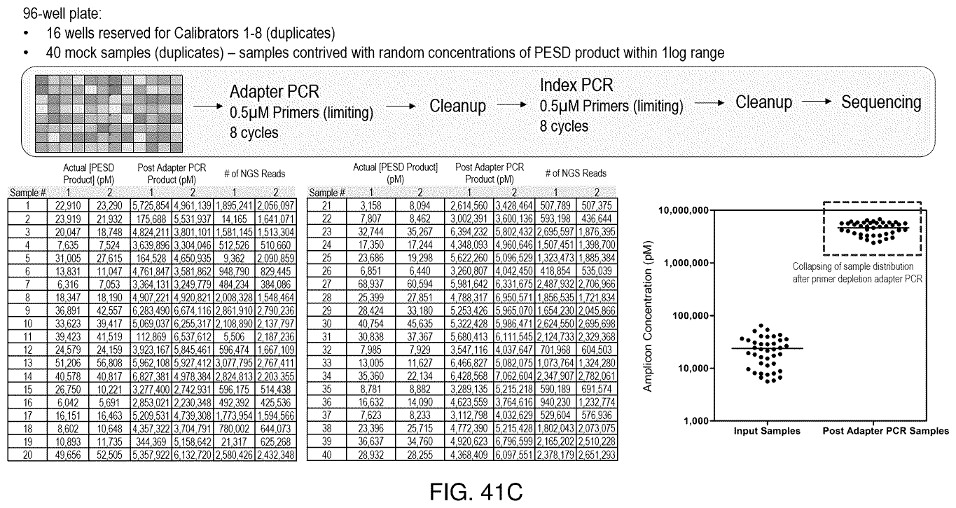

This non-limiting example shows the use of PCR compression to normalize the proportion of NGS reads used by high concentration standards or samples that have many elevated analytes. With an unrestricted adapter PCR reaction, in the multiplexing system described herein, ˜75% of the NGS reads would go to calibrators alone. This is reduced substantially with primer depletion normalization. For example, if all 80 patient samples and Cal 1-5 are normalized via primer depletion, then the read allocation for calibrators alone drops to <20% (FIG. 41A).

This PCR compression effect is produced by reducing the amount of primer in the well relative to the starting concentration of amplicon such that the primers are depleted in the concentrated samples part way through the reaction. Once primers begin to be depleted, amplification slows and when primers are completely depleted the amplification ceases. Low concentration samples continue to amplify linearly throughout the reaction.

PCR compression can be used such that the top 4 standards produce essentially the same concentration of indexed amplicon, and the overall range between standard 1 and standard 7 is reduced by more than 600-fold (FIGS. 41A-41B). PCR compression can also be used for sample balance (FIG. 41C). The saturation point and compression ratio can be tuned by varying the primer concentration and number of PCR cycles.

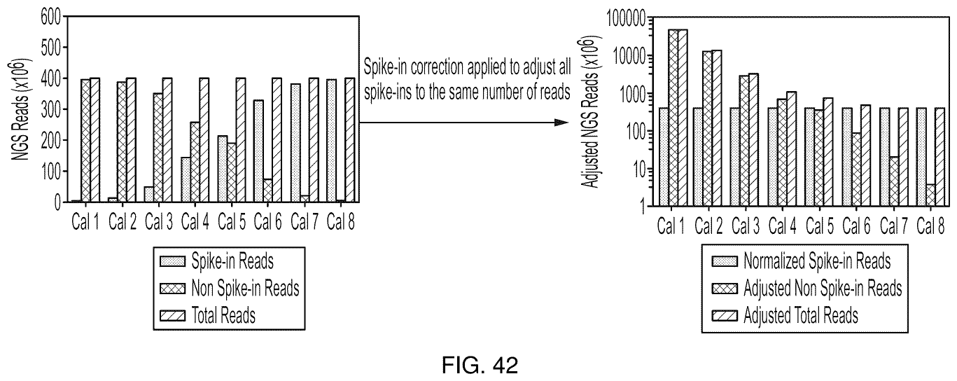

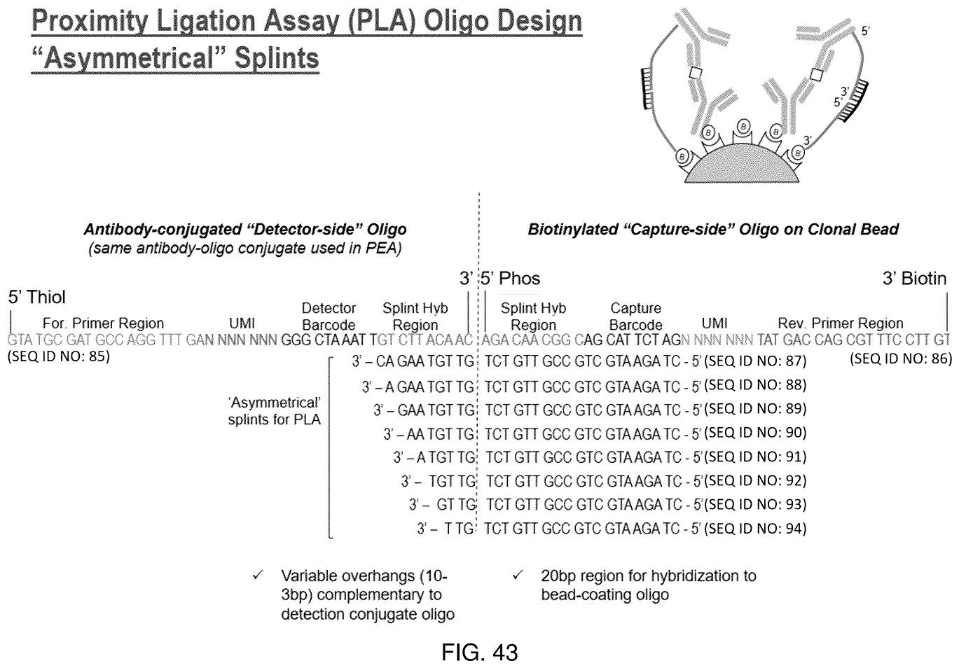

Since a synthetic spike-in oligo is included in the master mix of the adapter PCR at a fixed concentration for all samples/wells, this creates a reference level for all PESD product amplicons. The concentration of spike-in oligo used can be predicted based on known PCR amplification ratios to produce at least 1000 NGS counts in the most compressed wells. The primer-depletion normalization results in a different number of reads associated with the spike-in oligo across all indices. Thus, the spike counts will be high in the samples that started with low amplicon concentration and thus amplified linearly. On the other hand, spike counts will be low in samples that started with high amplicon concentration and thus had a high compression ratio. Spike normalized NGS reads will be proportional to the original amplicon concentrations in the PESD product. The ratio of spike-in oligo to analyte amplicon in each well is used to back calculate the actual number of reads and analyte molecules in a sample (FIG. 42). After normalization, standards can be fit with the 4PL curve and unknown samples can be back-fit to calculate concentrations. Alternatively, the ratio of spike-in oligo to analyte reads in each well can be calculated and use for quantitation.

### Example 18

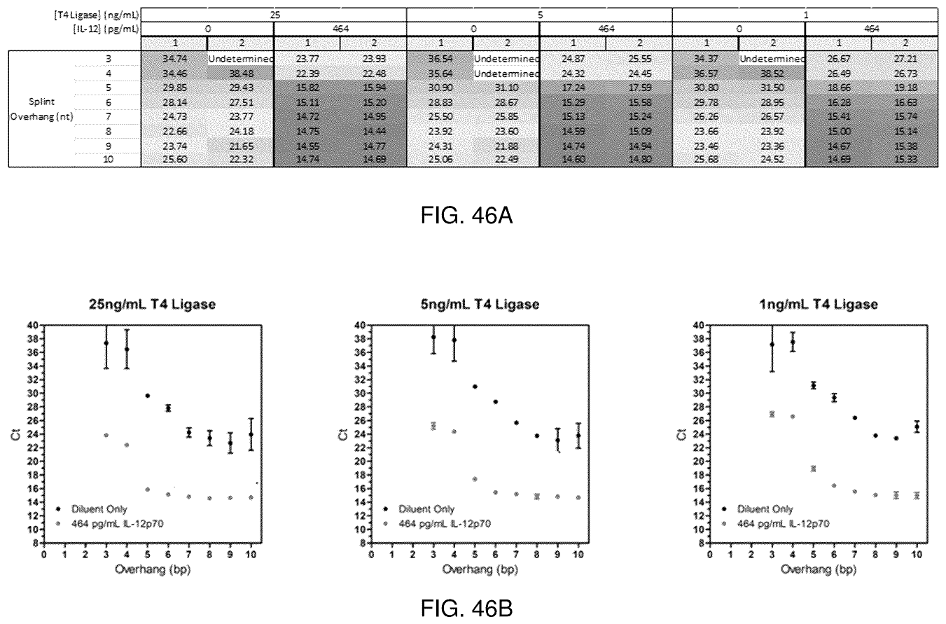

This non-limiting example shows a proximity ligation assay (PLA) with an asymmetrical splint.
1. Bead coating and barcoding:
   a. Streptavidin-coated magnetic beads were coated with a co-solution of biotin-Anti-IL-12 antibody and biotin PLA Capture Oligo.
      i. Coating conditions, per well: 1 ug of beads, 0.0625 pmol biotin-PLA Capture Oligo, 50 ng biotin-Anti-IL-12 antibody.
   b. After 2 h, beads were washed to remove unbound oligonucleotide and antibody.
2. Sample capture:
   a. Beads were added to wells containing standards of IL-12 as the analyte at concentrations varying from 0-464 pg/mL. Analyte was captured on beads for 2 h.
3. Detector incubation:
   a. Beads were washed to remove unbound IL-12 analyte.
   b. Beads were transferred to wells containing Anti-IL-12 detection antibody labeled with a barcoded oligonucleotide.
      i. This clone of Anti-IL-12 antibody is different from the antibody immobilized on the beads.
      ii. By design the oligonucleotide on the detection antibody has little/no complementarity to the oligonucleotide immobilized on the bead.
   c. Beads were incubated in detector conjugate for 1 h.
4. Splint addition
   a. Beads were washed to remove unbound Anti-IL-12 detector oligonucleotide conjugate.
   b. Beads were transferred to a solution containing the splints.
      i. The splints were designed such that they are: a portion of the splint is complementary to bases adjacent to ligation site on the bead-coating oligonucleotide, and a portion of the splint is complementary to the bases adjacent to the ligation site on the detection conjugate oligonucleotide.
      ii. The splint asymmetrically spans the ligation site with more base pairs on the side of the ligation site favoring the bead-coating oligonucleotide. By design, this was intended to be a stable 20 bp duplex formed between splint and bead-coating oligonucleotide. The remaining ‘overhang’ bases in the splint are complementary to the detection side oligonucleotide. In this experiment overhang was varied from 3-10 nt as shown in FIG. 46A.
   c. Beads were incubated in splint solutions for 30 min.
5. Ligation
   a. Beads were washed to remove unbound splint.
   b. Beads were transferred to solutions of ligase to ligate the two oligonucleotides mediated by the asymmetric splint.
   c. Ligation was performed for 1 h using T4 ligase at the concentrations shown in FIGS. 46A and 46B.
6. Release of product from beads
   a. Beads were washed to remove all enzyme and co-factors.
   b. Beads were transferred to Proteinase K solution to digest all proteinaceous material anchoring the oligonucleotide product to the beads.
   c. The protein digestion reaction was performed for 45 min at 40 C.
   d. Spent beads were removed from the system.
   e. Residual protease activity was destroyed by heat inactivating the reaction mixture for 15 min at 95 C.
7. Analysis of reaction products by qPCR.
   a. qPCR performed with 0.3 uM forward and reverse primer present in SYBR green master mix. Results are shown in FIGS. 46A and 46B. The following temperature program was used:
      i. UDG Activation: 50 C 2 min
      ii. Dual Lock DNA Polymerase: 95 C 2 min
      iii. Amplification (40 cycles)
         1. Denaturation: 95 C 3 sec
         2. Annealing+Extension: 60 C 30 sec

### Example 19

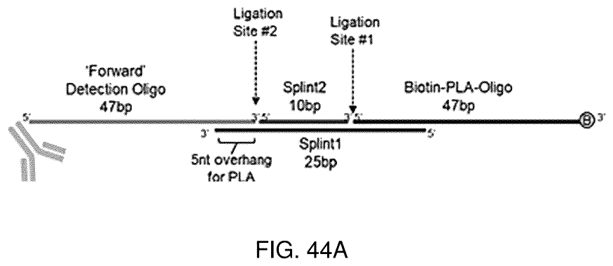

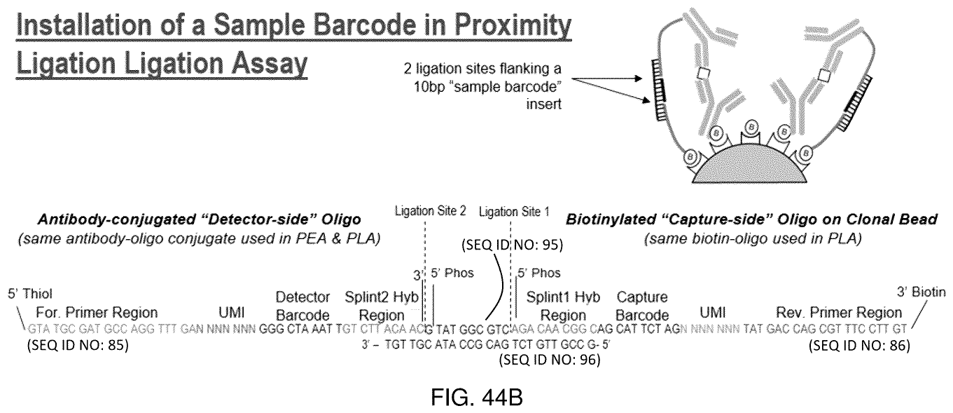

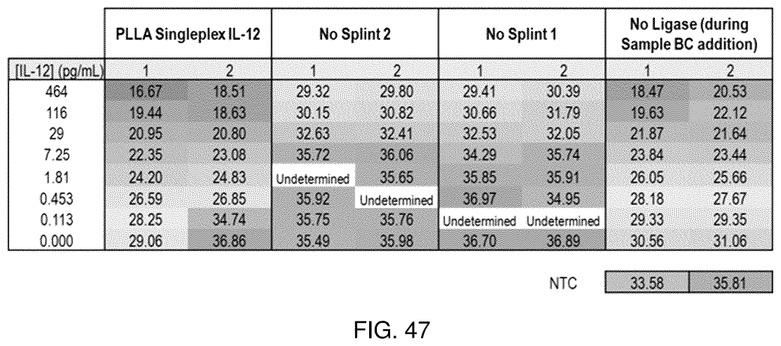

This non-limiting example shows a proximity ligation ligation assay (PLLA) in which an additional barcode is installed in the bead-coating amplicon prior to sample capture. This additional barcode may be used as a unique sample identifier, or any other additional code, to create a 3-barcode system, where two of those barcodes correspond to the capture and detection moieties. FIGS. 44A and 44B provide diagrams showing non-limiting arrangements of certain components of a PLLA.
1. Bead coating and barcoding:
   a. Streptavidin-coated magnetic beads were coated with a co-solution of biotin-Anti-IL-12 antibody and biotin PLA Capture Oligo (‘bead-coating oligonucleotide”)
      i. Coating conditions, per well: 1 ug of beads, 0.0625 pmol biotin-PLA Capture Oligo, 50 ng biotin-Anti-IL-12 antibody.
   b. After 2 h, beads were washed to remove unbound oligonucleotide and antibody
2. Addition of a sample barcode—first ligation
   a. Beads were transferred to wells containing the pre-hybridized splint, and sample barcode oligonucleotide. No splint 1 (i.e., sample barcode), and No splint 2 (i.e., ligation splint) negative controls were also included in this experiment. Splint 1 was used at 240 nM and Splint 2 was used at 300 nM.
   b. Incubation with the splint and sample barcode oligonucleotide duplex was performed for 45 min.
   c. Beads were washed to remove unbound splint and sample barcode, then beads released into ligase to ligate the sample barcode onto the bead-coating oligonucleotide for 45 min. A no ligase control was included in this step as a negative control.
3. Sample capture:
   a. Beads were added to wells containing standards of IL-12 as the analyte at concentrations varying from 0-464 pg/mL as shown in FIG. 47. Analyte was captured on beads for 2 h.
4. Detector incubation:
   a. Beads were washed to remove unbound IL-12 analyte.
   b. Beads were transferred to wells containing Anti-IL-12 detection antibody labeled with a barcoded oligonucleotide.
      i. This clone of Anti-IL-12 antibody is different from the antibody immobilized on the beads.
      ii. By design the oligonucleotide on the detection antibody has little/no complementarity to the oligonucleotide immobilized on the bead.
   c. Beads were incubated in detector conjugate for 1 h.
5. Ligation
   a. Beads were washed to remove unbound detection conjugate.
   b. Beads were transferred to solutions of ligase to ligate the oligonucleotides mediated by the splint.
   c. Ligation was performed for 1 h with 125 ng/mL of T4 ligase.
6. Release of product from beads
   a. Beads were washed to remove all enzyme and co-factors.
   b. Beads were transferred to Proteinase K solution to digest all proteinaceous material anchoring the oligonucleotide product to the beads.
   c. The protein digestion reaction was performed for 45 min at 40 C.
   d. Spent beads were removed from the system.
   e. Residual protease activity was destroyed by heat inactivating the reaction mixture for 15 min at 95 C.
7. Analysis of reaction products by qPCR.
   a. qPCR performed with 0.3 uM forward and reverse primer present in SYBR green master mix. Results of the qPCR are shown in FIG. 47. The following temperature program was used:
   b. UDG Activation: 50 C 2 min
   c. Dual Lock DNA Polymerase: 95 C 2 min
   d. Amplification (40 cycles)
      i. Denaturation: 95 C 3 sec
      ii. Annealing+Extension: 60 C 30 sec.

### Example 20

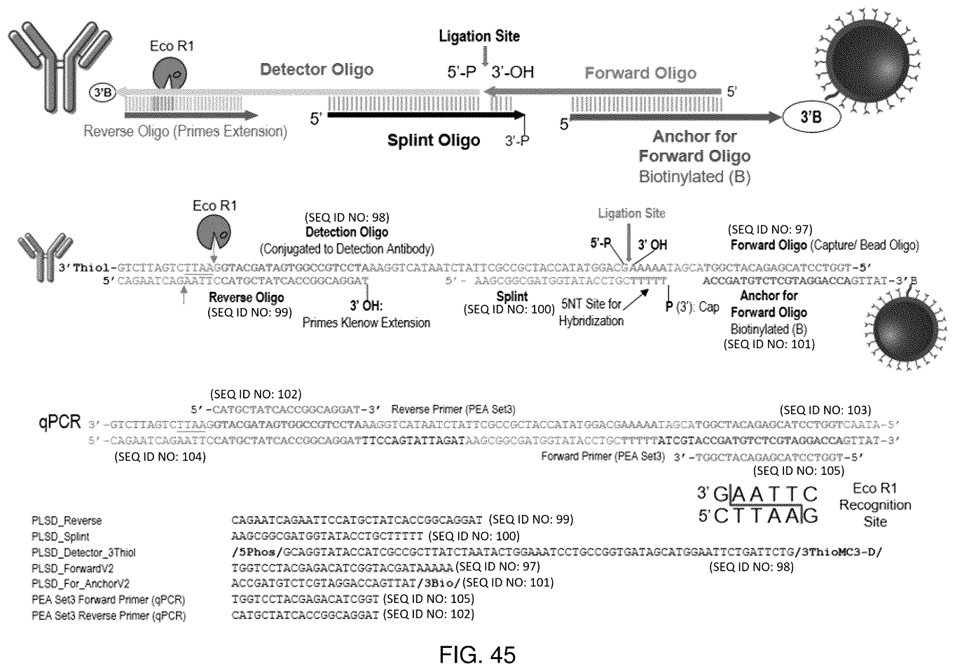

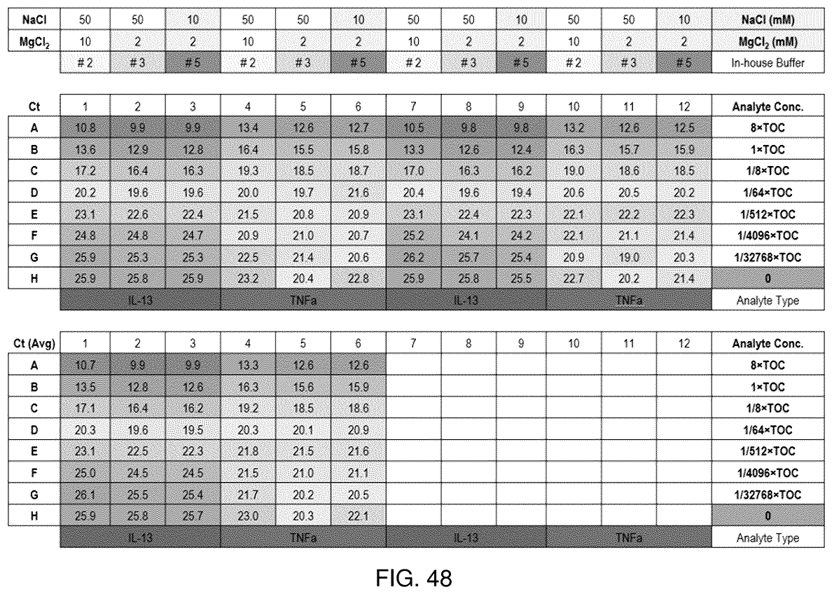

This non-limiting example shows a PLSD (Proximity Ligation Strand Displacement) assay. The inclusion of strand displacement functionality can limit the amount of unreacted product leftover in PLA that may otherwise interfere with sequencing downstream. Ligation was first performed followed by a restriction enzyme treatment and polymerase extension. In this example, the EcoRI restriction enzyme step and Klenow fragment extensions step were performed simultaneously. FIG. 45 provide diagrams showing non-limiting arrangements of certain components of PLSD.
1. Bead coating and barcoding
   a. Streptavidin-coated magnetic beads were coated with a co-solution of biotin-Anti-IL-13 antibody (or Anti-TNFa) and biotin anchor capture oligo (‘bead-coating oligonucleotide”)
   b. Coating conditions, per well: 1 ug of beads, 0.0625 pmol biotin-PLA Capture Oligo, 50 ng biotin-antibody.
   c. After overnight coating, beads were washed to remove unbound oligonucleotide and antibody
2. Sample Capture
   a. Beads were added to wells containing standards of IL-13 (or TNFa) as the analyte at concentrations varying from 0-3,856 pg/mL for IL-13, or 0-2,896 for TNFa.
   b. Analyte was captured on beads for 2 h.
3. Detector Incubation:
   a. Beads were washed to remove unbound analyte
   b. Beads were transferred to wells containing Anti-IL-13 (or Anti-TNFa) detection antibody labeled with a barcoded oligonucleotide. The detecton oligonucleotide was anchored to the detection antibody through a dsDNA duplex. This dsDNA duplex also contained a designed restriction enzyme cleavage site specific for the enzyme EcoRI
   c. A splint was stably prehybridized onto the detector-side oligonucleotide occupying the 20 nt closest to the restriction site. This splint also features a short 5 bp overhang that was complementary to the 5 bases adjacent to the ligation site on the bead-coating oligonucleotide.
   d. Beads were incubated in detector conjugate for 1 h.
4. Ligation
   a. Beads were washed to remove unbound detection conjugate.
   b. Beads were transferred to solutions of ligase to ligate the oligonucleotides mediated by the splint.
   c. Ligation was performed for 1 h with 25 ng/mL of T4 ligase.
5. Product Release
   a. Beads were washed to remove unbound enzyme and other soluble components.
   b. Beads were transferred to wells containing both EcoRI restriction enzyme and Klenow fragment polymerase. The reaction buffer was varied in this experiment to identify the optimal conditions to maximum both enzyme activities in their simultaneous use.
   c. Beads were incubated in those enzyme mixes for 1 h to release the products.
6. Analysis of reaction products by qPCR.
   a. qPCR performed with 0.3 uM forward and reverse primer present in SYBR green master mix. Results of the qPCR are shown in FIG. 48. The following temperature program was used:
   b. UDG Activation: 50 C 2 min
   c. Dual Lock DNA Polymerase: 95 C 2 min
   d. Amplification (40 cycles)
      i. Denaturation: 95 C 3 sec
      ii. Annealing+Extension: 60 C 30 sec

### Example 21

This non-limiting example shows PESD methods for antibody screening and characterization using a set of oligonucleotides selected according to the method described in Example 6.

Forty-nine monoclonal antibodies targeting the protein CEACAM5 were obtained and each antibody was labeled with a forward tether oligonucleotide by first activating with SM-PEG4 then reacting with thiol-modified tether oligonucleotide. Uniquely barcoded forward oligonucleotides were hybridized to each tether labeled antibody to form detection conjugates. After these labelling, the conjugates were pooled then unreacted tether oligo and unhybridized forward oligonucleotides were removed by MWCO spin filtration. Separately conjugates were purified before pooling but there were no significant differences in the results.

Forty-eight of the antibodies were biotinylated and a set of clonal capture beads was formed for each antibody by first coating Pierce streptavidin beads with reverse tether oligo at 0.00625 pmol/ug, followed by biotinylated antibody at a saturating concentration. The 49th antibody was omitted due to availability. Clonal beads were washed using the KingFisher APEX and pooled prior to sample capture. Beads were dispensed into each well of several 96 well plate. In some of these wells the amount of beads was titrated to produce a reduction in the amount of capture antibody in those wells. Each plate of beads was transferred using the KingFisher into a separate 96 well plate containing samples. These samples included serial dilutions of various CEACAM5 recombinant proteins and cell culture samples known to contain CEACAM5.

The bead and sample mixtures were incubated for 1 hour followed by several washes using the KingFisher to remove unbound analytes from the capture beads. Separate plates containing the pooled detector conjugates were prepared. In some of the wells of these plates, the detector pools were titrated to reduce the concentration of each detector antibody. The beads, with captured sample were transferred into the detector plates by kingfisher and incubated for 30 minutes to allow binding of the capture conjugates. Then the samples were washer by kingfisher. For some plates, the beads were transferred immediately after washing into the PESD extension buffer with strand displacing polymerase to produce the PESD amplicon. For other plates, the beads were first transferred into various buffers and incubated for varying times form 5 minutes to 1 hour to measure the off-rate of antibodies in these buffers. After the incubation in buffer, the beads were transferred to the PESD extension plate to produce PESD amplicon.

Following extension the amplicon in each well was quantified with qPCR and was also put through NGS library prep and sequenced as described in other examples. The NGS data was demultiplexed by sample, then barcodes for each clonal bead and detector conjugate were identified. For each capture antibody/detector antibody pair and sample combination, the number of counts was tabulated and compared with a comparable assay run using an established ECL-based sandwich assay system.

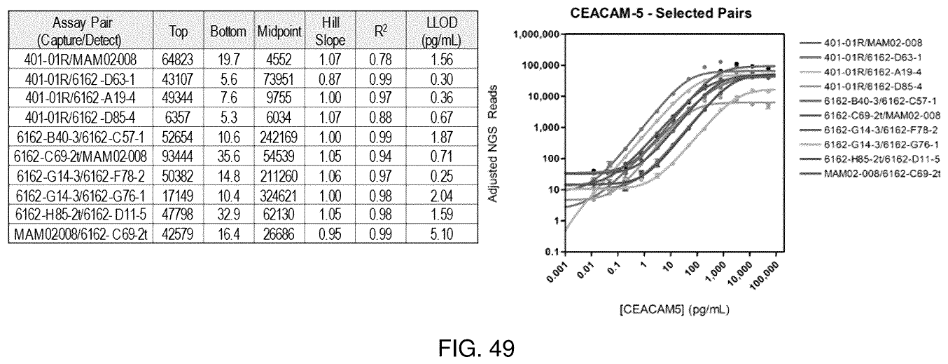

Results: The overall patterns of reactivity were strikingly similar when comparing the PESD and ECL measurements. PESD and ECL heatmaps for the entire 48×49 antibody matrix produced similar patterns. For each antibody pair were plotted the signal versus calibrator concentration and a 4PL fit was used to estimate the sensitivity of a sandwich assay using this capture and detection antibody pair. These are shown for 10-representative assays in FIG. 49.

Comparing various samples: We compared the reactivity of each antibody pair to the calibrator, which was also the immunogen used to produce the antibodies, against the reactivity to native CEACAM from a cancer cell lines. Many of the pairs that reacted to the immunogen did not react to the native CEACAM, suggesting that the immunogen was not a good match for the native analyte. Heatmaps for the immunogen and native protein showed marked differences.

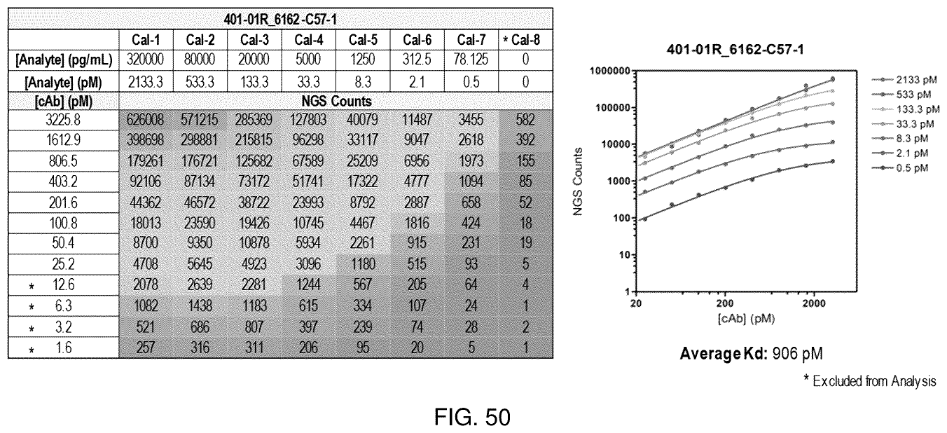

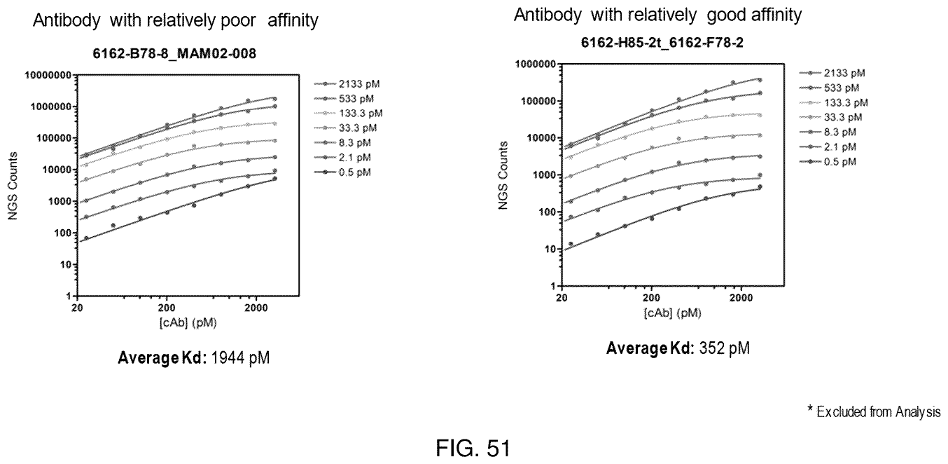

Calculating antibody affinities: The wells where capture bead amount was titrated were used to calculate the affinity of the capture antibodies as shown in FIG. 50. For analyte concentrations above Kd, the response if linear but below Kd, the response is hyperbolic and is well fit by a single site binding model, allowing extraction of a dissociation constant.

Measuring Off-rates: Off-rate off antibodies in a buffer was calculated from the data produced using various soak times after the detection step. FIG. 51 shows an example of calculating the signal loss and decay constants for 5 antibody pairs.

### Example 22

This non-limiting example of attenuation of PESD assay through deterministic or site specific modification. Example 13 showed that limiting the valency of oligonucleotide interactions between capture beads and detection conjugates, either by limiting the density of capture oligonucleotides on the beads or by reducing the stoichiometry of detector oligo to detector antibody, could minimize background, presumably by eliminating hybridization of multiple oligos on a single detector conjugate with multiple capture oligonucleotides on a bead.

Methods that use stochastic labeling of conjugates may be prone to this mechanism of background as they produce a degree-of-labeling that is poisson distributed, thus some fraction of each detector conjugate may have many oligonucleotides. Even for short hybridization regions, the sum of these interactions may result in unwanted pull-down of detector on the capture beads in an analyte independent fashion.

One way to eliminate these multivalent interaction is to ensure a uniform distribution of oligonucleotide labels on each detector through deterministic or site specific modification. Several methods for site-specific modification are known in the art, for example—Genovis GlyClick, which allows exactly 2 labels per antibody.

Detection conjugates are produced by labeling detection antibodies with exactly 2 forward tether oligonucleotides and then hybridizing forward oligonucleotides at a slight excess to the tether oligo. When these detection conjugates are used, the reverse oligonucleotide can be used at a higher surface density without producing analyte independent pull-down of detectors. This increases the specific signal without causing an unwanted elevation of background.

Alternatively, antigen binding fragments of antibodies (Fabs) can be used instead of full antibodies. These may also be deterministically labeled with a single tether oligonucleotide. Using a single labelled Fab instead of a doubly labeled antibody has a similar effect but may tolerate higher energy (longer) hybridization regions without producing detector pull-down, which can allow higher code-space and more orthogonal hybridization regions for higher multiplexing. Further, the fabs may have lower antibody-dependent non-specific binding to the capture antibody or bead surface due to their smaller size.

Although the foregoing has been described in some detail by way of illustrations and examples for purposes of clarity and understanding, it will be understood by those of skill in the art that modifications can be made without departing from the spirit of the present disclosure. Therefore, it should be understood that the forms disclosed herein are illustrative only and are not intended to limit the scope of the present disclosure, but rather to also cover all modification and alternatives coming with the true scope and spirit of the embodiments of the present disclosure.

It is contemplated that various combinations or subcombinations of the specific features and aspects of the embodiments disclosed above may be made. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed subject matter. Thus, it is intended that the scope of the present disclosure should not be limited by the particular disclosed embodiments described above. Moreover, while the disclosed subject matter is susceptible to various modifications, and alternative forms, specific examples thereof have been shown in the drawings and are herein described in detail. It should be understood, however, that the present disclosure is not to be limited to the particular forms or methods disclosed, but is to cover all modifications, equivalents, and alternatives falling within the spirit and scope of the various embodiments described and the appended claims.

Any methods disclosed herein need not be performed in the order recited unless explicitly stated or context dictates otherwise. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous includes 96%, 97%, 98%, 99%, and 100% homologous to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of analyzing a sample for an analyte, comprising:
a) combining:
i) a solid support comprising:
a capture moiety attached to the solid support, wherein the capture moiety specifically binds an analyte; and
a capture oligonucleotide attached to the solid support independently of the capture moiety, wherein the capture oligonucleotide comprises a 3′ hybridizing region;
ii) a detection conjugate comprising:
a detection moiety that specifically binds the analyte; and
a detection oligonucleotide attached to the detection moiety, wherein the detection oligonucleotide comprises a 3′ hybridizing region complementary to the 3′ hybridizing region of the capture oligonucleotide; and
iii) a sample;
wherein, if the analyte is present in the sample, it is bound to the solid support by the capture moiety and to the detection conjugate by the detection moiety to form a capture complex in which the capture oligonucleotide and the detection oligonucleotide are in proximity;
b) permitting the 3′ hybridizing region of the capture oligonucleotide and the 3′ hybridizing region of the detection oligonucleotide that are in proximity to hybridize to each other;
c) extending the hybridized capture oligonucleotide and/or the hybridized detection oligonucleotide to generate an on-target extension product;
d) releasing the on-target extension product from the solid support; and
e) determining the presence or the absence of the released on-target extension product to determine the presence or the absence of the analyte in the sample.

2-7. (canceled)

8. The method of claim 1, wherein the capture oligonucleotide comprises a first 5′ tethering region and is attached to the solid support via hybridization to a first tether oligonucleotide attached to the solid support, and the detection oligonucleotide comprises a second 5′ tethering region and is attached to the detection moiety via hybridization to a second tether oligonucleotide attached to the detection moiety.

9. The method of claim 8, wherein the hybridized capture oligonucleotide and the hybridized detection oligonucleotide are extended by a strand-displacing DNA polymerase, and the on-target extension product is released from the solid support by the strand-displacing DNA polymerase.

10-14. (canceled)

15. The method of claim 1, wherein the capture oligonucleotide comprises a first barcode sequence that identifies a binding target of the capture moiety, and wherein the detection oligonucleotide comprises a second barcode sequence that identifies a binding target of the detection moiety.

16-26. (canceled)

27. The method of claim 1, wherein determining the presence or the absence of the released on-target extension product comprises performing qPCR.

28. The method of claim 1, wherein determining the presence or the absence of the released on-target extension product comprises sequencing the released on-target extension product.

29. The method of claim 1, comprising providing a plurality of paired combinations of the solid support and the detection conjugate, wherein a binding target of the capture moiety and the detection moiety of each paired combination is the same, and wherein different paired combinations of the plurality of paired combinations have different binding targets.

30-32. (canceled)

33. The method of claim 29, wherein each capture oligonucleotide attached to a solid support of the plurality of the solid supports comprises a barcode sequence that identifies a binding target of the capture moiety attached to the respective solid support.

34. (canceled)

35. The method of claim 29, wherein the sample is divided into a plurality of subpools comprising at least a first subpool and a second subpool.

36-40. (canceled)

41. A method of analyzing a sample for an analyte, comprising:
a) combining:
i) a first conjugate comprising:
a first moiety that specifically binds an analyte;
a first tether region attached to the first moiety; and
a first splint oligonucleotide comprising a 5′ tethering region, a first moiety barcode, and a 3′ hybridizing region, wherein the 5′ tethering region of the first splint oligonucleotide is hybridized to the first tether region attached to the first moiety;
ii) a second conjugate comprising:
a second moiety that specifically binds the analyte;
a second tether region attached to the second moiety; and
a second splint oligonucleotide comprising a 5′ tethering region, a second moiety barcode, and a 3′ hybridizing region, wherein the 5′ tethering region of the second splint oligonucleotide is hybridized to the second tether region attached to the second moiety, and wherein the 3′ hybridizing region of the second splint oligonucleotide complementary to the 3′ hybridizing region of the first splint oligonucleotide; and
iii) a sample;
d) permitting the 3′ hybridizing region of the first splint oligonucleotide and the 3′ hybridizing region of the second splint oligonucleotide that are in proximity to hybridize to each other if the analyte is present in the sample;
e) extending the hybridized first splint oligonucleotide and the hybridized second splint oligonucleotide using a strand-displacing DNA polymerase to generate an on-target extension product that comprises the extended first splint oligonucleotide and/or the extended second splint oligonucleotide and release by strand displacement the on-target extension product from the first moiety and the second moiety; and
f) determining the presence or the absence of the on-target extension product to thereby determine the presence or the absence of the analyte in the sample.

42-46. (canceled)

47. A method of determining a pairwise combination of binding moieties that can simultaneously bind to a binding target, the method comprising:
a) combining
i) a plurality of different populations of solid supports, each solid support comprising:
a first binding moiety attached to the solid support; and
a capture oligonucleotide independently attached to the solid support, wherein the capture oligonucleotide comprises a 3′ hybridizing region and a capture barcode region, wherein the 3′ hybridizing regions of the capture oligonucleotides are the same in the plurality of different populations of solid supports, and wherein the capture barcode regions of the capture oligonucleotides are different for each different population of solid supports and the capture barcode region identifies the binding moiety attached to the same solid support, and;
ii) a plurality of different populations of detection conjugates comprising:
a second binding moiety; and
a detection oligonucleotide attached to the second binding moiety, wherein the detection oligonucleotide comprises a 3′ hybridizing region complementary to the 3′ hybridizing region of the capture oligonucleotide and a detector barcode region, wherein the detector barcode regions of the detection oligonucleotide are different for each different population of detection conjugates and identifies the second binding moiety to which it is attached; and
iii) one or more binding targets;
wherein, if a binding target is present in the sample, and the binding target is specifically bound by the first binding moiety and the second binding moiety, a capture complex is formed in which the capture oligonucleotide and the detection oligonucleotide are in proximity;
b) permitting the 3′ hybridizing region of the capture oligonucleotide and the 3′ hybridizing region of the detection oligonucleotide to hybridize to each other;
e) extending at least one of the hybridized capture oligonucleotide and the hybridized detection oligonucleotide to generate an extension product that comprises i) an extended capture oligonucleotide and a complement of at least a part of the detection oligonucleotide, and/or ii) an extended detection oligonucleotide and a complement of at least a part of the capture oligonucleotide;
f) releasing the extension product from the solid support; and
g) determining the capture barcode region and the detector barcode region of the released extension product to thereby determine a combination of binding moieties that can simultaneously bind to the binding target.

48. A method of determining interaction between two moieties, the method comprising:
a) combining a plurality of different populations of moieties, each moiety comprising a splint oligonucleotide attached to the moiety, wherein the splint oligonucleotide comprises a 3′ hybridizing region and a barcode region, wherein the 3′ hybridizing regions of the splint oligonucleotides are the same in the plurality of different populations of moieties, and wherein the barcode regions of the splint oligonucleotides are different for each different population of moieties and the barcode region identifies the moiety, and wherein, if a first moiety of the plurality of different populations of moieties interacts with a second moiety of the plurality of different populations of moieties, a capture complex is formed in which the splint oligonucleotide of the first moiety and the splint oligonucleotide of the second moiety are in proximity;
b) permitting the 3′ hybridizing region of the splint oligonucleotide of the first moiety and the 3′ hybridizing region of the splint oligonucleotide of the second moiety to hybridize to each other;
e) extending at least one of the splint oligonucleotide of the first moiety and the splint oligonucleotide of the second moiety to generate an extension product that comprises i) an extended splint oligonucleotide of the first moiety and a complement of at least a part of the splint oligonucleotide of the second moiety, and/or ii) an extended splint oligonucleotide of the second moiety and a complement of at least a part of the splint oligonucleotide of the first moiety;
f) releasing the extension product from the solid support; and
g) determining the barcode region of the splint oligonucleotide of the first moiety and the barcode region of the splint oligonucleotide of the second moiety of the released extension product to thereby determine an interaction between two moieties.

49-61. (canceled)

62. A composition comprising:
a) a first pool comprising at least 30 different populations of solid supports, each solid support comprising:
i) a capture moiety attached to the solid support, wherein the capture moiety specifically binds an analyte;
ii) a tether oligonucleotide attached to the solid support independent of the capture moiety; and
iii) a capture oligonucleotide comprising from 5′ to 3′: a tethering region, a capture barcode region, and a 3′ hybridizing region, wherein the capture oligonucleotide is attached to the solid support via hybridization of the tethering region to the tether oligonucleotide attached to the solid support; and
b) a second pool comprising at least 30 different populations of detection conjugates, each detection conjugate comprising:
i) a detection moiety that specifically binds the analyte;
ii) a tether oligonucleotide attached to the detection moiety; and
ii) a detection oligonucleotide comprising from 5′ to 3′: a tethering region, a detection barcode region, and a 3′ hybridizing region, wherein the detection oligonucleotide is attached to the detection moiety via hybridization of the tethering region to the tether oligonucleotide attached to the detection moiety,
wherein each of the at least 30 different populations of solid supports forms a paired combination with a corresponding detection conjugate of the at least 30 different populations of detection conjugates, wherein the analyte bound by the capture moiety and detection moiety of each paired combination is the same, wherein the analytes bound by different paired combinations are different, wherein the 3′ hybridizing region of the capture oligonucleotide and detection oligonucleotide of each paired combination is not complementary to the 3′ hybridizing region of the detection oligonucleotide and the capture oligonucleotide, respectively, of any other paired combination, and wherein each paired combination can be distinguished from any other paired combination by one or both of the sequence of the capture barcode region and/or the sequence of the detection barcode region.

63. The composition of claim 62, further comprising at least 30 different populations of blocker oligonucleotides, wherein each of the different populations of blocker oligonucleotides specifically hybridizes to the 3′ hybridizing region or a portion thereof of the capture oligonucleotide or the detection oligonucleotide of each paired combination.

64. The composition of claim 62 wherein the solid support is a magnetically responsive bead.

65. The composition of claim 62, wherein the capture moiety and the detection moiety are independently an antibody or an antibody fragment.

66. A composition comprising a plurality of partially double-stranded nucleic acids, each partially double-stranded nucleic acid comprising: a capture oligonucleotide hybridized at the 5′ end with a first tether oligonucleotide of 15-25 nucleotides in length and comprising a 3′ hybridizing region of at most 10 nucleotides; a detection oligonucleotide hybridized at the 5′ end with a second tether oligonucleotide of 15-25 nucleotides in length and comprising a 3′ hybridizing region of at most 10 nucleotides, wherein the 3′ hybridizing region of the capture oligonucleotide is hybridized to the 3′ hybridizing region of the detection oligonucleotide.

67. A method of analyzing a sample for an analyte, comprising:
a) combining:
i) a solid support comprising:
a capture moiety attached to the solid support, wherein the capture moiety specifically binds an analyte; and
a capture oligonucleotide attached to the solid support independently of the capture moiety;
ii) a detection conjugate comprising:
a detection moiety that specifically binds the analyte; and
a detection oligonucleotide attached to the detection moiety;
iii) a first splint oligonucleotide comprising:
a first portion complementary to A) a 3′ hybridization region of the capture oligonucleotide, or B) a 5′ hybridization region of the capture oligonucleotide; and
a second portion complementary to A) a 5′ hybridization region of the detection oligonucleotide if the first portion is complementary to the 3′ hybridization region of the capture oligonucleotide, or B) a 3′ hybridization region of the detection oligonucleotide if the first portion is complementary to the 5′ hybridization region of the capture oligonucleotide; and
iv) a sample;
wherein, if the analyte is present in the sample, it is bound to the solid support by the capture moiety and to the detection conjugate by the detection moiety to form a capture complex in which the capture oligonucleotide and the detection oligonucleotide are in proximity;
b) permitting i) the 3′ hybridizing region of the capture oligonucleotide and the 5′ hybridizing region of the detection oligonucleotide that are in proximity to hybridize to the first splint oligonucleotide, or ii) the 5′ hybridizing region of the capture oligonucleotide and the 3′ hybridizing region of the detection oligonucleotide that are in proximity to hybridize to the first splint oligonucleotide;
c) ligating the hybridized capture oligonucleotide and the hybridized detection oligonucleotide to generate an on-target ligation product that comprises the capture oligonucleotide and the detection oligonucleotide;
d) releasing the on-target ligation product from the solid support; and
e) determining the presence or the absence of the released on-target ligation product to determine the presence or the absence of the analyte in the sample.

68. A composition comprising:
a) a first pool comprising at least 30 different populations of solid supports, each solid support comprising:
i) a capture moiety attached to the solid support, wherein the capture moiety specifically binds an analyte;
ii) a tether oligonucleotide attached to the solid support independent of the capture moiety; and
iii) a capture oligonucleotide comprising A) from 5′ to 3′: a tethering region, a capture barcode region, and a 3′ hybridizing region, wherein the capture oligonucleotide is attached to the solid support via hybridization of the tethering region to the tether oligonucleotide attached to the solid support, or B) from 3′ to 5′: a tethering region, a capture barcode region, and a 5′ hybridizing region, wherein the capture oligonucleotide is attached to the solid support via hybridization of the tethering region to the tether oligonucleotide attached to the solid support; and
b) a second pool comprising at least 30 different populations of detection conjugates, each detection conjugate comprising:
i) a detection moiety that specifically binds the analyte;
ii) a tether oligonucleotide attached to the detection moiety; and
ii) a detection oligonucleotide comprising A) from 5′ to 3′: a tethering region, a detection barcode region, and a 3′ hybridizing region, or B) A) from 3′ to 5′: a tethering region, a detection barcode region, and a 5′ hybridizing region, wherein the detection oligonucleotide is attached to the detection moiety via hybridization of the tethering region to the tether oligonucleotide attached to the detection moiety,
c) a third pool comprising at least 30 different populations of first splint oligonucleotides, each first splint oligonucleotide comprising:
i) a first portion complementary to A) a 3′ hybridization region of the capture oligonucleotide, or B) a 5′ hybridization region of the capture oligonucleotide; and
ii) a second portion complementary to A) a 5′ hybridization region of the detection oligonucleotide if the first portion is complementary to the 3′ hybridization region of the capture oligonucleotide, or B) a 3′ hybridization region of the detection oligonucleotide if the first portion is complementary to the 5′ hybridization region of the capture oligonucleotide; and
wherein each of the at least 30 different populations of first splint oligonucleotides forms a triad combination with a corresponding solid support of the at least 30 different populations of solid supports and a corresponding detection conjugate of the at least 30 different populations of detection conjugates, wherein the analyte bound by the capture moiety and detection moiety of each triad combination is the same, wherein the analytes bound by different triad combinations are different, wherein the capture oligonucleotide and detection oligonucleotide of each paired combination are not complementary to the first splint oligonucleotide of any other triad combination, and wherein each triad combination can be distinguished from any other triad combination by one or both of the sequence of the capture barcode region and/or the sequence of the detection barcode region.

69. A method of analyzing a sample for an analyte, comprising:
a) combining:
i) a first conjugate comprising:
a first moiety that specifically binds an analyte;
a first tether region attached to the first moiety; and
a first splint oligonucleotide comprising A) a 5′ tethering region, a first moiety barcode, and a 3′ hybridizing region, wherein the 5′ tethering region of the first splint oligonucleotide is hybridized to the first tether region attached to the first moiety, or B) a 3′ tethering region, a first moiety barcode, and a 5′ hybridizing region, wherein the 3′ tethering region of the first splint oligonucleotide is hybridized to the first tether region attached to the first moiety;
ii) a second conjugate comprising:
a second moiety that specifically binds the analyte;
a second tether region attached to the second moiety; and
a second splint oligonucleotide comprising A) a 5′ tethering region, a second moiety barcode, and a 3′ hybridizing region, wherein the 5′ tethering region of the second splint oligonucleotide is hybridized to the second tether region attached to the second moiety, or B) a 3′ tethering region, a second moiety barcode, and a 5′ hybridizing region, wherein the 3′ tethering region of the second splint oligonucleotide is hybridized to the second tether region attached to the second moiety;
iii) a third splint oligonucleotide comprising:
a first portion complementary to A) a 3′ hybridization region of the capture oligonucleotide, or B) a 5′ hybridization region of the capture oligonucleotide; and
a second portion complementary to A) a 5′ hybridization region of the detection oligonucleotide if the first portion is complementary to the 3′ hybridization region of the capture oligonucleotide, or B) a 3′ hybridization region of the detection oligonucleotide if the first portion is complementary to the 5′ hybridization region of the capture oligonucleotide; and
iv) a sample;
b) permitting the first splint oligonucleotide and the second splint oligonucleotide that are in proximity to hybridize to the third splint oligonucleotide if the analyte is present in the sample;
c) ligating the hybridized first splint oligonucleotide and the hybridized second splint oligonucleotide to generate an on-target ligation product that comprises the first splint oligonucleotide and the extended second splint oligonucleotide;
d) releasing the on-target ligation product by treating the on-target ligation product with a restriction endonuclease and a strand-displacing DNA polymerase; and
e) determining the presence or the absence of the on-target ligation product to thereby determine the presence or the absence of the analyte in the sample.
